(19) United States
(12) Patent Application Publication
(10) Pub. No.: US 2023/0021500 A1
(43) Pub. Date: Jan. 26, 2023

(54) CYSTEINE ENGINEERED ANTIBODY-DRUG CONJUGATES WITH PEPTIDE-CONTAINING LINKERS

(71) Applicant: Mersana Therapeutics, Inc., Cambridge, MA (US)

(72) Inventors: Dorin TOADER, Cambridge, MA (US); Kalli CATCOTT, Boston, MA (US); Timothy B. LOWINGER, Carlisle, MA (US)

(21) Appl. No.: 17/289,992

(22) PCT Filed: Oct. 29, 2019
(86) PCT No.: PCT/US2019/058586

§ 371 Date: Apr. 29, 2021

Related U.S. Application Data
(60) Provisional application No. 62/751,945, filed on Oct. 29, 2018.

(51) Int. Cl. A61K 47/68

(52) U.S. Cl. CPC A61K 47/6889; A61K 47/6851; A61K 47/6803

(57) ABSTRACT

The present disclosure relates generally to cysteine engineered antibody-drug conjugates comprising peptide-containing linkers and to methods of using these conjugates as therapeutics and/or diagnostics.

### RELATED APPLICATIONS

This application claims priority to, and the benefit of, U.S. provisional application No. 62/751,945, filed Oct. 29, 2018, under 35 USC § 119(e). The content of this application is hereby incorporated by reference in its entirety.

### BACKGROUND

Traditionally, pharmaceuticals have primarily consisted of small molecules that are dispensed orally (as solid pills and liquids) or as injectables. Over the past three decades, formulations (i.e., compositions that control the route and/or rate of drug delivery and allow delivery of the therapeutic agent at the site where it is needed) have become increasingly common and complex. Nevertheless, many questions and challenges regarding the development of new treatments as well as the mechanisms with which to administer them remain to be addressed. In some embodiments, many drugs exhibit limited or otherwise reduced potencies and therapeutic effects because they are either generally subject to partial degradation before they reach a desired target in the body, or accumulate in tissues other than the target, or have a short half-life.

One objective in the field of drug delivery systems, therefore, is to deliver medications intact to specifically targeted areas of the body through a system that can stabilize the drug and/or extend the half-life and control the in vivo transfer of the therapeutic agent utilizing either physiological or chemical mechanisms, or both.

Antibody-drug conjugates have been developed as target-specific therapeutic agents. Antibodies against various cancer cell-surface antigens have been conjugated with different cytotoxic agents, including, but not limited to, microtubulin inhibitors (such as maytansinoids, auristatins, and taxanes, see, e.g., U.S. Pat. Nos. 5,208,020; 5,416,064; 6,333,410; 6,441,163; 6,340,701; 6,372,738; 6,436,931; 6,596,757; and 7,276,497); DNA (such as calicheamicin, doxorubicin, and CC-1065 analogs; see, e.g., U.S. Pat. Nos. 5,475,092; 5,585,499; 5,846,545; 6,534,660; 6,756,397; and 6,630,579). Antibody-drug conjugates with some of these cytotoxic drugs are actively being investigated in the clinic for cancer therapy (see, e.g., Ricart, A. D., and Tolcher, A. W., 2007, Nature Clinical Practice, 4, 245-255; Krop et al., 2010, J. Clin. Oncol., 28, 2698-2704). However, existing antibody-drug conjugates have exhibited a few limitations. A major limitation is their inability to deliver a sufficient concentration of drug to the target site because of the limited number of targeted antigens and/or the relatively moderate cytotoxicity of cancer drugs like auristatins, methotrexate, daunorubicin, maytansinoids, taxanes, and vincristine. Successful ADC development for a given target antigen depends on optimization of antibody selection, linker stability, cytotoxic drug potency and mode of linker-drug conjugation to the antibody.

Conjugating a drug moiety to an antibody through covalent bonds generally leads to a heterogeneous mixture of molecules where the drug moieties are attached at a number of sites on the antibody. In some embodiments, cytotoxic drugs have typically been conjugated to antibodies through the lysine or cysteine residues of the antibody thereby generating a heterogeneous antibody-drug conjugate mixture. Depending on the reaction conditions, the heterogeneous mixture typically contains a distribution of from 0 to about 8 drug moieties attached at various sites on the antibody. Analytical and preparative methods are inadequate to separate and characterize these antibody drug conjugate species molecules within the heterogeneous mixture resulting from a conjugation reaction. Additionally, the conjugation process may be nonreproducible due to difficulties in controlling the reaction conditions. Therefore, there is a need to reproducibly produce homogeneous antibody-drug conjugates in which the antibody drug conjugate species molecules can be characterized.

### SUMMARY

The present disclosure features a cysteine engineered targeting moiety-drug conjugate that exhibits high drug load, as well as strong binding to target antigen. In some embodiments, the cysteine engineered targeting moiety is a protein-based recognition-molecule (PBRM).

In some embodiments, the PBRM comprises an engineered cysteine prior to the conjugation. Preferably, the cysteine engineered PBRM substantially maintains one or more structural or functional characteristics of the PBRM without the engineered cysteine.

In some embodiments, the antibody or antibody fragment is an engineered antibody or antibody fragment. In some embodiments, the cysteine engineered PBRM is a cysteine engineered antibody or antibody fragment. In some embodiments, the antibody or antibody fragment comprises an engineered cysteine at a specific location, and the corresponding wild type antibody or antibody fragment does not comprise a cysteine at the same location.

In some embodiments, the PBRM is an immunoglobulin having an engineered cysteine (e.g., a cysteine introduced by engineering the immunoglobulin), and the engineered cysteine does not perturb the folding and assembly of the PBRM or alter antigen binding and effector functions of the PBRM.

In some embodiments, upon conjugation, the PBRM is conjugated to one or more drugs (e.g., cytotoxic drugs) through the engineered cysteine (e.g., through the thiol group of the engineered cysteine). In some embodiments, a Linker-Drug moiety is connected to the PBRM at the engineered cysteine (e.g., at the thiol group of the engineered cysteine). In some embodiments, one or more structural or functional characteristics of the PBRM is substantially maintained upon conjugation. In some embodiments, the PBRM is immunoglobulin, and the conjugation does not perturb immunoglobulin folding and assembly or alter antigen binding and effector functions of the PBRM. In some embodiments, the conjugate provides a homogeneous stoichiometry between the linker-drug moieties and the PBRM (e.g., up to two linker-drug moieties are conjugated to each PBRM having an engineered cysteine in each light chain).

In some embodiments, the PBRM is an IgG1, IgG2a or IgG2b antibody comprising an engineered cysteine. In some embodiments, the PBRM (e.g., the antibody) comprises one or more engineered cysteines at one or more locations of the PBRM and allows for drug attachment at those locations (e.g., the locations of the engineered cysteines in the light chain-Fab, heavy chain-Fab, or heavy chain-Fc). In some embodiments, at least one engineered cysteine is located in the heavy chain. In some embodiments, at least one engineered cysteine is located in the light chain. In some embodiments, the PBRM (e.g., the antibody) comprises at least one mutation in the light chain constant region at V205C (Kabat numbering).

In some aspects, the present disclosure relates to a conjugate comprising a cysteine engineered targeting moiety and one or more Linker-Drug moieties covalently bonded to the cysteine engineered targeting moiety, wherein

each Linker-Drug moiety includes a Multifunctional Linker that connects the cysteine engineered targeting moiety to one or more Drug Units through intermediacy of a Releasable Assembly Unit for each Drug Unit, and connects a hydrophilic group to the Drug Units of each Linker-Drug moiety,

wherein the Releasable Assembly units are capable of releasing free drug in proximity to a target site targeted by the targeting moiety, and

wherein the Multifunctional Linker comprises a peptide moiety between the cysteine engineered targeting moiety and the hydrophilic group, wherein the peptide moiety includes at least two amino acids.

In some aspects, the present disclosure relates to a conjugate comprising a targeting moiety and one or more Linker-Drug moieties covalently bonded to the cysteine engineered targeting moiety, wherein

each Linker-Drug moiety includes a Multifunctional Linker that connects the cysteine engineered targeting moiety to one or more Drug Units through intermediacy of a Releasable Assembly Unit for each Drug Unit, and connects a polyalcohol or a derivative thereof to the Drug Units of each Linker-Drug moiety,

wherein the Releasable Assembly units are capable of releasing free drug in proximity to a target site targeted by the targeting moiety.

In some aspects, the present disclosure relates to a conjugate of Formula (I):

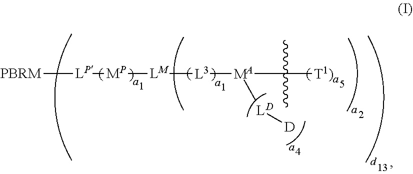

wherein

a₁, when present, is an integer from 0 to 1;

a₂ is an integer from 1 to 3;

a₃, when present, is an integer from 0 to 1;

a₄ is an integer from 1 to about 5;

a₅ is an integer from 1 to 3;

d₁₃ is an integer from 1 to about 6;

PBRM denotes a protein-based recognition-molecule, wherein the PBRM comprises an engineered cysteine;

L_{P′} is a divalent linker moiety connecting the engineered cysteine of the PBRM to M^{P}; of which the corresponding monovalent moiety L^{P} comprises a functional group W^{P} that is capable of forming a covalent bond with the engineered cysteine of the PBRM;

M^{P}, when present, is a Stretcher unit;

L^{M} is a bond, or a trivalent or tetravalent linker, and when L^{M} is a bond, a₂ is 1, when L^{M} is trivalent linker, a₂ is 2, or when L^{M} is a tetravalent linker, a₂ is 3;

L³, when present, is a carbonyl-containing moiety;

M^{A} comprises a peptide moiety that contains at least two amino acids;

T¹ is a hydrophilic group and the [symbol] between T¹ and M^{A} denotes direct or indirect attachment of T¹ and M^{A};

each occurrence of D is independently a therapeutic agent having a molecular weight ≤about 5 kDa; and

each occurrence of L^{D} is independently a divalent linker moiety connecting D to M^{A} and comprises at least one cleavable bond such that when the bond is broken, D is released in an active form for its intended therapeutic effect.

In some aspects, the disclosure relates to a peptide-containing scaffold, being any of Formulae (II)-(V):

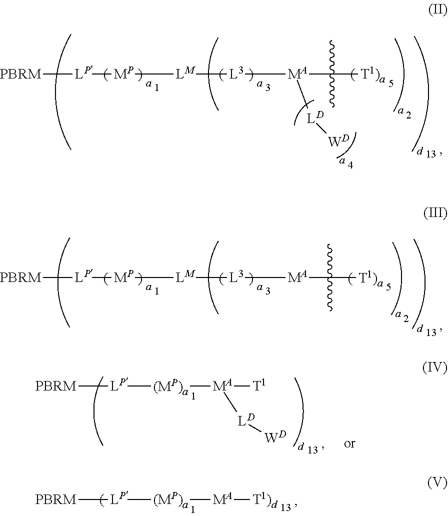

wherein:

a₁, when present, is an integer from 0 to 1;

a₂, when present, is an integer from 1 to 3;

a₃, when present, is an integer from 0 to 1;

a₄, when present, is an integer from 1 to about 5;

a₅, when present, is an integer from 1 to 3;

d₁₃ is an integer from 1 to about 6;

PBRM denotes a protein-based recognition-molecule, wherein the PBRM comprises an engineered cysteine;

L^{P′} is a divalent linker moiety connecting the cysteine engineered PBRM to M^{P}; of which the corresponding monovalent moiety L^{P} comprises a functional group W^{P} that is capable of forming a covalent bond with a functional group of engineered cysteine of the PBRM;

M^{P}, when present, is a Stretcher unit;

L^{M}, when present, is a bond, or a trivalent or tetravalent linker, and when L^{M} is a bond, a₂ is 1, when L^{M} is a trivalent linker, a₂ is 2, or when L^{M} is a tetravalent linker, a₂ is 3;

L³, when present, is a carbonyl-containing moiety;

M^{A} comprises a peptide moiety that contains at least two amino acids;

T¹ is a hydrophilic group and the [symbol] between T¹ and M^{A} denotes direct or indirect attachment of T¹ and M^{A};

each occurrence of W^{D}, when present, is independently a functional group that is capable of forming a covalent bond with a functional group of a therapeutic agent (“D”) having a molecular weight ≤about 5 kDa; and

each occurrence of L^{D} is independently a divalent linker moiety connecting W^{D} or D to M^{A} and L^{D} comprises at least one cleavable bond such that when the bond is broken, D is released in an active form for its intended therapeutic effect.

The conjugates and scaffolds of the disclosure can include one or more of the following features when applicable.

In some embodiments, each of the Drug Units and the hydrophilic group is connected to the Multifunctional Linker in parallel orientation.

In some embodiments, the cysteine engineered targeting moiety is a protein-based recognition-molecule (PBRM). In some embodiments, the PBRM is an antibody or antibody fragment.

In some embodiments, the PBRM comprises an engineered cysteine at V205 (Kabat numbering) of the light chain.

In some embodiments, the peptide moiety in the Multifunctional Linker comprises from three to about sixteen amino acids, e.g., about 3, about 4, about 5, about 6, about 7, about 8, about 9, about 10, about 11, about 12, about 13, about 14, about 15, or about 16 amino acids.

In some embodiments, the peptide moiety in the Multifunctional Linker comprises from three to about ten amino acids, e.g., about 3, about 4, about 5, about 6, about 7, about 8, about 9, or about 10 amino acids.

In some embodiments, the peptide moiety comprises from three to about ten amino acids selected from glycine, serine, glutamic acid, aspartic acid, lysine, cysteine, a stereoisomer thereof (e.g., isoglutamic acid or isoaspartic acid), and a combination thereof.

In some embodiments, the peptide moiety comprises at least four glycines and at least one serine.

In some embodiments, the peptide moiety comprises at least four glycines, at least one serine and at least one glutamic acid or isoglutamic acid.

In some embodiments, the peptide moiety comprises at least four glycines, and at least one glutamic acid.

In some embodiments, the hydrophilic group comprises a polyalcohol or a derivative thereof, a polyether or a derivative thereof, or a combination thereof.

In some embodiments, the hydrophilic group comprises an amino polyalcohol, e.g., glucamine or bis-glucamine.

In some embodiments, the hydrophilic group comprises:

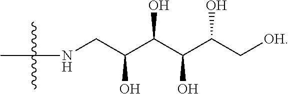 [SMILES: *NC[C@H](O)[C@@H](O)[C@H](O)[C@H](O)CO]

In some embodiments, the hydrophilic group comprises:

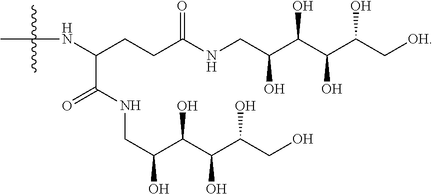 [SMILES: *NC(CCC(=O)NC[C@H](O)[C@@H](O)[C@H](O)[C@H](O)CO)C(=O)NC[C@H](O)[C@@H](O)[C@H](O)[C@H](O)CO]

In some embodiments, the amino polyalcohol is

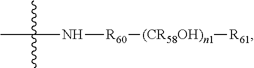

wherein

n₁ is an integer from 0 to about 6;

each R₅₈, when present, is independently hydrogen or C₁₋₈ alkyl;

R₆₀ is a bond, a C₁₋₆ alkyl linker, or —CHR₅₉— in which R₅₉ is —H, C₁₋₈ alkyl, cycloalkyl, or arylalkyl;

R₆₁ is CH₂OR₆₂, COOR₆₂, —(CH₂)ₙ₂COOR₆₂, or a heterocycloalkyl substituted with one or more hydroxyl;

R₆₂ is H or C₁₋₈ alkyl; and

n₂ is an integer from 1 to about 5.

In some embodiments, the hydrophilic group comprises

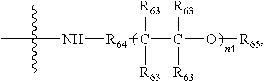

wherein

n₄ is an integer from 1 to about 25;

each R₆₃ is independently hydrogen or C₁₋₈ alkyl;

R₆₄ is a bond or a C₁₋₈ alkyl linker;

R₆₅ is H, C₁₋₈ alkyl, or —(CH₂)ₙ₂COOR₆₂;

R₆₂ is H or C₁₋₈ alkyl; and

n₂ is an integer from 1 to about 5.

In some embodiments, the hydrophilic group comprises polyethylene glycol, e.g., polyethylene glycol with from about 6 to about 24 PEG subunits.

In some embodiments, the hydrophilic group comprises a polyethylene glycol with from about 6 to about 12 PEG subunits.

In some embodiments, the hydrophilic group comprises a polyethylene glycol with from about 8 to about 12 PEG subunits.

In some embodiments, L, when present, comprises —X—C₁₋₁₀ alkylene-C(O)—, with X directly connected to L^{M}, in which X is CH₂, O, or NR₅, and R₅ is hydrogen, C₁₋₆ alkyl, C₆₋₁₀ aryl, C₃₋₈ cycloalkyl, COOH, or COO—C₁₋₆ alkyl.

In some embodiments, L³, when present, is —NR₅—(CH₂)ᵥ—C(O)— or —CH₂—(CH₂)ᵥ—C(O)—NR₅—(CH₂)ᵥ—C(O)—, in which each v independently is an integer from 1 to 10 (e.g., each v independently being an integer from 1 to 6, or from 2 to 4, or 2). In some embodiments, L³ is —NH—(CH₂)₂—C(O)— or —(CH₂)₂—C(O)—NH—(CH₂)₂—C(O)—.

In some embodiments, a₄ is 1, 2, or 3.

In some embodiments, d₁₃ is an integer from about 1 to about 6.

In some embodiments, d₁₃ is an integer from about 1 to about 4.

In some embodiments, d₁₃ is an integer from about 4 to about 6.

In some embodiments, d₁₃ is an integer from about 2 to about 4.

In some embodiments, d₁₃ is an integer from about 1 to about 2.

In some embodiments, d₁₃ is 2.

In some embodiments, each W^{P}, when present, is independently:

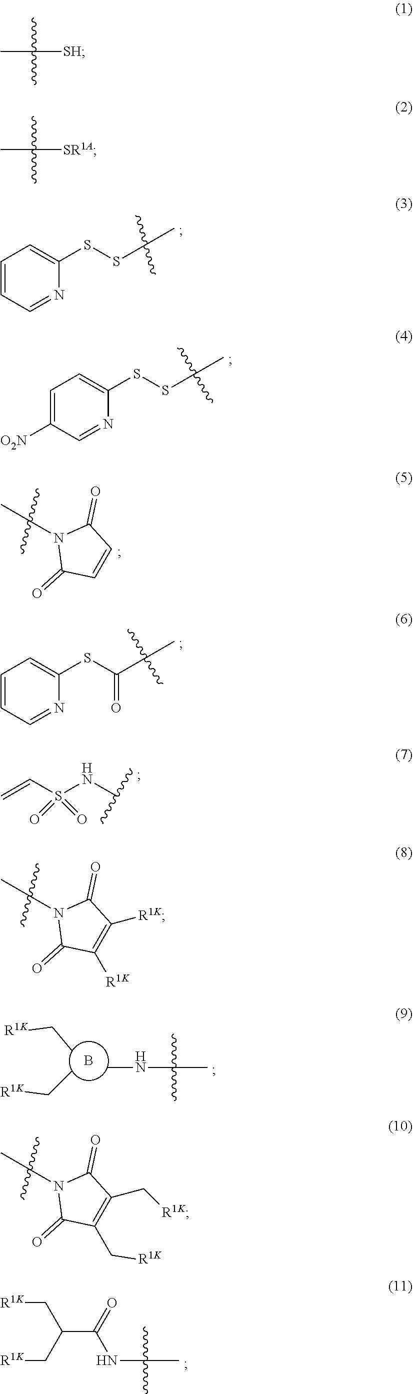

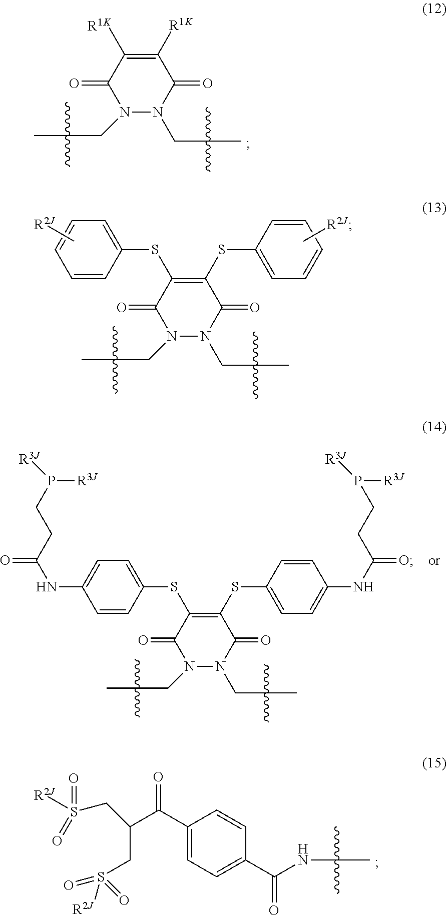

wherein

ring B is cycloalkyl, heterocycloalkyl, aryl, or heteroaryl;

R^{1K} is a leaving group;

R^{1A} is a sulfur protecting group;

R^{2J} is hydrogen, an aliphatic, aryl, heteroaliphatic, or carbocyclic moiety; and

R^{3J} is C₁₋₆ alkyl and each of Z₁, Z₂, Z₃, and Z₇ is independently a carbon or nitrogen atom.

In some embodiments, R^{K} is halo or RC(O)O— in which R is hydrogen, an aliphatic, heteroaliphatic, carbocyclic, or heterocycloalkyl moiety.

In some embodiments, R^{1A} is

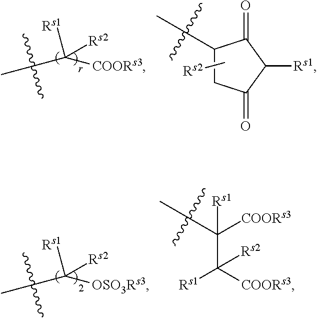

wherein r is 1 or 2 and each of R^{s1}, R^{s2}, and R^{s3} is independently hydrogen, an aliphatic moiety, a heteroaliphatic moiety, a carbocyclic moiety, or a heterocycloalkyl moiety.

In some embodiments, each W^{P} is independently

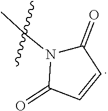 [SMILES: *N1C(=O)C=CC1=O]

In some embodiments, M^{P}, when present, is —(Z₄)—[(Z₅)—(Z₆)]_{z}—, with Z₄ connected to L^{P′} or L^{P} and Z₆ connected to L^{M}; wherein

z is 1, 2, or 3;

Z₄ is:

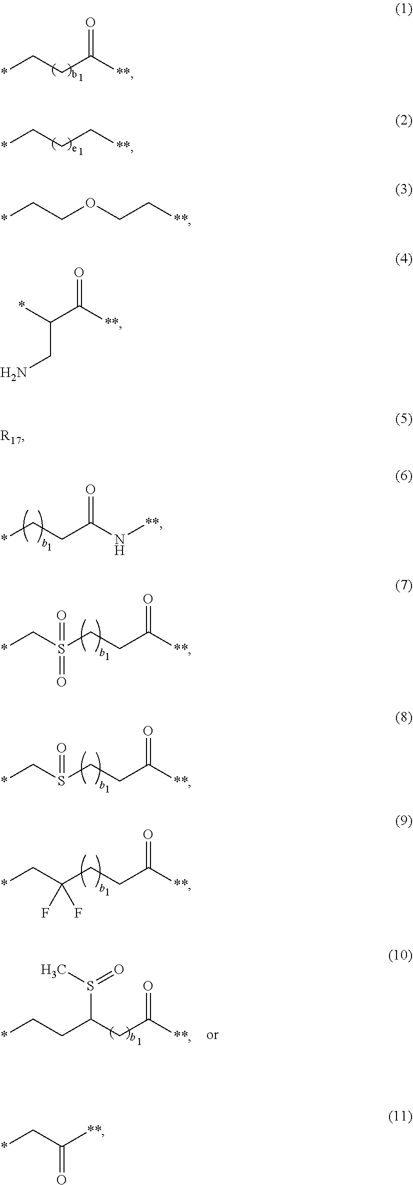

wherein * denotes attachment to L^{P′} or L^{P} and ** denotes attachment to Z₅ or Z₆, when present, or to L^{M} when Z₅ and Z₆ are both absent;

b₁ is an integer from 0 to 6;

e₁ is an integer from 0 to 8,

R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4- to 14-membered heterocycloalkylene, —C₁₋₁₀ alkylene-(4- to 14-membered heterocycloalkylene)-, -(4- to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₄ alkyl)-C(═O)—, -arylene-C(═O)—, —C₁₋₁₀ alkylene-arylene-C(═O)—, -arylene-C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4- to 14-membered heterocycloalkylene-C(═O)—, —C₁₋₁₀ alkylene-(4- to 14-membered heterocycloalkylene)-C(═O)—, -(4- to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4- to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁₋₁₀ alkylene-S—;

each Z₅ independently is absent, R₅₇—R₁₇ or a polyether unit;

each R₅₇ independently is a bond, NR₂₃, S or O;

each R₂₃ independently is hydrogen, C₁₋₆ alkyl, C₆₋₁₀ aryl, C₃₋₈ cycloalkyl, COOH, or COO—C₁₋₆ alkyl; and

each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S— or —(C₁₋₁₀ alkyl-R3)_{g1}—C₁₋₁₀ alkyl-C(O)—;

each R₃ independently is —C(O)—NR₅— or —NR₅—C(O)—;

each R₅ independently is hydrogen, C₁₋₆ alkyl, C₆₋₁₀ aryl, C₃₋₈ cycloalkyl, COOH, or COO—C₁₋₆ alkyl; and

g₁ is an integer from 1 to 4.

In some embodiments, M^{P}, when present, is

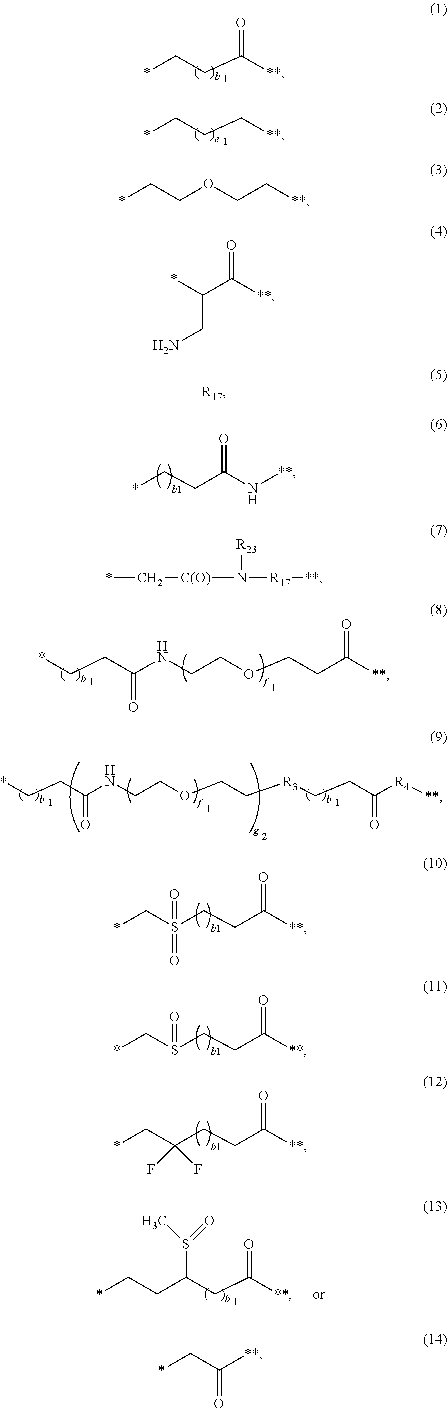

wherein * denotes attachment to L^{P′} or L^{P} and ** denotes attachment to L^{M};

R₃ is —C(O)—NR₅ or —NR₅—C(O)—;

R₄ is a bond or —NR₅—(CR₂₀R₂₁)—C(O)—;

R₅ is hydrogen, C₁₋₆ alkyl, C₆₋₁₀ aryl, C₃₋₈ cycloalkyl, —COOH, or —COO—C₁₋₆ alkyl;

R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4- to 14-membered heterocycloalkylene, —C₁₋₁₀ alkylene-(4- to 14-membered heterocycloalkylene)-, -(4- to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene-C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4- to 14-membered heterocycloalkylene-C(═O)—, —C₁₋₁₀ alkylene-(4- to 14-membered heterocycloalkylene)-C(═O)—, -(4- to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4- to 14-membered heterocycloalkylene-NH—, —C₁₋₁₀ alkylene-(4- to 14-membered heterocycloalkylene)-NH—, -(4- to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4- to 14-membered heterocycloalkylene-S—, —C₁₋₁₀ alkylene-(4- to 14-membered heterocycloalkylene)-S—, or -(4- to 14-membered heterocycloalkylene)-C₁₋₁₀ alkylene-S—;

each R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycle, C₃₋₈ cycloalkyl, hydroxylated C₃₋₈ cycloalkyl, polyhydroxylated C-s cycloalkyl or a side chain of a natural or unnatural amino acid;

each R₂ independently is hydrogen, C₁₋₆ alkyl, C₆₋₁₀ aryl, C₃₋₈ cycloalkyl, COOH, or COO—C₁₋₆ alkyl;

each b₁ independently is an integer from 0 to 6;

e₁ is an integer from 0 to 8;

each f₁ independently is an integer from 1 to 6; and

g₂ is an integer from 1 to 4.

In some embodiments, M^{P}, when present, is

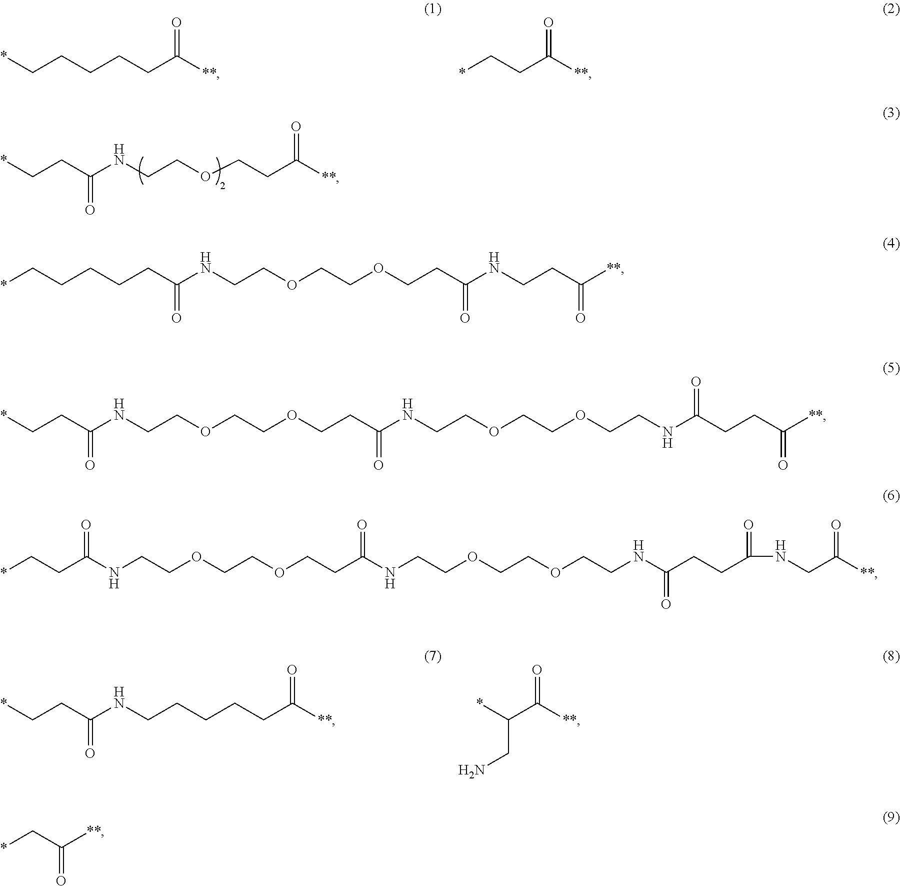

wherein * denotes attachment to L^{P′} or L^{P} and ** denotes attachment to L^{M}.

In some embodiments, L^{M} is a bond and a₂ is 1.

In some embodiments, a₂ is 2, and L^{M} is

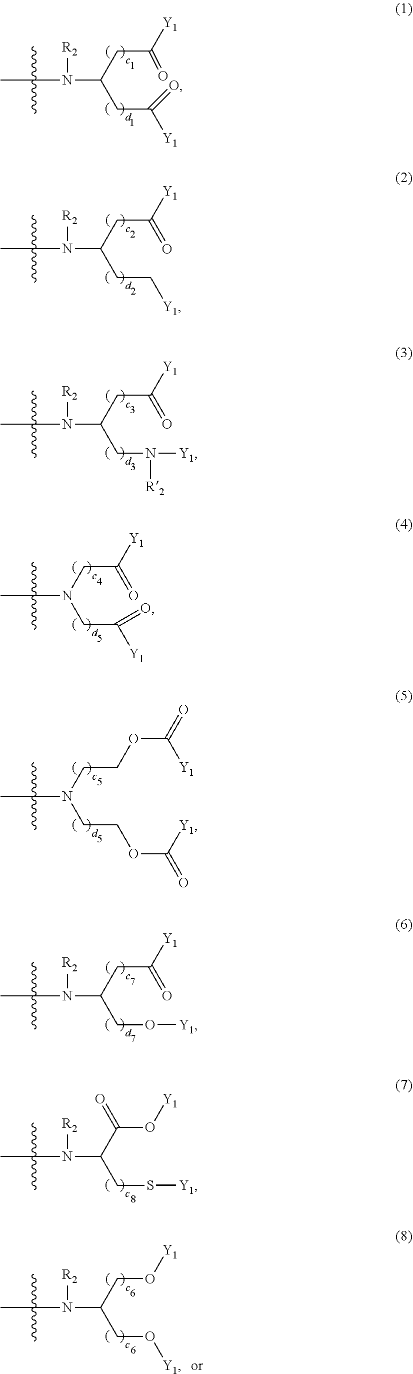

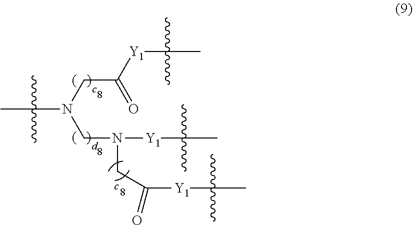

wherein

[symbol] denotes attachment to M^{P} when present or attachment to L^{P} or L^{P′} when M^{P} is absent:

Y₁ denotes attachment to L³ when present or attachment to M^{A} when L³ is absent:

R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxycarbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, COOH, or COO—C₁₋₆ alkyl;

each of c₁, c₂, c₃, c₄, c₅, c₇, and c₈ is an integer independently ranging between 0 and 10; and

each of d₁, d₂, d₃, d₄, d₅, and d₇ is an integer independently ranging between 0 and 10.

In some embodiments, a₂ is 2 and L^{M} is:

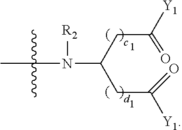

In some embodiments, a₂ is 3 and L^{M} is:

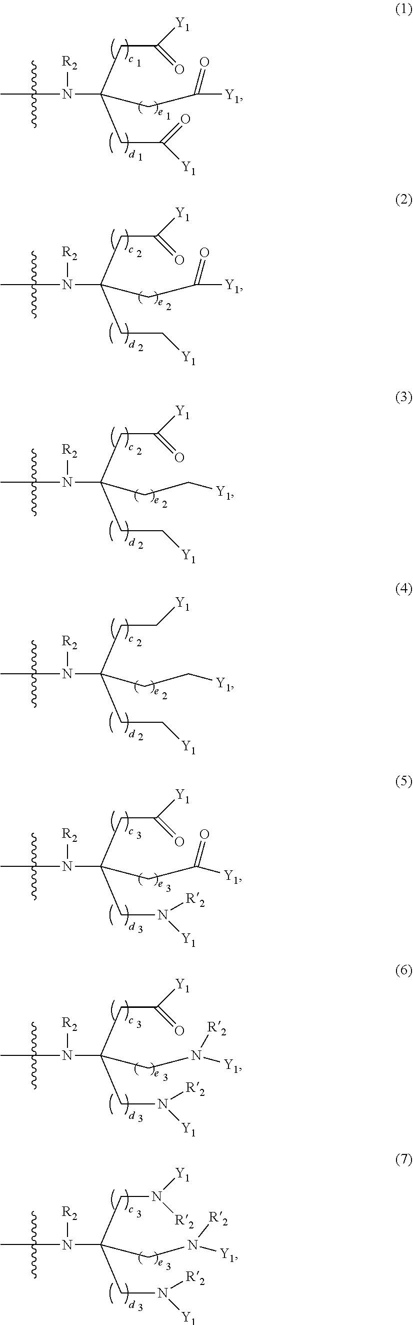

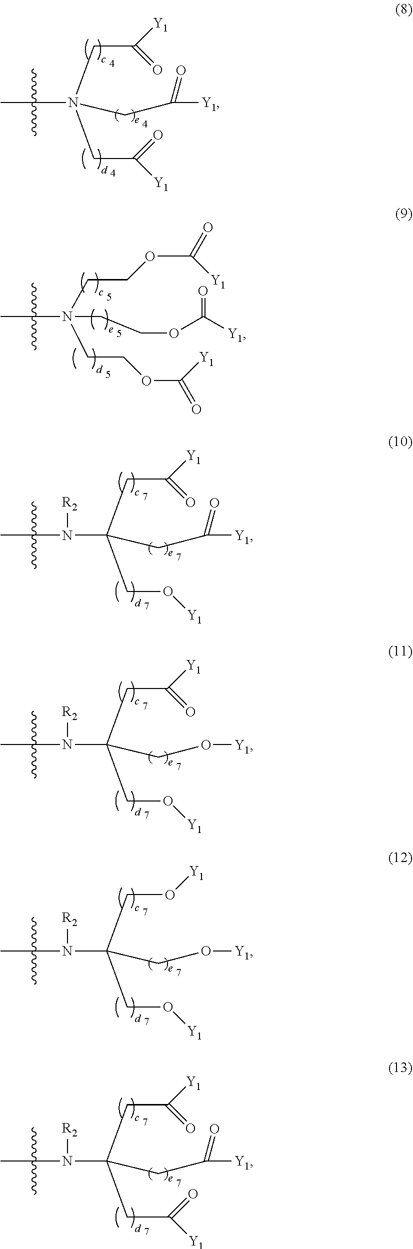

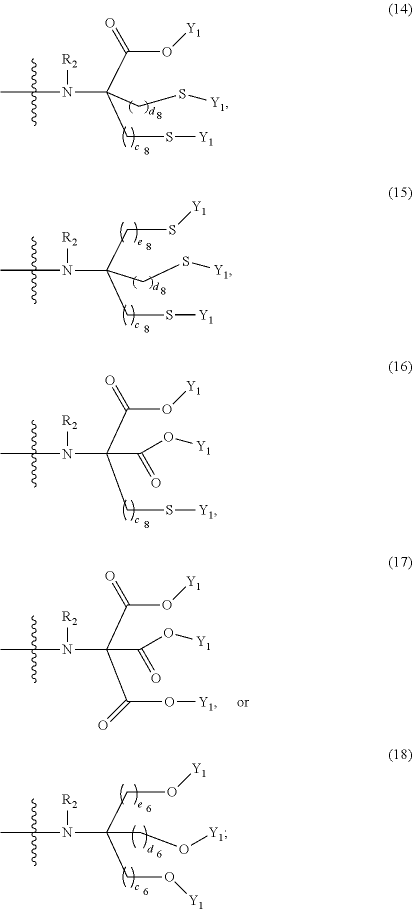

wherein

[symbol] denotes attachment to M^{P} when present or attachment to L^{P} or L^{P′} when M^{P} is absent;

Y₁ denotes attachment to L³ when present or attachment to M^{A} when L³ is absent;

R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxycarbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, —COOH, or —COO—C₁₋₆ alkyl;

each of c₁, c₂, c₃, c₄, c₅, c₆, c₇, and c₈ is an integer independently ranging between 0 and 10;

each of d₁, d₂, d₃, d₄, d₅, d₆, d₇ and d₈ is an integer independently ranging between 0 and 10; and

each of e₁, e₂, e₃, e₄, e₅, e₆, e₇, and e₈ is an integer independently ranging between 0 and 10.

In some embodiments, a₂ is 3 and L^{M} is

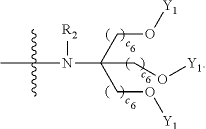

In some embodiments, M^{A} comprises a peptide moiety that comprises at least about five amino acids.

In some embodiments, M^{A} comprises a peptide moiety that comprises at most about sixteen amino acids.

In some embodiments, M^{A} comprises a peptide moiety that comprises about 4, about 5, about 6, about 7, about 8, about 9, about 10, about 11, about 12, about 13, about 14, about 15, or about 16 amino acids.

In some embodiments, M^{A} comprises a peptide moiety that comprises at most about ten amino acids.

In some embodiments, M^{A} comprises a peptide moiety that comprises about 4, about 5, about 6, about 7, about 8, about 9, or about 10 amino acids.

In some embodiments, M^{A} comprises a peptide moiety that comprises from about three to about ten amino acids selected from glycine, serine, glutamic acid, aspartic acid, lysine, cysteine, a stereoisomer thereof (e.g., isoglutamic acid or isoaspartic acid), and a combination thereof.

In some embodiments, M^{A} comprises a peptide moiety that comprises at least four glycines and at least one serine.

In some embodiments, M^{A} comprises a peptide moiety that comprises at least four glycines and at least one glutamic acid.

In some embodiments, M^{A} comprises a peptide moiety that comprises at least four glycines, at least one serine and at least one glutamic acid.

In some embodiments, the ratio between Linker-Drug moiety and PBRM or the ratio between Linker-Drug moiety and the cysteine engineered targeting moiety is between 2:1 and 4:1 or between 2:1 and 1:1. Examples of PBRM include but are not limited to, full length antibodies such as IgG and IgM, antibody fragments such as Fabs, scFv, camelids, Fab2, and the like, small proteins, and peptides.

In some embodiments, the ratio between Linker-Drug moiety and PBRM or the ratio between Linker-Drug moiety and the targeting moiety is about 6:1, about 5:1, about 4:1, about 3:1, about 2:1, or about 1:1.

In some embodiments, the ratio between Linker-Drug moiety and PBRM is about 6:1, about 5:1, about 4:1, about 3:1, about 2:1, or about 1:1.

In some embodiments, the ratio between Linker-Drug moiety and the targeting moiety is about 6:1, about 5:1, about 4:1, about 3:1, about 2:1, or about 1:1.

In some embodiments, the ratio between Linker-Drug moiety and PBRM or the ratio between Linker-Drug moiety and the targeting moiety is about 5:1, about 4:1, about 3:1, about 2:1, or about 1:1.

In some embodiments, the ratio between Linker-Drug moiety and PBRM is about 5:1, about 4:1, about 3:1, about 2:1, or about 1:1.

In some embodiments, the ratio between Linker-Drug moiety and the targeting moiety is about 5:1, about 4:1, about 3:1, about 2:1, or about 1:1.

In some embodiments, the ratio between Linker-Drug moiety and PBRM or the ratio between Linker-Drug moiety and the targeting moiety is about 4:1, about 3:1, about 2:1, or about 1:1.

In some embodiments, the ratio between Linker-Drug moiety and PBRM is about 4:1, about 3:1, about 2:1, or about 1:1.

In some embodiments, the ratio between Linker-Drug moiety and the targeting moiety is about 4:1, about 3:1, about 2:1, or about 1:1.

In some embodiments, the ratio between D and PBRM or the ratio between Drug Units and the targeting moiety is about 4:1, about 2:1, or about 1:1.

In some embodiments, the ratio between D and PBRM is about 4:1, about 2:1, or about 1:1.

In some embodiments, the ratio between Drug Units and the targeting moiety is about 4:1, about 2:1, or about 1:1.

In some embodiments, the ratio between Linker-Drug moiety and PBRM or the ratio between Linker-Drug moiety and the targeting moiety is about 6:1.

In some embodiments, the ratio between Linker-Drug moiety and PBRM is about 6:1.

In some embodiments, the ratio between Linker-Drug moiety and the targeting moiety is about 6:1.

In some embodiments, the ratio between Linker-Drug moiety and PBRM or the ratio between Linker-Drug moiety and the targeting moiety is about 4:1.

In some embodiments, the ratio between Linker-Drug moiety and PBRM is about 4:1.

In some embodiments, the ratio between Linker-Drug moiety and the targeting moiety is about 4:1.

In some embodiments, the ratio between Linker-Drug moiety and PBRM or the ratio between Linker-Drug moiety and the targeting moiety is about 2:1 or about 1:1.

In some embodiments, the ratio between Linker-Drug moiety and PBRM is about 2:1 or about 1:1.

In some embodiments, the ratio between Linker-Drug moiety and the targeting moiety is about 2:1 or about 1:1.

In some embodiments, the ratio between Linker-Drug moiety and PBRM or the ratio between Linker-Drug moiety and the targeting moiety is 2:1.

In some embodiments, the ratio between Linker-Drug moiety and PBRM is 2:1.

In some embodiments, the ratio between Linker-Drug moiety and the targeting moiety is 2:1.

In some embodiments, the ratio between Linker-Drug moiety and PBRM or the ratio between Linker-Drug moiety and the targeting moiety is 1:1.

In some embodiments, the ratio between Linker-Drug moiety and PBRM is 1:1.

In some embodiments, the ratio between Linker-Drug moiety and the targeting moiety is 1:1.

In some embodiments, the conjugate disclosed herein is used for the manufacture of a medicament useful for treating or lessening the severity of disorders, such as, characterized by abnormal growth of cells (e.g., cancer).

In some embodiments, the conjugate disclosed herein is used for the manufacture of a medicament useful for treating disorders, such as, characterized by abnormal growth of cells (e.g., cancer).

In some embodiments, the conjugate disclosed herein is used for the manufacture of a medicament useful for lessening the severity of disorders, such as, characterized by abnormal growth of cells (e.g., cancer).

In some embodiments, the Drug Unit or D is locally delivered to a specific target cell, tissue, or organ.

In some aspects, the present disclosure provides compositions comprising the conjugates, methods for their preparation, and methods of use thereof in the treatment of various disorders, including, but not limited to cancer.

In some aspects, the present disclosure relates to a pharmaceutical composition comprising a scaffold or conjugate described herein and a pharmaceutically acceptable carrier.

In some aspects, the present disclosure relates to a method of treating a disorder in a subject in need thereof, comprising administering to the subject an effective amount of a conjugate disclosed herein.

In some aspects, the present disclosure relates to a method of diagnosing a disorder in a subject suspected of having the disorder. The method comprises administering an effective amount of the conjugate described herein to the subject suspected of having the disorder or performing an assay to detect a target antigen/receptor in a sample from the subject so as to determine whether the subject expresses target antigen or receptor.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE FIGURES

FIG. 1 illustrates the anti-tumor efficacy of the Trastuzumab-drug conjugates, Conjugate 2, Conjugate 3, and Conjugate 4 as measured in a JIMT-1 mouse tumor xenograft model.

FIG. 2 depicts the exposure of the conjugated drug in a JIMT-1 mouse tumor xenograft model as measured after administration of Conjugate 2, Conjugate 3, and Conjugate 4 to mice.

### DETAILED DESCRIPTION

The present disclosure provides novel cysteine engineered targeting moiety-drug conjugates, synthetic methods for making the conjugates or scaffolds, pharmaceutical compositions containing them, and various uses of the conjugates.

### Definitions

Certain compounds of the present disclosure and definitions of specific functional groups are also described in more detail herein. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups.

The use of the articles “a”, “an”, and “the” in both the following description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted, permits but does not require the inclusion of additional elements or steps. In some embodiments, a scaffold of a certain formula includes all components shown in the formula and may also include additional component not shown in the formula. Additionally, whenever “comprising” or another open-ended term is used in an embodiment, it is to be understood that the same embodiment can be more narrowly claimed using the intermediate term “consisting essentially of” or the closed term “consisting of.”

As used herein, the expressions “one or more of A, B, or C,” “one or more A, B, or C,” “one or more of A, B, and C,” “one or more A, B, and C” and the like are used interchangeably and all refer to a selection from a group consisting of A, B, and/or C, i.e., one or more As, one or more Bs, one or more Cs, or any combination thereof.

The term “about”, “approximately”, or “approximate”, when used in connection with a numerical value, means that a collection or range of values is included. In some embodiments, “about X” includes a range of values that are ±25%, ±20%, ±15%, ±10%, ±5%, ±2%, ±1%, ±0.5%, ±0.2%, or ±0.1% of X, where X is a numerical value. In some embodiments, the term “about” refers to a range of values which are 5% more or less than the specified value. In some embodiments, the term “about” refers to a range of values which are 2% more or less than the specified value. In some embodiments, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. In some embodiments, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

“Protecting group”: as used herein, the term protecting group means that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. In some embodiments, in some embodiments, certain exemplary oxygen protecting groups may be utilized. These oxygen protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilyl ether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals. In certain other exemplary embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives. In yet other embodiments, certain exemplary sulphur protecting groups may be utilized. The sulfur protecting groups include, but are not limited to those oxygen protecting group describe above as well as aliphatic carboxylic acid (e.g., acrylic acid), maleimide, vinyl sulfonyl, and optionally substituted maleic acid. Certain other exemplary protecting groups are detailed herein, however, it will be appreciated that the present disclosure is not intended to be limited to these protecting groups; rather, a variety of additional equivalent protecting groups can be readily identified using the above criteria and utilized in the present disclosure. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

“Leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O— in which R is hydrogen, an aliphatic, heteroaliphatic, carbocyclic, or heterocycloalkyl moiety.

“Antibody” refers to a full-length antibody or functional fragment of an antibody comprising an immunoglobulin. By a “functional fragment” it is meant a sufficient portion of the immunoglobulin or antibody is provided that the moiety effectively binds or complexes with the cell surface molecule for its target cell population, e.g., human oncofetal antigen.

An immunoglobulin may be purified, generated recombinantly, generated synthetically, or combinations thereof, using techniques known to those of skill in the art. While immunoglobulins within or derived from IgG antibodies are particularly well-suited for use in the conjugates or scaffolds of this disclosure, immunoglobulins from any of the classes or subclasses may be selected, e.g., IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, intracellular antibodies (“intrabodies”), recombinant antibodies, anti-idiotypic antibodies, domain antibodies, linear antibody, multispecific antibody, antibody fragments, such as, Fv, Fab, F(ab)₂, F(ab)₃, Fab′, Fab′-SH, F(ab′)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, dual-affinity antibodies such as, dual-affinity retargeting proteins (DART™), divalent (or bivalent) single-chain variable fragments (di-scFvs, bi-scFvs) including but not limited to minibodies, diabodies, triabodies or tribodies, tetrabodies, and the like, and multivalent antibodies. “Antibody fragment” refers to at least a portion of the variable region of the immunoglobulin molecule that binds to its target, i.e., the antigen-binding region. As used herein, the term “antibody” refers to both the full-length antibody and antibody fragments unless otherwise specified.

“Protein-based recognition-molecule” or “PBRM” refers to a molecule that recognizes and binds to a cell surface marker or receptor such as, a transmembrane protein, surface immobilized protein, or proteoglycan. In some embodiments, the PBRM comprises an engineered cysteine. Examples of PBRMs include but are not limited to, antibodies (e.g., Trastuzumab, Cetuximab, Rituximab, Bevacizumab, Epratuzumab, Veltuzumab, Labetuzumab, B7-H4, B7-H3, CA125, CD33, CXCR2, EGFR, FGFR1, FGFR2, FGFR3, FGFR4, HER2, NaPi2b, c-Met, Mesothelin, NOTCH1, NOTCH2, NOTCH3, NOTCH4, PD-L1, c-Kit, MUC1, MUC13, Trop-2 and anti-5T4) or peptides (LHRH receptor targeting peptides, EC-1 peptide), lipocalins, such as, for example, anticalins, proteins such as, for example, interferons, lymphokines, growth factors, colony stimulating factors, and the like, peptides or peptide mimics, and the like. The protein-based recognition molecule, in addition to targeting the conjugate to a specific cell, tissue or location, may also have certain therapeutic effect such as antiproliferative (cytostatic and/or cytotoxic) activity against a target cell or pathway. The protein-based recognition molecule comprises or may be engineered to comprise at least one chemically reactive group such as, —COOH, primary amine, secondary amine —NHR, —SH, or a chemically reactive amino acid moiety or side chains such as, for example, tyrosine, histidine, cysteine, or lysine. In some embodiments, a PBRM may be a ligand (LG) or targeting moiety which specifically binds or complexes with a cell surface molecule, such as a cell surface receptor or antigen, for a given target cell population. Following specific binding or complexing of the ligand with its receptor, the cell is permissive for uptake of the ligand or ligand-drug-conjugate, which is then internalized into the cell. As used herein, a ligand that “specifically binds or complexes with” or “targets” a cell surface molecule preferentially associates with a cell surface molecule via intermolecular forces. In some embodiments, the ligand can preferentially associate with the cell surface molecule with a Kd of less than about 50 nM, less than about 5 nM, or less than 500 pM. Techniques for measuring binding affinity of a ligand to a cell surface molecule are well-known; for example, one suitable technique, is termed surface plasmon resonance (SPR). In some embodiments, the ligand is used for targeting and has no detectable therapeutic effect as separate from the drug which it delivers. In some embodiments, the ligand functions both as a targeting moiety and as a therapeutic or immunomodulatory agent (e.g., to enhance the activity of the active drug or prodrug).

“Engineered cysteine”, as used herein, refers to a cysteine amino acid being present in the cysteine engineered target moiety (e.g., the cysteine engineered PBRM). In some embodiments, the cysteine amino acid is introduced into the cysteine engineered target moiety by substituting a non-cysteine amino acid in the corresponding parent target moiety (e.g., the parent PBRM) with the cysteine amino acid. In some embodiments, the cysteine engineered target moiety is a cysteine engineered antibody or antibody fragment, and the cysteine amino acid is introduced by substituting a non-cysteine amino acid in the corresponding parent antibody or antibody fragment (e.g., at V205C (Kabat numbering) of the light chain constant region) with the cysteine amino acid. In some embodiments, the substitution is achieved by mutation.

“Parent target moiety”, as used herein, refers to the corresponding target moiety of the cysteine engineered target moiety prior to the engineering process (e.g., the engineering process introducing the engineered cysteine). It is understood that the parent target moiety may be wild type, mutated, or synthetic.

“Parent Protein-based recognition-molecule” or “Parent PBRM”, as used herein, refers to the corresponding protein-based recognition-molecule of the cysteine engineered protein-based recognition-molecule prior to the engineering process (e.g., the engineering process introducing the engineered cysteine). It is understood that the parent PBRM (e.g., parent antibody or antibody fragment) may be wild type, mutated, or synthetic.

“Cysteine engineered”, as used herein, refers to the feature of a target moiety (e.g., a PBRM (e.g., an antibody or antibody fragment)) as including at least one engineered cysteine.

“Biocompatible” as used herein is intended to describe compounds that exert minimal destructive or host response effects while in contact with body fluids or living cells or tissues. Thus a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “Biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. Preferably (with exception of compounds intended to be cytotoxic, such as, e.g., antineoplastic agents), compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. In the above sentence, the term “normal cells” refers to cells that are not intended to be destroyed or otherwise significantly affected by the compound being tested.

“Biodegradable”: As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable conjugates (or their components, e.g., the peptide-containing scaffolds and the linkers between the scaffolds and the antibody or the drug molecule) described herein, for example, include exposure of the biodegradable conjugates to water at a temperature and a pH of lysosomal intracellular compartment. Biodegradation of some conjugates (or their components, e.g., the peptide-containing scaffolds and the linkers between the scaffolds and the antibody or the drug molecule), can also be enhanced extracellularly, e.g., in low pH regions of the animal body, e.g., an inflamed area, in the close vicinity of activated macrophages or other cells releasing degradation facilitating factors. The integrity of the conjugates or scaffolds disclosed herein can be measured, for example, by size exclusion HPLC or LC/MS. Although faster degradation may be in some cases preferable, in general it may be more desirable that the conjugates or scaffolds disclosed herein degrade in cells with the rate that does not exceed the rate of metabolization or excretion of their fragments by the cells. In preferred embodiments, the biodegradation byproducts of conjugates or scaffolds disclosed herein are biocompatible.

“Bioavailability”: The term “bioavailability” refers to the systemic availability (i.e., blood/plasma levels) of a given amount of drug or compound administered to a subject. Bioavailability is an absolute term that indicates measurement of both the time (rate) and total amount (extent) of drug or compound that reaches the general circulation from an administered dosage form.

“Hydrophilic”: The term “hydrophilic” does not essentially differ from the common meaning of this term in the art, and denotes chemical moieties which contain ionizable, polar, or polarizable atoms, or which otherwise may be solvated by water molecules. Thus a hydrophilic moiety or group, as used herein, refers to an aliphatic, cycloalkyl, heteroaliphatic, heterocycloalkyl, aryl or heteroaryl moiety, which falls within the definition of the term hydrophilic, as defined above. Examples of particular hydrophilic organic moieties which are suitable include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters, polythioesters, polyalcohols and derivatives thereof. In some embodiments, hydrophilic substituents comprise a carboxyl group (COOH), an aldehyde group (CHO), a ketone group (COC₁₋₄ alkyl), a methylol (CH₂OH) or a glycol (for example, CHOH—CH₂OH or CH—(CH₂OH)₂), NH₂, F, cyano, SO₃H, PO₃H, and the like.

Hydrophilicity of the compounds (including drugs, conjugates and scaffolds) disclosed herein can be directly measured through determination of hydration energy, or determined through investigation between two liquid phases, or by HIC chromatography or by chromatography on solid phases with known hydrophobicity, such as, for example, C4 or C18.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Polysaccharide”, “carbohydrate” or “oligosaccharide”: The terms “polysaccharide”, “carbohydrate”, or “oligosaccharide” are known in the art and refer, generally, to substances having chemical formula (CH₂O)ₙ, where generally n>2, and their derivatives. Carbohydrates are polyhydroxyaldehydes or polyhydroxyketones, or change to such substances on simple chemical transformations, such as hydrolysis, oxidation or reduction. Typically, carbohydrates are present in the form of cyclic acetals or ketals (such as, glucose or fructose). These cyclic units (monosaccharides) may be connected to each other to form molecules with few (oligosaccharides) or several (polysaccharides) monosaccharide units. Often, carbohydrates with well defined number, types and positioning of monosaccharide units are called oligosaccharides, whereas carbohydrates consisting of mixtures of molecules of variable numbers and/or positioning of monosaccharide units are called polysaccharides. The terms “polysaccharide”, “carbohydrate”, and “oligosaccharide”, are used herein interchangeably. A polysaccharide may include natural sugars (e.g., glucose, fructose, galactose, mannose, arabinose, ribose, and xylose) and/or derivatives of naturally occurring sugars (e.g., 2′-fluororibose, 2′-deoxyribose, and hexose).

“Drug”: As used herein, the term “drug” refers to a compound which is biologically active and provides a desired physiological effect following administration to a subject in need thereof (e.g., an active pharmaceutical ingredient).

“Prodrug”: As used herein the term “prodrug” refers to a precursor of an active drug, that is, a compound that can be transformed to an active drug. Typically such a prodrug is subject to processing in vivo, which converts the drug to a physiologically active form. In some instances, a prodrug may itself have a desired physiologic effect. A desired physiologic effect may be, e.g., therapeutic, cytotoxic, immunomodulatory, or the like.

“Cytotoxic”: As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

“Cytostatic”: As used herein the term “cytostatic” refers to a drug or other compound which inhibits or stops cell growth and/or multiplication.

“Small molecule”: As used herein, the term “small molecule” refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. In certain preferred embodiments, the small molecule is a drug and the small molecule is referred to as “drug molecule” or “drug” or “therapeutic agent”. In some embodiments, the drug molecule has MW less than or equal to about 5 kDa. In other embodiments, the drug molecule has MW less than or equal to about 1.5 kDa. In embodiments, the drug molecule is selected from vinca alkaloids, auristatins, duocarmycins, kinase inhibitors, MEK inhibitors, KSP inhibitors, PI3 kinase inhibitors, calicheamicins, SN38, camptothecin, topoisomerase inhibitors, non-natural camptothecins, protein synthesis inhibitor, RNA polymerase inhibitor, pyrrolobenzodiazepines, maytansinoids, DNA-binding drugs, DNA intercalation drugs, NAMPT inhibitors, tubulysin, immunomodulatory compounds, and analogs thereof. Preferably, though not necessarily, the drug is one that has already been deemed safe and effective for use by an appropriate governmental agency or body, e.g., the FDA. In some embodiments, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered suitable for the methods, conjugates, and scaffolds disclosed herein. Classes of drug molecules that can be used in the practice of the present invention include, but are not limited to, anti-cancer substances, radionuclides, vitamins, anti-AIDS substances, antibiotics, immunosuppressants, immunomodulatory compounds, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents. Many large molecules are also drugs and such large molecules may be used in the conjugates and other constructs described herein. Examples of suitable large molecules include, e.g., amino acid-based molecules. Amino acid-based molecules may encompass, e.g., peptides, polypeptides, enzymes, antibodies, immunoglobulins, or functional fragments thereof, among others.

A more complete, although not exhaustive, listing of classes and specific drugs suitable for use in the present disclosure may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, Edited by Susan Budavari et al., CRC Press, 1996, both of which are incorporated herein by reference. In preferred embodiments, the drug used in this disclosure is a therapeutic agent that has antiproliferative (cytostatic and/or cytotoxic) activity against a target cell or pathway. The drug may have a chemically reactive group such as, for example, —COOH, primary amine, secondary amine —NHR, —OH, —SH, —C(O)H, —C(O)R, —C(O)NHR^{2b}, C(S)OH, —S(O)₂OR^{2b}, —P(O)₂OR^{2b}, —CN, —NC or —ONO, in which R is an aliphatic, heteroaliphatic, carbocyclic or heterocycloalkyl moiety and R^{2b} is a hydrogen, an aliphatic, heteroaliphatic, carbocyclic, or heterocyclic moiety.

“Active form” as used herein refers to a form of a compound that exhibits intended pharmaceutical efficacy in vivo or in vitro. In particular, when a drug molecule intended to be delivered by the conjugate of the disclosure is released from the conjugate, the active form can be the drug itself or its derivatives, which exhibit the intended therapeutic properties. The release of the drug from the conjugate can be achieved by cleavage of a biodegradable bond of the linker which attaches the drug to the scaffold or conjugate of the disclosure. The active drug derivatives accordingly can comprise a portion of the linker.

“Diagnostic label”: As used herein, the term diagnostic label refers to an atom, group of atoms, moiety or functional group, a nanocrystal, or other discrete element of a composition of matter, that can be detected in vivo or ex vivo using analytical methods known in the art. When associated with a conjugate of the present disclosure, such diagnostic labels permit the monitoring of the conjugate in vivo. Alternatively or additionally, constructs and compositions that include diagnostic labels can be used to monitor biological functions or structures. Examples of diagnostic labels include, without limitation, labels that can be used in medical diagnostic procedures, such as, radioactive isotopes (radionuclides) for gamma scintigraphy and Positron Emission Tomography (PET), contrast agents for Magnetic Resonance Imaging (MRI) (for example paramagnetic atoms and superparamagnetic nanocrystals), contrast agents for computed tomography and other X-ray-based imaging methods, agents for ultrasound-based diagnostic methods (sonography), agents for neutron activation (e.g., boron, gadolinium), fluorophores for various optical procedures, and, in general moieties which can emit, reflect, absorb, scatter or otherwise affect electromagnetic fields or waves (e.g., gamma-rays, X-rays, radiowaves, microwaves, light), particles (e.g., alpha particles, electrons, positrons, neutrons, protons) or other forms of radiation, e.g., ultrasound.

“Animal”: The term animal, as used herein, refers to humans as well as non-human animals, at any stage of development, including, for example, mammals, birds, reptiles, amphibians, fish, worms and single cells. Cell cultures and live tissue samples are considered to be pluralities of animals. Preferably, the non-human animal is a mammal (e.g., a rodent, a mouse, a rat, a rabbit, a monkey, a dog, a cat, a primate, or a pig). An animal may be a transgenic animal or a human clone. The term “subject” encompasses animals.

“Efficient amount”: In general, as it refers to an active agent or drug delivery device, the term “efficient amount” refers to the amount necessary to elicit the desired biological response. As will be appreciated by those of ordinary skill in this art, the efficient amount of an agent or device may vary depending on such factors as the desired biological endpoint, the agent to be delivered, the composition of the encapsulating matrix, the target tissue, etc. In some embodiments, the efficient amount of microparticles containing an antigen to be delivered to immunize an individual is the amount that results in an immune response sufficient to prevent infection with an organism having the administered antigen.

“Natural amino acid” as used herein refers to any one of the common, naturally occurring L-amino acids found in naturally occurring proteins, such as, glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys), methionine (Met) or a stereoisomer thereof, e.g., isoglutamic acid (iGlu) or isoaspartic acid (iAsp). Unless specified otherwise, a reference to an amino acid includes the amino acid itself and its stereoisomers. In some embodiments, the term “glutamic acid” includes both Glu and iGlu while the term “aspartic acid” includes both Asp and iAsp.

“Unnatural amino acid” as used herein refers to any amino acid which is not a natural amino acid. This includes, for example, amino acids that comprise α-, β-, γ-, D-, L-amino acyl residues. More generally, the unnatural amino acid comprises a residue of the general formula

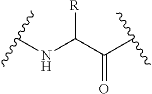

wherein the side chain R is other than the amino acid side chains occurring in nature. Exemplary unnatural amino acids, include, but are not limited to, sarcosine (N-methylglycine), citrulline (cit), homocitrulline, β-ureidoalanine, thiocitrulline, hydroxyproline, allothreonine, pipecolic acid (homoproline), α-aminoisobutyric acid, tert-butylglycine, tert-butylalanine, allo-isoleucine, norleucine, α-methylleucine, cyclohexylglycine, β-cyclohexylalanine, β-cyclopentylalanine, α-methylproline, phenylglycine, α-methylphenylalanine and homophenylalanine.

It is understood that “H”, “—H”, or “hydrogen”, as used herein, may be used interchangeably to refer to a hydrogen atom.

“Alkyl” by itself or as part of another term, as used herein, refers to a substituted or unsubstituted straight chain or branched, saturated or unsaturated hydrocarbon having the indicated number of carbon atoms (e.g., “—C₁₋₈ alkyl” or “—C₁₋₁₀ alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁₋₈ alkyl” groups include, but are not limited to, -methyl, -ethyl, -n-propyl, -n-butyl, -n-pentyl, -n-hexyl, -n-heptyl and -n-octyl; while branched—C₁₋₈ alkyls include, but are not limited to, -isopropyl, -sec-butyl, -isobutyl, -tert -butyl, -isopentyl, and -2-methylbutyl; unsaturated—C₂₋₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexyl, 2-hexyl, -3-hexyl, -acetylenyl, -propynyl, -1-butynyl, -2-butynyl, -1-pentynyl, -2-pentynyl and -3-methyl-1 butynyl. In some embodiments, an alkyl group is unsubstituted. An alkyl group can be substituted with one or more groups. In other aspects, an alkyl group will be saturated.

“Alkylene” by itself of as part of another term, as used herein, refers to a substituted or unsubstituted saturated or unsaturated branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like. In some embodiments, an alkylene is a branched or straight chain hydrocarbon (i.e., it is not a cyclic hydrocarbon). In any of the embodiments provided herein, the alkylene can be a saturated alkylene.

“Aryl” by itself or as part of another term, as used herein, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

“Arylene” by itself or as part of another term, as used herein, is an aryl group as defined above wherein one of the aryl group's hydrogen atoms is replaced with a bond (i.e., it is divalent) and can be in the ortho, meta, or para orientations as shown in the following structures, with phenyl as the exemplary group:

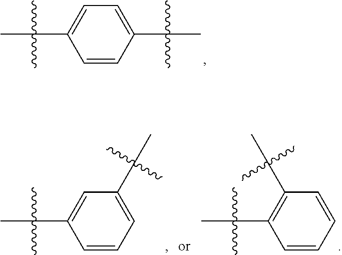 [SMILES: *c1ccc(*)cc1 ; *c1cccc(*)c1 ; *c1ccccc1*]

In some embodiments, e.g., when a Multifunctional Linker or Drug Unit, comprises an arylene, the arylene is an aryl group defined above wherein one or two of the aryl group's hydrogen atoms is replaced with a bond (i.e., the arylene can be divalent or trivalent).

“Heterocycle” by itself or as part of another term, as used herein, refers to a monovalent substituted or unsubstituted aromatic (“heteroaryl”) or non-aromatic (“heterocycloalkyl”) monocyclic, bicyclic, tricyclic, or tetracyclic ring system having a certain number of (e.g., from 3 to 8 or C₃₋₈) carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a heterocycle (e.g., C₃₋₈ heterocycle) include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

“Heterocyclo” or “Heterocyclo-” when used herein, refers to a heterocycle group (e.g., C₃₋₈ heterocycle) defined above wherein one or more of additional hydrogen atoms of the heterocycle are replaced with a bond (i.e., it is multivalent, such as divalent or trivalent). In some embodiments, when a hydrophilic group, Multifunctional Linker or Linker-Drug moiety comprises a heterocyclo, the heterocyclo is a heterocycle group defined above wherein one or two of the heterocycle group's hydrogen atoms is replaced with a bond (i.e., the heterocyclo can be divalent or trivalent).

“Carbocycle” by itself or as part of another term, when used herein, is monovalent, substituted or unsubstituted, aromatic (“aryl”) or saturated or unsaturated nonaromatic (“cycloalkyl”), monocyclic, bicyclic, tricyclic, or tetracyclic carbocyclic ring system having a certain number of (e.g., from 3 to 8 or C₃₋₈) carbon atoms (also referred to as ring members) derived by the removal of one hydrogen atom from a ring atom of a parent ring system. A carbocycle can be 3-, 4-, 5-, 6-, 7- or 8-membered. Representative C₃₋₈ carbocycles include, but are not limited to, cyclopropyl, cyclobutyl, cyclopentyl, cyclopentadienyl, cyclohexyl, cyclohexenyl, phenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

“Carbocyclo” or “Carbocyclo-” by itself or as part of another term, when used herein, refers to a C₃₋₈ carbocycle group defined above wherein another of the carbocycle groups' hydrogen atoms is replaced with a bond (i.e., it is divalent). In select embodiments, e.g., when a hydrophilic group, Multifunctional Linker or Linker-Drug moiety comprises a carbocyclo, the carbocyclo is a carbocycle group defined above wherein one or two of the carbocycle group's hydrogen atoms is replaced with a bond (i.e., the carbocyclo can be divalent or trivalent).

“Heteroalkyl” by itself or in combination with another term, when used herein, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₂, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. In preferred embodiments, a C₁₋₄ heteroalkyl or heteroalkylene has 1 to 4 carbon atoms and 1 or 2 heteroatoms and a C₁₋₃ heteroalkyl or heteroalkylene has 1 to 3 carbon atoms and 1 or 2 heteroatoms. In some aspects, a heteroalkyl or heteroalkylene is saturated.

“Heteroalkylene” by itself or as part of another substituent, when used herein, means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied. In select embodiments, e.g., when a hydrophilic group, Multifunctional Linker or Linker-Drug moiety comprises a heteroalkylene, the heteroalkylene is a heteroalkyl group defined above wherein one or two of the heteroalkyl group's hydrogen atoms is replaced with a bond (i.e., the heteroalkylene can be divalent or trivalent).

“Optionally substituted” when used herein, means that a chemical moiety (such as alkyl, heteroalkyl, carbocycle, and heterocycle, etc.) is either substituted or unsubstituted. Unless otherwise specified, the chemical moieties disclosed herein are optionally substituted. When a chemical moiety is substituted, one or more hydrogen atoms are each independently replaced with a substituent. Typical substituents include, but are not limited to, —X′, —R′, —O, —OR′, —SR′, —S—, —N(R′)₂, —N(R′)₃, ═NR′, —C(X′)₃, —CN, —OCN, —SCN, —N═C═O, —NCS, —NO, —NO₂, ═N₂, —N₃, —NR′C(═O)R′, —C(═O)R′, —C(═O)N(R′)₂, —SO₃—, —SO₃H, —S(═O)₂R′, —OS(═O)₂OR′, —S(═O)₂NR′, —S(═O)R′, —OP(═O)(OR′)₂, —P(═O)(OR′)₂, —PO₃—, —PO₃H₂, —AsO₂H₂, —C(═O)R′, —C(═O)X′, —C(═S)R′, —CO₂R′, —CO₂—, —C(═S)OR′, C(═O)SR′, C(═S)SR′, C(═O)N(R′)₂, C(═S)N(R′)₂, or C(═NR′)N(R′)₂, wherein each X′ is independently a halogen: —F, —Cl, —Br, or —I; and each R′ is independently —H, —C₁₋₂₀ alkyl, —C₆₋₂₀ aryl, —C₃-C14 heterocycle, a protecting group or a prodrug moiety. Typical substituents also include oxo (═O).

“Linker-Drug moiety” as used herein, refers to the non-targeting moiety portion of a conjugate disclosed herein. The Linker component of the Linker-Drug moiety has the release mechanism, which is referred to as the Releasable Assembly Unit, interposed between a Multifunctional Linker and a Drug Unit.

“Multifunctional Linker” as used herein, refers to a linker that connects one or more hydrophilic groups, one or more Drug Units, and a targeting moiety (e.g., a PBRM) to form a conjugate or scaffold as disclosed herein. The connection of these components to the Multifunctional Linker can either be parallel or serial. In some embodiments, the Multifunctional Linker comprises a peptide moiety between the targeting moiety and the hydrophilic group, wherein the peptide moiety includes at least two amino acids. In other embodiments, the Multifunctional Linker does not have to comprise a peptide moiety of at least two amino acids when the hydrophilic group is a polyalcohol or a derivative thereof. In other embodiments, the Multifunctional Linker does not have to comprise a peptide moiety of at least two amino acids when the hydrophilic group is a glucosyl-amine, a di-glucosyl-amine, a tri-glucosyl-amine or a derivative thereof.

As used herein, the phrase “parallel orientation”, “parallel placement”, “parallel connection” or like terms refer to a configuration wherein the parallel-placed or parallel-oriented or parallel-connected components are attached to the Multifunctional Linker in such a manner that each has one end tethered to the Multifunctional Linker and one free end. The term “parallel” is used herein is not being used to denote that two components are side-by-side in space or have the same distance between them throughout some or their entire lengths. In instances where a parallel-oriented component is itself branched and thus has multiple ends, it still has only one tethered end. In some embodiments, only those hydrophilic groups, required to mask hydrophobicity for a given Linker-Drug moiety are in parallel orientation to the Drug Unit, which does not necessarily require all of the Drug Units and hydrophilic groups connected to the Multifunctional Linker be in parallel orientations to one another. In other embodiments, all of the Drug Units and hydrophilic groups connected to the Multifunctional Linker are in parallel orientations to one another.

The phrase “serial orientation” or “serial placement” or “serial connection” or like terms refer to a configuration of a component in a conjugate or scaffold of the disclosure wherein the serially-oriented component is attached in such a manner that it has two tethered ends with each end connected to a different component of the conjugate or scaffold of the disclosure. In some embodiments, one or more (OCH₂CH₂) subunits, which characterize a PEG unit or subunit, are interposed between the Drug Unit and the targeting moiety.

“Free drug” as used herein, refers to a biologically active form of a drug moiety that is not covalently attached either directly or indirectly to a hydrophilic group or to a degradant product of a Ligand Unit. Free drug can refer to the drug, as it exists immediately upon cleavage from the Multifunctional Linker via the release mechanism, which is provided by the Releasable Assembly Unit in the Linker-Drug moiety, or, to subsequent intracellular conversion or metabolism. In some aspects, the free drug will have the form H-D or may exist a as a charged moiety. The free drug is a pharmacologically active species which can exert the desired biological effect. In some aspects, the pharmacologically active species may not be the parent drug and may include a component of the linker through which the drug is connected to the targeting moiety, which has not undergone subsequent intracellular metabolism.

Hydrophobicity can be measured using c log P. c log P is defined as the log of the octanol/water partition coefficient (including implicit hydrogens) and can be calculated using the program MOE™ from the Chemical Computing group (c log P values calculated using Wildman, S. A., Crippen, G. M.; Prediction of Physiochemical Parameters by Atomic Contributions; J. Chem. Inf. Comput. Sci. 39 No. 5 (1999) 868-873).

In some embodiments, the present disclosure provides a targeting moiety-drug conjugate composition comprising a population of targeting moiety-drug conjugates. The targeting moiety-drug conjugate comprises a targeting moiety unit and multiple Linker-Drug moieties attached thereto. Preferably, there is an average of from about 2 to about 6, from about 2 to about 4, or from about 1 to about 2, Linker-Drug moieties (e.g., do of Formula (I)) per targeting moiety in the conjugate. Exemplary attachment to the targeting moiety is via thioether linkages. Exemplary conjugation sites on a targeting moiety are the thiol groups obtained from reduction of interchain disulfide residues and/or thiol-containing residues introduced into the targeting moiety such as introduced cysteines. Attachment can be, for example, via thiol residues derived from an interchain disulfide and from 0 to 8 introduced cysteine residues.

As used herein, “molecular weight” or “MW” of a polymer refers to the weight average molecular weight unless otherwise specified.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include ¹³C and ¹⁴C.

The compound, scaffold, or conjugate of the present disclosure may exist in more than one isomeric form. It is understood that when a compound, scaffold, or conjugate is described herein, the disclosure refers to all isomers of the compound, scaffold, or conjugate. Such disclosure refers to, where applicable, regioisomers optical isomers and tautomeric isomers. In some embodiments, the optical isomers include enantiomers, diastereomers, chiral isomers, and non-chiral isomers. In some embodiments, the optical isomers include isolated optical isomers as well as mixtures of optical isomers including racemic and non-racemic mixtures. An isomer may be in isolated form or in a mixture with one or more other isomers. Unless stated otherwise, any compound, scaffold, or conjugate described herein is meant to refer to each isomer of the compound, scaffold, or conjugate, or any mixture thereof. When a compound, scaffold, or conjugate is depicted as a specific isomer, it is understood that the present disclosure is not limited to that specific isomer, but may refer to the specific isomer as an optional embodiment.

In some embodiments, the compound, scaffold, or conjugate of the present disclosure may exist as cis and/or trans isomers. Unless stated otherwise, any compound, scaffold, or conjugate described herein is meant to refer to the cis isomer or trans isomer of the compound, scaffold, or conjugate, as well as any mixture thereof. When a compound, scaffold, or conjugate is depicted as a cis or trans isomer, it is understood that the present disclosure is not limited to that specific cis or trans isomer, but may refer to the specific cis or trans isomer as an optional embodiment.

In some embodiments, the compound, scaffold, or conjugate of the present disclosure may exist as regioisomers. Unless stated otherwise, any compound, scaffold, or conjugate described herein is meant to refer to each regioisomer of the compound, scaffold, or conjugate, or any mixture thereof. When a compound, scaffold, or conjugate is depicted as a specific regioisomer, it is understood that the present disclosure is not limited to that specific regioisomer, but may refer to the specific regioisomer as an optional embodiment. Recitation or depiction of a compound, scaffold, or conjugate of the present disclosure without a specific stereoconfiguration designation, or with such a designation for less than all chiral centers, is intended to encompass, for such undesignated chiral centers, the racemate, racemic mixtures, each individual enantiomer, a diastereoisomeric mixture and each individual diastereomer of the compound wherein such forms are possible due to the presence of one or more asymmetric centers.

### Conjugates and Peptide-Containing Scaffolds

In some aspects, the disclosure relates to a conjugate of Formula (I) with a protein-based recognition-molecule (PBRM):

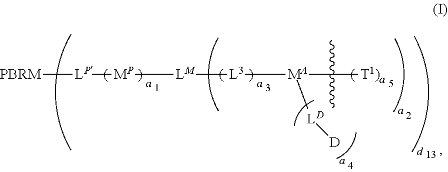

wherein

a₁, when present, is an integer from 0 to 1;

a₂ is an integer from 1 to 3;

a₃, when present, is an integer from 0 to 1;

a₄ is an integer from 1 to about 5;

a₅ is an integer from 1 to 3;

d₁₃ is an integer from 1 to about 6;

PBRM denotes a protein-based recognition-molecule, wherein the PBRM comprises an engineered cysteine;

L^{P′} is a divalent linker moiety connecting the engineered cysteine of the PBRM to M^{P}; of which the corresponding monovalent moiety L^{P} contains a functional group W^{P} that is capable of forming a covalent bond with the engineered cysteine of the PBRM;

M^{P}, when present, is a Stretcher unit;

L^{M} is a bond, or a trivalent or tetravalent linker, and when L^{M} is a bond, a₂ is 1, when L^{M} is trivalent linker, a₂ is 2, or when L^{M} is a tetravalent linker, a₂ is 3;

L³, when present, is a carbonyl-containing moiety;

M^{A} comprises a peptide moiety that contains at least two amino acids;

T¹ is a hydrophilic group and the [symbol] between T¹ and M^{A} denotes direct or indirect attachment of T¹ and M^{A};

each occurrence of D is independently a therapeutic agent having a molecular weight ≤about 5 kDa; and

each occurrence of L^{D} is independently a divalent linker moiety connecting D to M^{A} and comprises at least one cleavable bond such that when the bond is broken, D is released in an active form for its intended therapeutic effect.

In some aspects, the disclosure relates to a peptide-containing scaffold of any one of Formulae (II)-(V):

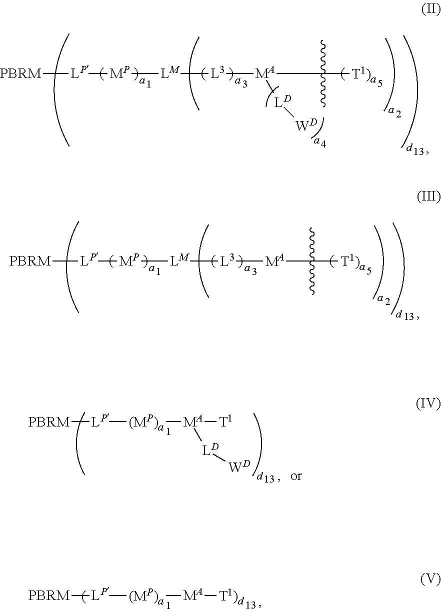

wherein

a₁, when present, is an integer from 0 to 1;

a₂, when present, is 3;

a₃, when present, is an integer from 0 to 1;

a₄, when present, is an integer from 1 to about 5;

a₅, when present, is an integer from 1 to 3;

d₁₃ is an integer from 1 to about 6;

PBRM denotes a protein-based recognition-molecule, wherein the PBRM comprises an engineered cysteine;

L^{P′} is a divalent linker moiety connecting the engineered cysteine of the PBRM to M^{P}; of which the corresponding monovalent moiety L^{P} contains a functional group W^{P} that is capable of forming a covalent bond with the engineered cysteine of the PBRM;

M^{P}, when present, is a Stretcher unit;

L^{M}, when present, is a bond, or a trivalent or tetravalent linker, and when L^{M} is a bond, a₂ is 1, when L^{M} is trivalent linker, a₂ is 2, or when L^{M} is a tetravalent linker, a₂ is 3;

L³, when present, is a carbonyl-containing moiety;

M^{A} comprises a peptide moiety that contains at least two amino acids;

T¹ is a hydrophilic group and the [symbol] between T¹ and M^{A} denotes direct or indirect attachment of T¹ and M^{A};

each occurrence of W^{D} is independently a functional group that is capable of forming a covalent bond with a functional group of a therapeutic agent (“D”) having a molecular weight ≤about 5 kDa; and

each occurrence of L^{D} is independently a divalent linker moiety connecting W^{D} or D to M^{A} and L^{D} comprises at least one cleavable bond such that when the bond is broken, D is released in an active form for its intended therapeutic effect.

The conjugates and scaffolds of the disclosure can include one or more of the following features when applicable.

In some embodiments, d₁₃ is an integer from about 1 to about 6 (e.g., d₁₃ is 1, 2, 3 4, 5 or 6).

In some embodiments, d₁₃ is an integer from about 2 to about 6 (e.g., d₁₃ is 2, 3, 4, 5 or 6).

In some embodiments, d₁₃ is an integer from about 4 to about 6 (e.g., d₁₃ is 4, 5 or 6).

In some embodiments, d₁₃ is an integer from about 1 to about 4 (e.g., d₁₃ is 1, 2, 3 or 4).

In some embodiments, d₁₃ is an integer from about 2 to about 4 (e.g., d₁₃ is 2, 3 or 4).

In some embodiments, d₁₃ is an integer from about 3 to about 4.

In some embodiments, d₁₃ is an integer from about 1 to about 2.

In some embodiments, d₁₃ is 1. In some embodiments, d₁₃ is 2. In some embodiments, dis is 3. In some embodiments, d₁₃ is 4. In some embodiments, d₁₃ is 5. In some embodiments, d₁₃ is 6.

In some embodiments, L³, when present, comprises —X—C₁₋₁₀ alkylene-C(O)—, with X directly connected to L^{M}, in which X is CH₂, O, or NR₅, and R₅ is hydrogen, C₁₋₆ alkyl, C₆₋₁₀ aryl, C₃₋₈ cycloalkyl, COOH, or COO—C₁₋₆ alkyl.

In some embodiments, L³ is —NR₅—(CH₂)ᵥ—C(O)— or —CH₂—(CH₂)ᵥ—C(O)—NR₅—(CH₂)ᵥ—C(O)—, in which each v independently is an integer from 1 to 10. In some embodiments, L³, when present, is —NH—(CH₂)₂—C(O)— or —(CH₂)₂—C(O)—NH—(CH₂)₂—C(O)—.

In some embodiments, each v independently is an integer from 1 to 6, or from 2 to 4, or v is 2.

In some embodiments, a₄ is 1.

In some embodiments, a₄ is 2. In some embodiments, a₄ is 3. In some embodiments, a₄ is 4.

In some embodiments, a₄ is 5.

### L^{P′}

In some embodiments, L^{P′} is a divalent linker moiety connecting the engineered cysteine of the PBRM to M^{P}, of which the corresponding monovalent moiety is L^{P}.

In some embodiments, L^{P} is the corresponding monovalent moiety of L^{P′} when not connected to the engineered cysteine of the PBRM. In some embodiments, L^{P} comprises a terminal group W^{P}, in which each W^{P} independently is:

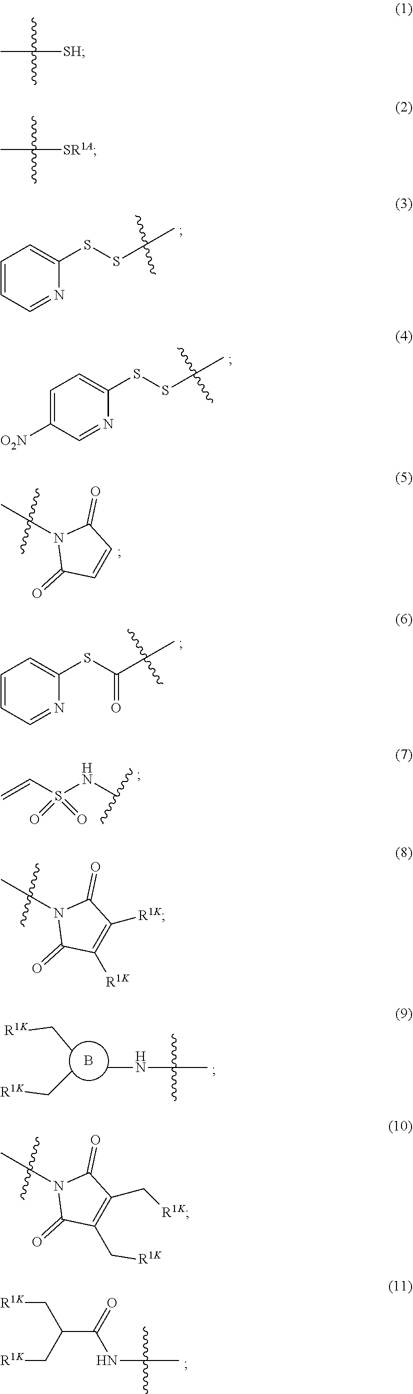

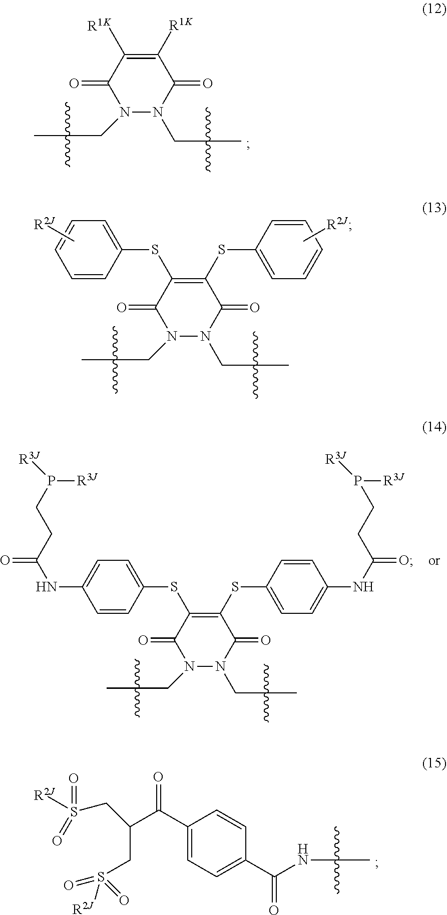

wherein

ring B is cycloalkyl, heterocycloalkyl, aryl, or heteroaryl;

R^{1K} is a leaving group;

R^{1A} is a sulfur protecting group;

R^{2J} is hydrogen, an aliphatic, aryl, heteroaliphatic, or carbocyclic moiety; and

R^{3J} is C₁₋₆ alkyl and each of Z₁, Z₂, Z₃, and Z₇ is independently a carbon or nitrogen atom.

In some embodiments, each R^{1K} is halo or RC(O)O— in which R is hydrogen, an aliphatic, heteroaliphatic, carbocyclic, or heterocycloalkyl moiety.

In some embodiments, each R^{1A} independently is

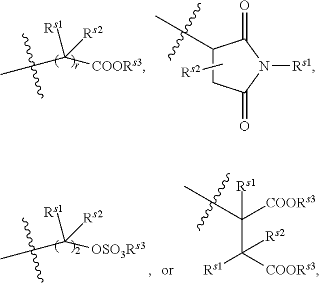

in which r is 1 or 2 and each of R^{s1}, R^{s2}, and R^{s3} is hydrogen, an aliphatic, heteroaliphatic, carbocyclic, or heterocycloalkyl moiety.

In some embodiments, W^{P} is

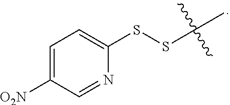 [SMILES: *SSc1ccc([N+](=O)[O-])cn1]

In some embodiments, W^{P} is

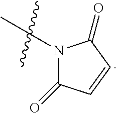 [SMILES: *N1C(=O)C=CC1=O]

In some embodiments, when W^{P} is

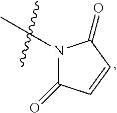 [SMILES: *N1C(=O)C=CC1=O]

L^{P′} comprises

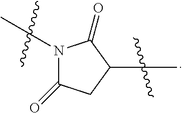 [SMILES: *C1CC(=O)N(*)C1=O]

In some embodiments, W^{P} is

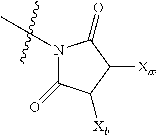

wherein one of Xₐ and X_{b} is H and the other is a maleimido blocking moiety. In some embodiments, a maleimido blocking compound (i.e., a compound that can react with maleimide to convert it to succinimide) may be used to quench the reaction between, e.g., the Linker-Drug moiety and the PBRM (e.g., the engineered cysteine of the PBRM), and a maleimido blocking moiety refers to the chemical moiety attached to the succinimide upon conversion. In some embodiments, the maleimido blocking moieties are moieties that can be covalently attached to one of the two olefin carbon atoms upon reaction of the maleimido group with a thiol-containing compound of Formula (II′):

R₉₀—(CH₂)_{d}—SH (II′)

wherein:

R₉₀ is NHR₉₁, OH, COOR₉₃, CH(NHR₉₁)COOR₉₃, or a substituted phenyl group;

R₉₃ is hydrogen or C₁₋₄ alkyl;

R₉₁ is hydrogen, CH₃, or CH₃CO; and

d is an integer from 1 to 3.

In some embodiments, the maleimido blocking compound can be cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is optionally substituted with one or more hydrophilic substituents, or 3-aminopropane-1-thiol. In some embodiments, the one or more hydrophilic substituents on phenyl comprise OH, SH, methoxy, ethoxy, COOH, CHO, COC₁₋₄ alkyl, NH₂, F, cyano, SO₃H, PO₃H, and the like.

In some embodiments, the maleimido blocking group is —S—(CH₂)_{d}—R₉₀, in which,

R₉₀ is OH, COOH, or CH(NHR₉₁)COOR₉₃;

R₉₃ is hydrogen or CH₃;

R₉₁ is hydrogen or CH₃CO; and

d is 1 or 2.

In some embodiments, the maleimido blocking group is —S—CH₂—CH(NH₂)COOH.

### Stretcher Unit M^{P}

In some embodiments, M^{P}, when present, is —(Z₄)—[(Z₅)—(Z₆)]_{z}—, wherein Z₄ is connected to L^{P′} or L^{P} and Z₆ is connected to L^{M}; in which

z is 1, 2, or 3;

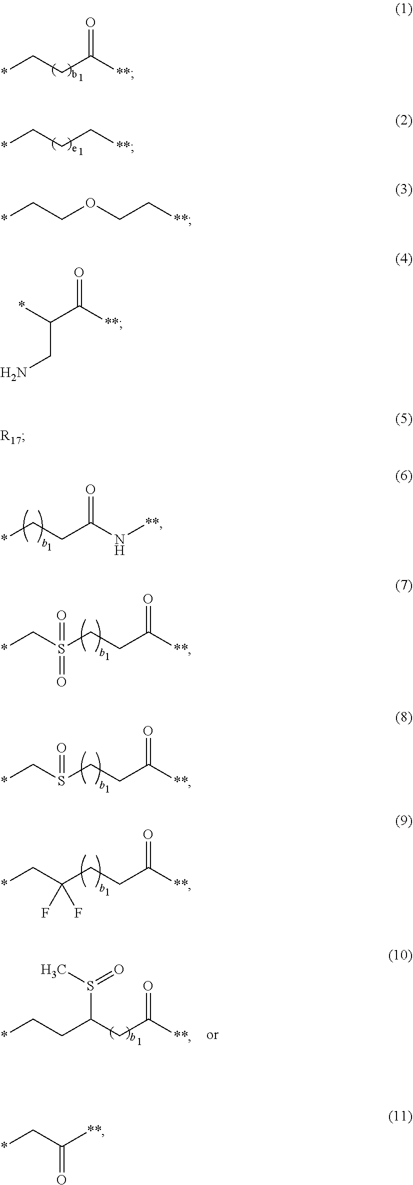

wherein * denotes attachment to L^{P′} or L^{P} and ** denotes attachment to Z₅ or Z₆, when present, or to L^{M} when Z₅ and Z₆ are both absent;

b₁ is an integer from 0 to 6;

e₁ is an integer from 0 to 8;

R₁₇ is C₁₋₁₀ alkylene, C₁₋₁₀ heteroalkylene, C₃₋₈ cycloalkylene, O—(C₁₋₈ alkylene), arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4- to 14-membered heterocycloalkylene, —C₁₋₁₀ alkylene-(4- to 14-membered heterocycloalkylene)-, -(4- to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4- to 14-membered heterocycloalkylene-C(═O)—, —C₁₋₁₀ alkylene-(4- to 14-membered heterocycloalkylene)-C(═O)—, -(4- to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4- to 14-membered heterocycloalkylene-NH—, —C₁₋₁₀ alkylene-(4- to 14-membered heterocycloalkylene)-NH—, -(4- to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4- to 14-membered heterocycloalkylene-S—, —C₁₋₁₀ alkylene-(4- to 14-membered heterocycloalkylene)-S—, or -(4- to 14-membered heterocycloalkylene)-C₁₋₁₀ alkylene-S—;

each Z₅ independently is absent, R₅₇—R₁₇, or a polyether unit;

each R⁵⁷ independently is a bond, NR₂₃, S, or O;

each R₂₃ independently is hydrogen, C₁₋₆ alkyl, C₆₋₁₀ aryl, C₃₋₈ cycloalkyl, COOH, or COO—C₁₋₆ alkyl;

each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—;

each R₃ independently is —C(O)—NR₅— or —NR₅—C(O)—;

each R₅ independently is hydrogen, C₁₋₆ alkyl, C₆₋₁₀ aryl, C₃₋₈ cycloalkyl, COOH, or COO—C₁₋₆ alkyl; and

g₁ is an integer from 1 to 4.

In some embodiments, Z₄ is

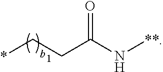

In some embodiments, Z₄ is

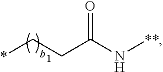

wherein b₁ is 1.

In some embodiments, Z₄ is

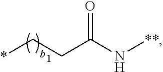

wherein b₁ is 4.

In some embodiments, Z₄ is

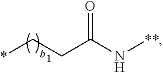

wherein b₁ is 4.

In some embodiments, Z₄ is

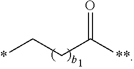

In some embodiments, Z₄ is

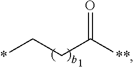

e.g., wherein b₁ is 4.

In some embodiments, Z₄ is

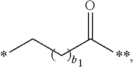

e.g., wherein b₁ is 0.

In some embodiments, Z₄ is

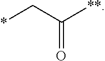

In some embodiments, Z₄ is

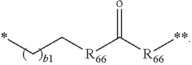

In some embodiments, b₁ is 0. In some embodiments, one of R₆₆ is O, and the other is NH.

In some embodiments, Z₄ is

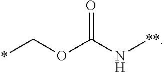

In some embodiments, Z₄ is

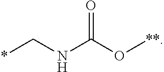

In some embodiments, each Z₅ independently is a polyalkylene glycol (PAO), including but are not limited to, polymers of lower alkylene oxides (e.g., polymers of ethylene oxide, such as, for example, propylene oxide, polypropylene glycols, polyethylene glycol (PEG), polyoxyethylenated polyols, copolymers thereof and block copolymers thereof). In some embodiments, the polyalkylene glycol is a polyethylene glycol (PEG) including, but not limited to, polydisperse PEG, monodisperse PEG and discrete PEG. In some embodiments, polydisperse PEGs are a heterogeneous mixture of sizes and molecular weights whereas monodisperse PEGs are purified from heterogeneous mixtures and therefore provide a single chain length and molecular weight. In some embodiments, the PEG units are discrete PEGs. In some embodiments, the discrete PEGs provide a single molecule with defined and specified chain length. In some embodiments, the PEG is mPEG.

As used herein a subunit when referring to the PEG unit refers to a polyethylene glycol subunit having the formula

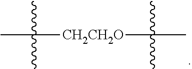

In some such embodiments, the PEG unit comprises multiple PEG subunits.

In some embodiments, when z is 2 or 3, at least one Z₅ is a polyalkylene glycol (PAO), e.g., a PEG unit.

In some embodiments, when z is 2, at least one Z₅ is a polyalkylene glycol (PAO), e.g., a PEG unit.

In some embodiments, when z is 3, at least one Z₅ is a polyalkylene glycol (PAO), e.g., a PEG unit.

In some embodiments, the PEG unit comprises 1 to 6 subunits.

In some embodiments, the PEG unit comprises 1 to 4 subunits.

In some embodiments, the PEG unit comprises 1 to 3 subunits.

In some embodiments, the PEG unit comprises 1 subunit.

In some embodiments, the PEG unit comprises 2 subunits.

In some embodiments, the PEG unit comprises 3 subunits.

In some embodiments, the PEG unit comprises 4 subunits.

In some embodiments, the PEG unit comprises 5 subunits.

In some embodiments, the PEG unit comprises 6 subunits.

In some embodiments, the PEG unit comprises one or multiple PEG subunits linked together by a PEG linking unit. In some embodiments, the PEG linking unit that connects one or more chains of repeating CH₂CH₂O— subunits is Z₆. In some embodiments, Z₆ is —C₁₋₁₀ alkyl-R₃—, —C₂₋₁₀ alkyl-NH—, —C₂₋₁₀ alkyl-C(O)—, —C₂₋₁₀ alkyl-O— or —C₁₋₁₀ alkyl-S, wherein R₃ is —C(O)—NR₅— or —NR₅—C(O)—.

In some embodiments, the PEG linking unit is —C₁₋₁₀ alkyl-C(O)—NH— or —C₁₋₁₀ alkyl-NH—C(O)—. In some embodiments, the PEG linking unit is —C₁₋₁₀ alkyl-C(O)—NH—. In some embodiments, the PEG linking unit is —C₁₋₁₀ alkyl-NH—C(O)—.

In some embodiments, the PEG linking unit is —(CH₂)₂—C(O)—NH—.

In some embodiments, each Z₅ is absent.

In some embodiments, when z is 2 or 3, at least one Z₅ is absent.

In some embodiments, when z is 2, at least one Z₅ is absent. In some embodiments, when z is 3, at least one Z₅ is absent.

In some embodiments, each Z₅ is —(CH₂—CH₂—O—)₂—.

In some embodiments, when z is 2 or 3, at least one Z₅ is —(CH₂—CH₂—O—)₂—. In some embodiments, when z is 2, at least one Z₅ is —(CH₂—CH₂—O—)₂—. In some embodiments, when z is 3, at least one Z₅ is —(CH₂—CH₂—O—)₂—.

In some embodiments, each Z₅ independently is R₅₇—R₁₇. In some embodiments, each Z₅ independently is R₁₇, NHR₁₇, OR₁₇, or SR₁₇.

In some embodiments, when z is 2 or 3, at least one Z₅ is R₅₇—R₁₇ (e.g., R₁₇, NHR₁₇, OR₁₇—, or SR₁₇).

In some embodiments, when z is 2, at least one Z₅ is R₅₇—R₁₇ (e.g., R₁₇, NHR₁₇, OR₁₇, or SR₁₇). In some embodiments, when z is 3, at least one Z₅ is R₅₇—R₁₇ (e.g., R₁₇, NHR₁₇, OR₁₇, or SR₁₇).

In some embodiments, each Z₆ is absent.

In some embodiments, when z is 2 or 3, at least one Z₆ is absent.

In some embodiments, when z is 2, at least one Z₆ is absent. In some embodiments, when z is 3, at least one Z₆ is absent.

In some embodiments, at least one of Z₅ and Z₆ is not absent.

In some embodiments, each Z₆ independently is —C₁₋₁₀ alkyl-R₃—, —C₁₋₁₀ alkyl-NH—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—. In some embodiments, g₁ is an integer from 1 to 4.

In some embodiments, when z is 2 or 3, at least one Z₆ is —C₁₋₁₀ alkyl-R₃—, —C₁₋₁₀ alkyl-NH—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—. In some embodiments, g₁ is an integer from 1 to 4.

In some embodiments, each Z₆ independently is —C₂₋₁₀ alkyl-C(O)— (e.g., —(CH₂)₂—C(O)—).

In some embodiments, at least one Z₆ is —C₂₋₁₀ alkyl-C(O)— (e.g., —(CH₂)₂—C(O)—).

In some embodiments, each Z₆ independently is —C₂₋₁₀ alkyl-R₃—C₂₋₁₀ alkyl-C(O)— (e.g., —(CH₂)₂—C(O)NH—(CH₂)₂—C(O)—).

In some embodiments, at least one Z₆ is —C₂₋₁₀ alkyl-R₃—C₂₋₁₀ alkyl-C(O)— (e.g., —(CH₂)₂—C(O)NH—(CH₂)₂—C(O)—).

In some embodiments, each Z₆ independently is —(C₂₋₁₀ alkyl-R₃)_{g1}—C₂₋₁₀ alkyl-C(O)— (e.g., —(CH₂)₂—C(O)NH—(CH₂)₂—NHC(O)—(CH₂)—C(O)—).

In some embodiments, at least one Z₆ is —(C₂₋₁₀ alkyl-R₃)_{g1}—C₂₋₁₀ alkyl-C(O)— (e.g., —(CH₂)₂—C(O)NH—(CH₂)₂—NHC(O)—(CH₂)—C(O)—) or —(CH₂)₂—NH—C(O)—(CH₂)₂—C(O)—NH—(CH₂)—C(O)—.

In some embodiments, each Z₆ independently is —(CH₂)₂—NH—C(O)—(CH₂)₂—C(O)—NH—CH₂—C(O)—.

In some embodiments, each Z₆ independently is —(CH₂)₂—C(O)—NH—(CH₂)₂—NH—C(O)—(CH₂)—C(O)— or —(CH₂)₂—NH—C(O)—CH₂)₂—C(O)—NH—(CH₂)—C(O)—.

In some embodiments, —[(Z₅)—(Z₆)]_{z}— is not absent.

In some embodiments, —[(Z₅)—(Z₆)]_{z}— is a bond.

In some embodiments, —[(Z₅)—(Z₆)]_{z}— is —(CH₂CH₂O)₂—(CH₂)₂—C(O)—.

In some embodiments, —[(Z₅)—(Z₆)]_{z}— is —(CH₂CH₂O)₂—(CH₂)₂—C(O)—NH—(CH₂CH₂O)₂—.

In some embodiments, —[(Z₅)—(Z₆)]_{z}— is —(CH₂CH₂O)₂—(CH₂)₂—C(O)—NH—(CH₂) C(O)—.

In some embodiments, —[(Z₅)—(Z₆)]_{z}— is —(CH₂CH₂O)₂—(CH₂)₂—NH—C(O)—.

In some embodiments, —[(Z₅)—(Z₆)]_{z}— is —(CH₂CH₂O)₂—(CH₂)₂—NH—C(O)—(CH₂)₂—C(O)—NH—(CH₂)—C(O)—.

In some embodiments, —[(Z₅)—(Z₆)]_{z}— is —(CH₂CH₂O)₂—(CH₂)₂—C(O)—NH—(CH₂CH₂O)₂—(CH₂)₂—NH—C(O)—(CH₂)₂—C(O)—NH—(CH₂)—C(O)—.

In some embodiments, M^{P}, when present, is

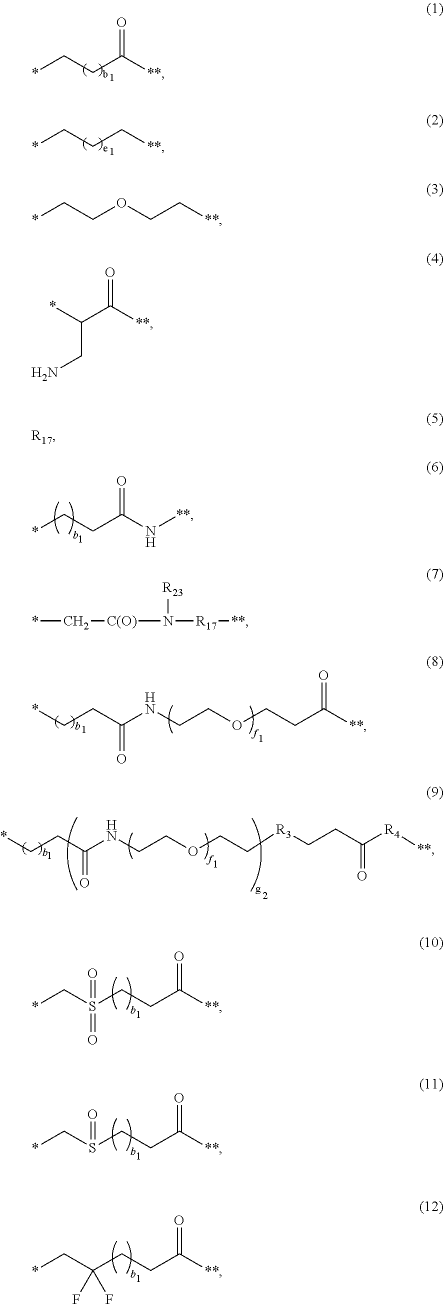

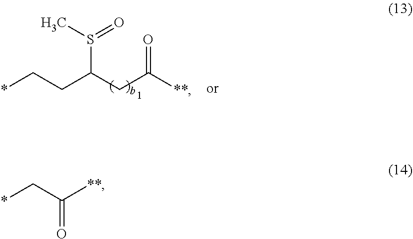

wherein * denotes attachment to L^{P′} or L^{P} and ** denotes attachment to L^{M}; and R₃, R₅, R₁₇, and R₂₃ are as defined herein;

R₄ is a bond or —NR₅—(CR₂₀R₂₁)—C(O)—;

each R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

each b₁ independently is an integer from 0 to 6;

e₁ is an integer from 0 to 8;

each f₁ independently is an integer from 1 to 6; and

g₂ is an integer from 1 to 4.

In some embodiments, b₁ is 1.

In some embodiments, b₁ is 0.

In some embodiments, each f₁ independently is 1 or 2. In some embodiments, f₁ is 1.

In some embodiments, f₁ is 2.

In some embodiments, g₂ is 1 or 2. In some embodiments, g₂ is 1.

In some embodiments, g₂ is 2.

In some embodiments, R₁₇ is unsubstituted.

In some embodiments, R₁₇ is optionally substituted.

In some embodiments, R₁₇ is substituted.

In some embodiments, R₁₇ is optionally substituted by a basic unit, e.g., —(CH₂)ₓNH₂, —(CH)ₓNHR^{a}, and —(CH₂)ₓN(R⁹)₂, wherein x is an integer from 1 to 4 and each R^{a} is independently selected from C₁₋₆ alkyl and C₁₋₆ haloalkyl, or two R^{a} groups are combined with the nitrogen to which they are attached to form an azetidinyl, pyrrolidinyl, or piperidinyl group.

In some embodiments, R₁₇ is substituted by a basic unit, e.g., —(CH₂)ₓNH₂, —(CH₂)ₓNHR^{a}, and —(CH₂)ₓN(R^{a})₂, wherein x is an integer from 1 to 4 and each R^{a} is independently selected from C₁₋₆ alkyl and C₁₋₆ haloalkyl, or two R^{a} groups are combined with the nitrogen to which they are attached to form an azetidinyl, pyrrolidinyl, or piperidinyl group.

In some embodiments, R₁₇ is —C₂₋₅ alkylene-C(═O)— wherein the alkylene is optionally substituted by a basic unit, e.g., —(CH₂)ₓNH₂, —(CH₂)ₓNHR³, and —(CH₂)ₓN(R^{a})₂, wherein x and R^{a} are as defined herein.

In some embodiments, R₁₇ is —C₂₋₅ alkylene-C(═O)— wherein the alkylene is substituted by a basic unit, e.g., —(CH₂)ₓNH₂, —(CH₂)ₓNHR^{a}, and —(CH₂)ₓN(R⁸)₂, wherein x and R are as defined herein.

In some embodiments, M^{P}, when present, is:

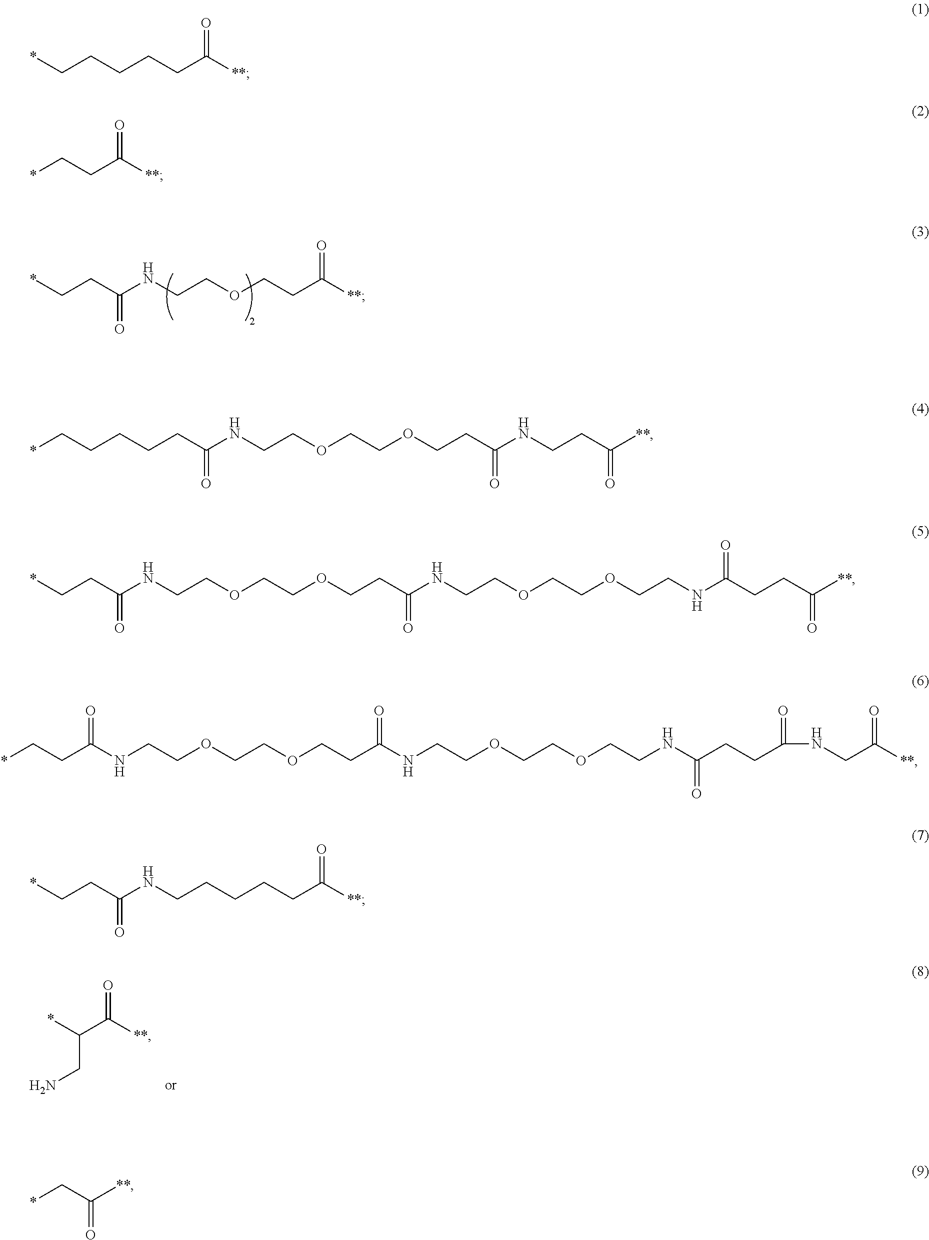

wherein * denotes attachment to L^{P′} or L^{P} and ** denotes attachment to L^{M}.

In some embodiments, M^{P}, when present, is:

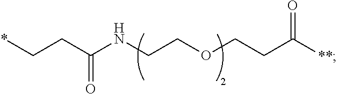

wherein * denotes attachment to L^{P′} or L^{P} and ** denotes attachment to L^{M}.

In some embodiments, M^{P}, when present, is:

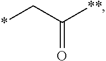

wherein * denotes attachment to L^{P′} or L^{P} and ** denotes attachment to L^{M}.

In some embodiments, M^{P}, when present, is:

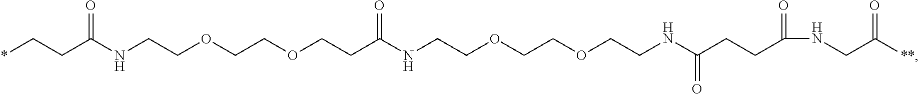

* denotes attachment to L^{P′} or L^{P} and ** denotes attachment to L^{M} or M^{A}.

In some embodiments, M^{P}, when present, is:

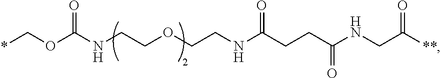

wherein * denotes attachment to L^{P′} or L^{P} and ** denotes attachment to L^{M} or M^{A}.

In some embodiments, M^{P}, when present, is:

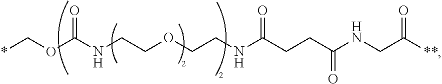

wherein * denotes attachment to L^{P′} or L^{P} and ** denotes attachment to L^{M} or M^{A}.

In some embodiments, M^{P}, when present, is:

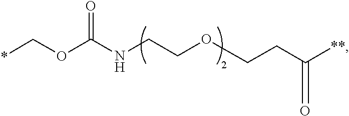

wherein * denotes attachment to L^{P′} or L^{P} and ** denotes attachment to L^{M} or M^{A}.

### L^{M}

In some embodiments, L^{M} is a bond or a multi-armed linker (e.g., trivalent or tetravalent or having 3 or 4 arms), wherein each arm maybe the same or different.

In some embodiments, L^{M} is a bond or a multi-armed linker (e.g., tetravalent or having 4 arms; or trivalent having 3 arms), wherein each arm maybe the same or different.

It is understood that the term “arm”, as used herein, refers to a portion of L^{M} which is (1) attached to M^{P} when present or attached to L^{P} or L^{P′} when M^{P} is absent, or (2) attached to L³ when present or attached to M^{A} when L³ is absent;

In some embodiments, a₂ is 2 and L^{M} is

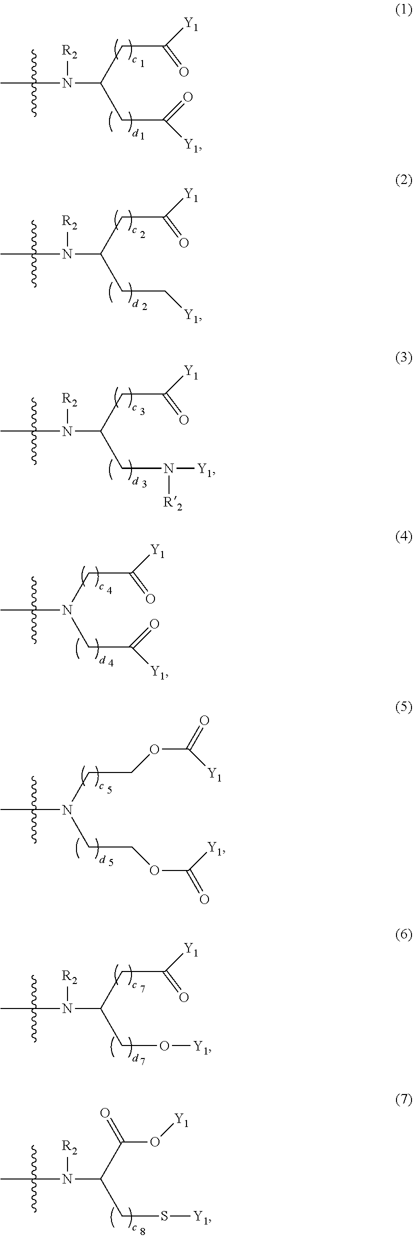

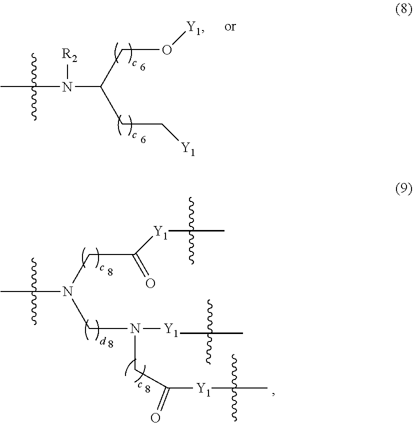

wherein

[symbol] denotes attachment to M^{P} when present or attachment to L^{P} or L^{P′} when M^{P} is absent;

Y₁ denotes attachment to L³ when present or attachment to M^{A} when L³ is absent;

R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxycarbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, COOH, or COO—C₁₋₆ alkyl;

each of c₁, c₂, c₃, c₄, c₅, c₇, and c₈ is an integer independently ranging between 0 and 10; and

each of d₁, d₂, d₃, d₄, d₅, and d₇ is an integer independently ranging between 0 and 10.

In some embodiments, a₂ is 2 and L^{M} is

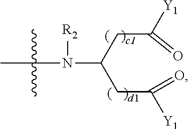

In some embodiments, a₂ is 2 and L^{M} is

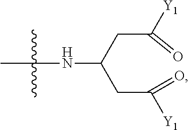

In some embodiments, c₁, c₂, c₃, c₄, c₅, c₇, and c₈ are each independently 0 or 1.

In some embodiments, c₁, c₂, c₃, c₄, c₅, c₇, and c₈ are each independently 1, 2, 3, 4, 5, 6, 7, 8, 9, or 10.

In some embodiments, c₁, c₂, c₃, c₄, c₅, c₇, and c₈ are each independently 0, 1, or 2.

In some embodiments, c₁, c₂, c₃, c₄, c₅, c₇, and c₈ are each independently 0. In some embodiments, c₁, c₂, c₃, c₄, c₅, c₇, and c₈ are each independently 1. In some embodiments, c₁, c₂, c₃, c₄, c₅, c₇, and c₈ are each independently 2.

In some embodiments, d₁, d₂, d₃, d₄, d₅, and d₇ are each independently 0 or 1.

In some embodiments, d₁, d₂, d₃, d₄, d₅, and d₇ are each independently 1, 2, 3, 4, 5, 6, 7, 8, 9, or 10.

In some embodiments, d₁, d₂, d₃, d₄, d₅, and d₇ are each independently 1, 2, 3 or 4.

In some embodiments, d₁, d₂, d₃, d₄, d₅, and d₇ are each independently 1. In some embodiments, d₁, d₂, d₃, d₄, d₅, and d₇ are each independently 2. In some embodiments, d₁, d₂, d₃, d₄, d₅, and d₇ are each independently 3. In some embodiments, d₁, d₂, d₃, d₄, d₅, and d₇ are each independently 4.

In some embodiments, R₂ and R′₂ are each independently hydrogen, C₁₋₆ alkyl, C₆₋₁₀ aryl, C₃₋₈ cycloalkyl, COOH, or COO—C₁₋₆ alkyl;

In some embodiments, R₂ and R′₂ are each independently hydrogen or C₁₋₆ alkyl.

In some embodiments, R₂ and R′₂ are each independently hydrogen.

In some embodiments, R₂ and R′₂ are each independently C₁₋₆ alkyl.

In some embodiments, L^{M} is:

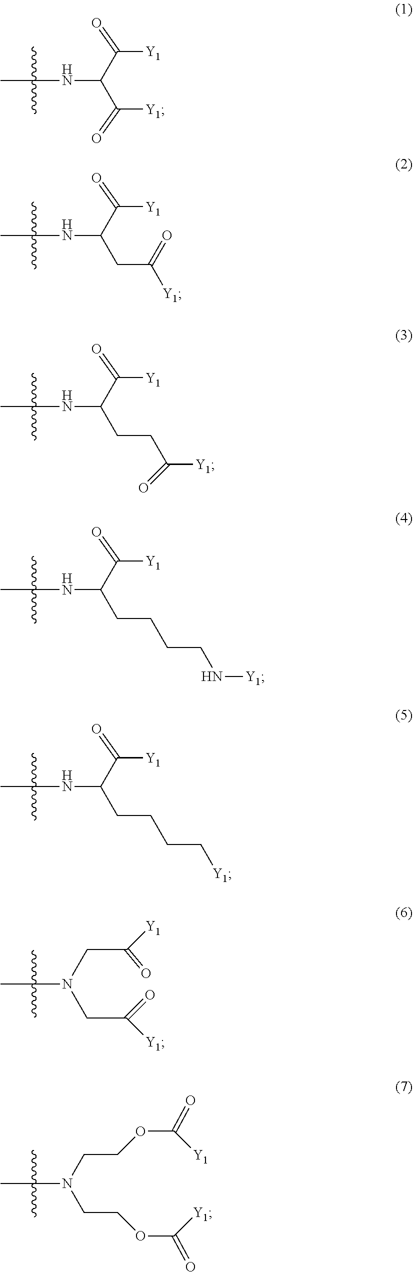

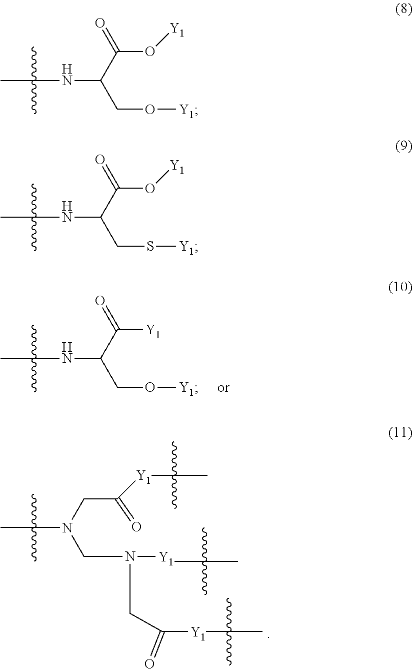

In some embodiments, a₂ is 3 and L^{M} is

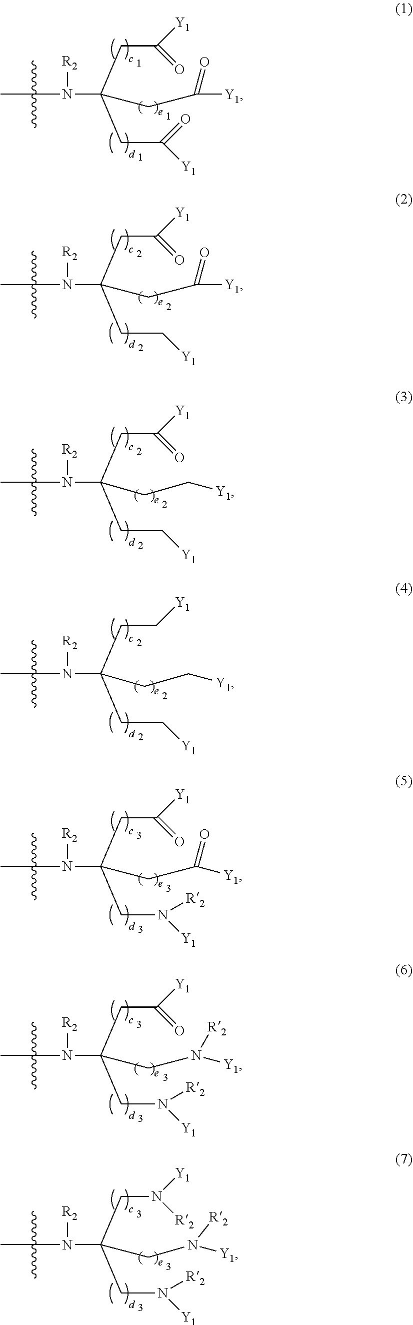

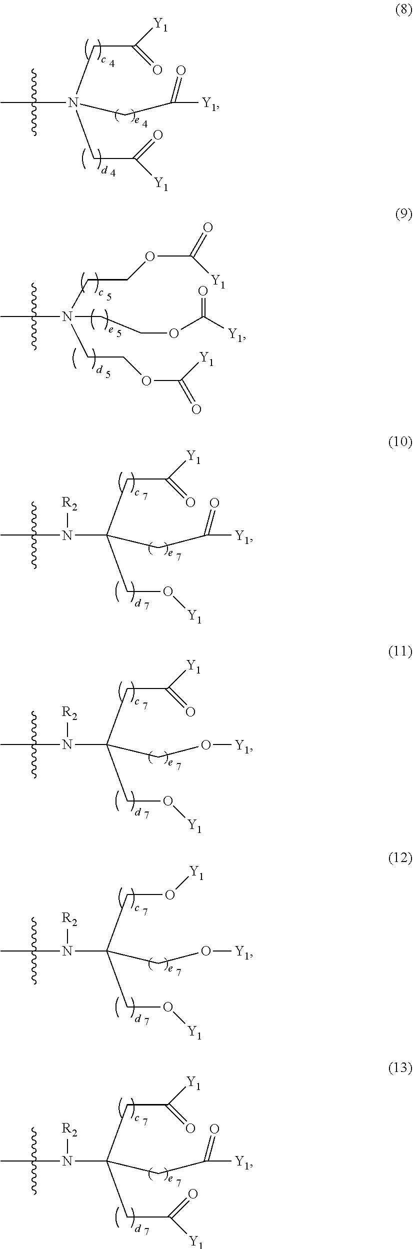

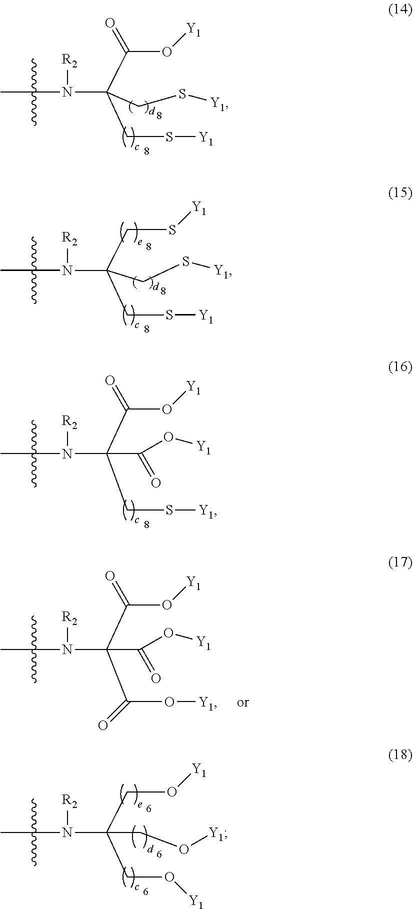

wherein:

[symbol] denotes attachment to M^{P} when present or attachment to L^{P} or L^{P′} when M^{P} is absent;

Y₁ denotes attachment to L³ when present or attachment to M^{A} when L³ is absent;

R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxycarbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, COOH, or COO—C₁₋₆ alkyl;

each of c₁, c₂, c₃, c₄, c₅, c₆, c₇, and c₈ is an integer independently ranging between 0 and 10;

each of d₁, d₂, d₃, d₄, d₅, d₆, d₇, and d₈ is an integer independently ranging between 0 and 10; and

each of e₁, e₂, e₃, e₄, e₅, e₆, e₇, and e₈ is an integer independently ranging between 0 and 10.

In some embodiments, a₂ is 3 and L^{M} is

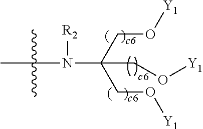

In some embodiments, a₂ is 3 and L^{M} is

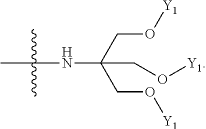

In some embodiments, -L^{M}-(L³)ₐ₂- is

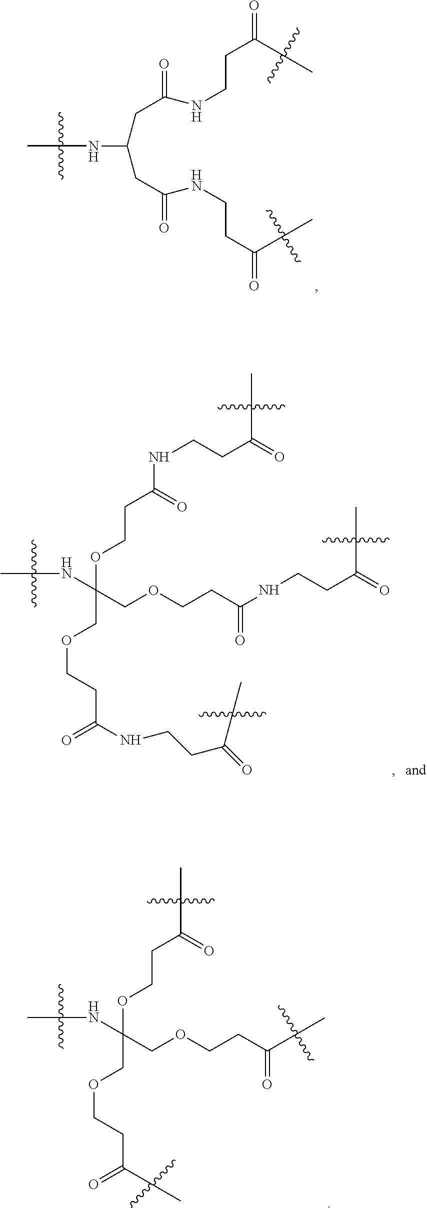 [SMILES: *NC(CC(=O)NCCC(*)=O)CC(=O)NCCC(*)=O ; *NC(COCCC(=O)NCCC(*)=O)(COCCC(=O)NCCC(*)=O)OCCC(=O)NCCC(*)=O ; *NC(COCCC(*)=O)(COCCC(*)=O)OCCC(*)=O]

In some embodiments, a₂ is 2 and L^{M} is selected from

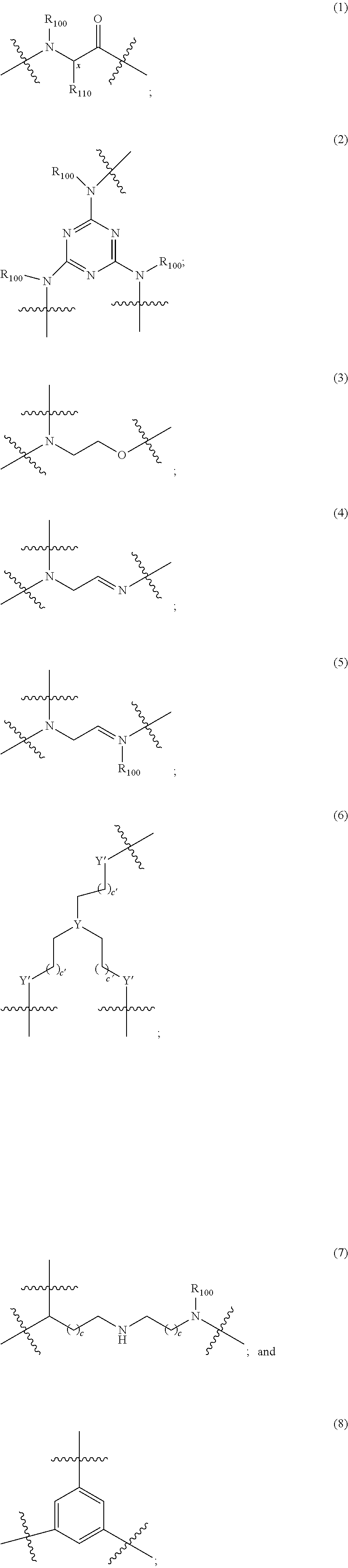

wherein

[symbol] indicated attachment sites within the conjugate of the disclosure or intermediates thereof;

R₁₁₀ is:

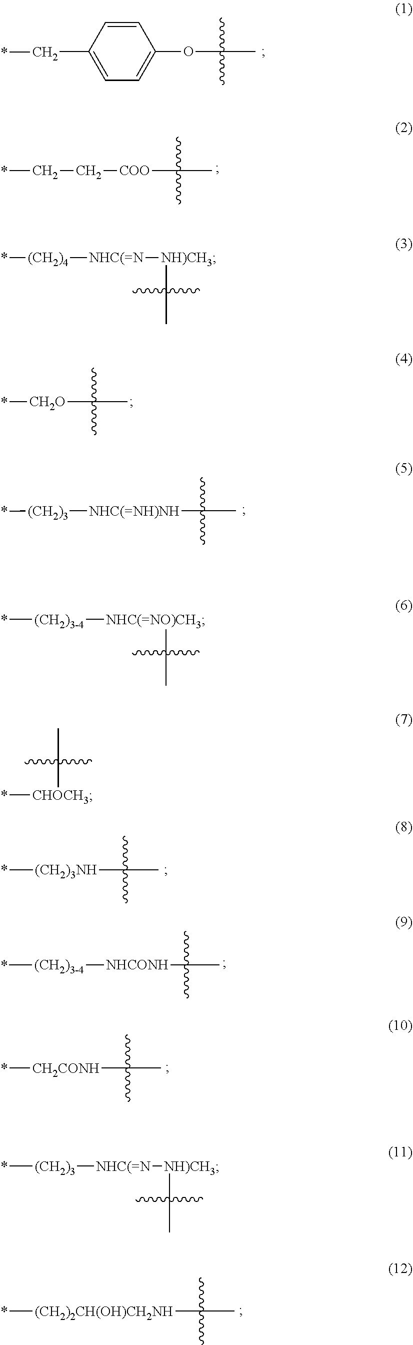

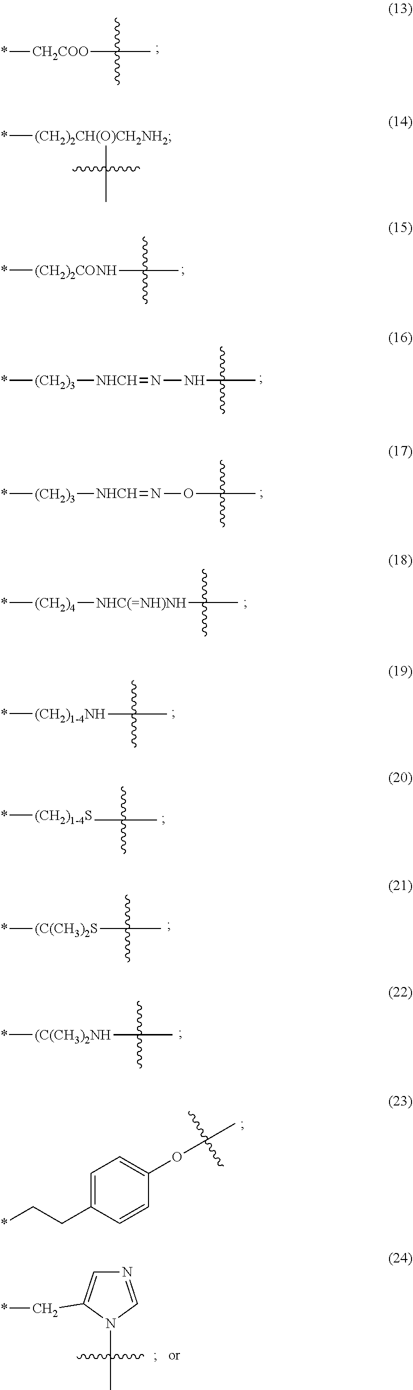

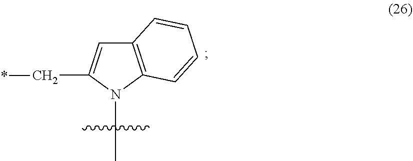 [SMILES: *Cc1cc2ccccc2n1*]

wherein the * indicates attachment to the carbon labeled x and the [symbol] indicates one of the three attachment sites;

R₁₀₀ is independently selected from hydrogen and —C₁₋₃ alkyl;

Y is N or CH;

each occurrence of Y′ is independently selected from NH, O, or S; and

each occurrence of c′ is independently an integer from 1 to 10.

In some embodiments, R₁₀₀ is independently selected from hydrogen and CH₃.

In some embodiments, R₁₀₀ is independently hydrogen.

In some embodiments, R₁₀₀ is independently CH₃.

In some embodiments, Y is N.

In some embodiments, Y is CH.

In some embodiments, R₁₀₀ is H or CH₃.

In some embodiments, R₁₀₀ is H. In some embodiments, R₁₀₀ is CH₃.

In some embodiments, each c′ is independently an integer from 1 to 3.

In some embodiments, R₁₁₀ is not

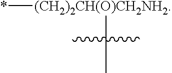

In some embodiments, wherein an AA unit has two attachment sites (i.e., a terminal drug unit) one of the attachment sites shown above can replaced, for example, by H, OH, or a C₁₋₃ unsubstituted alkyl group.

In some embodiments, when L^{M} is a multi-armed linker and not yet connected to the Stretcher unit M^{P}, W^{M} is a terminus of L^{M} and each occurrence of W^{M} is independently hydrogen, a protecting group, a leaving group, or a functional group that is capable of connecting L^{M} to M^{P} by forming a covalent bond. In some embodiments, W^{M} is an amine protecting group. In some embodiments, W^{M} is BOC.

In some embodiments, W^{M} is an amine protecting group, and L^{M} is

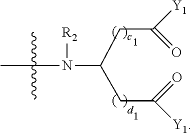

In some embodiments, W^{M} is BOC, and L^{M} is

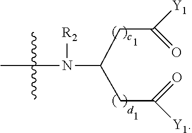

In some embodiments, W^{M} is an amine protecting group, and L^{M} is

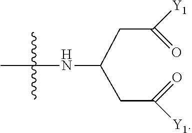

In some embodiments, W^{M} is BOC, and L^{M} is

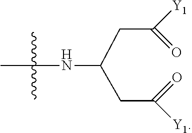

In some embodiments, W^{M} comprises an amine group in which w is an integer from 1 to 6.

In some embodiments, W^{M} comprises —C(O)—(CH₂)_{w}—NH₂, in which w is an integer from 1 to 6.

In some embodiments, W^{M} is —C(O)—CH₂—NH₂.

In some embodiments, W^{M} is —C(O)—CH₂—NH₂ and L^{M} is

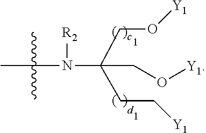

In some embodiments, W^{M} is —C(O)—CH₂—NH₂ and L^{M} is

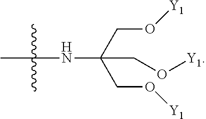

In some embodiments, W^{M} is hydrogen.

### L³

In some embodiments, each L³, when present, is a carbonyl-containing moiety.

In some embodiments, each L³, when present, independently is *—C₁₋₁₂ alkyl-C(O)—** or *—NH—C₁₋₁₂ alkyl-C(O)—**, wherein:

* indicates attachment to another L³ when present, or to L^{M}; and

** indicates attachment to another L³ when present, or to M^{A}.

In some embodiments, at least one L³ is *—C₁₋₁₂ alkyl-C(O)—**, wherein:

* indicates attachment to another L³ when present, or to L^{M}; and

** indicates attachment to another L³ when present, or to M^{A}.

In some embodiments, at least one L³ is *—CH₂CH₂—C(O)—**, wherein:

* indicates attachment to another L³ when present, or to L^{M}; and

** indicates attachment to another L³ when present, or to M^{A}.

In some embodiments, (L³)ₐ₃ is *—CH₂CH₂—C(O)—**, wherein:

* indicates attachment to L^{M}; and

** indicates attachment to M^{A}.

In some embodiments, at least one L³ is *—NH—C₁₋₁₂ alkyl-C(O)—**, wherein:

* indicates attachment to another L³ when present, or to L^{M}; and

** indicates attachment to another L³ when present, or to M^{A}.

In some embodiments, at least one L³ is *—NH—CH₂CH₂—C(O)—**, wherein:

* indicates attachment to another L³ when present, or to L^{M}; and

** indicates attachment to another L³ when present, or to M^{A}.

In some embodiments, at least one L³ is *—NH—CH₂CH₂—C(O)—**, wherein:

* indicates attachment to L^{M}; and

** indicates attachment to M^{A}.

In some embodiments, a₃ is 2 or greater, at least one L³ is *—C₁₋₁₂ alkyl-C(O)—**, and at least one L³ is *—NH—C₁₋₁₂ alkyl-C(O)—**.

In some embodiments, (L³)ₐ₃ is *—CH₂CH₂—C(O)—NH—CH₂CH₂—C(O)—**, wherein:

* indicates attachment to L^{M}; and

** indicates attachment to M^{A}.

In some embodiments, (L³)ₐ₃ is *NH—CH₂CH₂—C(O)—CH₂CH₂—C(O)—**, wherein:

* indicates attachment to L^{M}; and

** indicates attachment to M^{A}.

### M^{A}

In some embodiments, M^{A} is a linker moiety that is capable of connecting one or more drugs and one or more hydrophilic groups to L^{P} or L^{P′}. In some embodiments, M^{A} comprises a peptide moiety of at least two amino acids (AA's).

In some embodiments, the peptide moiety is a moiety that is capable of forming a covalent bond with a -L^{D}-D unit and allows for the attachment of multiple drugs. In some embodiments, peptide moiety comprises a single AA unit or has two or more AA units (e.g., from 2 to 10, from 2 to 6, or 2, 3, 4, 5 or 6) wherein the AA units are each independently a natural or non-natural amino acid, an amino alcohol, an amino aldehyde, a diamine, or a polyamine or combinations thereof. In some embodiments, in order to have the requisite number of attachments, at least one of AA units will have a functionalized side chain to provide for attachment of the -L^{D}-D unit. In some embodiments, exemplary functionalized AA units (e.g., amino acids, amino alcohols, or amino aldehydes) include, for example, azido or alkyne functionalized AA units (e.g., amino acid, amino alcohol, or amino aldehyde modified to have an azide group or alkyne group). In some embodiments, the azide group or alkyne group is for attachment using click chemistry.

In some embodiments, the peptide moiety has 2 to 12 AA units.

In some embodiments, the peptide moiety has 2 to 10 AA units.

In some embodiments, the peptide moiety has 2 to 6 AA units.

In yet some embodiments, the peptide moiety has 2, 3, 4, 5 or 6 AA units.

In yet some embodiments, the peptide moiety has 2 AA units. In yet some embodiments, the peptide moiety has 3 AA units. In yet some embodiments, the peptide moiety has 4 AA units. In yet some embodiments, the peptide moiety has 5 AA units. In yet some embodiments, the peptide moiety has 6 AA units.

In some embodiments, an AA unit has three attachment sites, (e.g., for attachment to L^{M}, the hydrophilic group or another AA unit, and to the -L^{D}-D unit). In some embodiments, the AA unit has the formula below:

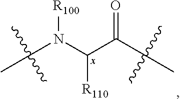

wherein [symbol] indicates attachment sites within the conjugate of the present disclosure or intermediates thereof; and R₁₀₀ and R₁₁₀ are as defined herein.

In some embodiments, an AA unit has two attachment sites (i.e., a terminal unit) and one of the attachment sites shown above can replaced, for example, by H, OH, or an unsubstituted C₁₋₃ alkyl group.

In some embodiments, the peptide moiety comprises at least two AA units of the following formula:

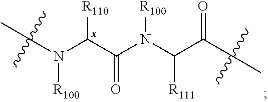

wherein:

each R₁₁₁ independently is H, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl,

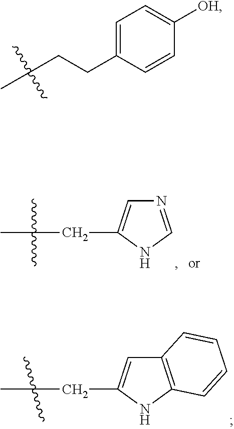 [SMILES: *CCc1ccc(O)cc1 ; *Cc1cnc[nH]1 ; *Cc1cc2ccccc2[nH]1]

the [symbol] indicates the attachment sites within the conjugate or intermediates thereof: and R₁₀₀ and R₁₁₀ are as defined herein.

In some embodiments, the peptide moiety comprises at least two AA units, e.g., cysteine-alanine as shown below:

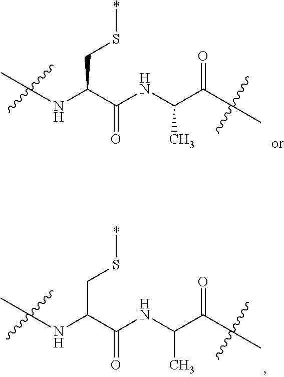 [SMILES: *N[C@@H](CS*)C(=O)N[C@@H](C)C(*)=O ; *NC(CS*)C(=O)NC(C)C(*)=O]

wherein the [symbol] and * indicate attachment sites within the conjugate or intermediates thereof. In some embodiments, * indicates attachment site of -L^{D}-D unit or a hydrophilic group. In some embodiments, the [symbol] next to the carbonyl group indicates attachment site of -L^{D}-D unit or a hydrophilic group. In some embodiments, the [symbol] next to the amine group indicates attachment site of -L^{D}-D unit or a hydrophilic group. In some embodiments, one or two of the [symbol] and * indicate attachment site(s) of one or more -L^{D}-D units or one or more hydrophilic groups.

In some embodiments, the peptide moiety comprises at least two AA units, which provide two attachment sites, e.g., cysteine-alanine as shown below:

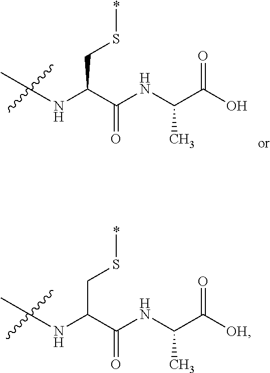 [SMILES: *N[C@@H](CS*)C(=O)N[C@@H](C)C(=O)O ; *NC(CS*)C(=O)N[C@@H](C)C(=O)O]

wherein the [symbol] and * indicate attachment sites within the conjugate or intermediates thereof. In some embodiments, * indicates attachment site of -L^{D}-D unit or a hydrophilic group. In some embodiments, the [symbol] indicates attachment site of -L^{D}-D unit or a hydrophilic group.

In some embodiments, one or more AA units (e.g., an amino acid, amino alcohol, amino aldehyde, or polyamine) of the peptide moiety can be replaced by an optionally substituted C₁₋₂₀ heteroalkylene (e.g., optionally substituted C₁₋₁₂ heteroalkylene), optionally substituted C₃₋₈ heterocycle, optionally substituted C₆₋₁₄ arylene, or optionally substituted C₃₋₈ carbocycle as described herein. In some embodiments, the optionally substituted heteroalkylene, heterocycle, arylene, or carbocycle may have one or more functional groups for attachment within a conjugate or intermediate thereof. In some embodiments, suitable substituents include, but are not limited to (═O), —R^{1C}, —R^{1B}, —OR^{1B}, —SR^{1B}, —N(R^{1B})₂, —N(R^{1B})₃, ═NR^{1B}, C(R^{1C})₃, CN, OCN, SCN, N═C═O, NCS, NO, NO₂, ═N₂, N₃, NR^{1B}C(═O)R^{1B}, —C(═O)R^{1B}, —C(═O)N(R^{1B})₂, SO₃—, SO₃H, S(═O)₂R^{1B}, —OS(═O)₂OR^{1B}, —S(═O)₂NR^{1B}, —S(═O)R^{1B}, —OP(═O)(OR^{1B})₂, —P(═O)(OR^{1B})₂, PO₃—, PO₃H₂, AsO₂H₂, C(═O)R^{1B}, C(═O)R^{1C}, C(═S)R^{1B}, CO₂R^{1B}, CO₂—, C(═S)ORB, C(═O)SR^{1B}, C(═S)SR^{1B}, C(═O)N(R^{1B})₂, C(═S)N(R^{1B})₂, and C(═NR^{1B})N(R^{1B})₂, where each R^{1C} is independently a halogen (e.g., —F, —Cl, —Br, or —I), and each R^{1B} is independently —H, C₁₋₂₀ alkyl, C₆₋₂₀ aryl, C₃₋₁₄ heterocycle, a protecting group, or a prodrug moiety.

In some embodiments, the one or more substituents for the heteroalkylene, heterocycle, arylene, or carbocycle are selected from (═O), R^{1C}, R^{1B}, OR^{1B}, SR^{1B}, and N(R^{1B})₂.

In some embodiments, the peptide moiety can be a straight chain or branched moiety. In some embodiments, the peptide moiety can be a straight chain or branched moiety having the Formula:

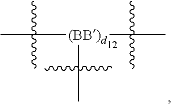

wherein:

each BB′ is independently an amino acid, optionally substituted C₁₋₂₀ heteroalkylene (e.g., optionally substituted C₁₋₁₂ heteroalkylene), optionally substituted C₃₋₈ heterocycle, optionally substituted C₆₋₁₄ arylene, or optionally substituted C₃-C₈ carbocycle;

d₁₂ is an integer from 1 to 10; and

the [symbol] indicates the covalent attachment sites within the conjugate or intermediate thereof.

In some embodiments, d₁₂ is an integer from 2 to 10.

In some embodiments, d₁₂ is an integer from 2 to 6.

In some embodiments, d₁₂ is an integer from 4, 5, or 6.

In some embodiments, d₁₂ is an integer from 5 or 6.

In some embodiments, d₁₂ is 4. In some embodiments, d₁₂ is 5. In some embodiments, d₁₂ is 6.

In some embodiments, the optionally substituted heteroalkylene, heterocycle, arylene, or carbocycle have functional groups for attachments between the BB′ subunits and/or for attachments within a conjugate or intermediates thereof disclosed herein.

In some embodiments, the peptide moiety comprises no more than 2 optionally substituted C₁₋₂₀ heteroalkylenes, optionally substituted C₃₋₁₈ heterocycles, optionally substituted C₆₋₁₄ arylenes, or optionally substituted C₃₋₈ carbocycles.

In some embodiments, the peptide moiety comprises 2 optionally substituted C₁₋₂₀ heteroalkylenes, optionally substituted C₃₋₁₈ heterocycles, optionally substituted C₆₋₁₄ arylenes, or optionally substituted C₃₋₈ carbocycles.

In other embodiments, the peptide moiety comprises no more than 1 optionally substituted C₁₋₂₀ heteroalkylene, optionally substituted C₃₋₁₈ heterocycle, optionally substituted C₆₋₁₄ arylene, or optionally substituted C₃₋₈ carbocycle.

In other embodiments, the peptide moiety comprises 1 optionally substituted C₁₋₂₀ heteroalkylene, optionally substituted C₃₋₁₈ heterocycle, optionally substituted C₆₋₁₄ arylene, or optionally substituted C₃₋₈ carbocycle.

In other embodiments, the optionally substituted heteroalkylene, heterocycle, arylene, or carbocyclo will have functional groups for attachment between the BB′ subunits and/or for attachments within a conjugate or intermediates thereof disclosed herein.

In some embodiments, at least one BB is an amino acid. In some embodiments, the amino acid can be an alpha, beta, or gamma amino acid, which can be natural or non-natural. The amino acid can be a D or L isomer.

In some embodiments, attachment within the peptide moiety or with the other components of the conjugate, intermediate thereof, or scaffold, can be, for example, via amino, carboxy, or other functionalities. In some embodiments, attachment within the peptide moiety or with the other components of the conjugate can be, for example, via amino, carboxy, or other functionalities. In some embodiments, each amino acid of the peptide moiety can be independently D or L isomer of a thiol containing amino acid. In some embodiments, each amino acid of the peptide moiety can be independently D isomer of a thiol containing amino acid. In some embodiments, each amino acid of the peptide moiety can be independently L isomer of a thiol containing amino acid. The thiol containing amino acid can be, for example, cysteine, homocysteine, or penicillamine.

In some embodiments, each amino acid that comprises the peptide moiety can be independently the L or D isomer of the following amino acids: alanine (including O-alanine), arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, citrulline, statine, diaminoalkanoic acid, stereoisomers thereof (e.g., isoaspartic acid and isoglutamic acid), or derivatives thereof.

In some embodiments, each amino acid that comprises the peptide moiety is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, alanine, or a stereoisomers thereof (e.g., isoaspartic acid and isoglutamic acid).

In some embodiments, the peptide moiety comprises a monopeptide, a dipeptide, tripeptide, tetrapeptide, or pentapeptide.

In some embodiments, the peptide moiety comprises at least about five amino acids (e.g., 5, 6, 7, 8, 9, or 10 amino acids).

In some embodiments, the peptide moiety comprises at most about ten amino acids.

In some embodiments, the peptide moiety comprises a pentapeptide.

In some embodiments, each amino acid that comprises the peptide moiety is independently glycine, serine, glutamic acid, lysine, aspartic acid, and cysteine.

In some embodiments, the peptide moiety comprises at least four glycines and at least one serine, e.g., (glycine)₄ and serine wherein the serine is at any position along the peptide chain, such as, for example, (serine)-(glycine)₄; (glycine)-(serine)-(glycine)₃; (glycine)₂-(serine)-(glycine)₂; (glycine)₃-(serine)-(glycine); or (glycine)₄-(serine).

In some embodiments, the peptide moiety comprises (glycine)₄-(serine) or (serine)-(glycine)₄. In some embodiments, the peptide moiety comprises (glycine)₄-(serine). In some embodiments, the peptide moiety comprises (serine)-(glycine)₄.

In some embodiments, the peptide moiety comprises at least four glycines and at least one glutamic acid e.g., (glycine)₄ and glutamic acid wherein the glutamic acid is at any position along the peptide chain, such as, for example, (glutamic acid)-(glycine)₄; (glycine)-(glutamic acid)-(glycine)₃; (glycine)₂-(glutamic acid)-(glycine)₂; (glycine)₃-(glutamic acid)-(glycine); or (glycine)₄-(glutamic acid).

In some embodiments, the peptide moiety comprises (glutamic acid)-(glycine)₄; or (glycine)₄-(glutamic acid).

In some embodiments, the peptide moiety comprises (β-alanine)-(glycine)₄-(serine) wherein the serine is at any position along the peptide chain, such as, for example, (β-alanine)-(serine)-(glycine)₄; (β-alanine)-(glycine)-(serine)-(glycine)₃; (β-alanine)-(glycine)₂-(serine)-(glycine)₂; (β-alanine)-(glycine)₃-(serine)-(glycine); or (β-alanine)-(glycine)₄-(serine).

In some embodiments, the peptide moiety comprises (glycine)₄-(serine)-(glutamic acid) wherein the serine is at any position along the peptide chain, such as, for example, (serine)-(glycine)₄-(glutamic acid); (glycine)-(serine)-(glycine)₃-(glutamic acid); (glycine)₂-(serine)-(glycine)₂-(glutamic acid); (glycine)₃-(serine)-(glycine)-(glutamic acid); or (glycine)₄-(serine)-(glutamic acid). In some embodiments, the peptide moiety comprises (β-alanine)-(glycine)₄-(serine)-(glutamic acid) wherein the serine is at any position along the peptide chain, such as, for example, (β-alanine)-(serine)-(glycine)₄-(glutamic acid); (β-alanine)-(glycine)-(serine)-(glycine)₃-(glutamic acid); (β-alanine)-(glycine)₂-(serine)-(glycine)₂-(glutamic acid); (β-alanine)-(glycine)₃-(serine)-(glycine)-(glutamic acid); or (β-alanine)-(glycine)₄-(serine)-(glutamic acid).

In some embodiments, the peptide moiety comprises (glycine)₁₋₄-(serine), wherein:

the peptide moiety is attached to L³ when present, or to L^{M} when L³ is absent, via one of the glycine;

the peptide moiety is attached to T¹ when present, via the serine; and

the peptide moiety is attached to L^{D} when present, via the serine.

In some embodiments, the peptide moiety comprises (glycine)₁₋₄-(serine), wherein:

the peptide moiety is attached to L³ when present, or to L^{M} when L³ is absent, via the serine;

the peptide moiety is attached to T¹ when present, via the glycine; and

the peptide moiety is attached to L^{D} when present, via the serine.

In some embodiments, the peptide moiety comprises

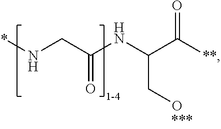

wherein:

* indicates attachment to L³ when present, or to L^{M} when L³ is absent:

** indicates attachment to T¹ when present, or —OH when T¹ is absent; and

*** indicates attachment to L^{D} when present, or —H when L^{D} is absent.

In some embodiments, the peptide moiety comprises (glycine)-(serine), wherein:

the peptide moiety is attached to L³ when present, or to L^{M} when L³ is absent, via the glycine;

the peptide moiety is attached to T¹ when present, via the serine; and

the peptide moiety is attached to L^{D} when present, via the serine.

In some embodiments, the peptide moiety comprises

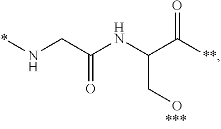

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (glycine)₄-(serine), wherein:

the peptide moiety is attached to L³ when present, or to L^{M} when L³ is absent, via one of the glycine;

the peptide moiety is attached to T¹ when present, via the serine; and

the peptide moiety is attached to L^{D} when present, via the serine.

In some embodiments, the peptide moiety comprises

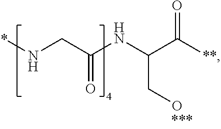

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (serine)-(glycine)₄, wherein:

the peptide moiety is attached to L³ when present, or to L^{M} when L³ is absent, via the serine;

the peptide moiety is attached to T¹ when present, via one of the glycine; and

the peptide moiety is attached to L^{D} when present, via the serine.

In some embodiments, the peptide moiety comprises

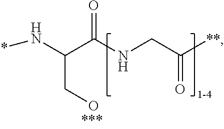

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent

In some embodiments, the peptide moiety comprises

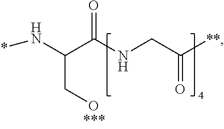

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (β-alanine)-(glycine)₁₋₄-(serine), wherein:

the peptide moiety is attached to L³ when present, or to L^{M} when L³ is absent, via the β-alanine;

the peptide moiety is attached to T¹ when present, via the serine; and

the peptide moiety is attached to L^{D} when present, via the serine.

In some embodiments, the peptide moiety comprises

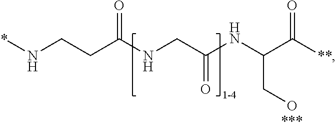

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (β-alanine)-(glycine)₄-(serine),

wherein:

the peptide moiety is attached to L³ when present, or to L^{M} when L³ is absent, via the β-alanine;

the peptide moiety is attached to T¹ when present, via the serine; and

the peptide moiety is attached to L^{D} when present, via the serine.

In some embodiments, the peptide moiety comprises

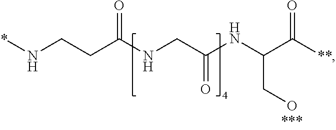

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (glycine)₁₋₄-(glutamic acid), wherein:

the peptide moiety is attached to L³ when present, or to L^{M} when L³ is absent, via one of the glycine;

the peptide moiety is attached to T¹ when present, via the glutamic acid; and

the peptide moiety is attached to L^{D} when present, via the glutamic acid.

In some embodiments, the peptide moiety comprises (glycine)₁₋₄-(glutamic acid, wherein:

the peptide moiety is attached to L³ when present, or to L^{M} when L³ is absent, via the glutamic acid;

the peptide moiety is attached to T¹ when present, via the glycine; and

the peptide moiety is attached to L^{D} when present, via the glutamic acid.

In some embodiments, the peptide moiety comprises

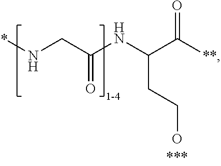

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (glycine)-(glutamic acid), wherein:

the peptide moiety is attached to L³ when present, or to L^{M} when L³ is absent, via the glycine;

the peptide moiety is attached to T¹ when present, via the glutamic acid; and

the peptide moiety is attached to L^{D} when present, via the glutamic acid.

In some embodiments, the peptide moiety comprises

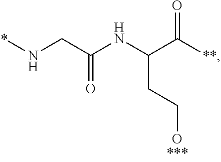

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (glycine)₄-(glutamic acid), wherein:

the peptide moiety is attached to L³ when present, or to L^{M} when L³ is absent, via one of the glycine;

the peptide moiety is attached to T¹ when present, via the glutamic acid; and

the peptide moiety is attached to L^{D} when present, via the glutamic acid.

In some embodiments, the peptide moiety comprises

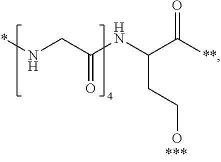

wherein:

* indicates attachment to L³ when present, or to L^{M} when L³ is absent;

** indicates attachment to T¹ when present, or —OH when T is absent; and

*** indicates attachment to L^{D} when present, or —H when L^{D} is absent

In some embodiments, the peptide moiety comprises (glutamic acid)-(glycine)₄, wherein:

the peptide moiety is attached to L³ when present, or to L^{M} when L³ is absent, via the glutamic acid;

the peptide moiety is attached to T¹ when present, via one of the glycine; and

the peptide moiety is attached to L^{D} when present, via the glutamic acid.

In some embodiments, the peptide moiety comprises

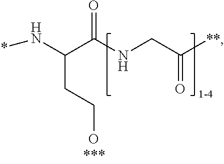

wherein:

* indicates attachment to L³ when present, or to L^{M} when L is absent;

** indicates attachment to T¹ when present, or —OH when T is absent; and

*** indicates attachment to L^{D} when present, or —H when L^{D} is absent.

In some embodiments, the peptide moiety comprises

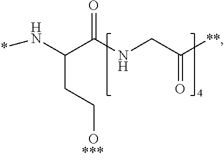

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (β-alanine)-(glycine)₁₋₄-(glutamic acid),

wherein:

the peptide moiety is attached to L³ when present, or to L^{M} when L³ is absent, via the β-alanine;

the peptide moiety is attached to T¹ when present, via the glutamic acid; and

the peptide moiety is attached to L^{D} when present, via the glutamic acid.

In some embodiments, the peptide moiety comprises

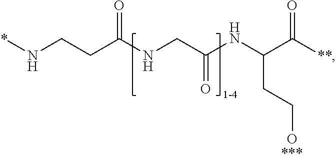

wherein:

* indicates attachment to L³ when present, or to L^{M} when L³ is absent;

** indicates attachment to T¹ when present, or —OH when T¹ is absent; and

*** indicates attachment to L^{D} when present, or —H when L^{D} is absent.

In some embodiments, the peptide moiety comprises (β-alanine)-(glycine)₄-(glutamic acid),

wherein:

the peptide moiety is attached to L³ when present, or to L^{M} when L³ is absent, via the β-alanine;

the peptide moiety is attached to T¹ when present, via the glutamic acid; and

the peptide moiety is attached to L^{D} when present, via the glutamic acid.

In some embodiments, the peptide moiety comprises

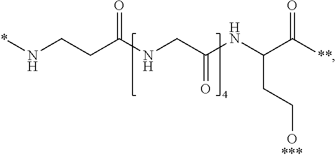

wherein:

* indicates attachment to L³ when present, or to L^{M} when L³ is absent;

** indicates attachment to T¹ when present, or —OH when T is absent; and

*** indicates attachment to L^{D} when present, or —H when L^{D} is absent.

In some embodiments, when at least one of the hydrophilic groups (or T¹) is a polyalcohol or derivative thereof (e.g., an amino polyalcohol), a glucosyl-amine, a di-glucosyl-amine, or a tri-glucosyl-amine, M^{A} does not have to comprise a peptide moiety, e.g., M^{A} comprising those multi-armed linkers as listed herein for L^{M}. In some embodiments, M^{A} comprises one or more of the following:

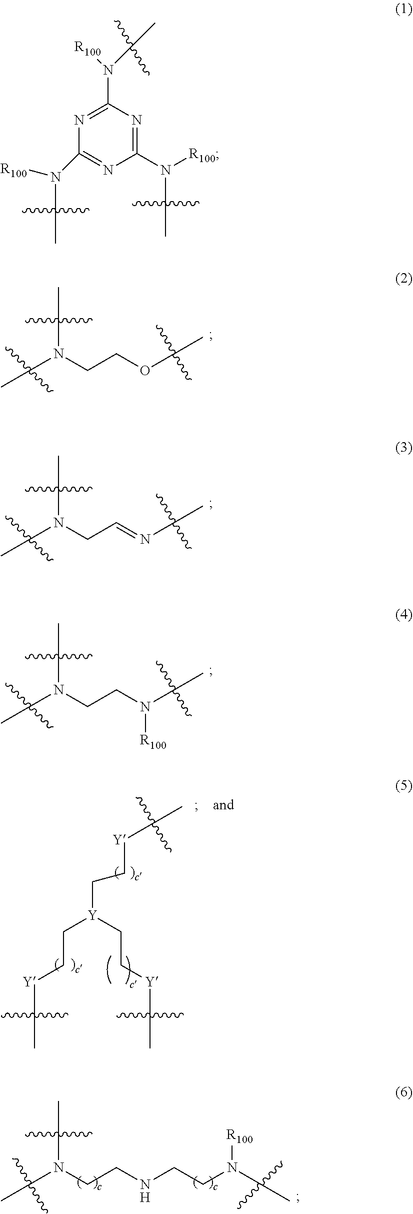

wherein

the [symbol] indicates attachment sites within the conjugate of the disclosure or intermediates thereof, and R₁₀₀ and R₁₁₀ are as defined herein.

In some embodiments, R₁₁₀ is:

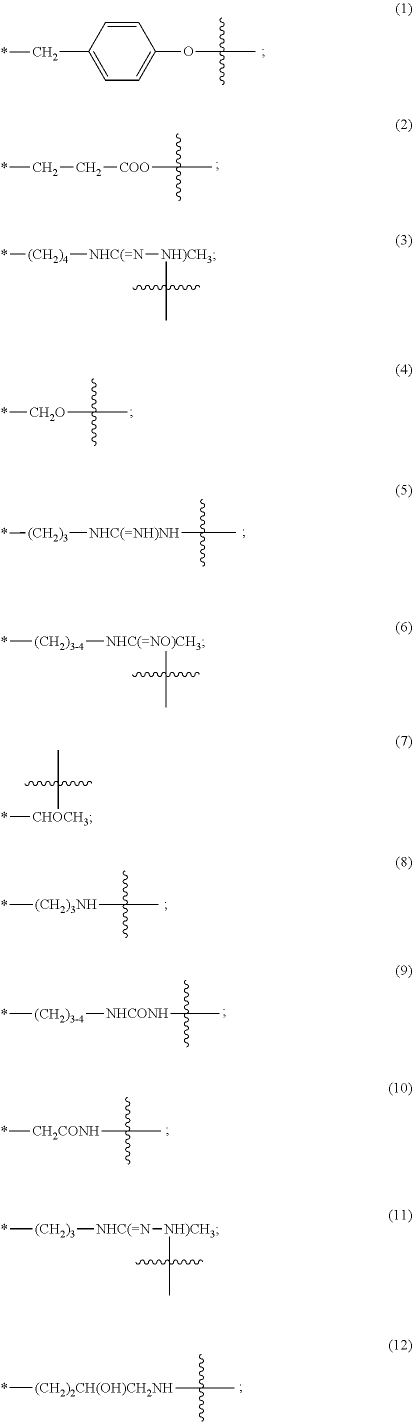

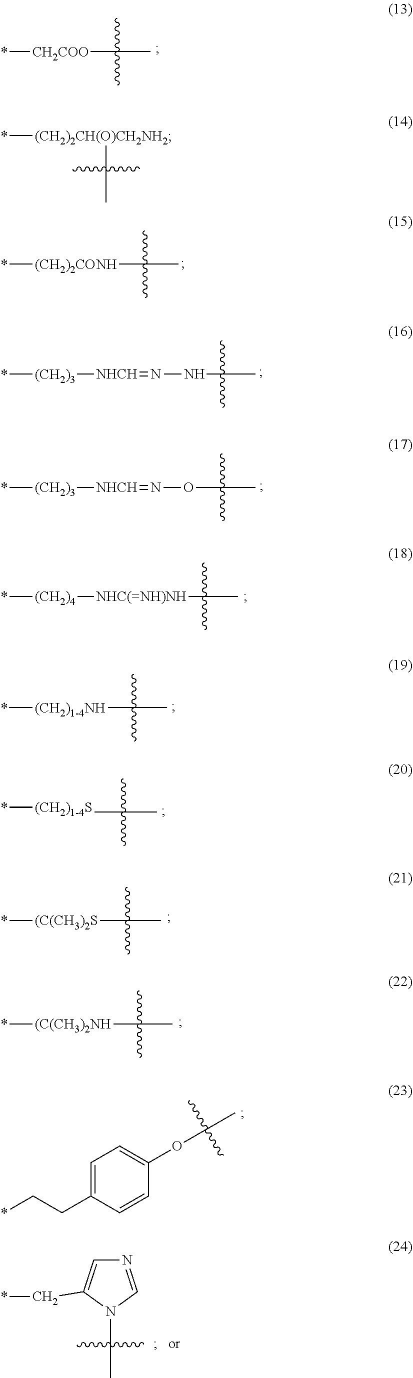

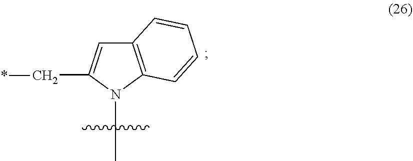 [SMILES: *Cc1cc2ccccc2n1*]

wherein the * indicates attachment to the carbon labeled x and the [symbol] indicates one of the three attachment sites.

In some embodiments, R₁₀₀ is independently selected from hydrogen and CH₃.

In some embodiments, R₁₀₀ is independently hydrogen. In some embodiments, R₁₀₀ is independently CH₃.

In some embodiments, Y is N. In some embodiments, Y is CH.

In some embodiments, R₁₀₀ is H or CH₃. In some embodiments, R₁₀₀ is H. In some embodiments, R₁₀₀ is CH₃.

In some embodiments, each c′ is independently an integer from 1 to 3.

In some embodiments, R₁₁₀ is not

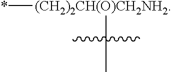

L^{D} and W^{D} In some embodiments, each occurrence of L^{D} is independently a divalent linker moiety connecting D to M^{A} and comprises at least one cleavable bond such that when the bond is cleaved, D is released in an active form for its intended therapeutic effect.

In some embodiments, L^{D} is a component of the Releasable Assembly Unit. In other embodiments, L^{D} is the Releasable Assembly Unit.

In some embodiments, L^{D} comprises one cleavable bond.

In some embodiments, L^{D} comprises multiple cleavage sites or bonds.

In some embodiments, functional groups for forming a cleavable bond can include, for example, sulfhydryl groups to form disulfide bonds, aldehyde, ketone, or hydrazine groups to form hydrazone bonds, hydroxylamine groups to form oxime bonds, carboxylic or amino groups to form peptide bonds, carboxylic or hydroxy groups to form ester bonds, and sugars to form glycosidic bonds. In some embodiments, L^{D} comprises a disulfide bond that is cleavable through disulfide exchange, an acid-labile bond that is cleavable at acidic pH, and/or bonds that are cleavable by hydrolases (e.g., peptidases, esterases, and glucuronidases). In some embodiments, L^{D} comprises a carbamate bond (i.e., —O—C(O)—NR—, in which R is H or alkyl or the like).

In some embodiments, the structure and sequence of the cleavable bond in L^{D} can be such that the bond is cleaved by the action of enzymes present at the target site. In other embodiments, the cleavable bond can be cleavable by other mechanisms.

In some embodiments, the structure and sequence of the cleavable bonds in L^{D} can be such that the bonds are cleaved by the action of enzymes present at the target site. In other embodiments, the cleavable bonds can be cleavable by other mechanisms.

In some embodiments, the cleavable bond(s) can be enzymatically cleaved by one or more enzymes, including a tumor-associated protease, to liberate the Drug unit or D, wherein the conjugate of the present disclosure, or intermediate, or scaffold thereof, is protonated in vivo upon release to provide a Drug unit or D.

In some embodiments, L^{D} can comprise one or more amino acids. In some embodiments, for example, each amino acid in L^{D} can be natural or unnatural and/or a D or L isomer, provided that there is a cleavable bond. In some embodiments, L^{D} comprises an alpha, beta, or gamma amino acid that can be natural or non-natural. In some embodiments, L^{D}comprises 1 to 12 (e.g., 1 to 6, or 1 to 4, or 1 to 3, or 1, 2, 3, 4, 5, 6, 7, 8, 9, 10, 11, or 12) amino acids in contiguous sequence.

In some embodiments, L^{D} can comprise only natural amino acids. In some embodiments, L^{D} can comprise only non-natural amino acids. In some embodiments, L^{D} can comprise a natural amino acid linked to a non-natural amino acid. In some embodiments, L^{D} can comprise a natural amino acid linked to a D-isomer of a natural amino acid. In some embodiments, L^{D} comprises a dipeptide such as -Val-Cit-, -Phe-Lys-, or -Val-Ala-.

In some embodiments, L^{D} comprises a monopeptide, a dipeptide, a tripeptide, a tetrapeptide, a pentapeptide, a hexapeptide, a heptapeptide, an octapeptide, a nonapeptide, a decapeptide, an undecapeptide, or a dodecapeptide unit.

In some embodiments, L^{D} comprises a peptide (e.g., of 1 to 12 amino acids), which is conjugated directly to the drug unit. In some such embodiments, the peptide is a single amino acid or a dipeptide. In some such embodiments, the peptide is a single amino acid. In some such embodiments, the peptide is a dipeptide.

In some embodiments, each amino acid in L^{D} is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, each amino acid is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, citrulline, and selenocysteine.

In some embodiments, each amino acid is independently selected from the group consisting of alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline, and derivatives thereof.

In some embodiments, each amino acid is selected from the proteinogenic or the non-proteinogenic amino acids.

In some embodiments, each amino acid in L^{D} can be independently selected from L or D isomers of the following amino acids: alanine, β-alanine, arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, citrulline, statine, diaminoalkanoic acid, valine, citrulline, and derivatives thereof.

In some embodiments, each amino acid in L^{D} is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, citrulline, or alanine.

In some embodiments, each amino acid in L^{D} is independently selected from L-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in L^{D} is independently selected from D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in L^{D} independently is L- or D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, or valine.

In some embodiments, each amino acid in L^{D} is alanine, β-alanine, glutamic acid, isoglutamic acid, isoaspartic acid, valine citrulline, or aspartic acid.

In some embodiments, L^{D} comprises β-alanine. In some embodiments, L^{D} comprises (β-alanine)-(alanine). In some embodiments, L^{D} comprises (β-alanine)-(glutamic acid). In some embodiments, L^{D} comprises (β-alanine)-(isoglutamic acid). In some embodiments, L^{D} comprises (β-alanine)-(aspartic acid). In some embodiments, L^{D} comprises (β-alanine)-(isoaspartic acid). In some embodiments, L^{D} comprises (β-alanine)-(valine). In some embodiments, L^{D} comprises (β-alanine)-(valine)-(alanine). In some embodiments, L^{D} comprises (β-alanine)-(alanine)-(alanine). In some embodiments, L^{D} comprises (β-alanine)-(valine)-(citrulline).

In some embodiments, L^{D} comprises a carbamate bond in addition to one or more amino acids.

In some embodiments, L^{D} can be designed and optimized in selectivity for enzymatic cleavage by a particular enzyme. In some embodiments, the particular enzyme is a tumor-associated protease.

In some embodiments, L^{D} comprises a bond whose cleavage is catalyzed by cathepsin B, C and D, or a plasmin protease.

In some embodiments, L^{D} comprises a sugar cleavage site. In some embodiments, L^{D} comprises a sugar moiety (Su) linked via an oxygen glycosidic bond to a self-immolative group. In some embodiments, a “self-immolative group” can be a tri-functional chemical moiety that is capable of covalently linking together three spaced chemical moieties (i.e., the sugar moiety (via a glycosidic bond), a drug unit (directly or indirectly), and M^{A} (directly or indirectly). In some embodiments, the glycosidic bond can be cleaved at the target site to initiate a self-immolative reaction sequence that leads to a release of the drug.

In some embodiments, L^{D} comprises a sugar moiety (Su) linked via a glycoside bond (—O′—) to a self-immolative group (K) of the formula:

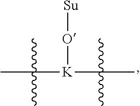

wherein the self-immolative group (K) forms a covalent bond with the drug unit (directly or indirectly) and also forms a covalent bond with M^{A} (directly or indirectly). In some embodiments, examples of self-immolative groups are described in WO 2015/057699, the contents of which are hereby incorporated by reference in its entirety.

In some embodiments, L^{D}, when not connected to or prior to connecting to a drug, comprises a functional group W^{D}. In some embodiments, each W^{D} independently can be a functional group as listed for W^{P}. In some embodiments, each W^{D} independently is

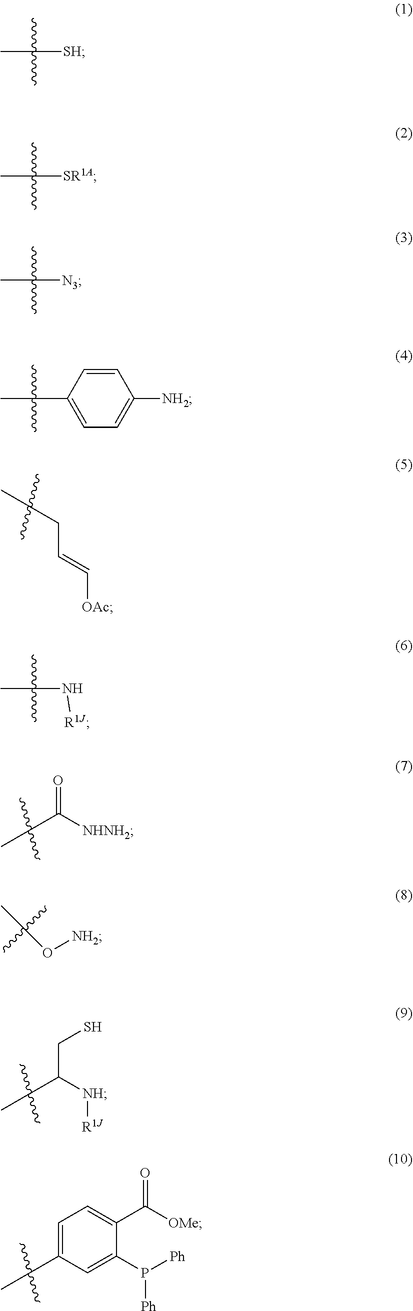

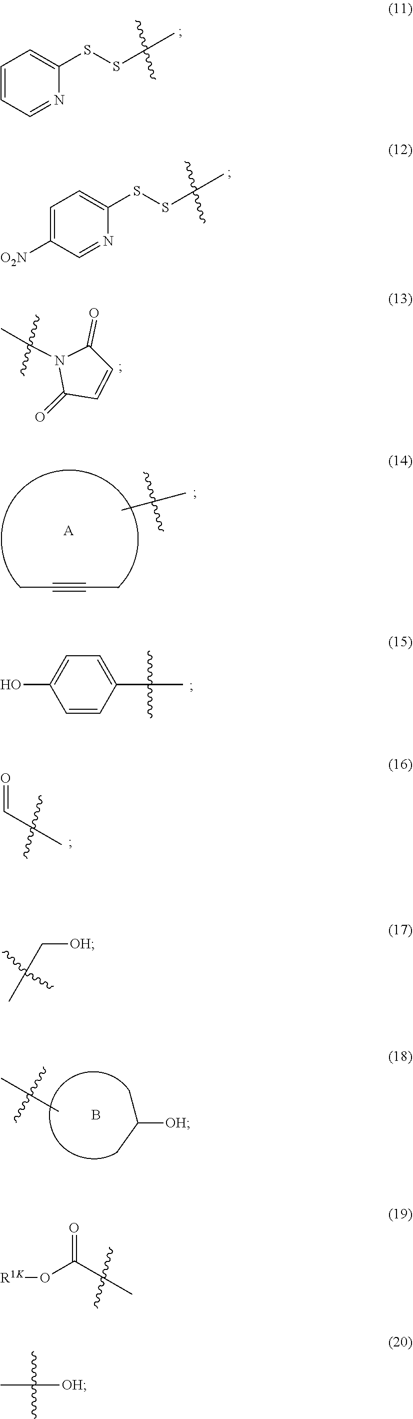

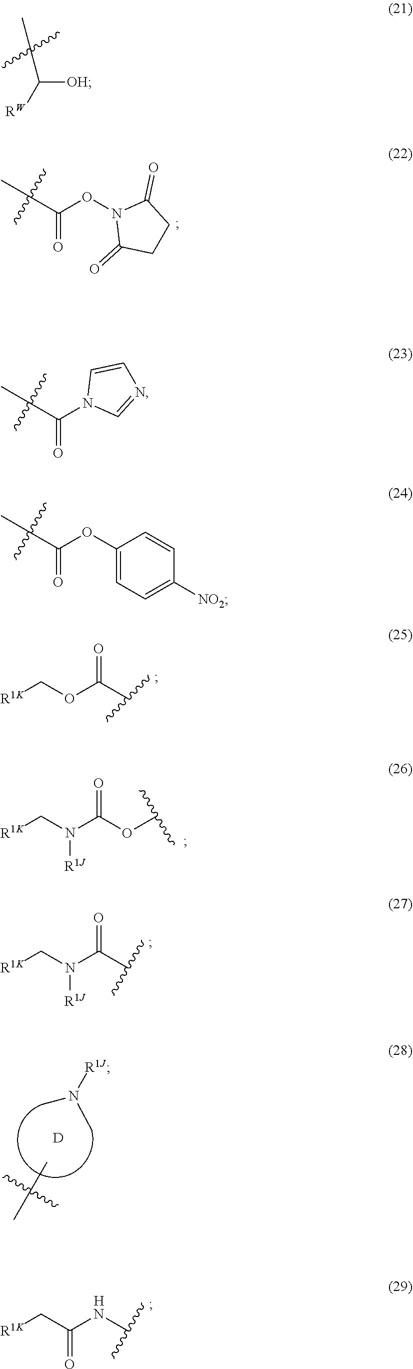

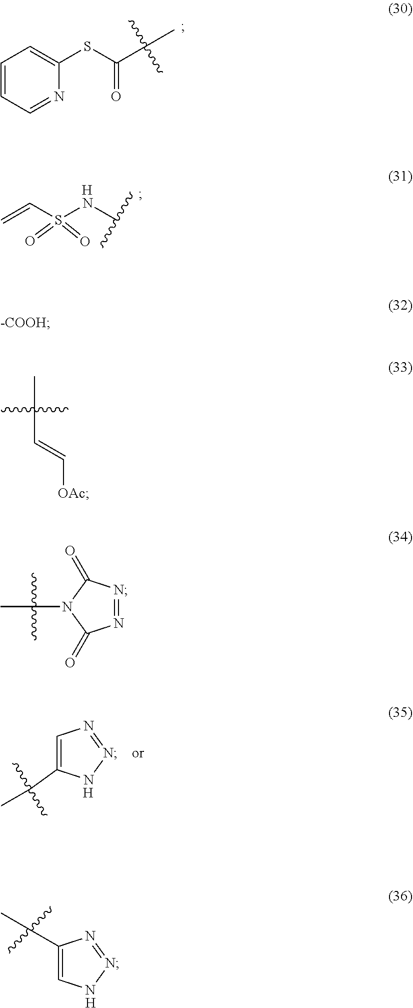

in which R^{1A} is a sulfur protecting group, each of ring A and B, independently, is cycloalkyl or heterocycloalkyl; R^{W} is an aliphatic, heteroaliphatic, carbocyclic, or heterocycloalkyl moiety; ring D is heterocycloalkyl; R^{1J} is hydrogen, an aliphatic, heteroaliphatic, carbocyclic, or heterocycloalkyl moiety; and R^{1K} is a leaving group (e.g., halide or RC(O)O— in which R is hydrogen, an aliphatic, heteroaliphatic, carbocyclic, or heterocycloalkyl moiety).

In some embodiments, W^{D} is

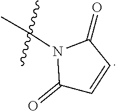 [SMILES: *N1C(=O)C=CC1=O]

In some embodiments, W^{D} is

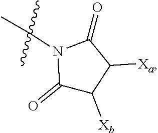

wherein one of Xₐ and X_{b} is H and the other is a maleimido blocking moiety.

In some embodiments, W^{D} is

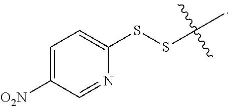 [SMILES: *SSc1ccc([N+](=O)[O-])cn1]

### Therapeutic Agents, Drug Unit, or D

In some embodiments, the therapeutic agent is a small molecule having a molecular weight ≤about 5 kDa. In some embodiments, the therapeutic agent is a small molecule having a molecular weight ≤about 4 kDa. In some embodiments, the therapeutic agent is a small molecule having a molecular weight ≤about 3 kDa. In some embodiments, the therapeutic agent is a small molecule having a molecular weight ≤about 1.5 kDa. In some embodiments, the therapeutic agent is a small molecule having a molecular weight ≤about 1 kDa.

In some embodiments, the therapeutic agent has an IC₅₀ of about less than 1 nM. In some embodiments, the therapeutic agent has an IC₅₀ of less than 1 nM.

In some embodiments, the therapeutic agent has an IC₅₀ of about greater than 1 nM, for example, the therapeutic agent has an IC₅₀ of about 1 to 50 nM.

In some embodiments, the therapeutic agent has an IC₅₀ of about greater than 1 nM. In some embodiments, the therapeutic agent has an IC₅₀ of about 1 to 50 nM.

In some embodiments, the therapeutic agent has an IC₅₀ of greater than 1 nM, for example, the therapeutic agent has an IC₅₀ of 1 to 50 nM.

In some embodiments, the therapeutic agent has an IC₅₀ of greater than 1 nM. In some embodiments, the therapeutic agent has an IC₅₀ of 1 to 50 nM.

In some embodiments, some therapeutic agents having an IC₅₀ of greater than about 1 nM (e.g., “less potent drugs”) are unsuitable for conjugation with an antibody using art-recognized conjugation techniques. Without wishing to be bound by theory, such therapeutic agents have a potency that is insufficient for use in targeted antibody-drug conjugates using conventional techniques as sufficient copies of the drug (i.e., more than 8) cannot be conjugated using art-recognized techniques without resulting in diminished pharmacokinetic and physiochemical properties of the conjugate. However sufficiently high loadings of these less potent drugs can be achieved using the conjugation strategies described herein thereby resulting in high loadings of the therapeutic agent while maintaining the desirable pharmacokinetic and physiochemical properties. Thus, the disclosure also relates to an antibody-drug conjugate which includes an antibody, a scaffold and at least eight therapeutic agent moieties, wherein the therapeutic agent has an IC₅₀ of greater than about 1 nM.

The small molecule therapeutic agents used in this disclosure (e.g., antiproliferative (cytotoxic and cytostatic) agents capable of being linked to a targeting moiety via the linker(s) of the disclosure) include cytotoxic compounds (e.g., broad spectrum), angiogenesis inhibitors, cell cycle progression inhibitors, PI3K/m-TOR/AKT pathway inhibitors, MAPK signaling pathway inhibitors, kinase inhibitors, protein chaperones inhibitors, HDAC inhibitors, PARP inhibitors, nicotinamide phosphoribosyl transferase (NAMPT) inhibitors, tubulysins, immunomodulatory compounds, Wnt/Hedgehog signaling pathway inhibitors and RNA polymerase inhibitors.

Broad spectrum cytotoxins include, but are not limited to, DNA-binding, intercalating or alkylating drugs, microtubule stabilizing and destabilizing agents, platinum compounds, topoisomerase I inhibitors, and protein synthesis inhibitors.

Exemplary DNA-binding, intercalation or alkylating drugs include, CC-1065 and its analogs, anthracyclines (doxorubicin, epirubicin, idarubicin, daunorubicin, nemorubicin and its derivatives, PNU-159682), bisnapththalimide compounds such as elinafide (LU79553). and its analogs, alkylating agents, such as calicheamicins, dactinomycins, mitomycins, pyrrolobenzodiazepines, and the like. Exemplary CC-1065 analogs include duocarmycin SA, duocarmycin A, duocarmycin C1, duocarmycin C2, duocarmycin B1, duocarmycin B2, duocarmycin D, DU-86, KW-2189, adozelesin, bizelesin, carzelesin, seco-adozelesin, and related analogs and prodrug forms, examples of which are described in U.S. Pat. Nos. 5,475,092; 5,595,499; 5,846,545; 6,534,660; 6,586,618; 6,756,397; and 7,049,316. Doxorubicin and its analogs include those described in U.S. Pat. No. 6,630,579. Calicheamicins include, e.g., enediynes, e.g., esperamicin, and those described in U.S. Pat. Nos. 5,714,586 and 5,739,116. Duocarmycins include those described in U.S. Pat. Nos. 5,070,092; 5,101,038; 5,187,186; 6,548,530; 6,660,742; and 7,553,816 B2; and Li et al., Tet Letts., 50:2932-2935 (2009).

Pyrrolobenzodiazepines (PBD) and analogs thereof include those described in Denny, Exp. Opin. Ther. Patents., 10(4):459-474 (2000) and Antonow and Thurston, Chem Rev., 2815-2864 (2010).

Exemplary microtubule stabilizing and destabilizing agents include taxane compounds, such as paclitaxel, docetaxel, tesetaxel and carbazitaxel; maytansinoids, auristatins and analogs thereof, vinca alkaloid derivatives, epothilones, and cryptophycins.

Exemplary maytansinoids or maytansinoid analogs include maytansinol and maytansinol analogs, maytansine or DM-1 and DM-4 are those described in U.S. Pat. Nos. 5,208,020; 5,416,064; 6,333.410; 6,441,163; 6,716,821; RE39,151; and 7,276,497. In some embodiments, the cytotoxic agent is a maytansinoid, another group of anti-tubulin agents (ImmunoGen, Inc.; see also Chari et al., 1992, Cancer Res. 52:127-131), maytansinoids or maytansinoid analogs. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F, auristatin F phenylenediamine (AFP), auristatin F hydroxylpropylamide (AF-HPA), monomethyl auristatin F hydroxylpropylamide (MMAF-HPA), and dolastatin. Suitable auristatins are also described in U.S. Publication Nos. 2003/0083263, 2011/0020343, and 2011/0070248; PCT Application Publication Nos. WO 09/117531, WO 2005/081711, WO 04/010957; WO 02/088172; and WO 01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of which are incorporated herein by reference in their entirety.

Exemplary vinca alkaloids include vincristine, vinblastine, vindesine, and navelbine (vinorelbine). Suitable Vinca alkaloids that can be used in the present disclosure are also disclosed in U.S. Publication Nos. 2002/0103136 and 2010/0305149, and in U.S. Pat. No. 7,303,749 B1, the disclosures of which are incorporated herein by reference in their entirety.

Exemplary epothilone compounds include epothilone A, B, C, D, E and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO 97/19086; WO 98/08849; WO 98/22461; WO 98/25929; WO 98/38192: WO 99/01124; WO 99/02514; WO 99/03848; WO 99/07692; WO 99/27890; and WO 99/28324; the disclosures of which are incorporated herein by reference in their entirety.

Exemplary cryptophycin compounds are described in U.S. Pat. Nos. 6,680,311 and 6,747,021.

Exemplary platinum compounds include cisplatin (PLATINOL®), carboplatin (PARAPLATIN®), oxaliplatin (ELOXATINE®), iproplatin, ormaplatin, and tetraplatin.

Still other classes of compounds or compounds with these or other cytotoxic modes of action may be selected, including, e.g., mitomycin C, mitomycin A, daunorubicin, doxorubicin, morpholino-doxorubicin, cyanomorpholino-doxorubicin, aminopterin, bleomycin, 1-(chloromethyl)-2,3-dihydro-1H-benzo[e]indol-5-ol, pyrrolobenzodiazepine (PBD) polyamide and dimers thereof. Other suitable cytotoxic agents include, for example, puromycins, topotecan, rhizoxin, echinomycin, combretastatin, netropsin, estramustine, cryptophysins, cemadotin, discodermolide, eleutherobin, and mitoxantrone.

Exemplary topoisomerase I inhibitors include camptothecin, camptothecin derivatives, camptothecin analogs and non-natural camptothecins, such as, for example, CPT-11 (irinotecan), SN-38, GI-147211C, topotecan, 9-aminocamptothecin, 7-hydroxymethyl camptothecin, 7-aminomethyl camptothecin, 10-hydroxycamptothecin, (20S)-camptothecin, rubitecan, gimatecan, karenitecin, silatecan, lurtotecan, exatecan, diflomotecan, belotecan, lurtotecan and S39625. Other camptothecin compounds that can be used in the present disclosure include those described in, for example, J. Med. Chem., 29:2358-2363 (1986); J. Med. Chem., 23:554 (1980); J. Med. Chem., 30:1774 (1987).

Angiogenesis inhibitors include, but are not limited, MetAP2 inhibitors, VEGF inhibitors, PIGF inhibitors, VEGFR inhibitors, PDGFR inhibitors, MetAP2 inhibitors. Exemplary VEGFR and PDGFR inhibitors include sorafenib (Nexavar), sunitinib (Sutent) and vatalanib. Exemplary MetAP2 inhibitors include fumagillol analogs, meaning any compound that includes the fumagillin core structure, including fumagillamine, that inhibits the ability of MetAP-2 to remove NH₂-terminal methionines from proteins as described in Rodeschini et al., J. Org. Chem., 69, 357-373, 2004 and Liu, et al., Science 282, 1324-1327, 1998. Non limiting examples of “fumagillol analogs” are disclosed in J. Org. Chem., 69, 357, 2004; J. Org. Chem., 70, 6870, 2005; European Patent Application 0 354 787; J. Med. Chem., 49, 5645, 2006; Bioorg. Med. Chem., 11, 5051, 2003; Bioorg. Med. Chem., 14, 91, 2004; Tet. Lett. 40, 4797, 1999; WO99/61432; U.S. Pat. Nos. 6,603,812; 5,789,405; 5,767,293; 6,566,541; and 6,207,704.

Exemplary cell cycle progression inhibitors include CDK inhibitors such as, for example, BMS-387032 and PD0332991; Rho-kinase inhibitors such as, for example GSK429286; checkpoint kinase inhibitors such as, for example, AZD7762; aurora kinase inhibitors such as, for example, AZD1152, MLN8054 and MLN8237; PLK inhibitors such as, for example, BI 2536, B16727 (Volasertib), GSK461364, ON-01910 (Estybon); and KSP inhibitors such as, for example, SB 743921, SB 715992 (ispinesib), MK-0731, AZD8477, AZ3146, and ARRY-520.

Exemplary PI3K/m-TOR/AKT signaling pathway inhibitors include phosphoinositide 3-kinase (PI3K) inhibitors, GSK-3 inhibitors, ATM inhibitors, DNA-PK inhibitors, and PDK-1 inhibitors.

Exemplary PI3 kinase inhibitors are disclosed in U.S. Pat. No. 6,608,053, and include BEZ235, BGT226, BKM120, CAL101, CAL263, demethoxyviridin, GDC-0941, GSK615, IC87114, LY294002, Palomid 529, perifosine, PI-103, PF-04691502, PX-866, SAR245408, SAR245409, SF1126, Wortmannin, XL147, and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,994 and include GDC-0973, GSK1120212, MSC1936369B, AS703026, RO5126766 and RO4987655, PD0325901, AZD6244, AZD 8330, and GDC-0973.

Exemplary B-raf inhibitors include CDC-0879, PLX-4032, and SB590885.

Exemplary B p38 MAPK inhibitors include BIRB 796, LY2228820, and SB 202190.

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR, and RET receptor family receptors. Exemplary specific RTK targets include ErbB2, FLT-3, c-Kit, and c-Met.

Exemplary inhibitors of ErbB2 receptor (EGFR family) include but not limited to AEE788 (NVP-AEE 788), BIBW2992, (Afatinib), Lapatinib, Erlotinib (Tarceva), and Gefitinib (Iressa).

Exemplary RTK inhibitors targeting more than one signaling pathway (multitargeted kinase inhibitors) include AP24534 (Ponatinib) that targets FGFR, FLT-3, VEGFR-PDGFR and Bcr-Abl receptors; ABT-869 (Linifanib) that targets FLT-3 and VEGFR-PDGFR receptors; AZD2171 that targets VEGFR-PDGFR, Flt-1 and VEGF receptors; CHR-258 (Dovitinib) that targets VEGFR-PDGFR, FGFR, Flt-3, and c-Kit receptors; Sunitinib (Sutent) that targets VEGFR, PDGFR, KIT, FLT-3 and CSF-IR; Sorafenib (Nexavar) and Vatalanib that target VEGFR, PDGFR as well as intracellular serine/threonine kinases in the Raf/Mek/Erk pathway.

Exemplary protein chaperon inhibitors include HSP90 inhibitors. Exemplary HSP90 inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MCI568, MGCD0103 (Mocetinostat), MS-275 (Entinostat), PCI-24781, Pyroxamide (NSC 696085), SB939, Trichostatin A, and Vorinostat (SAHA).

Exemplary PARP inhibitors include iniparib (BSI 201), olaparib (AZD-2281), ABT-888 (Veliparib), AG014699, CEP 9722, MK 4827, KU-0059436 (AZD2281), LT-673, 3-aminobenzamide, A-966492, and AZD2461.

Exemplary NAMPT inhibitors include FK866 (AP0866) and CHS828, GPP78, GMX1778 (CHS828), STF-118804, STF-31, CB 300919, CB 30865, GNE-617, IS001, TP201565, Nampt-IN-1, P7C3, MPC-9528, CB30865, MPI0479883, and (E)-N-(5-((4-(((2-(1H-Indol-3-yl)ethyl)(isopropyl)amino)methyl)phenyl)amino)pentyl)-3-(pyridin-3-yl)acrylamide.

Exemplary Wnt/Hedgehog signaling pathway inhibitors include vismodegib (RG3616/GDC-0449), cyclopamine (11-deoxojervine) (Hedgehog pathway inhibitors), and XAV-939 (Wnt pathway inhibitor).

Exemplary RNA polymerase inhibitors include amatoxins. Exemplary amatoxins include α-amanitins, β-amanitins, γ-amanitins, ε-amanitins, amanullin, amanullic acid, amaninamide, amanin, and proamanullin.

Exemplary protein synthesis inhibitors include trichothecene compounds.

In some embodiments, the drug is a topoisomerase inhibitor (such as, for example, a non-natural camptothecin compound), vinca alkaloid, kinase inhibitor (e.g., PI3 kinase inhibitor (GDC-0941 and PI-103)), MEK inhibitor, KSP inhibitor, RNA polymerase inhibitor, protein synthesis inhibitor, PARP inhibitor, NAMPT inhibitor, tubulysins, immunomodulatory compound, docetaxel, paclitaxel, doxorubicin, duocarmycin, auristatin, dolastatin, calicheamicins, topotecan, SN38, camptothecin, exatecan, nemorubicin and its derivatives, PNU-159682, CC1065, elinafide, trichothecene, pyrrolobenzodiazepines, maytansinoids, DNA-binding drugs or a platinum compound, and analogs thereof. In specific embodiments, the drug is a derivative of SN-38, camptothecin, topotecan, exatecan, calicheamicin, nemorubicin, PNU-159682, anthracycline, maytansinoid, taxane, trichothecene, CC1065, elinafide, vindesine, vinblastine, PI-103, AZD 8330, dolastatin, auristatin E, auristatin F, a duocarmycin compound, ispinesib, pyrrolobenzodiazepine, ARRY-520 and stereoisomers, isosteres and analogs thereof.

In some embodiments, the drug is a derivative of (a) an auristatin compound; (b) a calicheamicin compound; (c) a duocarmycin compound; (d) SN38, (e) a pyrrolobenzodiazepine; (f) a vinca compound; (g) a tubulysin compound; (h) a non-natural camptothecin compound; (i) a maytansinoid compound; (j) a DNA binding drug; (k) a kinase inhibitor; (l) a MEK inhibitor; (m) a KSP inhibitor; (n) a topoisomerase inhibitor; (o) a DNA-alkylating drug; (p) a RNA polymerase; (q) a PARP inhibitor; (r) a NAMPT inhibitor; (s) a topoisomerase inhibitor; (t) a protein synthesis inhibitor; (u) a DNA-binding drug: (v) a DNA intercalation drug; or (w) an immunomodulatory compound.

In some embodiments, the drug is a derivative of an auristatin compound. In some embodiments, the drug is a derivative of a calicheamicin compound. In some embodiments, the drug is a derivative of a duocarmycin compound. In some embodiments, the drug is a derivative of SN38. In some embodiments, the drug is a derivative of a pyrrolobenzodiazepine. In some embodiments, the drug is a derivative of a vinca compound. In some embodiments, the drug is a derivative of a tubulysin compound. In some embodiments, the drug is a derivative of a non-natural camptothecin compound. In some embodiments, the drug is a derivative of a maytansinoid compound. In some embodiments, the drug is a derivative of a DNA binding drug. In some embodiments, the drug is a derivative of a kinase inhibitor. In some embodiments, the drug is a derivative of a MEK inhibitor. In some embodiments, the drug is a derivative of a KSP inhibitor. In some embodiments, the drug is a derivative of a topoisomerase inhibitor. In some embodiments, the drug is a derivative of a DNA-alkylating drug. In some embodiments, the drug is a derivative of a RNA polymerase. In some embodiments, the drug is a derivative of a PARP inhibitor. In some embodiments, the drug is a derivative of a NAMPT inhibitor. In some embodiments, the drug is a derivative of a topoisomerase inhibitor. In some embodiments, the drug is a derivative of a protein synthesis inhibitor. In some embodiments, the drug is a derivative of a DNA-binding drug. In some embodiments, the drug is a derivative of a DNA intercalation drug. In some embodiments, the drug is a derivative of an immunomodulatory compound.

In some embodiments, the drug used in the disclosure is a combination of two or more drugs, such as, for example, PI3 kinase inhibitors and MEK inhibitors; broad spectrum cytotoxic compounds and platinum compounds; PARP inhibitors, NAMPT inhibitors and platinum compounds; broad spectrum cytotoxic compounds and PARP inhibitors.

In yet some embodiments, the drug used in the disclosure is auristatin F-hydroxypropylamide-L-alanine.

In some embodiments, the Vinca alkaloid is a compound of Formula (V1),

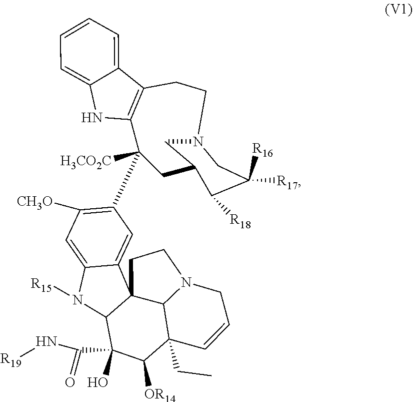

wherein:

R₁₄ is hydrogen, —C(O)—C₁₋₃ alkyl, or —C(O)-chloro substituted C₁₋₃ alkyl;

R₁₅ is hydrogen, —CH₃, or —CHO;

when R₁₇ and R₁₈ are taken independently, R₁₈ is hydrogen, and either R₁₆ or R₁₇ is ethyl and the other is hydroxyl;

when R₁₇ and R₁₈ are taken together with the carbon to which they are attached to form an oxiran ring, R₁₆ is ethyl;

R₁₉ is —H, OH, amino group, C₁₋₈ alkyl amino, or —[C(R₂₀R₂₁)]ₐ—R₂₂;

each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)ᵣ—N(H)(R₂₃), or —R₈₂—(C(O)—CH(X²)—NH)_{d}—R₇₇;

each R₂₃ independently is hydrogen, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is hydrogen or X² and NR₇₇ form a nitrogen containing heterocyclic moiety;

R₈₂ is —NR₂₃ or oxygen;

a is an integer from 1 to 6;

c is an integer from 0 to 3;

d is an integer from 1 to 3; and

f is an integer from 1 to 12.

Further examples of Vinca alkaloids are described in U.S. Pat. No. 8,524,214B2 and US 2002/0103136.

In some embodiments the Vinca alkaloid of Formula (V1) is a compound of Formula (VI1):

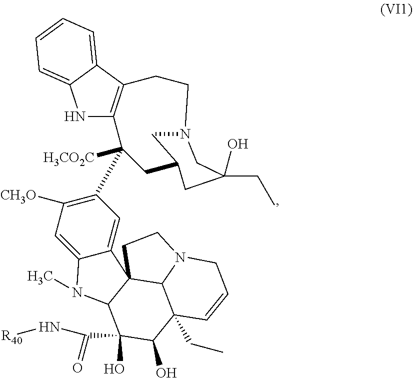

wherein:

R₄₀ is hydrogen, —OH, —NH₂, or an of the following structures:

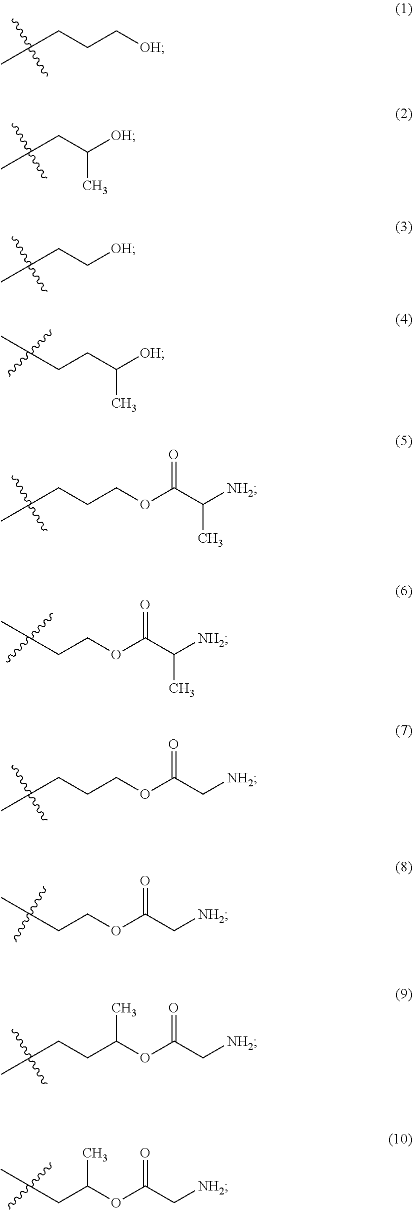

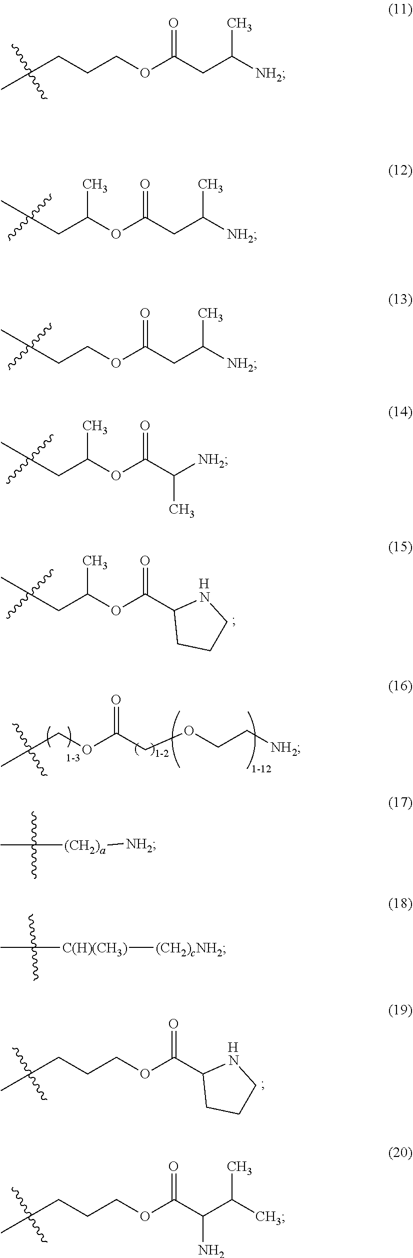

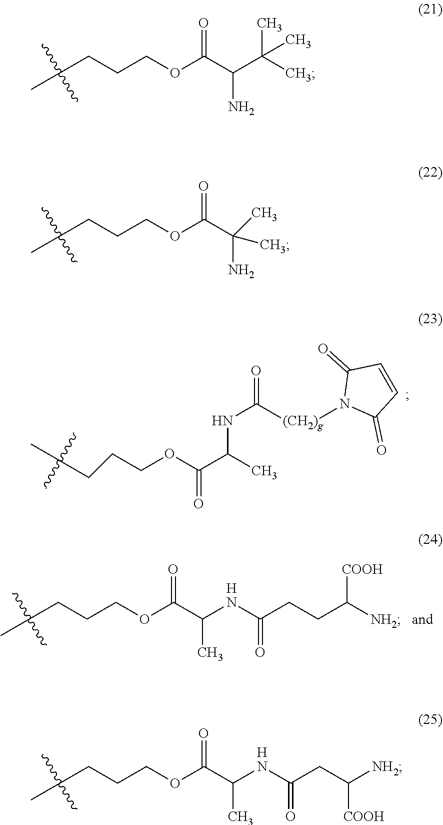

wherein:

a is an integer from 1 to 6;

g is an integer from 2 to 6; and

c is an integer from 0 to 3.

In some embodiments, in Formula (VI1), R₄₀ is

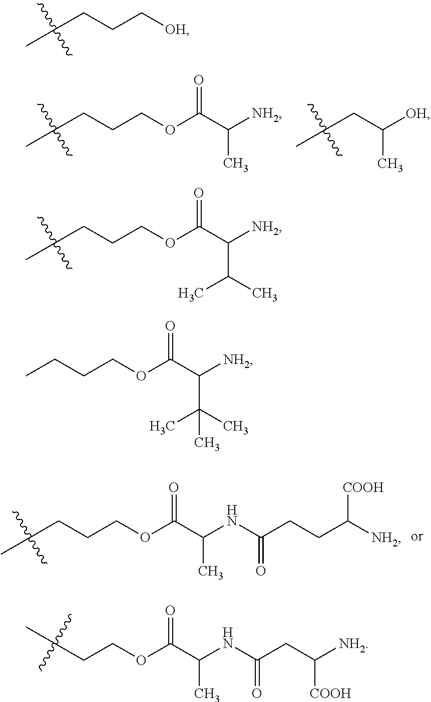

In some embodiments, R₄₀ is

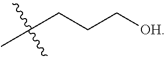 [SMILES: *CCCO]

In some embodiments, R₄₀ is

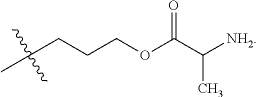 [SMILES: *CCCOC(=O)C(C)N]

In some embodiments, R₄₀ is

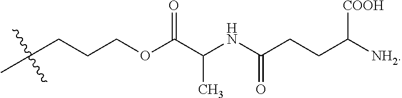 [SMILES: *CCCOC(=O)C(C)NC(=O)CCC(N)C(=O)O]

In some embodiments, R₄₀ is

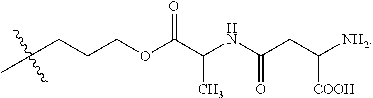 [SMILES: *CCCOC(=O)C(C)NC(=O)CC(N)C(=O)O]

In some embodiments, the compound of Formula (VI1) is a compound of Formula (VIa), (VIb), (VIc), (VId), (VIe) or (VIf):

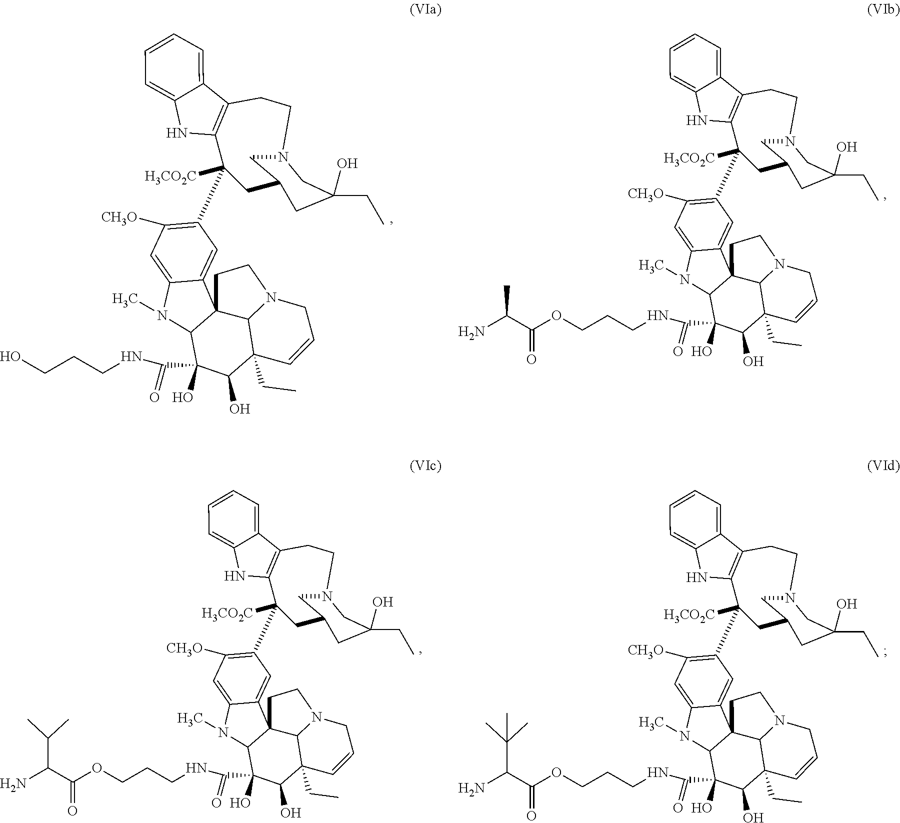

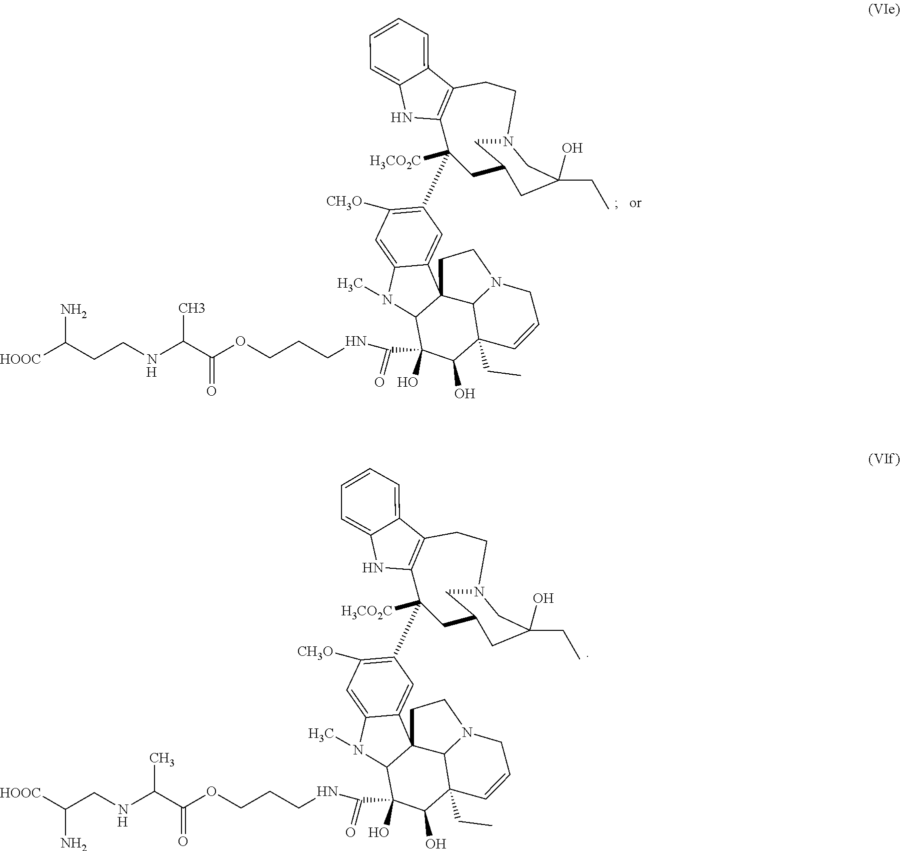

In some embodiments, the topoisomerase inhibitor is a camptothecin compound of Formula (VII1):

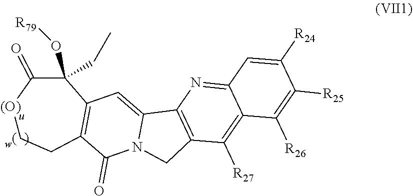

wherein:

R₂₄ is —H, —Cl, —F, —OH, or alkyl; or R₂₄ and R₂₅, may be taken together to form an optionally substituted five- or six-membered ring;

R₂₅ is —H, —F, —OH, —CH₃, —CH═N—O-t-Butyl, —CH₂CH₂Si(CH₃)₃, —Si((CH₃)₂)-t-butyl, or —O—C(O)—R₂₉;

R₂₉ is —NH₂, —R₂₈—C₁₋₆ alkyl-R₂₂, 5- to 12-membered heterocycloalkyl, R₂₈—C₅₋₁₂ heterocycloalkyl-C₁₋₆ alkyl-R₂₂, or —R₂₈—C₁₋₆ alkyl-C₆₋₁₂ aryl-C₁₋₆ alkyl-R₂₂; or R₂₉ is R₄₇ as defined herein;

R₂₆ is —H, —CH₂—N(CH₃)₂, NH₂, or NO₂;

R₂₇ is —H, ethyl, N-methyl piperidine, cycloalkyl, —CH₂OH, —CH₂CH₂NHCH(CH₃)₂, or —N-4-methylcyclohexylamine;

R₇₉ is —H or —C(O)—R₂₈—[C(R₂₀R₂₁)]—R₂₂;

each of R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

or R₂₆ and R₂₇ when taken together with the two carbon atoms to which they attach and the third carbon atom connecting the two carbon atoms form an optionally substituted six-membered ring;

R₂₈ is absent, NR₂₃, or oxygen;

a is an integer from 1 to 6;

c is an integer from 0 to 3;

d is an integer from 1 to 3;

f is an integer from 1 to 12;

u is an integer 0 or 1; and

w is an integer 0 or 1;

with the proviso that the compound of Formula (VIII) must contain at least one of R₂₉ and R₇₉.

In some embodiments the camptothecin compound of Formula (VII1) is a compound of Formula (VIII1), (VIIIa), or (VIIIb), or Formula (XXV) or (XXVa):

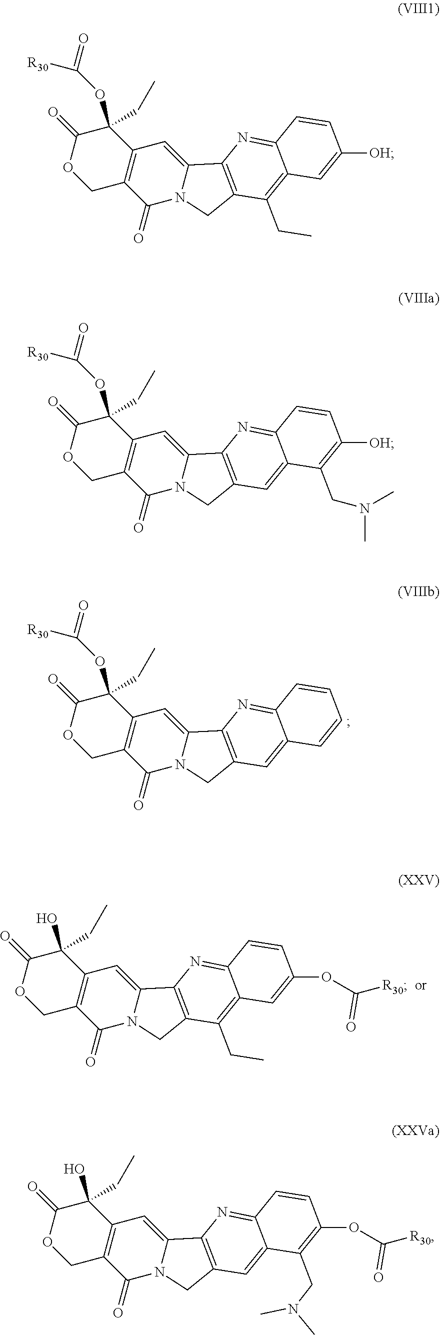

wherein:

R₃₀ is —NH₂, —R₂₈—[C(R₂₀R₂₁)]ₐ—R₂₂, —R₂₈—C₁₋₆ alkyl-R₂₂, 5- to 12-membered heterocycloalkyl, R₂₈—C₅₋₁₂ heterocycloalkyl-C₁₋₆ alkyl-R₂₂, or —R₂₈—C₁₋₆ alkyl-C₆₋₁₂ aryl-C₁₋₆ alkyl-R₂₂;

R₂₈ is absent, NR₂₃, or oxygen;

each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NR₂₃)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound:

R₈₂ is —NR₂₃ or oxygen;

a is an integer from 1 to 6;

c is an integer from 0 to 3;

d is an integer from 1 to 3; and

f is an integer from 1 to 12.

In some embodiments, R₃₀ is any one of the following structures:

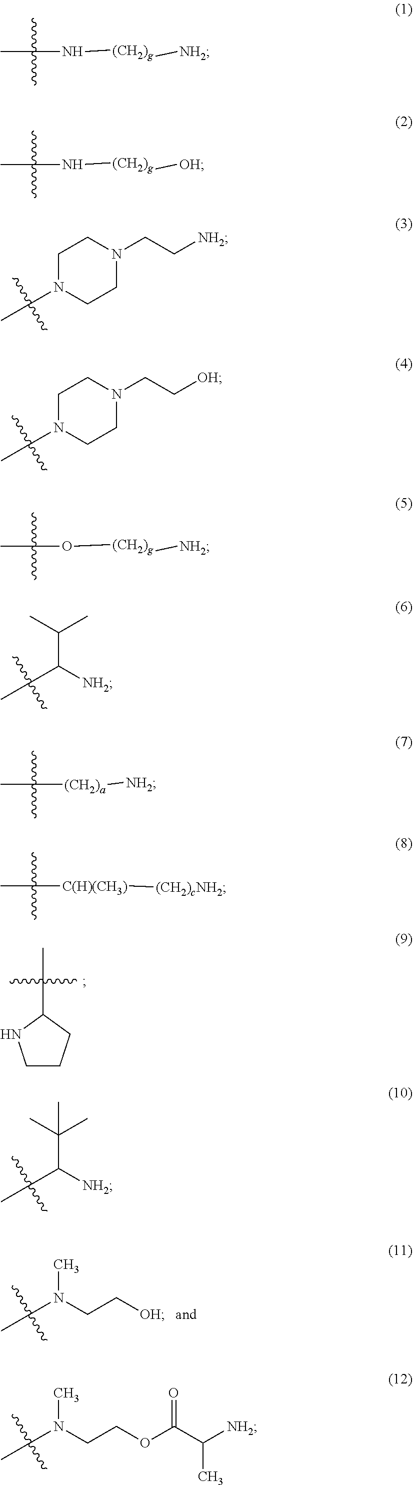

wherein:

a is an integer from 1 to 6;

c is an integer from 0 to 3; and

g is an integer from 2 to 6.

In some embodiments, in Formula (VII1), R₃₀ is:

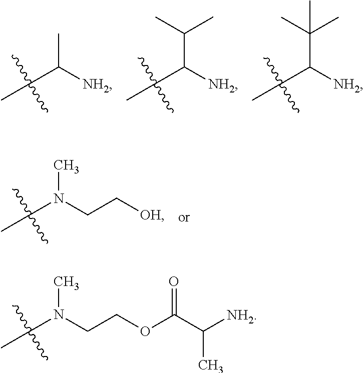

In some embodiments, the compound of Formula (VII1) is a compound of Formula (VIIa), (VIIb), (VIIc), (VIId), (VIIe), (VIIf), (VIIg), (VIIh), (VIIi), or (VIIj):

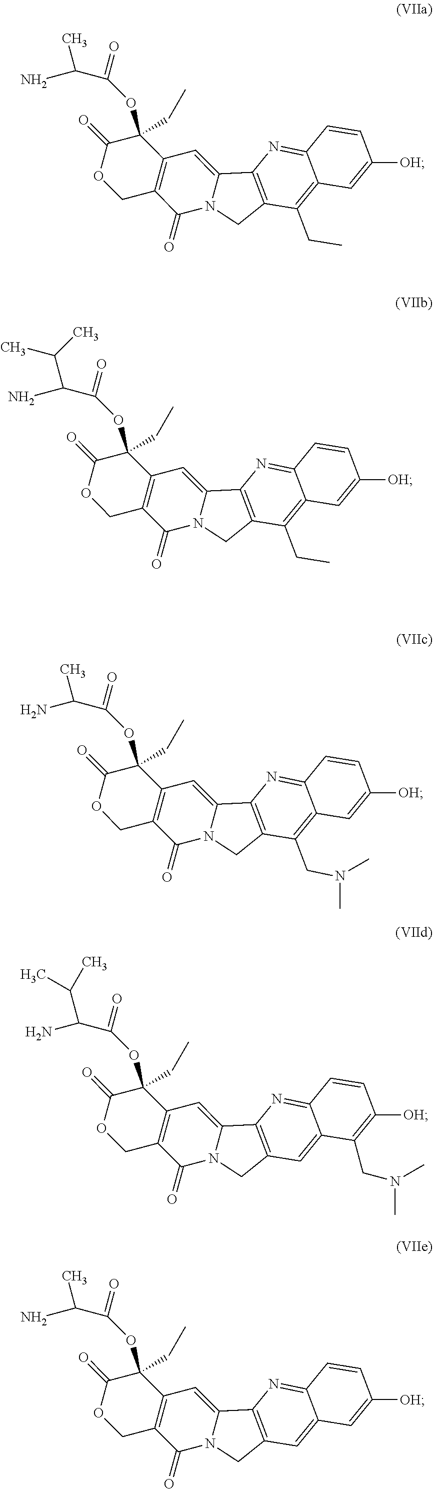 [SMILES: CCc1c2c(nc3ccc(O)cc13)-c1cc3c(c(=O)n1C2)COC(=O)[C@@]3(CC)OC(=O)C(C)N ; CCc1c2c(nc3ccc(O)cc13)-c1cc3c(c(=O)n1C2)COC(=O)[C@@]3(CC)OC(=O)C(N)C(C)C ; CC[C@@]1(OC(=O)C(C)N)C(=O)OCc2c1cc1n(c2=O)Cc2c-1nc1ccc(O)cc1c2CN(C)C ; CC[C@@]1(OC(=O)C(N)C(C)C)C(=O)OCc2c1cc1n(c2=O)Cc2cc3c(CN(C)C)c(O)ccc3nc2-1 ; CC[C@@]1(OC(=O)C(C)N)C(=O)OCc2c1cc1n(c2=O)Cc2cc3cc(O)ccc3nc2-1]

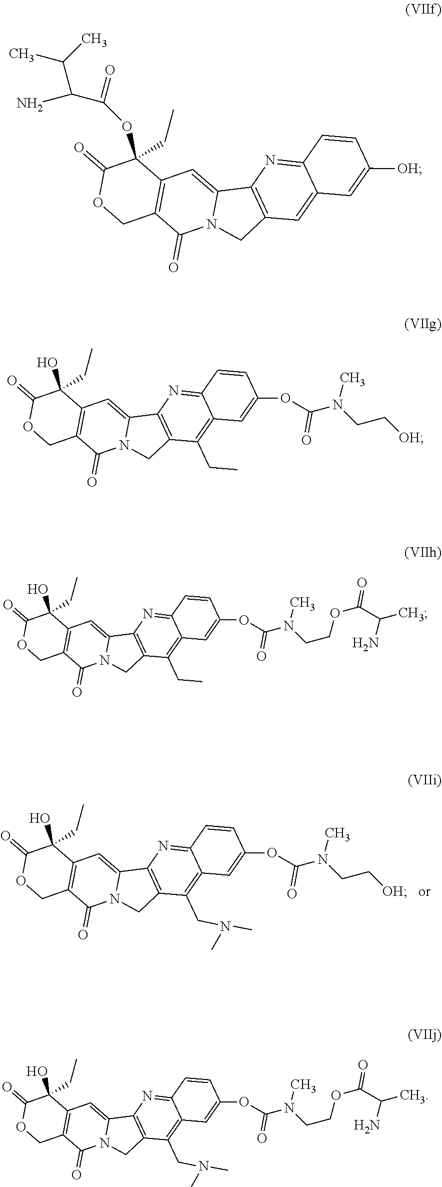

In some embodiments the PI3 kinase inhibitor is a compound of Formula (IX1):

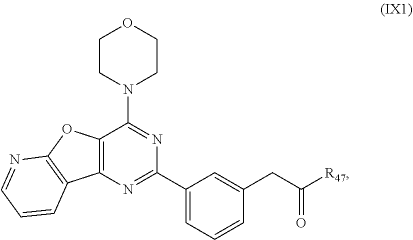

wherein

R₄₇ is an amino group, —R₉—[C(R₂₀R₂₁)]ₐ—R₁₀, —R₉—C₅₋₁₂ heterocycloalkyl-C₁₋₆ alkyl-R₁₀, 5 to 12-membered heterocycloalkyl, or —R₉—C₆₋₁₀ aryl;

each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₁₀ is —OH, —NHR₈₃, —N—(R₈₃)R₁₁, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), —R₈₂—(C(O)—CH(X²)—NH)_{d}—R₇₇, or —R₈₂—C(O)—[C(R₂₀R₂₁)]ₐ—R₈₂—R₈₃;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

R₉ is absent, N—(R₈₃) or oxygen;

R₈₃ is —H or CH₃; and

R₁₁ is:

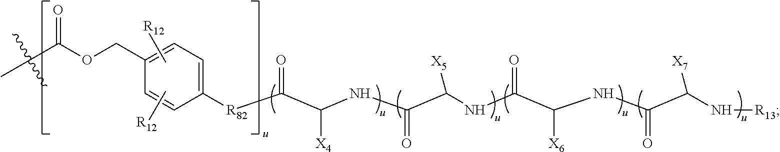

each R₁₂ independently is hydrogen, chloride, —CH₃, or —OCH₃;

R₁₃ is —H or —C(O)—(CH₂)_{d}—(O—CH₂—CH₂)_{f}—NH₂;

R₈₂ is —NR₂₃ or oxygen;

X₄ is the side chain of lysine, arginine, citrulline, alanine, or glycine;

X₅ is the side chain of phenylalanine, valine, leucine, isoleucine, or tryptophan;

each of X₆ and X₇ is independently the side chain of glycine, alanine, serine, valine, or proline;

a is an integer from 1 to 6;

c is an integer from 0 to 3;

d is an integer from 1 to 3;

f is an integer from 1 to 12; and

each u independently is an integer 0 or 1;

or R₁₁ is —Yᵤ—W_{q}—R₈₈,

wherein:

Y is any one of the following structures:

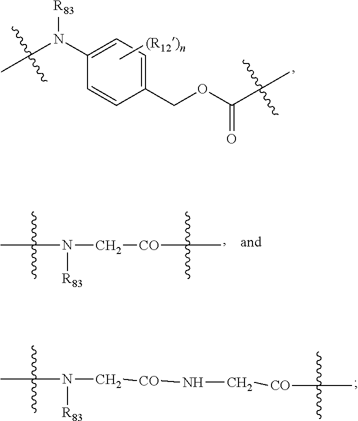

in each of which the terminal NR₈₃ group of Y is proximal to R₈₈;

R₈₃ is —H or CH₃;

each W is an amino acid unit;

each R₁₂′ independently is halogen, —C₁₋₈ alkyl, —O—C₁₋₈ alkyl, nitro, or cyano;

R₈₈ is —H or —C(O)—(CH₂)_{ff}—(NH—C(O))ₐₐ-E-(CH₂)_{bb}—R₈₅;

R₈₅ is NH₂ or OH;

E is —CH₂— or —CH₂CH₂O—;

u is an integer 0 or 1;

q is an integer from 0 to 12;

aa is an integer 0 or 1;

bb is an integer 0 or 2;

ff is an integer from 0 to 10;

h is an integer from 0 to 4;

j is an integer from 0 to 12; and

when E is —CH₂—, bb is 0 and j is an integer from 0 to 10; and when E is —CH₂CH₂—O—, bb is 2 and j is an integer from 1 to 12;

or R₁₁ is:

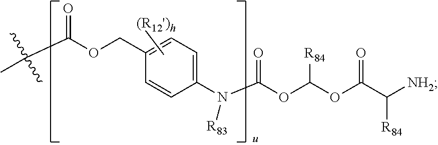

wherein:

R₈₃ is —H or CH₃.

R₈₄ is C₁₋₆ alkyl or C₆₋₁₀ aryl;

each R₁₂′ independently is halogen, —C₁₋₈ alkyl, —O—C₁₋₈ alkyl, nitro, or cyano;

h is an integer from 0 to 4; and

u is an integer 0 or 1.

In some embodiments, R₁₁ is:

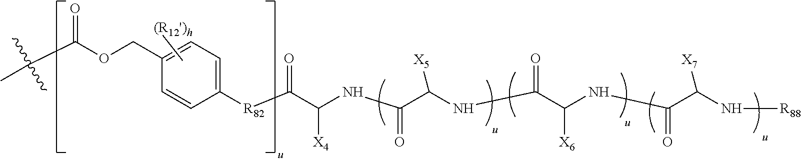

wherein:

each R₁₂′ independently is chloride, —CH₃, or —OCH₃;

R₈₈ is —H or —C(O)—(CH₂)_{ff}—(CH₂—CH₂O)ⱼ—CH₂—CH₂—NH₂;

R₈₂ is —NR₂₃ or oxygen;

X₄ is the side chain of lysine, arginine, citrulline, alanine, or glycine;

X₅ is the side chain of phenylalanine, valine, leucine, isoleucine, or tryptophan;

each of X₆ and X₇ is independently the side chain of glycine, alanine, serine, valine, or proline;

ff is an integer from 1 to 3;

j is an integer from 1 to 12;

h is an integer from 0 to 4; and

each u independently is an integer 0 or 1.

In some embodiments,

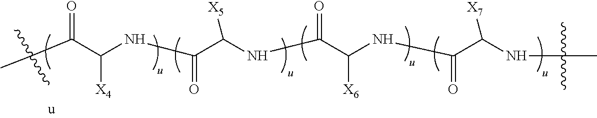

is citrulline-valine; lysine-phenylalanine; citrulline-phenylalanine; citrulline-leucine; citrulline-valine-glycine-glycine; glycine-phenylalanine-glycine-glycine; valine; proline; leucine; or isoleucine.

In some embodiments, R₁₁ is any one of the following structures:

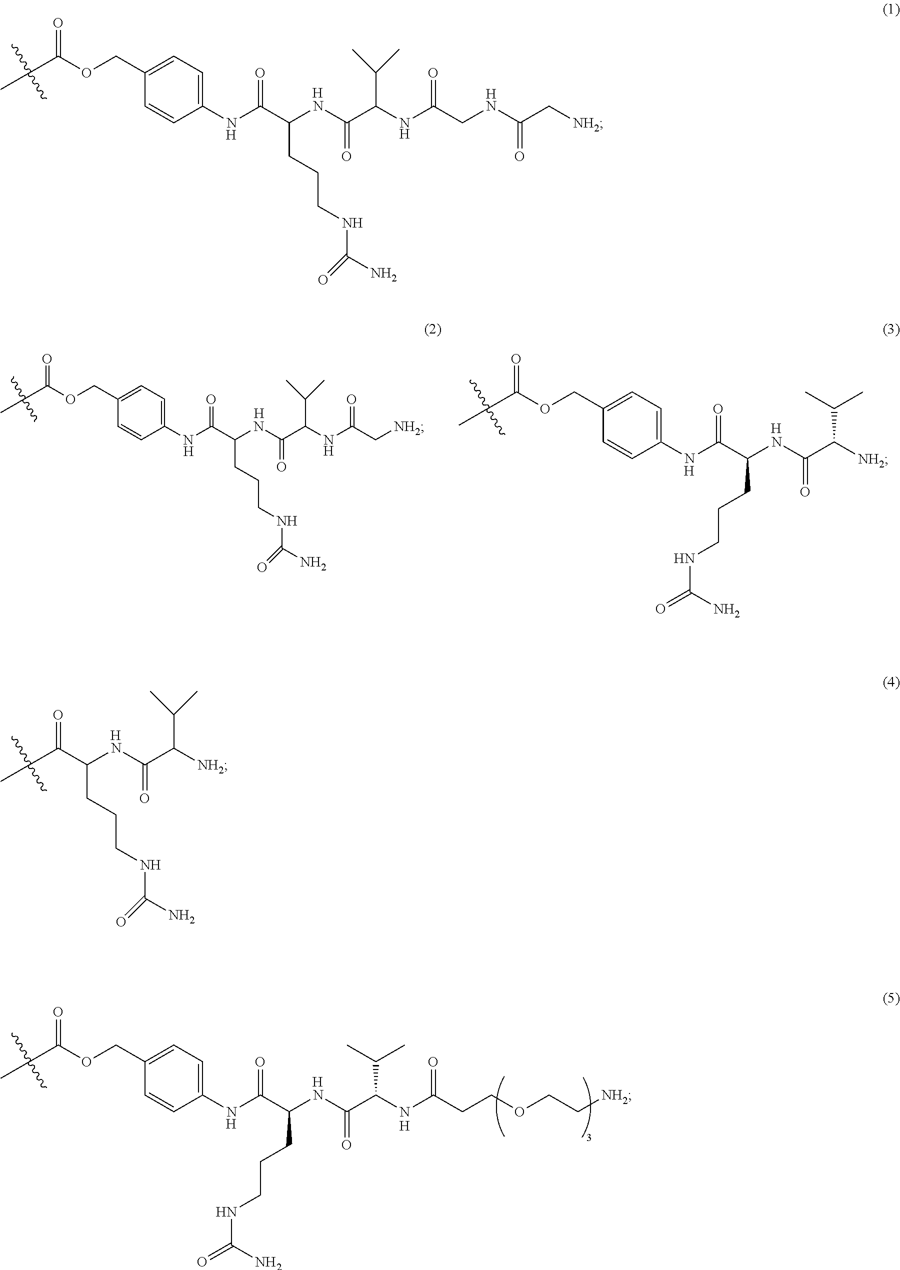

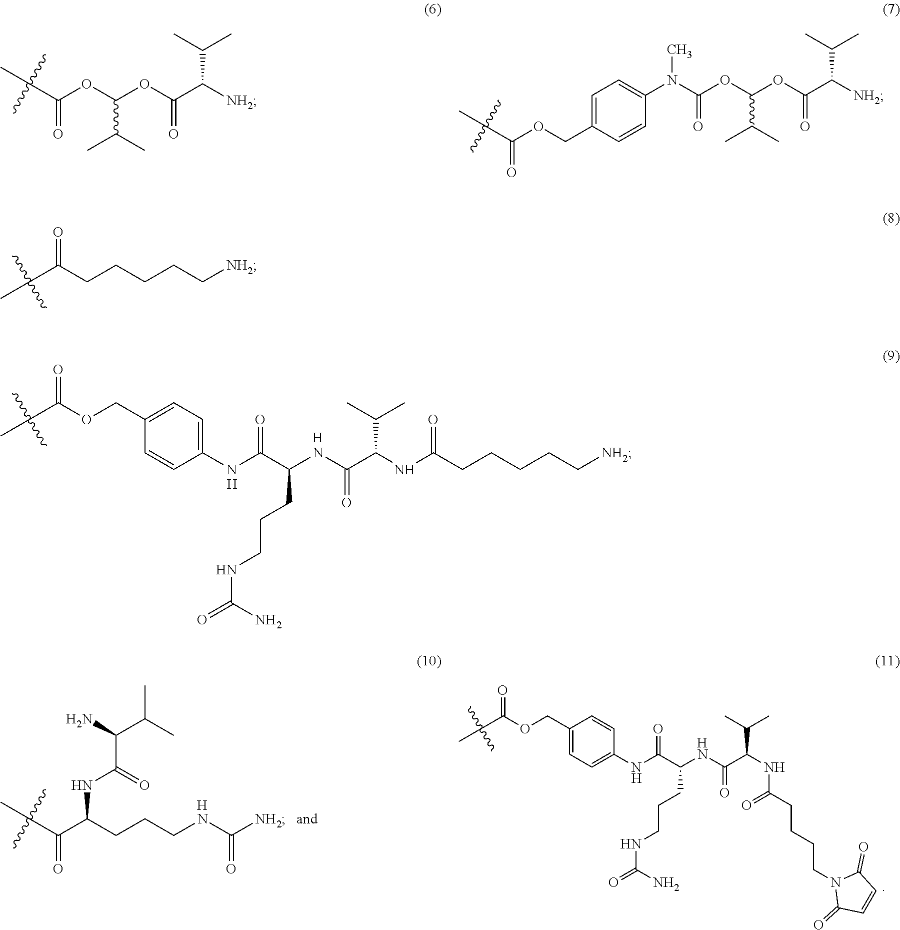

In some embodiments, R₄₇ is any one of the following structures:

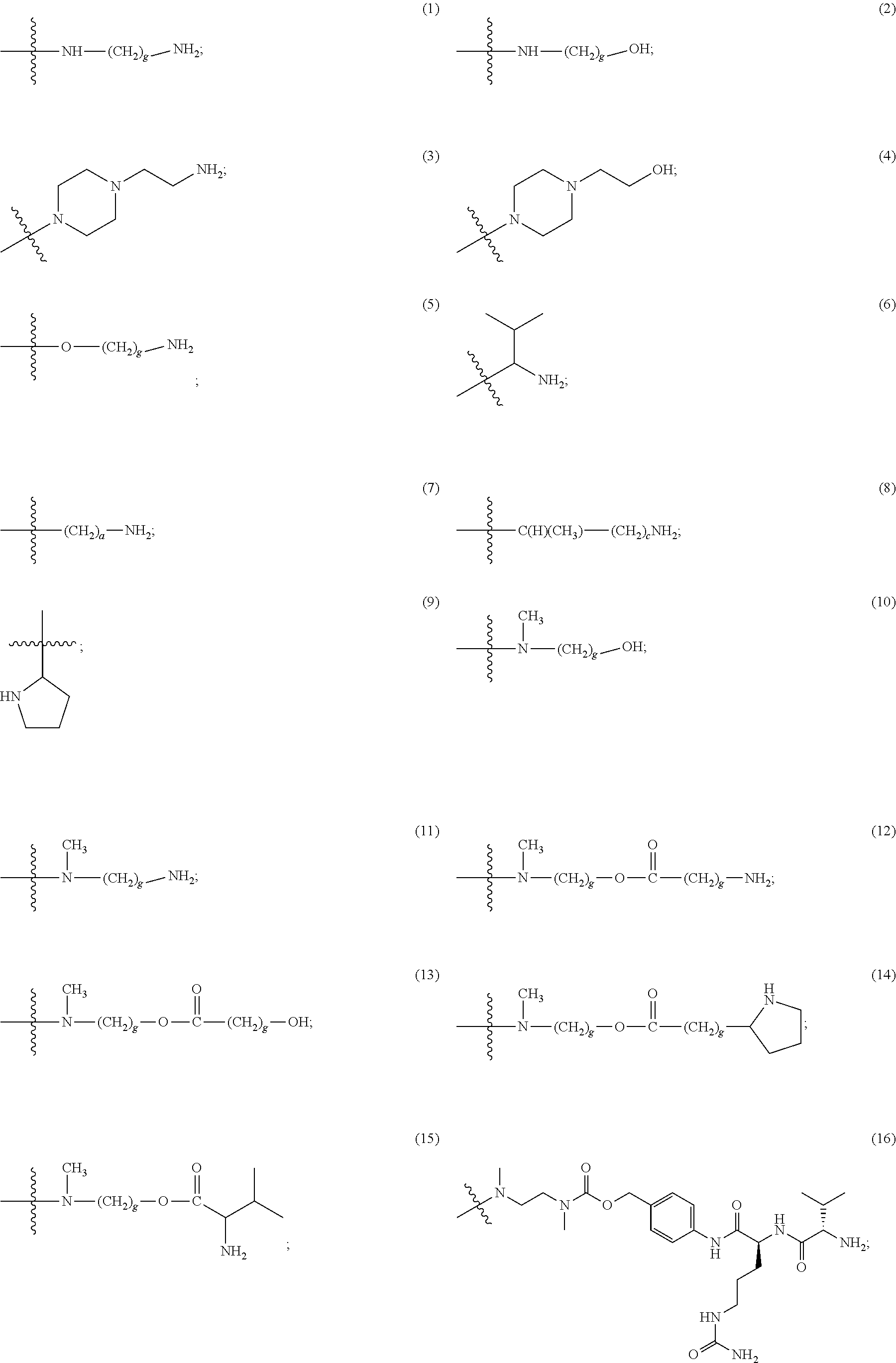

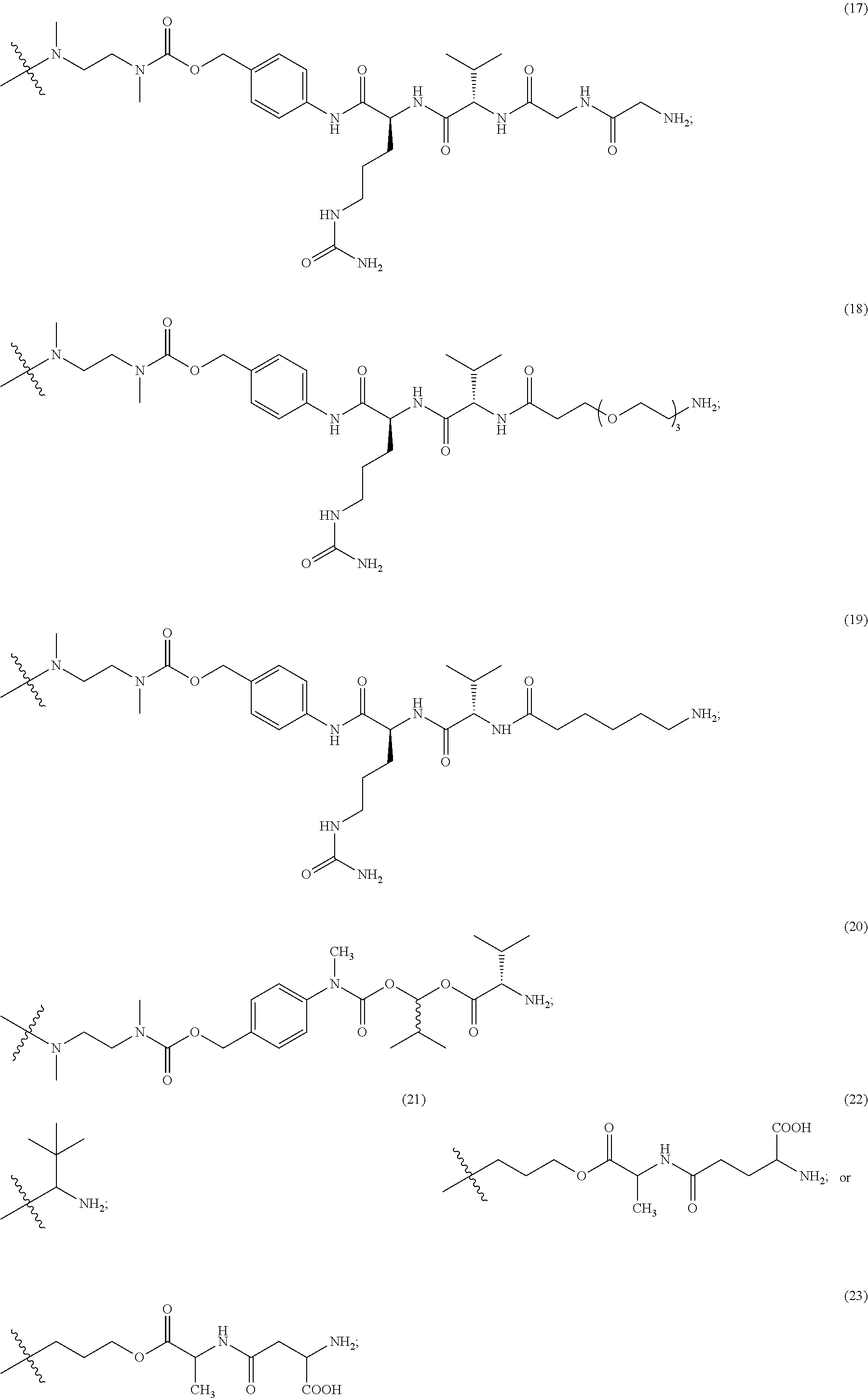

wherein:

a is an integer from 1 to 6;

c is an integer from 0 to 3; and

g is an integer from 2 to 6.

In some embodiments the auristatin is a compound of Formula (X):

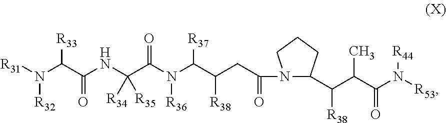

wherein:

each of R₃₁ and R₃₂ independently is —H or C₁₋₈ alkyl and at most one of R₃₁ and R₃₂ is —H;

R₃₃ is —H, C₁₋₈ alkyl, C₃₋₈ carbocycle, C₆₋₁₀ aryl, C₁₋₈ alkyl-C₆₋₁₀ aryl, X¹—(C₃₋₈ carbocycle), C₃₋₈ heterocycle, or X¹—(C₃₋₈ heterocycle);

R₃₄ is —H, C₁₋₈ alkyl, C₃₋₈ carbocycle, C₆₋₁₀ aryl, X¹—C₆₋₁₀ aryl, X¹—(C₃₋₈ carbocycle), C₃₋₈ heterocycle, or X¹—(C₃₋₈ heterocycle);

R₃₅ is —H or methyl;

or R₃₄ and R₃₅, together with the carbon atom to which they attach form a carbocyclic ring having the formula —(CR₅₅R₄₁)_{b}— wherein each of R₅₅ and R₄₁ independently is —H or C₁₋₈ alkyl and b is an integer from 3 to 7;

R₃₆ is —H or C₁₋₈ alkyl;

R₃₇ is —H, C₁₋₈ alkyl, C₃₋₈ carbocycle, C₆₋₁₀ aryl, —X¹—C₆₋₁₀ aryl, —X¹—(C₃₋₈ carbocycle), C₃₋₈ heterocycle, or —X¹—(C₃₋₈ heterocycle);

each Ria independently is —H, OH, C₁₋₈ alkyl, C₃₋₈ carbocycle, or O—(C₁₋₈ alkyl);

R₅₃ is:

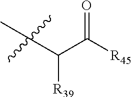

### or R₄;

R₃₉ is —H, C₁₋₈ alkyl, C₆₋₁₀ aryl, —X¹—C₆₋₁₀ aryl, C₃₋₈ carbocycle, C₃₋₈ heterocycle, —X—C₃₋₈ heterocycle, —C₁₋₈ alkylene-NH₂, or (CH₂)₂SCH₃;

each X¹ independently is C₁₋₁₀ alkylene, or C₃₋₁₀ cycloalkylene;

R₄₄ is —H or C₁₋₈ alkyl;

R₄₅ is X³—R₄₂ or NH—R₁₉;

X³ is O or S;

R₁₉ is —H, OH, amino group, Cj-s alkyl amino, or —[C(R₂₀R₂₁)]ₐ—R₂₂;

R₄₂ is an amino group, C₁₋₆ alkyl amino, or —[C(R₂₀R₂₁)]ₐ—R₂₂;

each of R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

R₄ is —C(R₅₆)₂—C(R₅₆)₂—C₆₋₁₀ aryl, —C(R₅₆)₂—C(R₅₆)₂—C₃₋₈ heterocycle, or —C(R₅₆)₂—C(R₅₆)₂—C₃₋₈ carbocycle;

R₅₆ is independently selected from H, OH, C₁₋₈ alkyl, C₃₋₈ carbocycle, —O—C₁₋₈ alkyl, —O—C(O)—R₂₉, and —O—R₂₃—O—C₁₋₆ alkyl-NH₂;

R₂₉ is an amino group, 5- to 12-membered heterocycloalkyl, —R₂₈—C₁₋₆ alkyl-R₂₂, R₂₈—C₅₋₁₂ heterocycloalkyl-C₁₋₆ alkyl-R₂₂, —[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;

R₂₈ is absent, NR₂₃, or oxygen;

a is an integer from 1 to 6;

c is an integer from 0 to 3;

d is an integer from 1 to 3; and

f is an integer from 1 to 12.

In some embodiments, in the auristatin compound of Formula (X):

R₃₉ is benzyl or

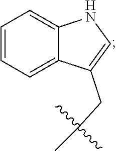 [SMILES: *Cc1c[nH]c2ccccc12]

and

R₄₄ is hydrogen.

In some embodiments the auristatin is a compound of Formula (Xa):

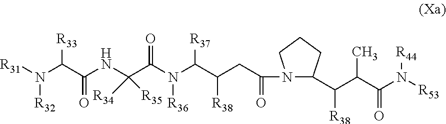

wherein:

R₃₃ through R₃₈, and R₄₄ are as defined herein,

one of R₃₁ and R₃₂ is hydrogen or C₁₋₈ alkyl and the other is:

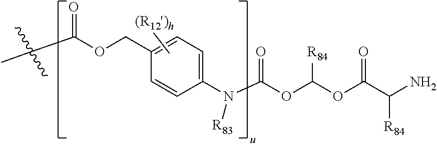

wherein:

R₈₃ is —H or CH₃;

R₈₄ is C₁₋₆ alkyl or C₆₋₁₀ aryl;

each R₁₂′ independently is halogen, —C₁₋₈ alkyl, —O—C₁₋₈ alkyl, nitro, or cyano;

h is an integer from 0 to 4;

u is an integer 0 or 1;

R₅₃ is:

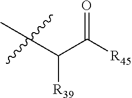

### or R₅₄

R₃₉ is H, C₁₋₈ alkyl, C₆₋₁₀ aryl, —X¹—C₆₋₁₀ aryl, C₃₋₈ carbocycle, C₃₋₈ heterocycle, —X¹—C₃₋₈ heterocycle, —C₁₋₈ alkylene-NH₂, or (CH₂)₂SCH₃,

each X¹ independently is C₁₋₁₀ alkylene or C₃₋₁₀ cycloalkylene;

R₄₅ is X³—R₄₂ or NH—R₁₉;

X³ is O or S;

R₁₉ is —H, OH, amino group, C₁₋₈ alkyl amino, or —[C(R₂₀R₂₁)]ₐ—R₂₂;

R₄₂ is —H, an amino group, C₁₋₆ alkyl amino, or —[C(R₂₀R₂₁)]ₐ—R₂₂;

each of R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(O—CH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

R₅₄ is —C(R₅₆)₂—C(R₅₆)₂—C₆₋₁₀ aryl, —C(R₅₆)₂—C(R₅₆)₂—C₃₋₈ heterocycle, or —C(R₅₆)₂—C(R₅₆)₂—C₃₋₈ carbocycle;

R₅₆ is independently selected from H, OH, C₁₋₈ alkyl, C₃₋₈ carbocycle, —O—C₁₋₈ alkyl, —O—C(O)—R₂₉, and —O—R₂₃—O—C₁₋₆ alkyl-NH₂;

R₂₉ is an amino group, 5 to 12-membered heterocycloalkyl, —R₂₈—C₁₋₆ alkyl-R₂₂, R₂₈—C₅₋₁₂ heterocycloalkyl-C₁₋₆ alkyl-R₂₂, —[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;

R₂₈ is absent, NR₂₃, or oxygen;

a is an integer from 1 to 6;

c is an integer from 0 to 3;

d is an integer from 1 to 3; and

f is an integer from 1 to 12.

In some embodiments, the auristatin compound of Formula (Xa) is a compound of Formula (XIa) or Formula (XIb):

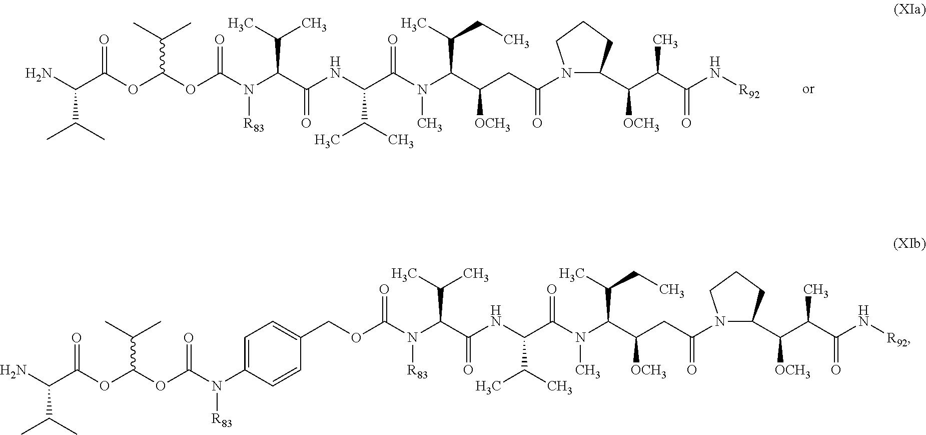

wherein:

R₉₂ is:

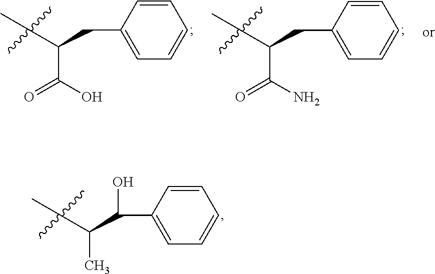 [SMILES: *[C@@H](Cc1ccccc1)C(=O)O ; *[C@@H](Cc1ccccc1)C(N)=O ; *[C@H](C)C(O)c1ccccc1]

and

R₈₃ is hydrogen or CH₃.

In some embodiments the auristatin of Formula (X) is a compound of Formula (XI), Formula (XII) or Formula (XIII):

wherein the compound of Formula (XI) is:

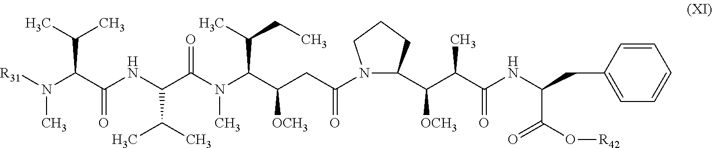

wherein R₃₁ is H or CH₃ and R₄₂ is —CH₃ or any one of the following structures:

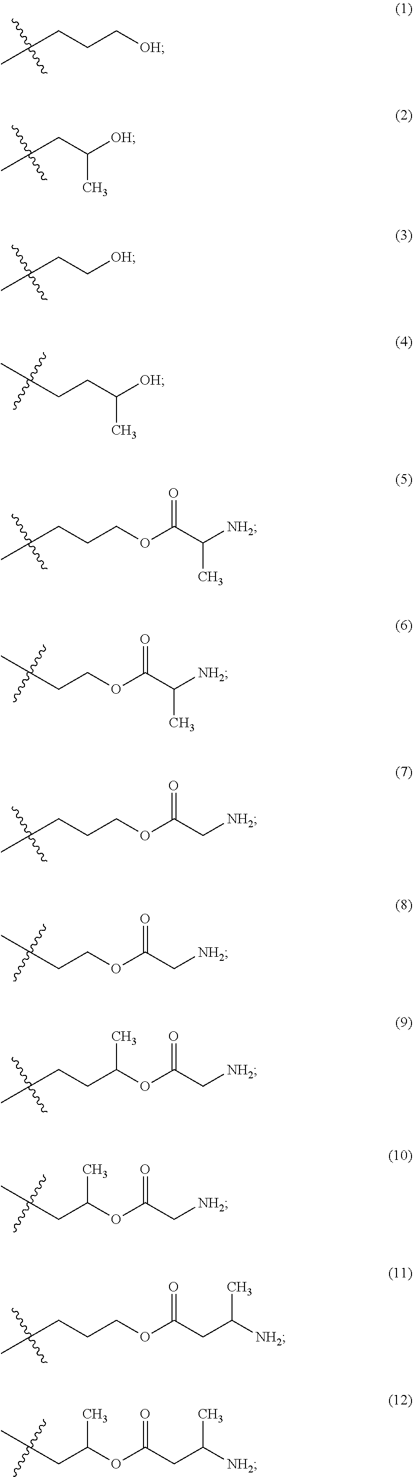

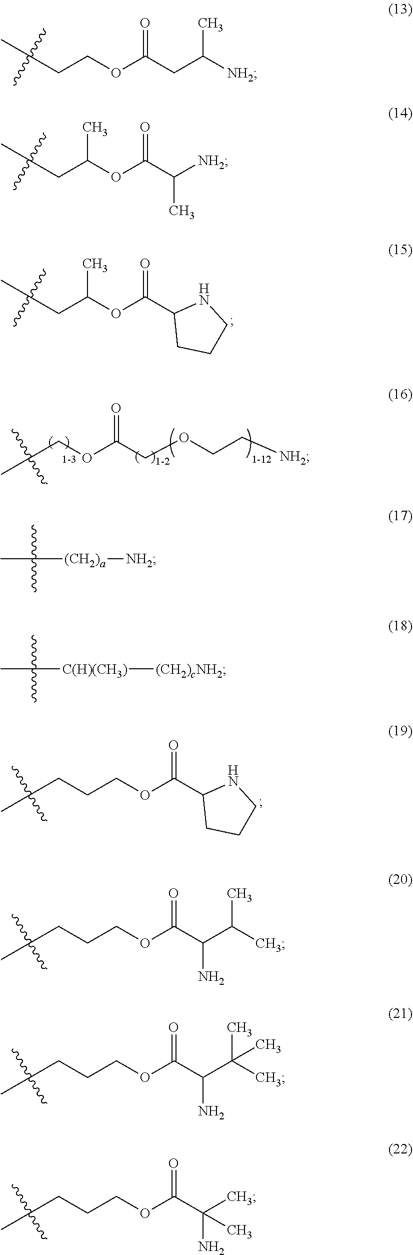

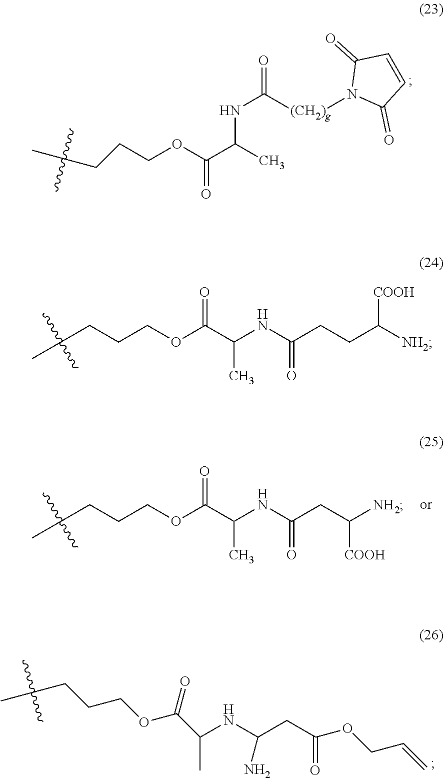

wherein:

a is an integer from 1 to 6;

c is an integer from 0 to 3; and

g is an integer from 2 to 6;

wherein the compound of Formula (XII) is:

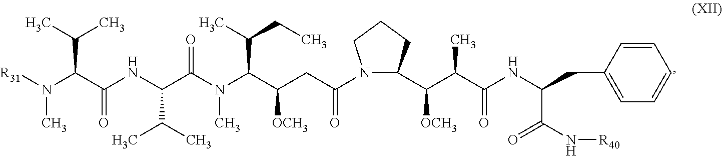

wherein R₃₁ is H or CH₃ and R₄₀ is hydrogen, —OH, —NH₂, or any of the following structures:

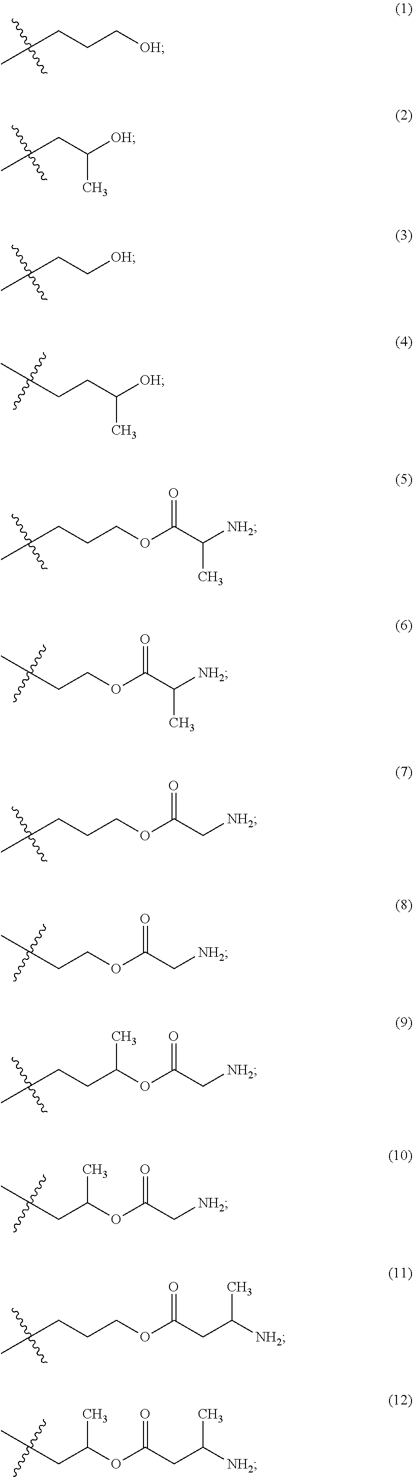

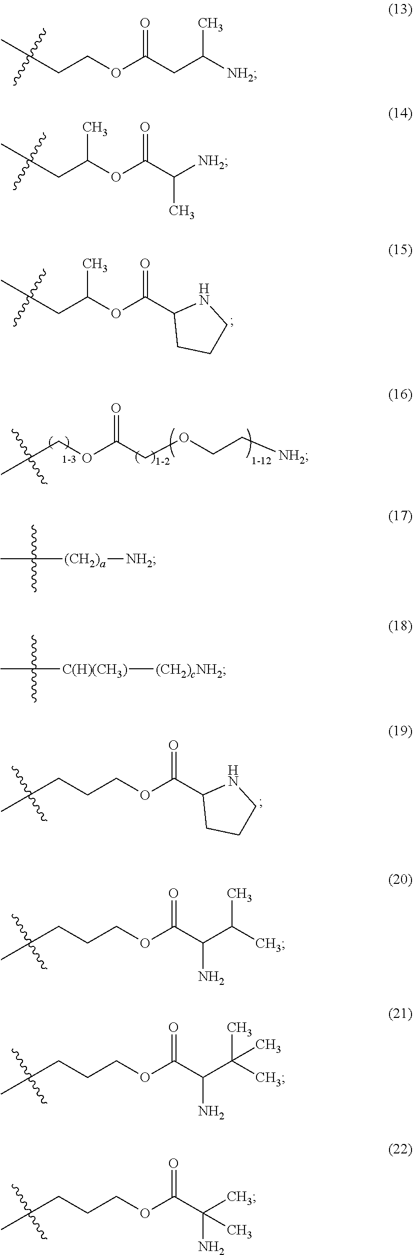

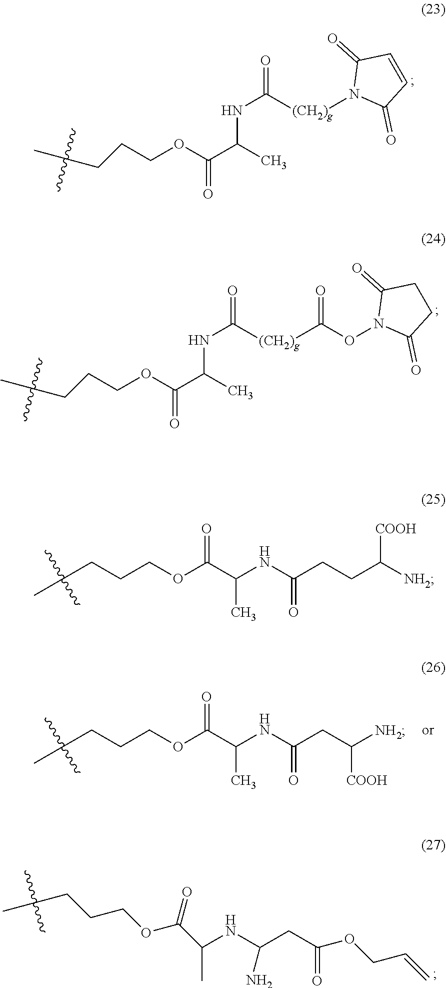

wherein:

a is an integer from 1 to 6;

g is an integer from 2 to 6; and

c is an integer from 0 to 3;

wherein the compound of Formula XIII is:

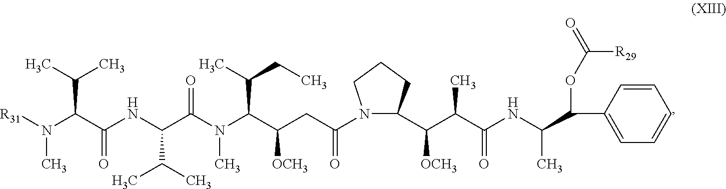

wherein:

R₃₁ is H or CH₃;

R₂₉ is an amino group, 5 to 12-membered heterocycloalkyl, —R₂₈—C₁₋₆ alkyl-R₂₂, R₂₈—C₅₋₁₂ heterocycloalkyl-C₁₋₆ alkyl-R₂₂, —R₂₈—[C(R₂₀R₂₁)]ₐ—R₂₂, or —R₂₈—C₁₋₆ alkyl-C₆₋₁₂ aryl-C₁₋₆ alkyl-R₂₂; or R₂₉ is R₄₇ as defined herein;

each of R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C-s cycloalkyl, or a side chain of a natural or unnatural amino acid;

R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

R₂₈ is absent, NR₂₃ or oxygen;

a is an integer from 1 to 6;

c is an integer from 0 to 3;

d is an integer from 1 to 3; and

f is an integer from 1 to 12.

In some embodiments, in Formula (XII), R₄₀ is

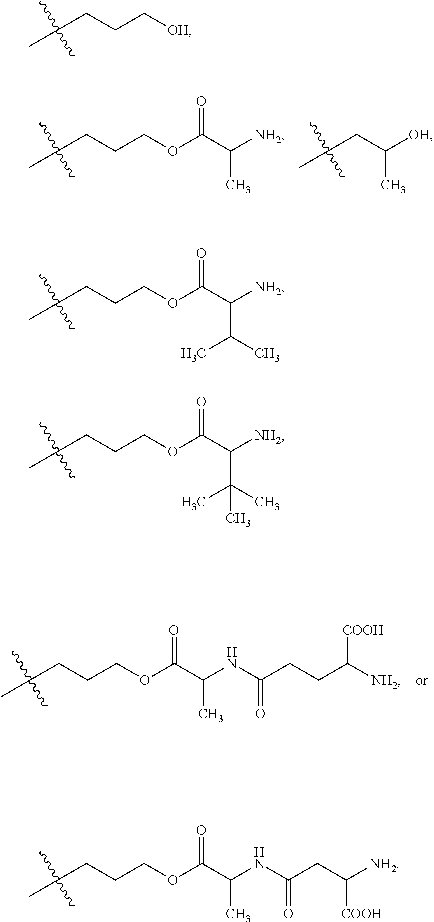

In some embodiments, the compound of Formula (XII) is a compound of Formula (XIIa), (XIIb), (XIIc), (XIId), (XIIe), (XIIf), (XIIg) or (XIIh):

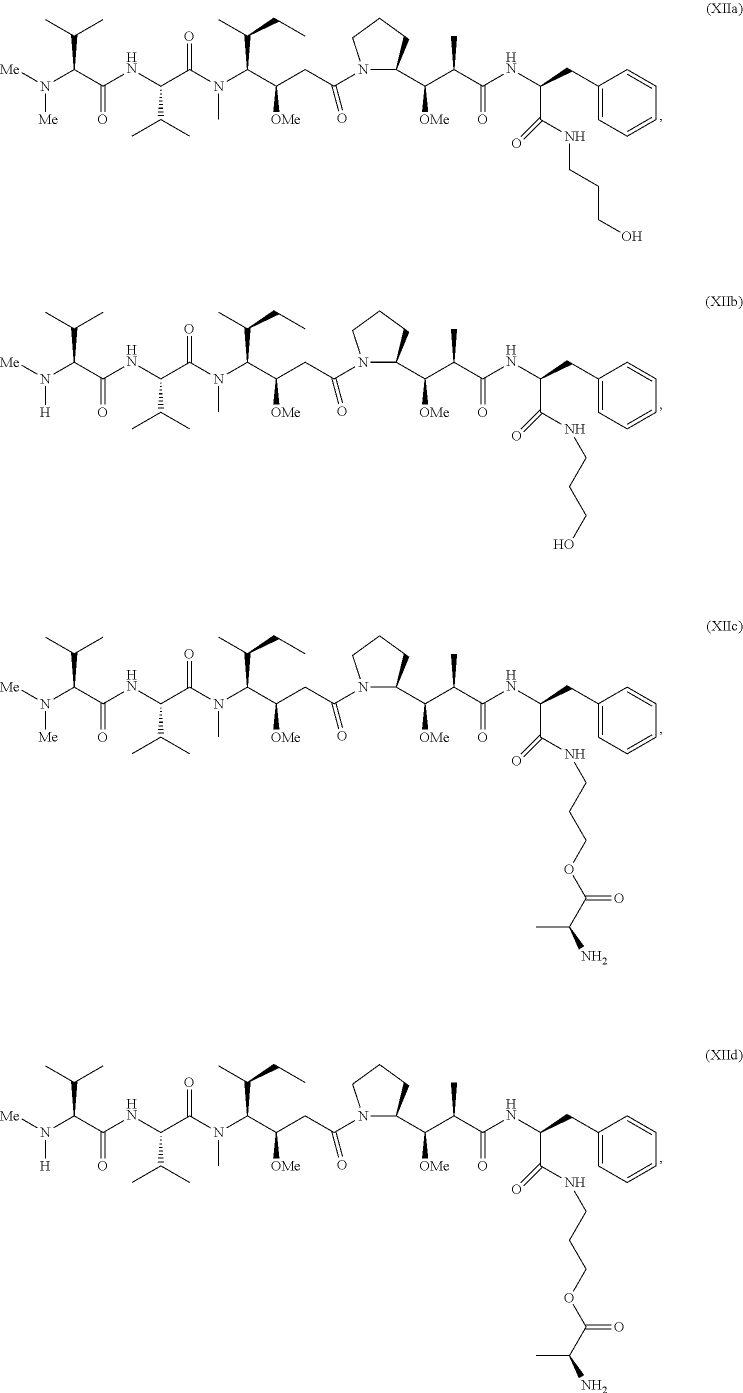

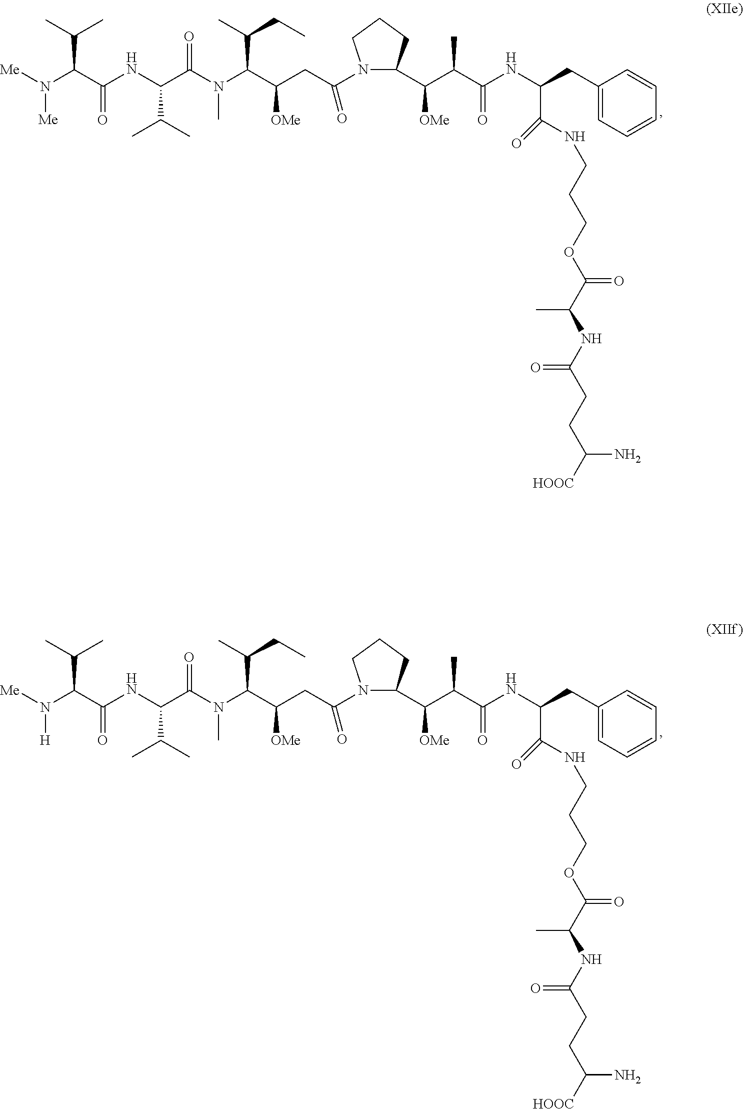 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)NC(=O)CCC(N)C(=O)O)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)NC(=O)CCC(N)C(=O)O)OC)N(C)C(=O)[C@@H](NC(=O)[C@@H](NC)C(C)C)C(C)C]

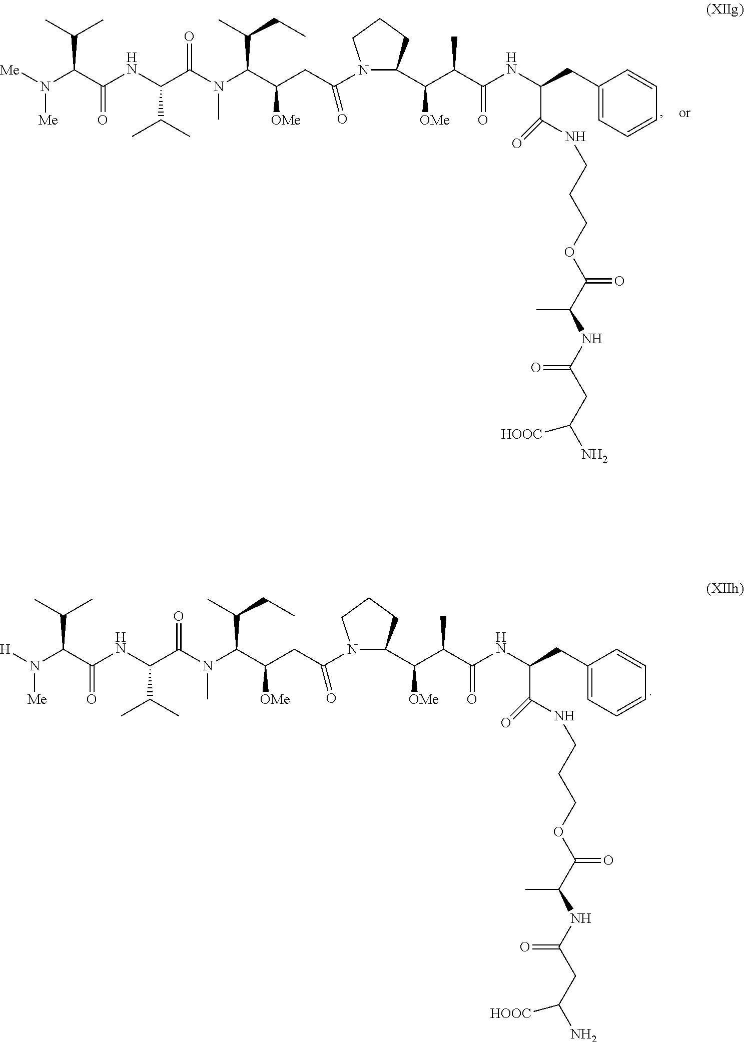 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)NC(=O)CC(N)C(=O)O)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@H](C)NC(=O)CC(N)C(=O)O)OC)N(C)C(=O)[C@@H](NC(=O)[C@@H](NC)C(C)C)C(C)C]

In some embodiments in the compound of Formula (XIII), R₂₉ is —NH₂, 5-membered heterocycloalkyl, —R₂₈—C₁₋₆ alkyl-R₂₂, R₂₈—C₅₋₁₂ heterocycloalkyl-C₁₋₆ alkyl-R₂₂, or —R₂₈—C₁₋₆ alkyl-C₆₋₁₂ aryl-C₁₋₆ alkyl-R₂₂; or R₂₉ is R₄₇ as defined herein;

R₂₈ is absent, NR₂₃, or oxygen;

R₂₂ is —OH, —NHR₂₃, —COOH, —R₈₂—C(O)(CH₂)_{c}—C(H)(R₂₃)—N(H)(R₂₃), —R₈₂—C(O)(CH₂)_{d}—(OCH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR-3 or oxygen;

c is an integer from 0 to 3;

d is an integer from 1 to 3; and

f is an integer from 1 to 12.

In some embodiments, R₂₉ is any one of the following structures:

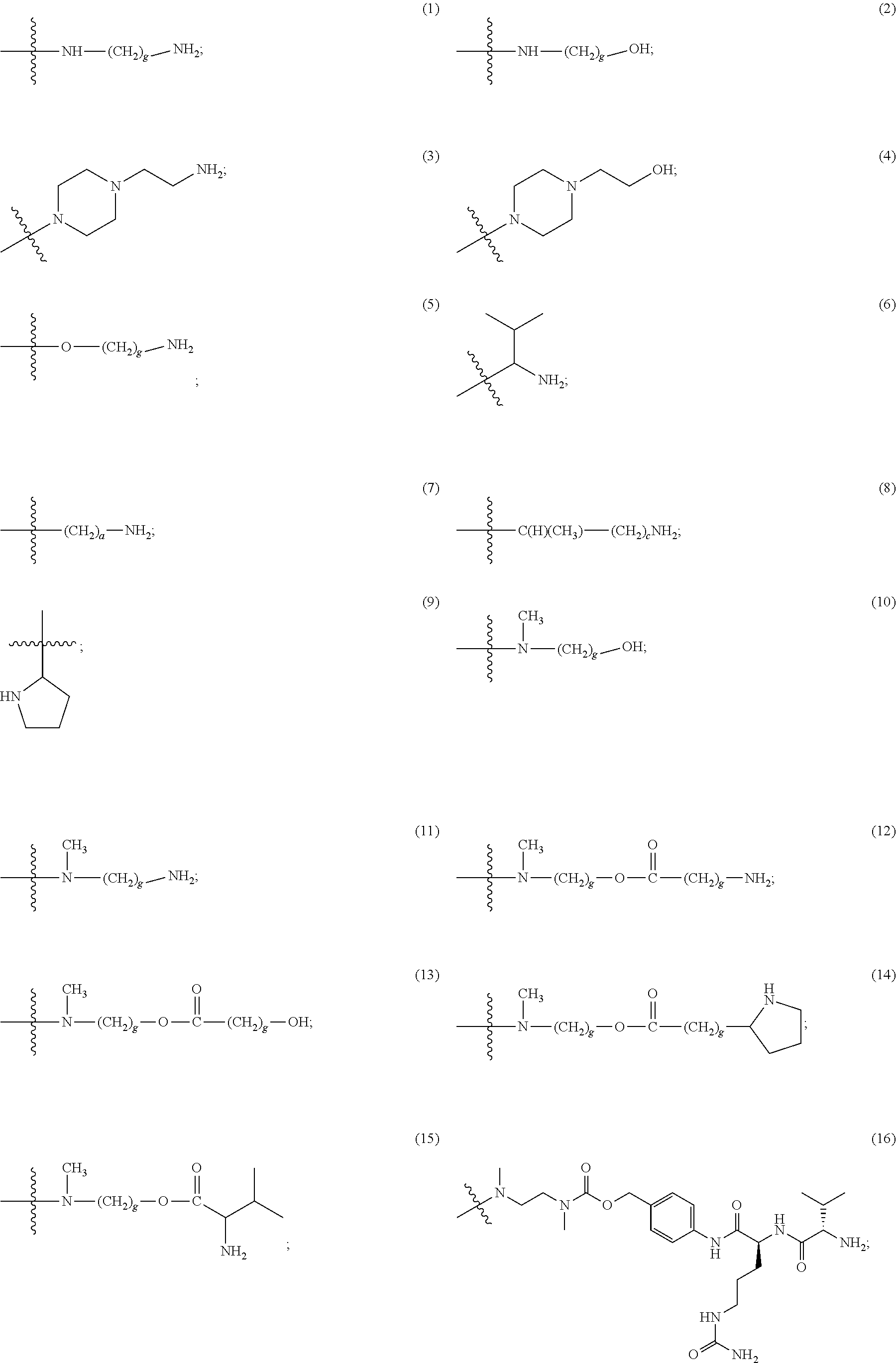

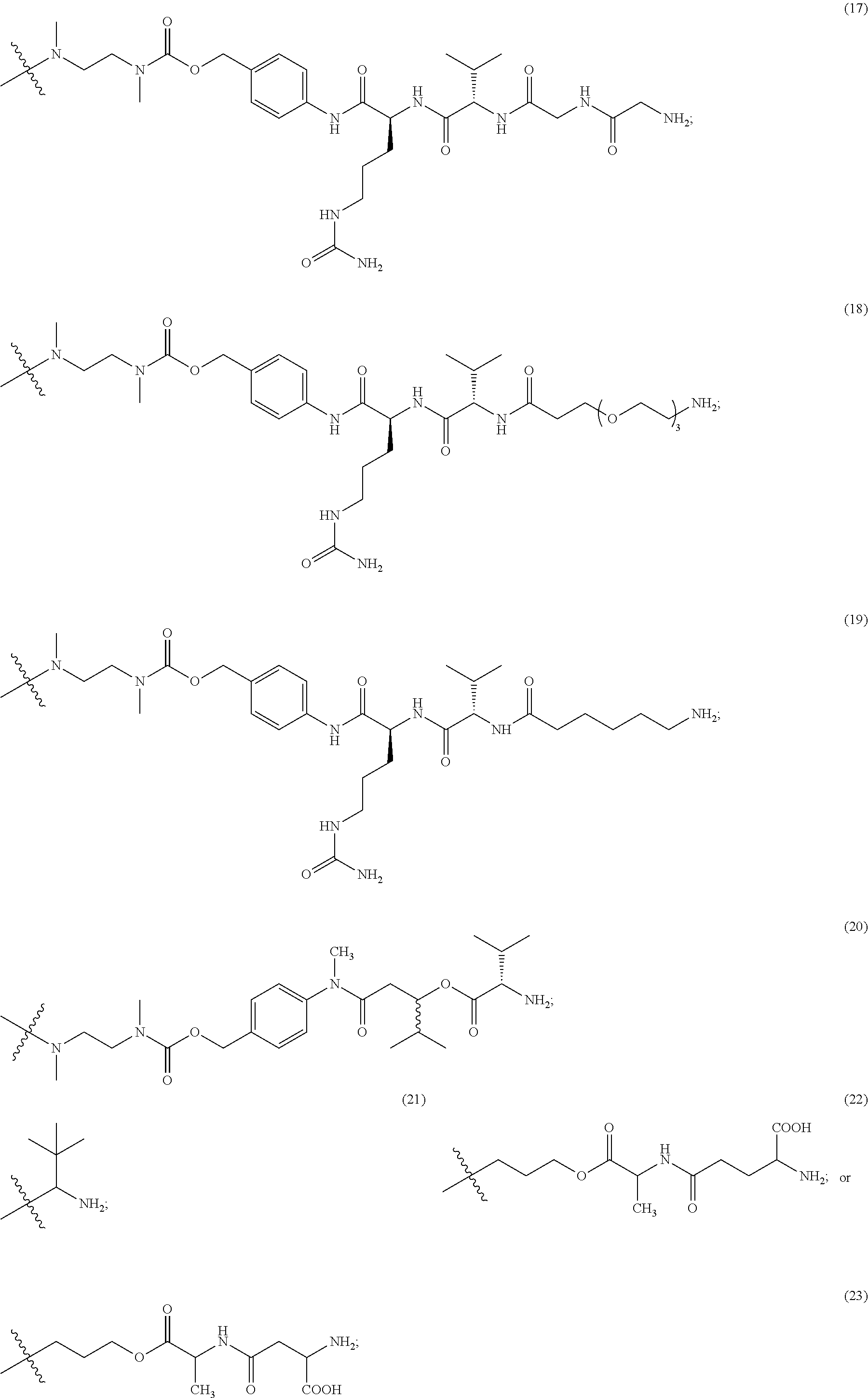

wherein:

a is an integer from 1 to 6;

c is an integer from 0 to 3; and

g is an integer from 2 to 6.

In some embodiments, the MEK inhibitor is a compound of Formula (XIV):

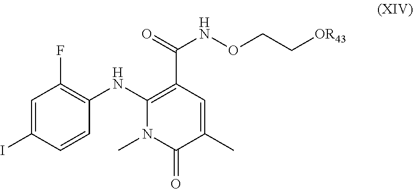

wherein:

R₄₃ is —H or —R₄₆—R₄₇;

each of R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C-s cycloalkyl, or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

R₄₆ is —C(O)—, —C(O)—O—, —C(O)—NH—, or absent:

R₄₇ is as defined herein;

a is an integer from 1 to 6;

c is an integer from 0 to 3;

d is an integer from 1 to 3; and

f is an integer from 1 to 12.

Further examples of the MEK inhibitor are disclosed in U.S. Pat. No. 7,517,994 B2.

In some embodiments, R₄₃ is —C(O)—(CH₂)ₐ—NH₂ or —C(O)—C(H)(CH₃)—(CH₂)_{c}—NH₂; in which a is an integer from 1 to 6; and c is an integer from 0 to 3.

In some embodiments, the duocarmycin compound is a compound of Formula (XV):

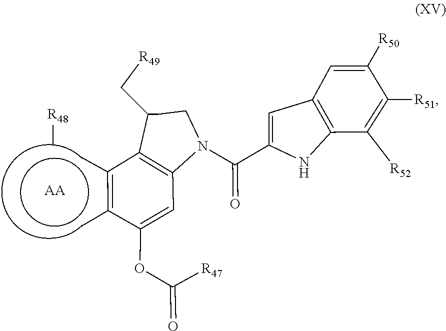

wherein:

R₄₇ is as defined herein;

R₄₈ is hydrogen, —COOC₁₋₆ alkyl, —COOH, —NH₂, or —CH₃;

R₄₉ is Cl, Br, or —OH;

R₅₀ is —H, —OCH₃,

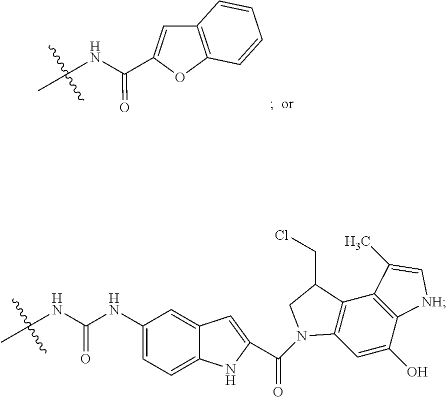 [SMILES: *NC(=O)c1cc2ccccc2o1 ; *NC(=O)Nc1ccc2[nH]c(C(=O)N3CC(CCl)c4c3cc(O)c3[nH]cc(C)c43)cc2c1]

each of R₅₁ and R₅₂ independently is —H or —OCH₃; and

ring AA is either a phenyl or pyrrolyl ring.

Further examples of duocarmycin compounds are disclosed in U.S. Pat. No. 7,553,816.

In some embodiments the duocarmycin compound of Formula (XV) is a compound of Formula (XVI), (XVII), (XVIII) or (XIX):

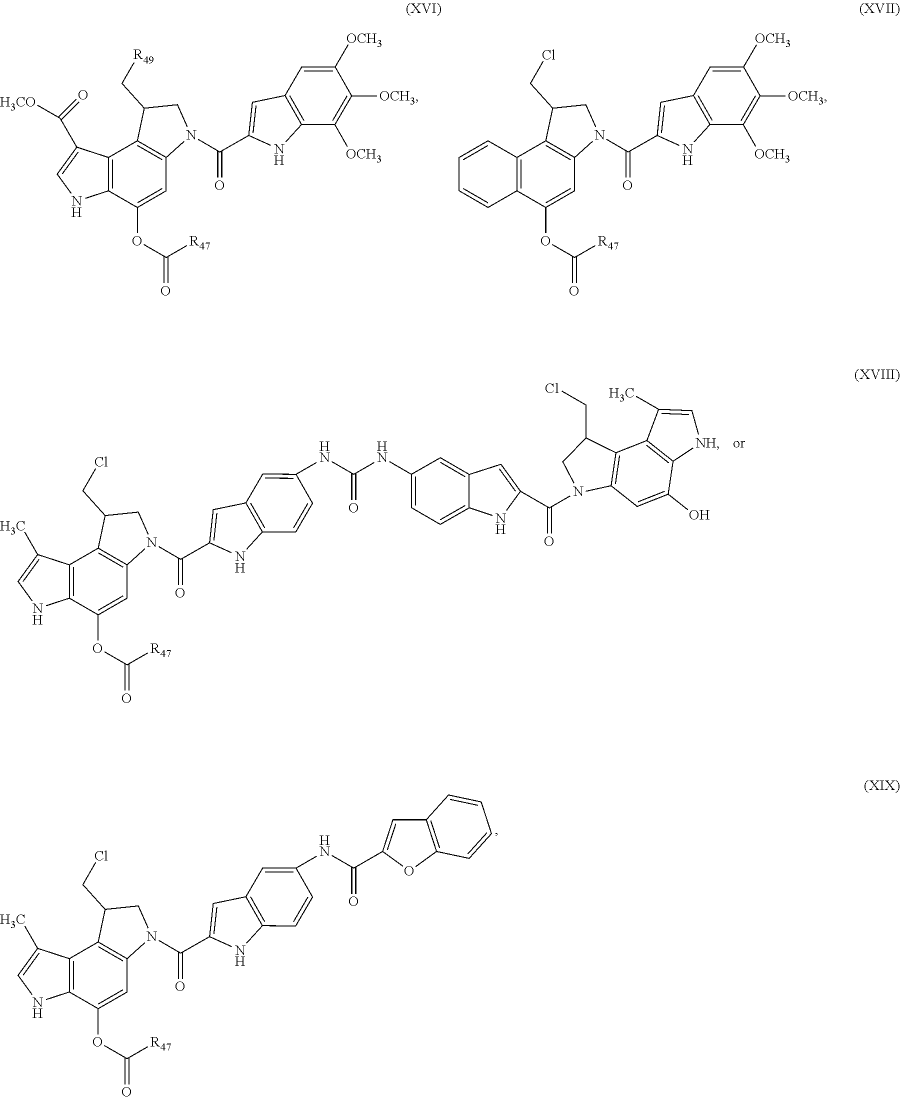

wherein:

R₄₉ is Cl, Br, or —OH; and

R₄₇ is as defined herein.

In some embodiments, the duocarmycin compound is a duocarmycin SA compound of Formula (XX) or (XXI):

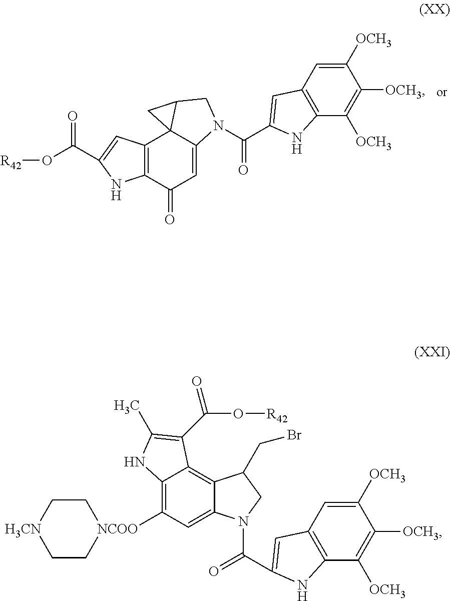

wherein:

R₄₂ is C₁₋₆ alkyl amino or —[C(R₂₀R₂₁)]ₐ—R₂₂;

each of R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl, or a side chain of a natural or unnatural amino acid;

R₂₂ is —OH, —NH₂, —COOH, —R₈₂—C(O)(CH₂)_{c}—C(H)(R₂₃)—N(H)(R₂₃), —R₈₂—C(O)(CH₂)_{d}—(O CH₂—CH₂)_{f}—N(H)(R₂₃), or —R₈₂—(C(O)—CH(X²)—NH)_{d}—R₇₇;

each R₂₃ independently is —H, C₁₋₆ alkyl, C₆₋₁₀ aryl, C₃₋₈ cycloalkyl, —COOH, or —COO—C₁₋₆ alkyl;

X² is a side chain of a natural or unnatural amino acid;

R₇₇ is a —H or X² and NR₇₇ form a nitrogen containing cyclic compound;

R₈₂ is —NR₂₃ or oxygen;

a is an integer from 1 to 6;

c is an integer from 0 to 3;

d is an integer from 1 to 3; and

f is an integer from 1 to 12.

In some embodiments, R₄₂ is any one of the following structures:

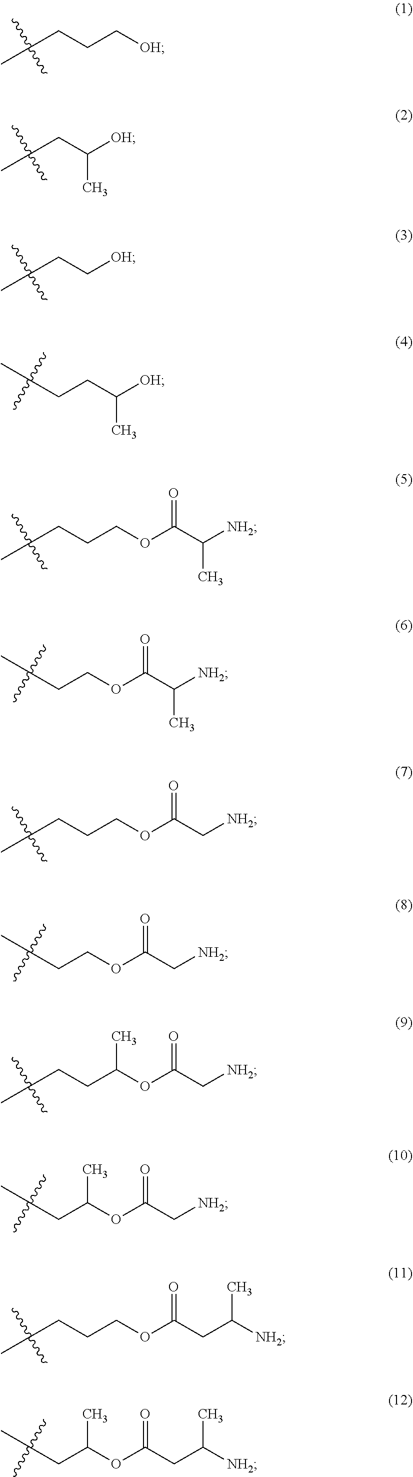

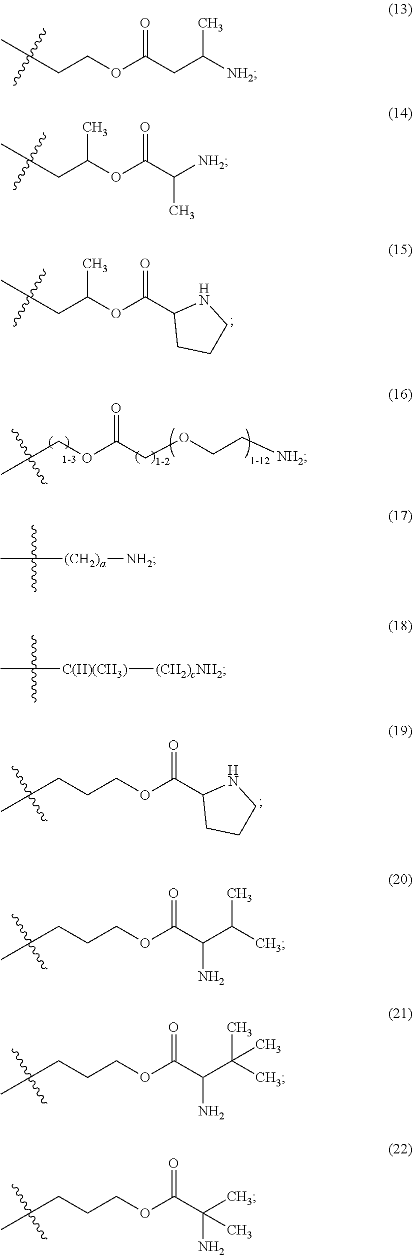

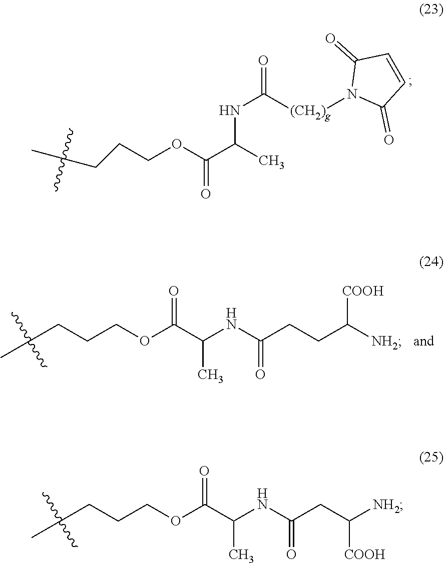

wherein:

a is an integer from 1 to 6;

g is an integer from 2 to 6; and

c is an integer from 0 to 3.

In some embodiments, the KSP inhibitor compound is a compound of Formula (XXVI):

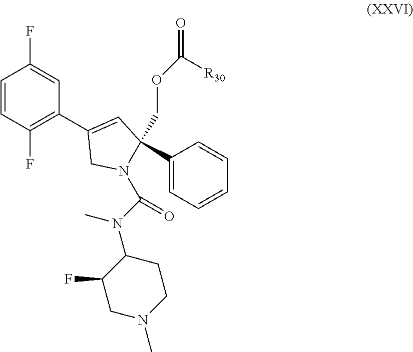

wherein R₃₀ is as defined herein.

In some embodiments, R₃₀ is:

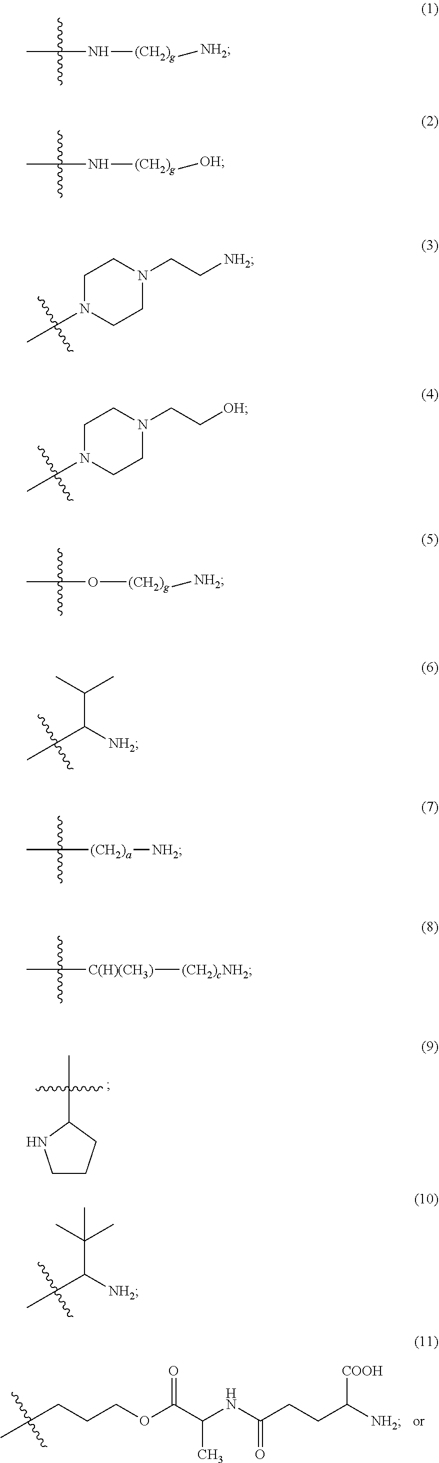

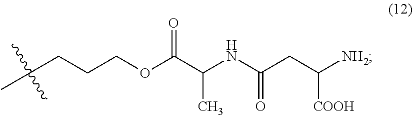

wherein:

a is an integer from 1 to 6;

c is an integer from 0 to 3; and

g is an integer from 2 to 6.

In some embodiments, the duocarmycin compound is Duocarmycin A, Duocarmycin B1, Duocarmycin B2, Duocarmycin C1, Duocarmycin C2, Duocarmycin D, CC-1065, Adozelesin, Bizelesin, or Carzelesin. Additional duocarmycin compounds suitable for the conjugates, scaffolds, and methods of the disclosure are described in U.S. Pat. No. 5,101,038.

In some embodiments the KSP inhibitor compound is a compound of Formula (XXVII), (XXVII), or (XXIX):

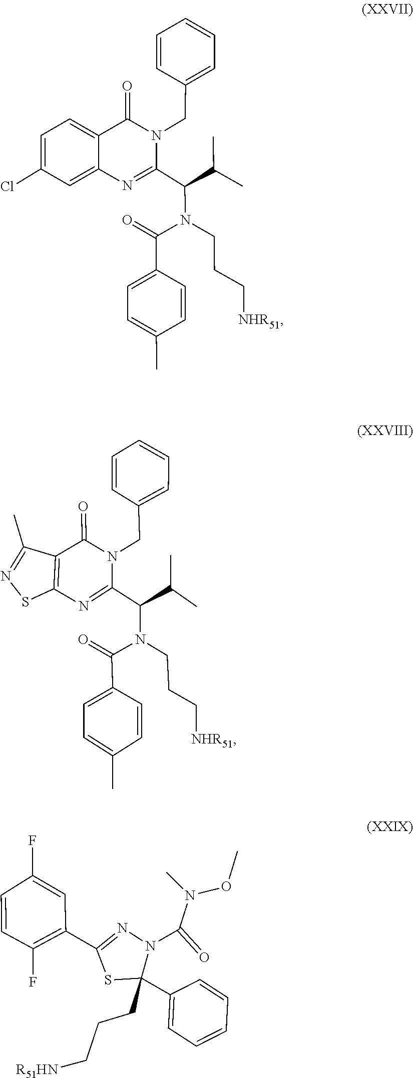

wherein:

R₅₁ is a bond, —C(O)—(CH₂)—C(O)NH—(CH₂)₂—NH—, —C(O)—(CH₂O—CH₂)—C(O)NH—(CH₂)₂—NH—, or R₁₁ is as defined herein.

One skilled in the art of therapeutic agents will readily understand that each of the therapeutic agents described herein can be modified in such a manner that the resulting compound still retains the specificity and/or activity of the original compound. The skilled artisan will also understand that many of these compounds can be used in place of the therapeutic agents described herein. Thus, the therapeutic agents disclosed herein include analogs and derivatives of the compounds described herein.

Table A below provides more examples of the therapeutic agents and derivatives thereof suitable for conjugation to form the antibody-drug conjugates or drug-carrying scaffolds of the disclosure. Spectral data of certain compounds are also provided (ND in the table means “not determined”). These examples may also be the active form of the drug when it is released from the conjugates in vitro or in vivo.

TABLE A
(VI1)

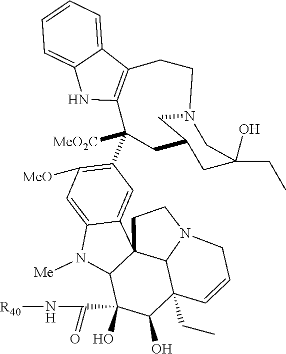

R₄₀

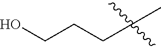 [SMILES: *CCCO]

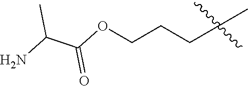 [SMILES: *CCCOC(=O)C(C)N]

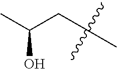 [SMILES: *C[C@H](C)O]

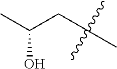 [SMILES: *C[C@@H](C)O]

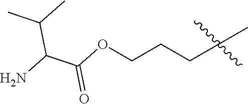 [SMILES: *CCCOC(=O)C(N)C(C)C]

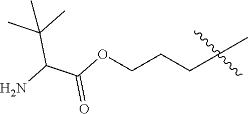 [SMILES: *CCCOC(=O)C(N)C(C)(C)C]

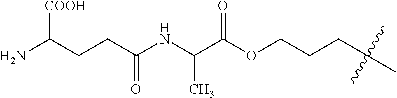 [SMILES: *CCCOC(=O)C(C)NC(=O)CCC(N)C(=O)O]

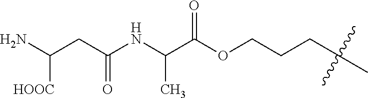 [SMILES: *CCCOC(=O)C(C)NC(=O)CC(N)C(=O)O]

(IX1)

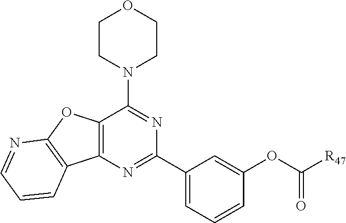

| R₄₇ | m/z |
|---|---|
| 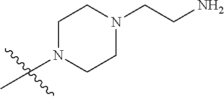 [SMILES: *N1CCN(CCN)CC1] | ND |
| 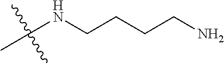 [SMILES: *NCCCCN] | ND |
| 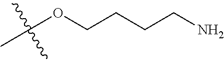 [SMILES: *OCCCCN] | ND |
| 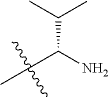 [SMILES: *[C@@H](N)C(C)C] | ND |

(XI)

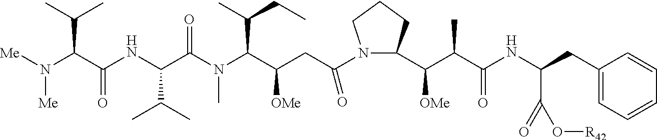

| R₄₂ | m/z |
|---|---|
| H | |
| —CH₃ | 760 |
| 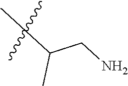 [SMILES: *C(C)CN] | 802.6 |
| 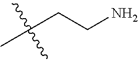 [SMILES: *CCN] | 790 |
| 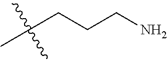 [SMILES: *CCCN] | 804 |

(XII)

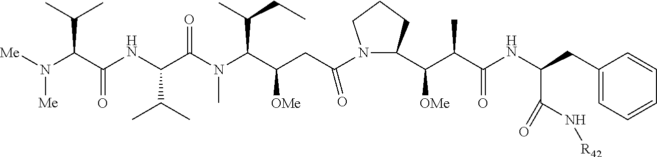

| R₄₀ | m/z |
|---|---|
| —H | |
| 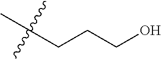 [SMILES: *CCCO] | 803.5 |
| 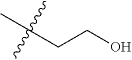 [SMILES: *CCO] | 789.1 |
| 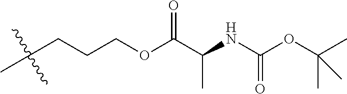 [SMILES: *CCCOC(=O)[C@H](C)NC(=O)OC(C)(C)C] | 974.2 |
| 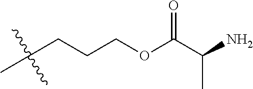 [SMILES: *CCCOC(=O)[C@H](C)N] | 874.5 |
| 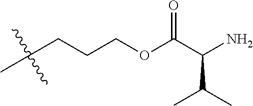 [SMILES: *CCCOC(=O)[C@@H](N)C(C)C] | 902.2 |
| 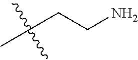 [SMILES: *CCN] | ND |
| 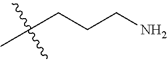 [SMILES: *CCCN] | ND |
| —OH | 788 |
| 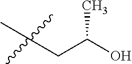 [SMILES: *C[C@H](C)O] | 803.4 |
| 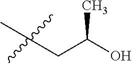 [SMILES: *C[C@@H](C)O] | 803.4 |
| 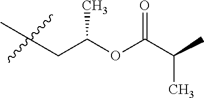 | 874.4 |
| 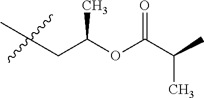 | 874.4 |
| 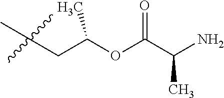 [SMILES: *C[C@H](C)OC(=O)[C@H](C)N] | 874.4 |
| 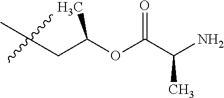 [SMILES: *C[C@@H](C)OC(=O)[C@H](C)N] | 874.4 |
| 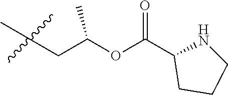 [SMILES: *C[C@H](C)OC(=O)[C@H]1CCCN1] | 900.2 |
| 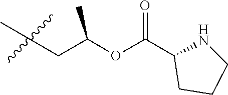 [SMILES: *C[C@@H](C)OC(=O)[C@H]1CCCN1] | 900.2 |
| 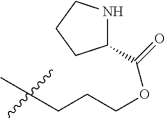 [SMILES: *CCCOC(=O)[C@@H]1CCCN1] | 900.5 |
| 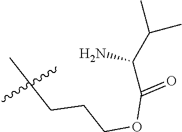 [SMILES: *CCCOC(=O)[C@H](N)C(C)C] | 900.5 |
| 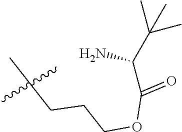 [SMILES: *CCCOC(=O)[C@H](N)C(C)(C)C] | 1016.6 |
| 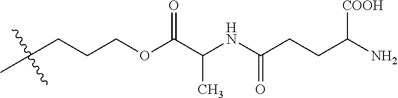 [SMILES: *CCCOC(=O)C(C)NC(=O)CCC(N)C(=O)O] | 989.5 |
| 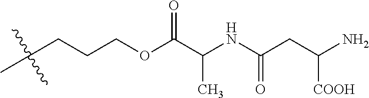 [SMILES: *CCCOC(=O)C(C)NC(=O)CC(N)C(=O)O] | 975.5 |

(XIII)

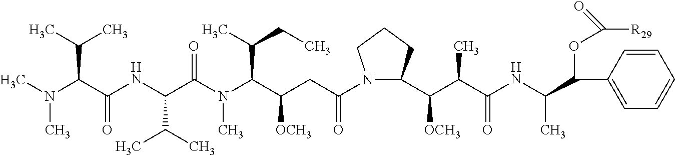

| —C(O)—R₂₉ | m/z |
|---|---|
| 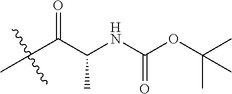 [SMILES: *C(=O)[C@@H](C)NC(=O)OC(C)(C)C] | 903.2 |
| 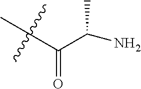 [SMILES: *C(=O)[C@H](C)N] | 803.1 |
| 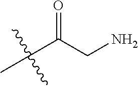 [SMILES: *C(=O)CN] | 790 |
| 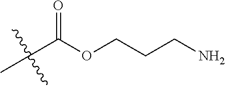 [SMILES: *C(=O)OCCCN] | 832.6 |
| 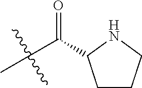 | 829.1 |
| 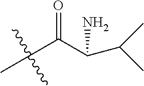 [SMILES: *C(=O)[C@@H](N)C(C)C] | 802 |

(XIV)

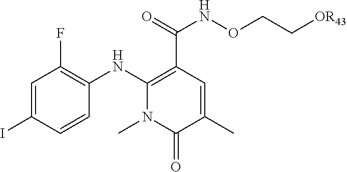

| R₄₃ | m/z |
|---|---|
| 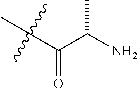 [SMILES: *C(=O)[C@H](C)N] | ND |
| 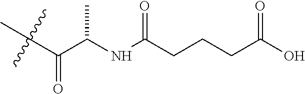 [SMILES: *C(=O)[C@H](C)NC(=O)CCCC(=O)O] | 644.9 |

(XVII)

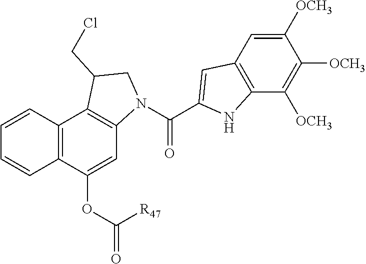

| R₄₇ | m/z |
|---|---|
| 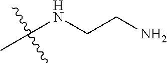 [SMILES: *NCCN] | 553.1 |
| 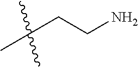 [SMILES: *CCN] | 538.1 |
| 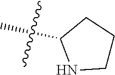 | 564.1 |
| 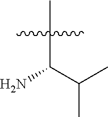 [SMILES: *[C@@H](N)C(C)C] | 566.1 |
| 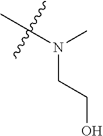 [SMILES: *N(C)CCO] | 568.1 |
| 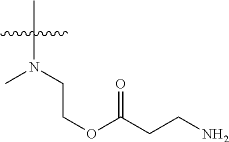 [SMILES: *N(C)CCOC(=O)CCN] | ND |
| 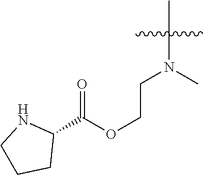 [SMILES: *N(C)CCOC(=O)[C@@H]1CCCN1] | ND |
| 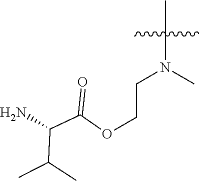 [SMILES: *N(C)CCOC(=O)[C@@H](N)C(C)C] | 667.2 |
| 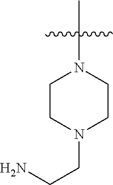 [SMILES: *N1CCN(CCN)CC1] | 622.2 |
| 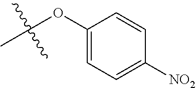 [SMILES: *Oc1ccc([N+](=O)[O-])cc1] | 632.02 |
| 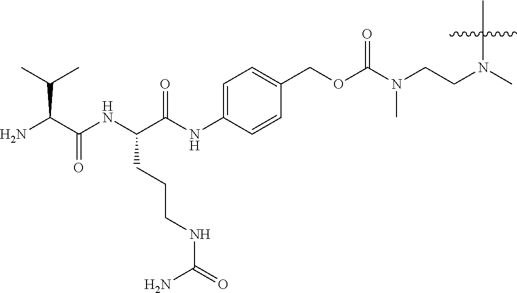 [SMILES: *N(C)CCN(C)C(=O)OCc1ccc(NC(=O)[C@H](CCCNC(N)=O)NC(=O)[C@@H](N)C(C)C)cc1] | 986.2 |
| 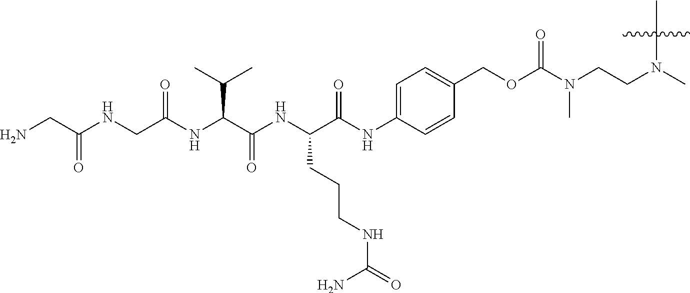 [SMILES: *N(C)CCN(C)C(=O)OCc1ccc(NC(=O)[C@H](CCCNC(N)=O)NC(=O)[C@@H](NC(=O)CNC(=O)CN)C(C)C)cc1] | ND |
| 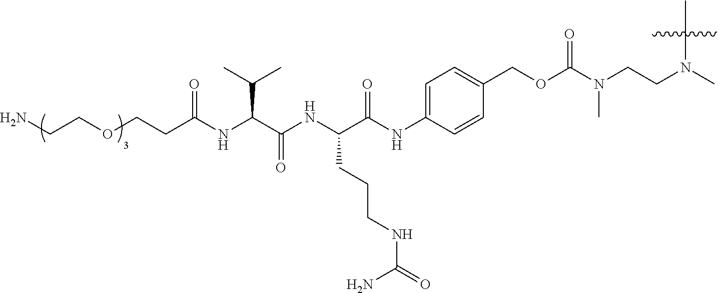 | ND |

(XXVII)

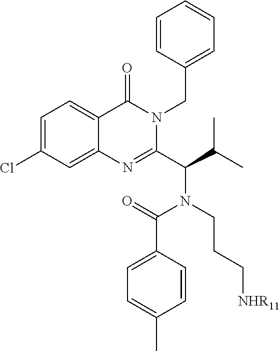

(XXVIII)

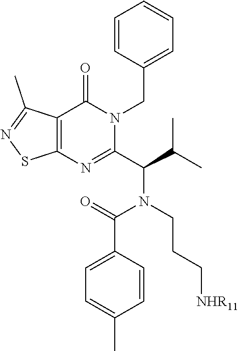

(XXIX)

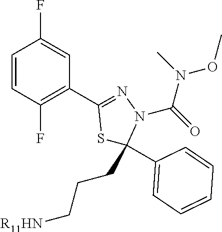

| R₁₁ | m/z (XXVII) |
|---|---|
| 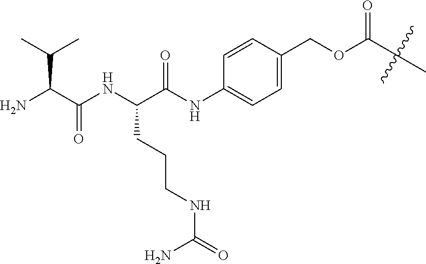 [SMILES: *C(=O)OCc1ccc(NC(=O)[C@H](CCCNC(N)=O)NC(=O)[C@@H](N)C(C)C)cc1] | 922.3 |
| 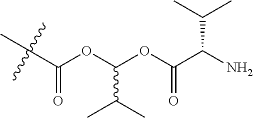 [SMILES: *C(=O)OC(OC(=O)[C@@H](N)C(C)C)C(C)C] | 732.2 |
| 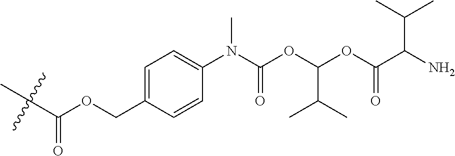 [SMILES: *C(=O)OCc1ccc(N(C)C(=O)OC(OC(=O)C(N)C(C)C)C(C)C)cc1] | ND |
| 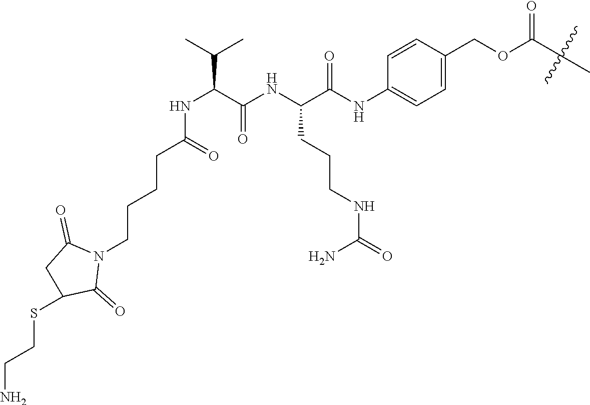 [SMILES: *C(=O)OCc1ccc(NC(=O)[C@H](CCCNC(N)=O)NC(=O)[C@@H](NC(=O)CCCCN2C(=O)CC(SCCN)C2=O)C(C)C)cc1] | ND |
| 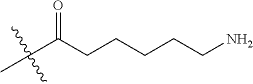 [SMILES: *C(=O)CCCCCN] | ND |
| 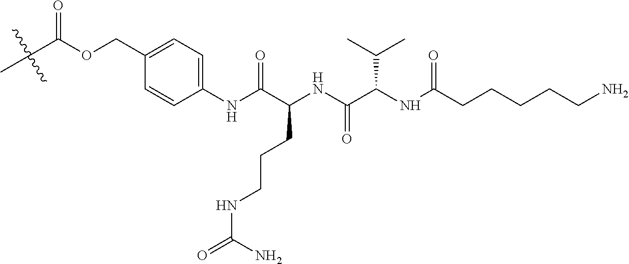 [SMILES: *C(=O)OCc1ccc(NC(=O)[C@H](CCCNC(N)=O)NC(=O)[C@@H](NC(=O)CCCCCN)C(C)C)cc1] | ND |
| 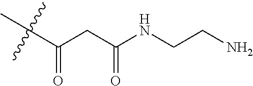 [SMILES: *C(=O)CC(=O)NCCN] | |
| 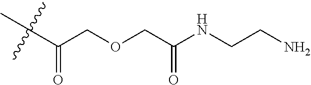 [SMILES: *C(=O)COCC(=O)NCCN] | |

### Hydrophilic Group or T¹

In some embodiments, the hydrophilic group included in the conjugates or scaffolds of the disclosure is a water-soluble and substantially non-antigenic polymer. Examples of the hydrophilic group, include, but are not limited to, polyalcohols, polyethers, polyanions, polycations, polyphosphoric acids, polyamines, polysaccharides, polyhydroxy compounds, polylysines, and derivatives thereof. In some embodiments, one end of the hydrophilic group can be functionalized so that it can be covalently attached to the Multifunctional Linker or M^{A} linker (e.g., to an amino acid in the M^{A} linker) by means of a non-cleavable linkage or via a cleavable linkage. In some embodiments, functionalization can be, for example, via an amine, thiol, NHS ester, maleimide, alkyne, azide, carbonyl, or other functional group. In some embodiments, the other terminus (or termini) of the hydrophilic group will be free and untethered. In some embodiments, by “untethered”, it is meant that the hydrophilic group will not be attached to another moiety, such as D or a Drug Unit, Releasable Assembly Unit, or other components of the conjugates or scaffolds of the disclosure. In some embodiments, the free and untethered end of the hydrophilic group may include a methoxy, carboxylic acid, alcohol or other suitable functional group. In some embodiments, the methoxy, carboxylic acid, alcohol, or other suitable functional group acts as a cap for the terminus or termini of the hydrophilic group.

In some embodiments, a cleavable linkage refers to a linkage that is not substantially sensitive to cleavage while circulating in the plasma but is sensitive to cleavage in an intracellular or intratumoral environment. In some embodiments, a non-cleavable linkage is one that is not substantially sensitive to cleavage in any biological environment. In some embodiments, chemical hydrolysis of a hydrazone, reduction of a disulfide, and enzymatic cleavage of a peptide bond or glycosidic linkage are examples of cleavable linkages. In some embodiments, exemplary attachments of the hydrophilic group are via amide linkages, ether linkages, ester linkages, hydrazone linkages, oxime linkages, disulfide linkages, peptide linkages, or triazole linkages. In some embodiments, the attachment of the hydrophilic group to the Multifunctional Linker or M^{A} linker (e.g., to an amino acid in the M^{A} linker) is via an amide linkage.

In some embodiments wherein the conjugate or scaffold of the disclosure comprises more than one hydrophilic groups, the multiple hydrophilic groups may be the same or different chemical moieties (e.g., hydrophilic groups of different molecular weight, number of subunits, or chemical structure). In some embodiments, the multiple hydrophilic groups can be attached to the Multifunctional Linker or M^{A} linker at a single attachment site or different sites.

In some embodiments, the addition of the hydrophilic group may have two potential impacts upon the pharmacokinetics of the resulting conjugate. In some embodiments, the desired impact is the decrease in clearance (and consequent in increase in exposure) that arises from the reduction in non-specific interactions induced by the exposed hydrophobic elements of the drug or drug-linker. In some embodiments, the undesired impact is the decrease in volume and rate of distribution that may arise from the increase in the molecular weight of the conjugate. In some embodiments, increasing the molecular weight of the hydrophilic group increases the hydrodynamic radius of a conjugate, resulting in decreased diffusivity that may diminish the ability of the conjugate to penetrate into a tumor. Because of these two competing pharmacokinetic effects, it may be desirable to use a hydrophilic group that is sufficiently large to decrease the conjugate clearance thus increasing plasma exposure, but not so large as to greatly diminish its diffusivity, which may reduce the ability of the conjugate to reach the intended target cell population.

In some embodiments, the hydrophilic group, includes, but is not limited to, a sugar alcohol (also known as polyalcohol, polyhydric alcohol, alditol or glycitol, such as inositol, glycerol, erythritol, threitol, arabitol, xylitol, ribitol, galactitol, mannitol, sorbitol, and the like) or a derivative thereof (e.g., amino polyalcohol), carbohydrate (e.g., a saccharide), a polyvinyl alcohol, a carbohydrate-based polymer (e.g., dextrans), a hydroxypropylmethacrylamide (HPMA), a polyalkylene oxide, and/or a copolymer thereof.

In some embodiments, the hydrophilic group comprises a plurality of hydroxyl (“—OH”) groups, such as moieties that incorporate monosaccharides, oligosaccharides, polysaccharides, and the like. In some embodiments the hydrophilic group comprises a plurality of —(CR₅₈OH)— groups, wherein R₅₈ is —H or C₁₋₈ alkyl.

In some embodiments, the hydrophilic group comprises one or more of the following fragments of the formula:

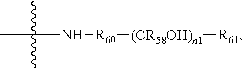

in which:

n₁ is an integer from 0 to about 6;

each R₅₈ is independently —H or C₁₋₈ alkyl;

R₆₀ is a bond, a C₁₋₆ alkyl linker, or —CHR₅₉— in which R₅₉ is —H, C₁₋₈ alkyl, cycloalkyl, or arylalkyl;

R₆₁ is CH₂OR₆₂, COOR₆₂, —(CH₂)ₙ₂COOR₆₂, or a heterocycloalkyl substituted with one or more hydroxyl;

R₆₂ is —H or C₁₋₈ alkyl; and

n₂ is an integer from 1 to about 5.

In some embodiments, R₅₈ is —H; R₆₀ is a bond or a C₁₋₆ alkyl linker; n₁ is an integer from 1 to about 6; and R₆₁ is CH₂OH or COOH. In some embodiments, R₅₈ is —H; R₆₀ is —CHR₅₉—; n₁ is 0; and R₆₁ is a heterocycloalkyl substituted with one or more hydroxyl, e.g., a monosaccharide.

In some embodiments, the hydrophilic group comprises a glucosyl-amine, a diamine, or a tri-amine.

In some embodiments, the hydrophilic group comprises one or more of the following fragments or a stereoisomer thereof;

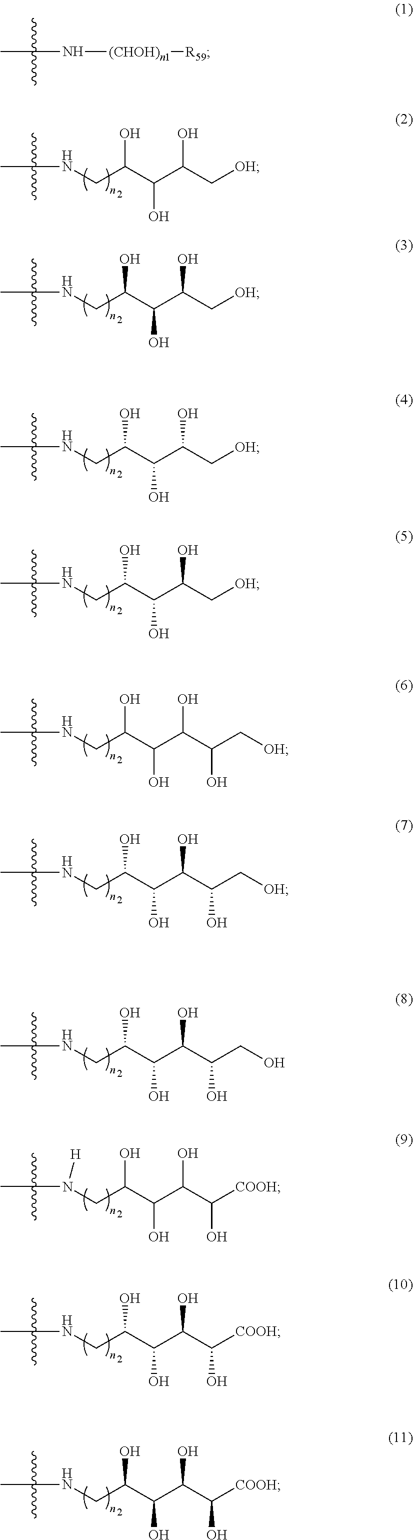

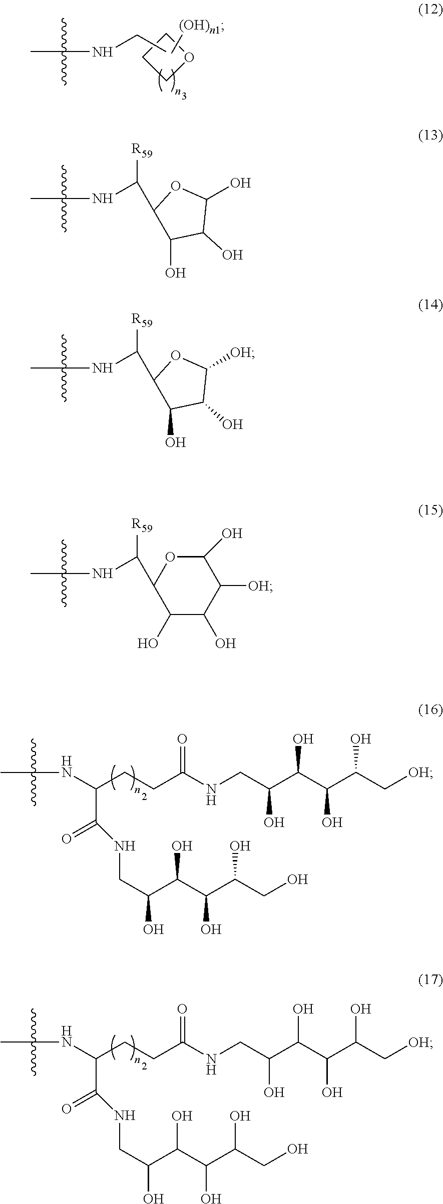

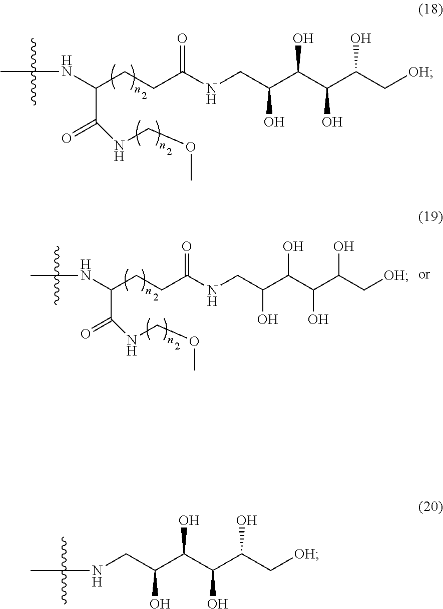

wherein:

R₅₉ is —H, C₁₋₈ alkyl, cycloalkyl, or arylalkyl;

n₁ is an integer from 1 to about 6;

n₂ is an integer from 1 to about 5; and

n₃ is an integer from about 1 to about 3.

It is understood that all stereochemical forms of the hydrophilic groups are contemplated herein. For example, in the above formula, the hydrophilic group may be derived from ribose, xylose, glucose, mannose, galactose, or other sugar and retain the stereochemical arrangements of pendant hydroxyl and alkyl groups present on those molecules. In some embodiments, it is to be understood that in the foregoing formulae, various deoxy compounds are also contemplated. Illustratively, one or more of the following features are contemplated for the hydrophilic groups when applicable:

In some embodiments, n₃ is 2 or 3.

In some embodiments, n₁ is 1, 2, or 3.

In some embodiments, n₂ is 1.

In some embodiments, R₅₉ is hydrogen.

In some embodiments, the hydrophilic group comprises:

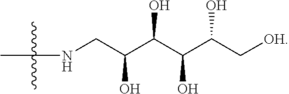 [SMILES: *NC[C@H](O)[C@@H](O)[C@H](O)[C@H](O)CO]

In some embodiments, the hydrophilic group comprises:

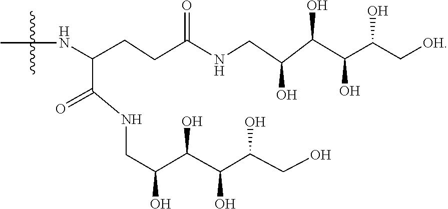 [SMILES: *NC(CCCC(=O)NC[C@H](O)[C@@H](O)[C@H](O)[C@H](O)CO)C(=O)NC[C@H](O)[C@@H](O)[C@H](O)[C@H](O)CO]

In some embodiments, the hydrophilic group comprises:

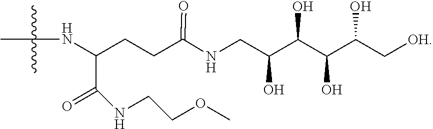 [SMILES: *NC(CCC(=O)NC[C@H](O)[C@@H](O)[C@H](O)[C@H](O)CO)C(=O)NCCOC]

In some embodiments, the hydrophilic group comprises

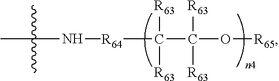

in which

n₄ is an integer from 1 to about 25;

each R₆₃ is independently —H or C₁₋₈ alkyl;

R₆₄ is a bond or a C₁₋₈ alkyl linker;

R₆₅ is —H, C₁₋₈ alkyl, or —(CH₂)ₙ₂COOR₂;

R₆₂ is —H or C₁₋₈ alkyl; and

n₂ is an integer from 1 to about 5.

In some embodiments, the hydrophilic group comprises:

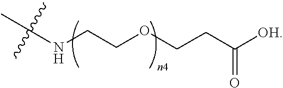

In some embodiments, n₄ is an integer from about 2 to about 20, from about 4 to about 16, from about 6 to about 12, or from about 8 to about 12.

In some embodiments, n₄ is an integer from about 2 to about 20. In some embodiments, n₄ is an integer from about 4 to about 16. In some embodiments, n₄ is an integer from about 6 to about 12. In some embodiments, n₄ is an integer from about 8 to about 12.

In some embodiments, n₄ is 6, 7, 8, 9, 10, 11, or 12.

In some embodiments, the hydrophilic group comprises a polyether, e.g., a polyalkylene glycol (PAO). PAO includes but is not limited to, polymers of lower alkylene oxides, in particular polymers of ethylene oxide, such as, for example, propylene oxide, polypropylene glycols, polyethylene glycol (PEG), polyoxyethylenated polyols, copolymers thereof, and block copolymers thereof. In other embodiments the polyalkylene glycol is a polyethylene glycol (PEG) including, but not limited to, polydisperse PEG, monodisperse PEG, and discrete PEG. Polydisperse PEGs are a heterogeneous mixture of sizes and molecular weights whereas monodisperse PEGs are typically purified from heterogeneous mixtures and are therefore provide a single chain length and molecular weight. In some embodiments, the PEG units are discrete PEGs provide a single molecule with defined and specified chain length. In some embodiments, the polyethylene glycol is mPEG.

In some embodiments, the hydrophilic group comprises a PEG unit which comprises one or multiple PEG chains. The PEG chains can be linked together, for example, in a linear, branched or star shaped configuration. The PEG unit, in addition to comprising repeating PEG subunits, may also comprise non-PEG material (e.g., to facilitate coupling of multiple PEG chains to each other or to facilitate coupling to the amino acid). Non-PEG material refers to the atoms in the PEG chain that are not part of the repeating —CH₂CH₂O— subunits. In some embodiments, the PEG chain can comprise two monomeric PEG chains linked to each other via non-PEG elements. In some embodiments, the PEG Unit can comprise two linear PEG chains attached to a central core that is attached to the amino acid (i.e., the PEG unit itself is branched).

The PEG unit may be covalently bound to the Multifunctional Linker or M_{A} linker (e.g., to an amino acid in the M^{A} linker) via a reactive group. Reactive groups are those to which an activated PEG molecule may be bound (e.g., a free amino or carboxyl group). In some embodiments, N-terminal amino acids and lysines (K) have a free amino group; and C-terminal amino acid residues have a free carboxyl group. Sulfhydryl groups (e.g., as found on cysteine residues) may also be used as a reactive group for attaching PEG.

In some embodiments, the PEG unit may be attached to the Multifunctional Linker or M^{A} linker (e.g., to an amino acid in the M^{A} linker) by using methoxylated PEG (“mPEG”) having different reactive moieties, including, but not limited to, succinimidyl succinate (SS), succinimidyl carbonate (SC), mPEG-imidate, para-nitrophenylcarbonate (NPC), succinimidyl propionate (SPA), and cyanuric chloride. Examples of mPEGs include, but are not limited to, mPEG-succinimidyl succinate (mPEG-SS), mPEG₂-succinimidyl succinate (mPEG₂-SS), mPEG-succinimidyl carbonate (mPEG-SC), mPEG₂-succinimidyl carbonate (mPEG₂-SC), mPEG-imidate, mPEG-para-nitrophenylcarbonate (mPEG-NPC), mPEG-imidate, mPEG₂-para-nitrophenylcarbonate (mPEG₂-NPC), mPEG-succinimidyl propionate (mPEG-SPA), mPEG₂-succinimidyl propionate (mPEG₂-SPA), mPEG-N-hydroxy-succinimide (mPEG-NHS), mPEG₂-N-hydroxy-succinimide (mPEG₂-NHS), mPEG-cyanuric chloride, mPEG₂-cyanuric chloride, mPEG₂-Lysinol-NPC, and mPEG₂-Lys-NHS. A wide variety of PEG species can be used, and substantially any suitable reactive PEG reagent can be used. In some embodiments, the reactive PEG reagent will result in formation of a carbamate or amide bond upon attachment to the Multifunctional Linker or M^{A} linker (e.g., to an amino acid in the M^{A} linker). The reactive PEG reagents include, but are not limited to, mPEG₂-N-hydroxy-succinimide (mPEG₂-NHS), bifunctional PEG propionaldehyde (mPEG₂-ALD), multi-Arm PEG, maleimide-containing PEG (mPEG(MAL)₂, mPEG₂(MAL)), mPEG-NH₂, mPEG-succinimidyl propionate (mPEG-SPA), succinimide of mPEG butanoate acid (mPEG-SBA), mPEG-thioesters, mPEG-double Esters, mPEG-BTC, mPEG-ButyrALD, mPEG-acetaldehyde diethyl acetal (mPEG-ACET), heterofunctional PEGs (e.g., NH₂-PEG-COOH, Boc-PEG-NHS, Fmoc-PEG-NHS, NHS-PEG-vinylsulfone (NHS-PEG-VS), or NHS-PEG-MAL), PEG acrylates (ACRL-PEG-NHS), PEG-phospholipids (e.g., mPEG-DSPE), multi-armed PEGs of the SUNBRITE™ series including the glycerine-based PEGs activated by a chemistry chosen by those skilled in the art, any SUNBRITE activated PEGs (including but not limited to carboxyl-PEGs, p-NP-PEGs, Tresyl-PEGs, aldehyde PEGs, acetal-PEGs, amino-PEGs, thiol-PEGs, maleimido-PEGs, hydroxyl-PEG-amine, amino-PEG-COOK hydroxyl-PEG-aldehyde, carboxylic anhydride type-PEG, functionalized PEG-phospholipid, and other similar and/or suitable reactive PEGs.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some such embodiments, the PEG unit comprises no more than about 72 subunits.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, or at least 20 subunits.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, or at least 18 subunits.

In some embodiments, the PEG unit comprises at least 6 subunits, at least 7 subunits, at least 8 subunits, at least 9 subunits, at least 10 subunits, at least 11 subunits, or at least 12 subunits.

In some embodiments, the PEG unit comprises at least 8 subunits, at least 9 subunits, at least 10 subunits, at least 11 subunits, or at least 12 subunits.

In some embodiments, the PEG unit comprises at least 6 subunits, at least 7 subunits, or at least 8 subunits.

In some embodiments, the PEG unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG unit comprises a combined total of at least 6 subunits, at least 8, at least 10 subunits, or at least 12 subunits. In some such embodiments, the PEG unit comprises no more than a combined total of about 72 subunits. In some such embodiments, the PEG unit comprises no more than a combined total of about 36 subunits.

In some embodiments, the PEG unit comprises a combined total of from 4 to 72, 4 to 60, 4 to 48, 4 to 36, or 4 to 24 subunits; from 5 to 72, 5 to 60, 5 to 48, 5 to 36, or 5 to 24 subunits; from 6 to 72, 6 to 60, 6 to 48, 6 to 36, or from 6 to 24 subunits; from 7 to 72, 7 to 60, 7 to 48, 7 to 36, or 7 to 24 subunits; from 8 to 72, 8 to 60, 8 to 48, 8 to 36, or 8 to 24 subunits; from 9 to 72, 9 to 60, 9 to 48, 9 to 36, or 9 to 24 subunits; from 10 to 72, 10 to 60, 10 to 48, 10 to 36, or 10 to 24 subunits; from 11 to 72, 11 to 60, 11 to 48, 11 to 36, or 11 to 24 subunits; from 12 to 72, 12 to 60, 12 to 48, 12 to 36, or 12 to 24 subunits; from 13 to 72, 13 to 60, 13 to 48, 13 to 36, or 13 to 24 subunits; from 14 to 72, 14 to 60, 14 to 48, 14 to 36, or 14 to 24 subunits; from 15 to 72, 15 to 60, 15 to 48, 15 to 36, or 15 to 24 subunits; from 16 to 72, 16 to 60, 16 to 48, 16 to 36, or 16 to 24 subunits; from 17 to 72, 17 to 60, 17 to 48, 17 to 36, or 17 to 24 subunits; from 18 to 72, 18 to 60, 18 to 48, 18 to 36, or 18 to 24 subunits; from 19 to 72, 19 to 60, 19 to 48, 19 to 36, or 19 to 24 subunits; from 20 to 72, 20 to 60, 20 to 48, 20 to 36, or 20 to 24 subunits; from 21 to 72, 21 to 60, 21 to 48, 21 to 36, or 21 to 24 subunits; from 22 to 72, 22 to 60, 22 to 48, 22 to 36, or 22 to 24 subunits; from 23 to 72, 23 to 60, 23 to 48, 23 to 36, or 23 to 24 subunits; or from 24 to 72, 24 to 60, 24 to 48, 24 to 36 subunits.

In some embodiments, the PEG unit comprises one or more linear PEG chains having a combined total of from 4 to 72, 4 to 60, 4 to 48, 4 to 36, or 4 to 24 subunits; from 5 to 72, 5 to 60, 5 to 48, 5 to 36, or 5 to 24 subunits; from 6 to 72, 6 to 60, 6 to 48, 6 to 36, or 6 to 24 subunits; from 7 to 72, 7 to 60, 7 to 48, 7 to 36, or 7 to 24 subunits; from 8 to 72, 8 to 60, 8 to 48, 8 to 36, or 8 to 24 subunits; from 9 to 72, 9 to 60, 9 to 48, 9 to 36, or 9 to 24 subunits; from 10 to 72, 10 to 60, 10 to 48, 10 to 36, or 10 to 24 subunits; from 11 to 72, 11 to 60, 11 to 48, 11 to 36, or 11 to 24 subunits; from 12 to 72, 12 to 60, 12 to 48, 12 to 36, or 12 to 24 subunits; from 13 to 72, 13 to 60, 13 to 48, 13 to 36, or 13 to 24 subunits; from 14 to 72, 14 to 60, 14 to 48, 14 to 36, or 14 to 24 subunits; from 15 to 72, 15 to 60, 15 to 48, 15 to 36, or 15 to 24 subunits; from 16 to 72, 16 to 60, 16 to 48, 16 to 36, or 16 to 24 subunits; from 17 to 72, 17 to 60, 17 to 48, 17 to 36, or 17 to 24 subunits; from 18 to 72, 18 to 60, 18 to 48, 18 to 36, or 18 to 24 subunits; from 19 to 72, 19 to 60, 19 to 48, 19 to 36, or 19 to 24 subunits; from 20 to 72, 20 to 60, 20 to 48, 20 to 36, or 20 to 24 subunits; from 21 to 72, 21 to 60, 21 to 48, 21 to 36, or 21 to 24 subunits; from 22 to 72, 22 to 60, 22 to 48, 22 to 36, or 22 to 24 subunits; from 23 to 72, 23 to 60, 23 to 48, 23 to 36, or 23 to 24 subunits; or from 24 to 72, 24 to 60, 24 to 48, 24 to 36 subunits.

In some embodiments, the PEG unit is a derivatized linear single PEG chain having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits.

In some embodiments, the PEG unit is a derivatized linear single PEG chain having from 6 to 72, 6 to 60, 6 to 48, 6 to 36, or 6 to 24 subunits; from 7 to 72, 7 to 60, 7 to 48, 7 to 36, or 7 to 24 subunits; from 8 to 72, 8 to 60, 8 to 48, 8 to 36, or 8 to 24 subunits; from 9 to 72, 9 to 60, 9 to 48, 9 to 36, or 9 to 24 subunits; from 10 to 72, 10 to 60, 10 to 48, 10 to 36, or 10 to 24 subunits; from 11 to 72, 11 to 60, 11 to 48, 11 to 36, or 11 to 24 subunits; from 12 to 72, 12 to 60, 12 to 48, 12 to 36, or 12 to 24 subunits; from 13 to 72, 13 to 60, 13 to 48, 13 to 36, or 13 to 24 subunits; from 14 to 72, 14 to 60, 14 to 48, 14 to 36, or 14 to 24 subunits; from 15 to 72, 15 to 60, 15 to 48, 15 to 36, or 15 to 24 subunits; from 16 to 72, 16 to 60, 16 to 48, 16 to 36, or 16 to 24 subunits; from 17 to 72, 17 to 60, 17 to 48, 17 to 36, or 17 to 24 subunits; from 18 to 72, 18 to 60, 18 to 48, 18 to 36, or 18 to 24 subunits; from 19 to 72, 19 to 60, 19 to 48, 19 to 36, or 19 to 24 subunits; from 20 to 72, 20 to 60, 20 to 48, 20 to 36, or 20 to 24 subunits; from 21 to 72, 21 to 60, 21 to 48, 21 to 36, or 21 to 24 subunits; from 22 to 72, 22 to 60, 22 to 48, 22 to 36, or 22 to 24 subunits; from 23 to 72, 23 to 60, 23 to 48, 23 to 36, or 23 to 24 subunits; or from 24 to 72, 24 to 60, 24 to 48, 24 to 36 subunits.

In some embodiments, the PEG unit is a derivatized linear single PEG chain having from 2 to 72, 2 to 60, 2 to 48, 2 to 36, or 2 to 24 subunits; from 2 to 72, 2 to 60, 2 to 48, 2 to 36, or 2 to 24 subunits; from 3 to 72, 3 to 60, 3 to 48, 3 to 36, or 3 to 24 subunits; from 3 to 72, 3 to 60, 3 to 48, 3 to 36, or 3 to 24 subunits; from 4 to 72, 4 to 60, 4 to 48, 4 to 36, or 4 to 24 subunits; or from 5 to 72, 5 to 60, 5 to 48, 5 to 36, or 5 to 24 subunits.

In some embodiments, a linear PEG unit is:

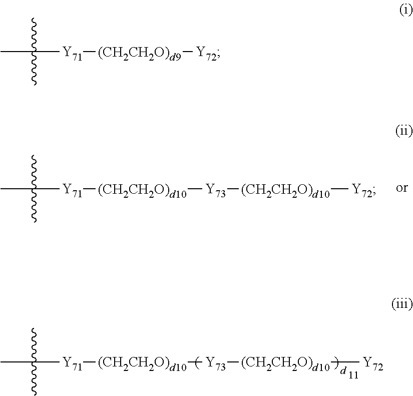

wherein;

[symbol] indicates site of attachment to the Multifunctional Linker or M^{A} linker (e.g., to an amino acid in the M^{A} linker);

Y₇₁ is a PEG attachment unit;

Y₇₂ is a PEG capping unit;

Y₇₃ is an PEG coupling unit (i.e., for coupling multiple PEG subunit chains together);

d₉ is an integer from 2 to 72;

each d₁₀ is independently an integer from 1 to 72.

d₁₁ is an integer from 2 to 5.

In some embodiments, d₉ is an integer from 2 to 72. In some embodiments, d₉ is an integer from 4 to 72. In some embodiments, d₉ is an integer from 6 to 72, from 8 to 72, from 10 to 72, from 12 to 72, or from 6 to 24.

In some embodiments, d₉ is an integer from 6 to 72. In some embodiments, d₉ is an integer from 8 to 72. In some embodiments, d₉ is an integer from 10 to 72. In some embodiments, d₉ is an integer from 12 to 72. In some embodiments, d₉ is an integer from 6 to 24.

In some embodiments, there are at least 6 PEG subunits in the PEG unit. In some embodiments, there are no more than 72 or 36 PEG subunits in the PEG unit.

In some embodiments, there are at least 8 PEG subunits in the PEG unit. In some embodiments, there are at least 10 PEG subunits in the PEG unit. In some embodiments, there are at least 12 PEG subunits in the PEG unit.

In some embodiments, d₉ is 8 or about 8, 12 or about 12, 24 or about 24.

In some embodiments, each Y₇₂ is independently —C₁₋₁₀ alkyl, —C₂₋₁₀ alkyl-CO₂H, —C₂₋₁₀ alkyl-OH, —C₂₋₁₀ alkyl-NH₂, —C₂₋₁₀ alkyl-NH(C₁₋₃ alkyl), or C₂₋₁₀ alkyl-N(C₁₋₃ alkyl)₂.

In some embodiments, Y₇₂ is —C₁₋₁₀ alkyl, —C₂₋₁₀ alkyl-CO₂H, —C₂₋₁₀ alkyl-OH, or —C₂₋₁₀ alkyl-NH₂.

In some embodiments, the PEG coupling unit is part of the PEG unit and is non-PEG material that acts to connect two or more chains of repeating CH₂CH₂O— subunits. In some embodiments, the PEG coupling unit Y₇₃ is —C₂₋₁₀ alkyl-C(O)—NH—, —C₂₋₁₀ alkyl-NH—C(O)—, —C₂₋₁₀ alkyl-NH—, —C₂₋₁₀ alkyl-C(O)—, —C₂₋₁₀ alkyl-O—, or —C₂₋₁₀ alkyl-S—.

In some embodiments, each Y₇₃ is independently —C₁₋₁₀ alkyl-C(O)—NH—, —C₁₋₁₀ alkyl-NH—C(O)—, —C₂₋₁₀ alkyl-NH—, —C₂₋₁₀ alkyl-O—, —C₁₋₁₀ alkyl-S—, or —C₁₋₁₀ alkyl-NH—.

In some embodiments, the PEG attachment unit is part of the PEG unit and acts to link the PEG unit to the Multifunctional Linker or M^{A} linker (e.g., to an amino acid in the M^{A} linker). In some embodiments, the amino acid has a functional group that forms a bond with the PEG Unit. In some embodiments, the functional groups for attachment of the PEG unit to the amino acid include sulfhydryl groups to form disulfide bonds or thioether bonds, aldehyde, ketone, or hydrazine groups to form hydrazone bonds, hydroxylamine to form oxime bonds, carboxylic or amino groups to form peptide bonds, carboxylic or hydroxy groups to form ester bonds, sulfonic acids to form sulfonamide bonds, alcohols to form carbamate bonds, and amines to form sulfonamide bonds or carbamate bonds or amide bonds. In some embodiments, the PEG unit can be attached to the amino acid, for example, via a disulfide, thioether, hydrazone, oxime, peptide, ester, sulfonamide, carbamate, or amide bond. In some embodiments, the reaction for attaching the PEG unit can be a cycloaddition, addition, addition/elimination or substitution reaction, or a combination thereof when applicable.

In some embodiments, the PEG attachment unit Y₇₁ is a bond, —C(O)—, —O—, —S—, —S(O)—, —S(O)₂—, —NR₅—, —C(O)O—, —C(O)—C₁₋₁₀ alkyl, —C(O)—C₁₋₁₀ alkyl-O—, —C(O)—C₁₋₁₀ alkyl-CO₂—, —C(O)—C₁₋₁₀ alkyl-NR₅—, —C(O)—C₁₋₁₀ alkyl-S—, —C(O)—C₁₋₁₀ alkyl-C(O)—NR—, —C(O)—C₁₋₁₀ alkyl-NR₅—C(O)—, —C₁₋₁₀ alkyl, —C₁₋₁₀ alkyl-O—, —C₁₋₁₀ alkyl-CO₂—, —C₁₋₁₀ alkyl-NR₅—, —C₁₋₁₀ alkyl-S—, —C₁₋₁₀ alkyl-C(O)—NR₅—, —C₁₋₁₀ alkyl-NR₅—C(O)—, —CH₂CH₂SO₂—C₁₋₁₀ alkyl-, —CH₂C(O)—C₁₋₁₀ alkyl-, ═N—(O or N)—C₁₋₁₀ alkyl-O—, ═N—(O or N)—C₁₋₁₀ alkyl-NR₅—, ═N—(O or N)—C₁₋₁₀ alkyl-CO₂—, ═N—(O or N)—C₁₋₁₀ alkyl-S—,

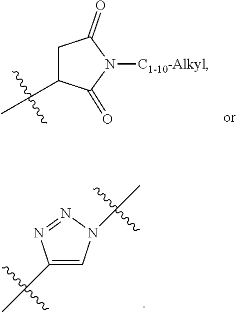

In some embodiments, Y₇₁ is —NH—, —C(O)—, a triazole group, —S—, or a maleimido-group such as

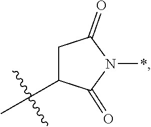

wherein [symbol] indicates attachment to the Multifunctional Linker or M^{A} linker (e.g., to an amino acid in the M^{A} linker) and the * indicates the site of attachment within the PEG Unit.

Examples of linear PEG units include:

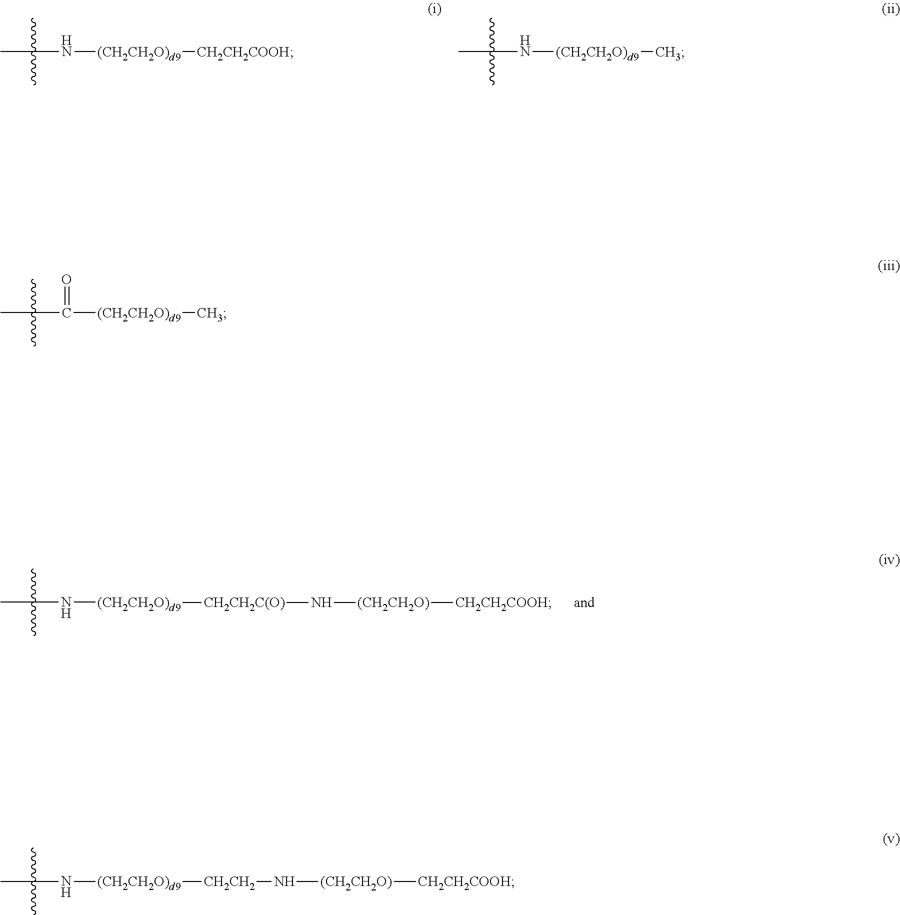

wherein

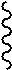

indicates site of attachment to the Multifunctional Linker or M^{A} linker (e.g., to an amino acid in the M^{A} linker), and each d₉ is independently an integer from 4 to 24, 6 to 24, 8 to 24, 10 to 24, 12 to 24, 14 to 24, or 16 to 24.

In some embodiments, d₉ is about 8, about 12, or about 24.

In some embodiments, the PEG unit is from about 300 Da to about 5 kDa; from about 300 Da to about 4 kDa; from about 300 Da to about 3 kDa; from about 300 Da to about 2 kDa; or from about 300 Da to about 1 kDa. In some embodiments, the PEG unit has at least 6 subunits or at least 8, 10 or 12 subunits. In some embodiments, the PEG unit has at least 6 subunits or at least 8, 10 or 12 subunits but no more than 72 subunits. In some embodiments, the PEG unit has at least 6 subunits or at least 8, 10 or 12 subunits but no more than 36 subunits.

In some embodiments, suitable polyethylene glycols may have a free hydroxy group at each end of the polymer molecule, or may have one hydroxy group etherified with a lower alkyl, e.g., a methyl group. In some embodiments suitable for the practice of the present disclosure are derivatives of polyethylene glycols having esterifiable carboxy groups. In some embodiments, polyethylene glycols are commercially available under the trade name PEG, usually as mixtures of polymers characterized by an average molecular weight. In some embodiments, polyethylene glycols having an average molecular weight from about 300 to about 5000. In some embodiments, polyethylene glycols having an average molecular weight from about 600 to about 1000.

In some embodiments, examples of hydrophilic groups that are suitable for the conjugates, scaffolds, and methods disclosed herein can be found in e.g., U.S. Pat. No. 8,367,065 column 13; U.S. Pat. No. 8,524,696 column 6; WO2015/057699 and WO 2014/062697, the contents of each of which are hereby incorporated by reference in their entireties.

### Cysteine Engineered Targeting Moieties

In some embodiments, the cysteine engineered targeting moiety directs the conjugates comprising a peptide linker to specific tissues, cells, or locations in a cell. In some embodiments, the cysteine engineered targeting moiety comprises an engineered cysteine.

In some embodiments, the cysteine engineered targeting moiety is a protein-based recognition molecule (PBRM).

In some embodiments, the cysteine engineered protein-based recognition molecule directs the conjugates comprising a peptide linker to specific tissues, cells, or locations in a cell. In some embodiments, the cysteine engineered protein-based recognition molecule can direct the conjugate in culture or in a whole organism, or both. In each case, the cysteine engineered protein-based recognition molecule may have a ligand that is present on the cell surface of the targeted cell(s) to which it binds with an effective specificity, affinity, and avidity. In some embodiments, the cysteine engineered protein-based recognition molecule targets the conjugate to tissues other than the liver. In some embodiments the cysteine engineered protein-based recognition molecule targets the conjugate to a specific tissue such as the liver, kidney, lung, or pancreas. The cysteine engineered protein-based recognition molecule can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. Cysteine engineered protein-based recognition molecules can direct the conjugate to specific types of cells such as specific targeting to hepatocytes in the liver as opposed to Kupffer cells. In some embodiments, cysteine engineered protein-based recognition molecules can direct the conjugate to cells of the reticular endothelial or lymphatic system, or to professional phagocytic cells such as macrophages or eosinophils. In some embodiments, the conjugate itself may also be an effective delivery system, without the need for specific targeting.

In some embodiments, the cysteine engineered protein-based recognition molecule can target the conjugate to a location within the cell, such as the nucleus, the cytoplasm, or the endosome, for example. In some embodiments, the cysteine engineered protein-based recognition molecule can enhance cellular binding to receptors, or cytoplasmic transport to the nucleus and nuclear entry or release from endosomes or other intracellular vesicles.

In some embodiments, the cysteine engineered protein-based recognition molecule is an antibody, an antibody fragment, a protein, a peptide, or a peptide mimic.

In some embodiments, the cysteine engineered protein-based recognition molecule is an antibody. In some embodiments, the cysteine engineered protein-based recognition molecule is an antibody fragment. In some embodiments, the cysteine engineered protein-based recognition molecule is a protein. In some embodiments, the cysteine engineered protein-based recognition molecule is a peptide. In some embodiments, the cysteine engineered protein-based recognition molecule is a peptide mimic.

In some embodiments, the cysteine engineered antibody or antibody fragment is an antibody or antibody fragment in which one or more amino acids of the corresponding parent antibody or antibody fragment (e.g., the corresponding wild type antibody or antibody fragment) are substituted with cysteines (e.g., engineered cysteine). In some embodiments, the parent antibody or antibody fragment may be wild type or mutated.

In some embodiments, the cysteine engineered antibody or antibody fragment may be a mutated antibody or antibody fragment. In some embodiments, a monoclonal antibody known in the art is engineered to form the cysteine engineered antibody. In some embodiments, an antibody fragment (e.g., a Fab antibody fragment) known in the art is engineered to form the cysteine engineered antibody fragment (e.g., a cysteine engineered Fab antibody fragment). In some embodiments, a single site mutation of a Fab gives a single cysteine engineered residue in a Fab whereas a single site mutation in an antibody yields two cysteine engineered amino acids in the resulting antibody due to the dimeric nature of the IgG antibody.

In some embodiments, the cysteine engineered antibody or antibody fragment retains the antigen binding capability of its corresponding wild type antibody or antibody fragment. In some embodiments, the cysteine engineered antibody or antibody fragment is capable of binding to the one or more antigens for its corresponding wild type antibody or antibody fragment.

In some embodiments, the engineered cysteine is not a part of an intrachain or interchain disulfide unit. In some embodiments, the engineered cysteine contains a free thiol group that is reactive with an electrophilic functionality. In some embodiments, the engineered cysteine (e.g., the free thiol group thereof) on the antibody or antibody fragment surface may allow for conjugation of the antibody or antibody fragment with a Linker-Drug moiety comprising a thiol-reactive group (e.g., a maleimide or a haloacetyl).

It is understood that substituting one or more non-cysteine amino acids in an antibody or antibody fragment with cysteines may create one or more engineered cysteines as available sites for conjugation. In some embodiments, by substituting a non-cysteine amino acid in an antibody or antibody fragment with cysteine, a reactive thiol group is positioned as an accessible site of the antibody or antibody fragment and may be used to conjugate the antibody or antibody fragment to other moieties (e.g., drug moieties, or Linker-Drug moieties), and to create the conjugate of the present disclosure. In some embodiments, the amino acid at V205 (Kabat numbering) of the light chain of a parent antibody or antibody fragment is substituted with cysteine. In some embodiments, cysteine engineered antibodies may be generated as described, e.g., in U.S. Pat. No. 7,521,541.

In some embodiments, the cysteine engineered protein-based recognition molecule comprises an engineered cysteine, and the cysteine engineered protein-based recognition molecule is conjugated to the Linker-Drug moiety by forming a covalent bond via the sulfhydryl group of the engineered cysteine and a functional group of the Linker-Drug moiety.

In some embodiments, exemplary cysteine engineered antibodies or antibodies derived from Fab, Fab2, scFv or camel antibody heavy-chain fragments specific to the cell surface markers, include, but are not limited to, 5T4, AOC3, ALK, AXL, C242, C4.4a, CA-125, CCLI 1, CCR 5, CD2, CD3, CD4, CD5, CD15, CA15-3, CD18, CD19, CA19-9, CDH6, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD 154, CD326, CEA, CEACAM-5, clumping factor, CTLA-4, CXCR2, EGFR (HER1), ErbB1, ErbB2, ErbB3, EpCAM, EPHA2, EPHB2, EPHB4, FGFR (i.e. FGFR1, FGFR2, FGFR3, FGFR4), FLT3, folate receptor, FAP, GD2, GD3, GPNMB, GCC (GUCY2C), HGF, HER2, HER3, HMI.24, ICAM, ICOS-L, IGF-1 receptor, VEGFR1, EphA2, TRPV1, CFTR, gpNMB, CA9, Cripto, c-KIT, c-MET, ACE, APP, adrenergic receptor-beta2, Claudine 3, LIV1, LY6E, Mesothelin, MUC1, MUC13, NaPi2b, NOTCH1, NOTCH2, NOTCH3, NOTCH4, RON, ROR1, PD-L1, PD-L2, PTK7, B7-H3, B7-B4, IL-2 receptor, IL-4 receptor, IL-13 receptor, TROP-2, frizzled-7, integrins (including α₄, αᵥβ₃, αᵥβ₅, αᵥβ₆, α₁β₄, α₄β₁, α₄β₇, α₅β₁, α₆β₄, α_{IIIb}β₃ integrins), IFN-α, IFN-γ, IgE, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, myostatin, NCA-90, NGF, PDGFRα, phosphatidylserine, prostatic carcinoma cell, Pseudomonas aeruginosa, rabies, RANKL, respiratory syncytial virus, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGF-A, VEGFR2, vimentin, and the like.

In some embodiments the cysteine engineered antibodies or cysteine engineered antibody derived from Fab, Fab2, scFv or camel antibody heavy-chain fragments specific to the cell surface markers include CA-125, C242, CD3, CD19, CD22, CD25, CD30, CD31, CD33, CD37, CD40, CD44, CD51, CD54, CD56, CD62E, CD62P, CD62L, CD70, CD138, CD141, CD326, CEA, CTLA-4, EGFR (HER1), ErbB2, ErbB3, FAP, folate receptor, IGF-1 receptor, GD3, GPNMB, HGF, HER2, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mucin, MUC1, NaPi2b, B7H3, B7H4, C4.4a, CEACAM-5, MUC13, TROP-2, frizzled-7, Mesothelin, IL-2 receptor, IL-4 receptor, IL-13 receptor and integrins (including αᵥβ₃, αᵥβ₅, αᵥβ₆, α₁β₄, α₄β₁, α₅β₁, α₆β₄ integrins), tenascin C, TRAIL-R2, and vimentin.

Exemplary cysteine engineered antibodies include 3F8, abagovomab, abciximab (REOPRO®), adalimumab (HUMIRA®), adecatumumab, afelimomab, afutuzumab, alacizumab, ALD518, alemtuzumab (CAMPATH®), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN®), belimumab (BENLYSTA®), benralizumab, bertilimumab, besilesomab (SCINITIMUN®), bevacizumab (AVASTIN®), biciromab (FIBRISCINT®), bivatuzumab, blinatumomab, brentuximab, briakinumab, canakinumab (ILARIS), cantuzumab, capromab, catumaxomab (REMOVAB®), CC49, cedelizumab, certolizumab, cetuximab (ERBITUX®), citatuzumab, cixutumumab, clenoliximab, clivatuzumab, conatumumab, CR6261, dacetuzumab, daclizumab (ZENAPAX®), daratumumab, denosumab (PROLIA®), detumomab, dorlimomab, dorlixizumab, ecromeximab, eculizumab (SOLIRIS), edobacomab, edrecolomab (PANOREX®), efalizumab (RAPTIVA®), efungumab (MYCOGRAB®), elotuzumab, elsilimomab, enlimomab, epitumomab, epratuzumab, erlizumab, ertumaxomab (REXOMUN®), etaracizumab (ABEGRIN®), exbivirumab, fanolesomab (NEUTROSPEC®), faralimomab, farletuzumab, felvizumab, fezakinumab, figitumumab, fontolizumab (HuZAF), foravirumab, fresolimumab, galiximab, gantenerumab, gavilimomab, gemtuzumab, girentuximab, glembatumumab, golimumab (SIMPONI®), gomiliximab, ibalizumab, ibritumomab, igovomab (INDIMACIS-125), imciromab (MYOSCINT®), infliximab (REMICADE®), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA®), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI®), nebacumab, necitumumab, nerelimomab, nimotuzumab (THERACIM®), nofetumomab, ocrelizumab, odulimomab, ofatumumab (ARZERRA®), olaratumab, omalizumab (XOLAIR®), ontecizumab, oportuzumab, oregovomab (OVAREX®), otelixizumab, pagibaximab, palivizumab (SYNAGIS®), panitumumab (VECTIBIX), panobacumab, pascolizumab, pemtumomab (THERAGYN®), pertuzumab (OMNITARG®), pexelizumab, pintumomab, priliximab, pritumumab, PRO 140, rafivirumab, ramucirumab, ranibizumab (LUCENTIS®), raxibacumab, regavirumab, reslizumab, rilotumumab, rituximab (RITUXAN®), robatumumab, rontalizumab, rovelizumab (LEUKARREST®), ruplizumab (ANTOVA®), satumomab pendetide, sevirumab, sibrotuzumab, sifalimumab, siltuximab, siplizumab, solanezumab, sonepcizumab, sontuzumab, stamulumab, sulesomab (LEUKOSCAN®), tacatuzumab (AFP-CIDE®), tetraxetan, tadocizumab, talizumab, tanezumab, taplitumomab paptox, tefibazumab (AUREXIS®), telimomab, tenatumomab, teneliximab, teplizumab, TGN1412, ticilimumab (tremelimumab), tigatuzumab, TNX-650, tocilizumab (atlizumab, ACTEMRA®), toralizumab, tositumomab (BEXXAR®), trastuzumab (HERCEPTIN®), tremelimumab, tucotuzumab, tuvirumab, urtoxazumab, ustekinumab (STELERA®), vapaliximab, vedolizumab, veltuzumab, vepalimomab, visilizumab (NUVION®), volociximab (HUMASPECT®), votumumab, zalutumumab (HuMEX-EGFr), zanolimumab (HuMAX-CD4), ziralimumab, and zolimomab.

In some embodiments, the cysteine engineered antibodies are directed to cell surface markers for 5T4, CA-125, CEA, CDH6, CD3, CD19, CD20, CD22, CD30, CD33, CD40, CD44, CD51, CTLA-4, CEACAM5, EpCAM, HER2, EGFR (HER1), FAP, folate receptor, GCC (GUCY2C), HGF, integrin αᵥβ₃, integrin α₅β₁, IGF-1 receptor, GD3, GPNMB, mucin, LIV1, LY6E, mesothelin, MUC1, MUC13, PTK7, phosphatidylserine, prostatic carcinoma cells, PDGFR α, TAG-72, tenascin C, TRAIL-R2, VEGF-A and VEGFR2. In this embodiment the antibodies are abagovomab, adecatumumab, alacizumab, altumomab, anatumomab, arcitumomab, bavituximab, bevacizumab (AVASTIN®), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN®), rilotumumab, robatumumab, satumomab, sibrotuzumab, taplitumomab, tenatumomab, tenatumomab, ticilimumab (tremelimumab), tigatuzumab, trastuzumab (HERCEPTIN®), tositumomab, tremelimumab, tucotuzumab celmoleukin, volociximab, and zalutumumab.

In specific embodiments the cysteine engineered antibodies directed to cell surface markers for HER2 are pertuzumab or trastuzumab and for EGFR (HER1) the antibody is cetuximab or panitumumab; and for CD20 the antibody is rituximab and for VEGF-A is bevacizumab and for CD-22 the antibody is epratuzumab or veltuzumab and for CEA the antibody is labetuzumab.

Exemplary cysteine engineered peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In some embodiments, the cysteine engineered peptides or peptide mimics are LHRH receptor targeting peptides and ErbB2 (HER2) receptor targeting peptides

Exemplary cysteine engineered proteins comprise insulin, transferrin, fibrinogen-gamma fragment, thrombospondin, claudin, apolipoprotein E, Affibody molecules such as, for example, ABY-025, Ankyrin repeat proteins, ankyrin-like repeats proteins and synthetic peptides.

In some embodiments, the cysteine engineered protein-drug conjugates comprise broad spectrum cytotoxins in combination with cell surface markers for HER2, such as, for example, pertuzumab or trastuzumab; for EGFR such as cetuximab and panitumumab; for CEA such as labetuzumab; for CD20 such as rituximab; for VEGF-A such as bevacizumab; or for CD-22 such as epratuzumab or veltuzumab.

In some embodiments, the cysteine engineered protein-drug conjugates or cysteine engineered protein conjugates used in the disclosure comprise combinations of two or more cysteine engineered protein-based recognition molecules, such as, for example, combination of bispecific antibodies directed to the EGF receptor (EGFR) on tumor cells and to CD3 and CD28 on T cells; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3-CD19 plus CD28-CD22 bispecific antibodies.

In some embodiments, the cysteine engineered protein-drug conjugates or cysteine engineered protein conjugates used in the disclosure comprise protein-based recognition molecules are antibodies against antigens, such as, for example, Trastuzumab, Cetuximab, Rituximab, Bevacizumab, Epratuzumab, Veltuzumab, Labetuzumab, B7-H4, B7-H3, CA125, CDH6, CD33, CXCR2, CEACAM5, EGFR, FGFR1, FGFR2, FGFR3, FGFR4, GCC (GUCY2C), HER2, LIV1, LY6E, NaPi2b, c-Met, mesothelin, NOTCH1, NOTCH2, NOTCH3, NOTCH4, PD-L1, PTK7, c-Kit, MUC1, MUC13. and 5T4.

In some embodiments, the cysteine engineered protein-drug conjugates or cysteine engineered protein conjugates of the disclosure comprise protein-based recognition molecules which are antibodies against 5T4, such as, for example a humanized anti-5T4 scFvFc antibody.

Examples of suitable 5T4 targeting ligands or immunoglobulins include those which are commercially available, or have been described in the patent or non-patent literature, e.g., U.S. Pat. Nos. 8,044,178, 8,309,094, 7,514,546, EP1036091 (commercially available as TroVax™, Oxford Biomedica), EP2368914A1, WO 2013041687 A1 (Amgen), US 2010/0173382, and P. Sapra, et al., Mol. Cancer Ther. 2013, 12:38-47. An anti-5T4 antibody is disclosed in U.S. Provisional Application No. 61/877,439, filed Sep. 13, 2013 and U.S. Provisional Application No. 61/835,858, filed Jun. 17, 2013. The contents of each of the patent documents and scientific publications are herein incorporated by reference in their entireties.

As used herein, the term “5T4 antigen-binding portion” refers to a polypeptide sequence capable of selectively binding to a 5T4 antigen. In exemplary conjugates, the 5T4 antigen-binding portion generally comprises a single chain scFv-Fc form engineered from an anti-5T4 antibody. A single-chain variable fragment (scFv-Fc) is a fusion protein of the variable regions of the heavy (VH) and light chains (VL) of an immunoglobulin, connected with a linker peptide, and further connected to an Fc region comprising a hinge region and CH2 and CH3 regions of an antibody (any such combinations of antibody portions with each other or with other peptide sequences is sometimes referred to herein as an “immunofusion” molecule). Within such a scFvFc molecule, the scFv section may be C-terminally linked to the N-terminus of the Fc section by a linker peptide.

In some embodiments, the cysteine engineered protein-drug conjugates or cysteine engineered protein conjugates of the disclosure comprise protein-based recognition molecules which are Her-2 or NaPi2b antibodies.

For example the cysteine engineered Her-2 antibody suitable for the conjugate or scaffold of the disclosure comprises a variable heavy chain complementarity determining region 1 (CDRH1) comprising the amino acid sequence FTFSSYSMN (SEQ ID NO: 25); a variable heavy chain complementarity determining region 2 (CDRH2) comprising the amino acid sequence YISSSSSTIYYADSVKG (SEQ ID NO: 26); a variable heavy chain complementarity determining region 3 (CDRH3) comprising the amino acid sequence GGHGYFDL (SEQ ID NO: 27); a variable light chain complementarity determining region 1 (CDRL1) comprising the amino acid sequence RASQSVSSSYLA (SEQ ID NO: 28); a variable light chain complementarity determining region 2 (CDRL2) comprising the amino acid sequence GASSRAT (SEQ ID NO: 21); and a variable light chain complementarity determining region 3 (CDRL3) comprising the amino acid sequence QQYHHSPLT (SEQ ID NO: 29) (see, e.g., U.S. Pat. No. 9,738,720 issued Aug. 22, 2107).

In some embodiments, the cysteine engineered NaPi2b antibody suitable for the conjugate or scaffold of the disclosure comprises a variable light chain complementarity determining region 1 (CDRL1) comprising the amino acid sequence SASQDIGNFLN (SEQ ID NO: 8); a variable light chain complementarity determining region 2 (CDRL2) comprising the amino acid sequence YTSSLYS (SEQ ID NO: 9); a variable light chain complementarity determining region 3 (CDRL3) comprising the amino acid sequence QQYSKLPLT (SEQ ID NO: 10); a variable heavy chain complementarity determining region 1 (CDRH1) comprising the amino acid sequence GYTFTGYNIH (SEQ ID NO: 5); a variable heavy chain complementarity determining region 2 (CDRH2) comprising the amino acid sequence AIYPGNGDTSYKQKFRG (SEQ ID NO: 6); and a variable heavy chain complementarity determining region 3 (CDRH3) comprising the amino acid sequence GETARATFAY (SEQ ID NO: 7) (see, e.g., co-pending application U.S. Ser. No. 15/457,574 filed Mar. 13, 2017).

### Cysteine Engineered PBRM-Drug Conjugates

In some embodiments, conjugates of the disclosure comprise one or more occurrences of D, wherein D is a therapeutic agent, e.g., a drug, wherein the one or more occurrences of D may be the same or different.

In some embodiments, one or more occurrences of engineered cysteine of the cysteine engineered PBRM (e.g., at light chain V205C) is attached to the Linker-Drug moiety, wherein the one or more occurrences of cysteine engineered PBRM may be the same or different. In some embodiments, one or more Linker-Drug moieties that comprises one or more occurrences of D are connected to one cysteine engineered PBRM (e.g., a cysteine engineered antibody).

In some embodiments, D is (a) an auristatin compound; (b) a calicheamicin compound; (c) a duocarmycin compound; (d) SN38; (e) a pyrrolobenzodiazepine; (f) a vinca compound; (g) a tubulysin compound; (h) a non-natural camptothecin compound; (i) a maytansinoid compound: (j) a DNA binding drug; (k) a kinase inhibitor; (l) a MEK inhibitor; (m) a KSP inhibitor; (n) a topoisomerase inhibitor; (o) a DNA-alkylating drug; (p) a RNA polymerase; (q) a PARP inhibitor; (r) a NAMPT inhibitor; (s) a topoisomerase inhibitor; (t) a protein synthesis inhibitor; (u) a DNA-binding drug; (v) a DNA intercalation drug; or (w) an immunomodulatory compound.

In some embodiments, D is (a) an auristatin compound; (b) a calicheamicin compound; (c) a duocarmycin compound; (d) a topoisomerase inhibitor; (e) a pyrrolobenzodiazepine compound; (f) a vinca compound; (g) a protein synthesis inhibitor; (h) a RNA polymerase inhibitor; (i) a tubulin binding compound; or (j) a NAMPT inhibitor, or an analog thereof.

In some embodiments, D is (a) an auristatin compound: (b) a calicheamicin compound; (c) a duocarmycin compound; (d) a camptothecin compound; (e) a pyrrolobenzodiazepine compound; or (f) a vinca compound; or an analog thereof.

In some embodiments, the auristatin compound is auristatin, dolastatin, monomethylauristatin E (MMAE), monomethylauristatin F (MMAF), auristatin F, AF-HPA, MMA-HPA, or phenylenediamine (AFP).

In some embodiments, the duocarmycin or an analog thereof is duocarmycin A, duocarmycin B1, duocarmycin B2, duocarmycin C1, duocarmycin C2, duocarmycin D, duocarmycin SA, CC-1065, adozelesin, bizelesin, or carzelesin.

In some embodiments, the camptothecin compound is camptothecin, CPT-11 (irinotecan), SN-38, or topotecan.

In some embodiments, the pyrrolobenzodiazepine compound is a pyrrolobenzodiazepine monomer, a symmetrical pyrrolobenzodiazepine dimer, or an unsymmetrical pyrrolobenzodiazepine dimer.

The PBRM-drug conjugate of the disclosure comprise a cysteine engineered PBRM that has a molecular weight of about 40 kDa or greater (e.g., about 60 kDa or greater; about 80 kDa or greater; about 100 kDa or greater; about 120 kDa or greater; about 140 kDa or greater; about 160 kDa or greater; about 180 kDa or greater; or about 200 kDa or greater, or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa).

In some embodiments, the cysteine engineered PBRM has a molecular weight of about 40 kDa or greater (e.g., about 60 kDa or greater; about 80 kDa or greater; about 100 kDa or greater; about 120 kDa or greater; about 140 kDa or greater; about 160 kDa or greater; about 180 kDa or greater; or about 200 kDa or greater; or about 40-200 kDa, about 40-180 kDa, about 40-140 kDa, about 60-200 kDa, about 60-180 kDa, about 60-140 kDa, about 80-200 kDa, about about 80-180 kDa, about 80-140 kDa, about 100-200 kDa, about 100-180 kDa, or about 100-140 kDa) and has a sulfhydryl (i.e., —SH or thiol) group.

In some embodiments, the total number of sulfide bonds formed between the Linker-drug moieties and the cysteine engineered PBRM (or total number of attachment points) is 10 or less (e.g., 2 or less).

In some embodiments, for conjugation with one or more Linker-Drug moieties, the cysteine engineered PBRM has a molecular weight of about 40 kDa or greater (e.g., about 60 kDa or greater, about 80 kDa or greater, about 100 kDa or greater, about 120 kDa or greater, about 140 kDa or greater, about 160 kDa or greater, or about 180 kDa or greater; or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa).

In some embodiments, for conjugation with one or more Linker-Drug moieties, the cysteine engineered PBRM has a molecular weight of about 40 kDa to about 200 kDa.

In some embodiments, for conjugation with one or more Linker-Drug moieties, the cysteine engineered PBRM has a molecular weight of about 40 kDa to about 80 kDa.

In some embodiments, for conjugation with one or more Linker-Drug moieties, the cysteine engineered PBRM has a molecular weight of 40 kDa to 200 kDa.

In some embodiments, for conjugation with one or more Linker-Drug moieties, the PBRM has a molecular weight of 40 kDa to 80 kDa.

In some embodiments, cysteine engineered PBRMs in this molecular weight range include, but are not limited to, for example, antibody fragments, such as, for example, Fabs.

In some embodiments, for conjugation with one or more Linker-Drug moieties, the cysteine engineered PBRM has a molecular weight of about 60 kDa to about 120 kDa.

In some embodiments, for conjugation with one or more Linker-Drug moieties, the cysteine engineered PBRM has a molecular weight of 60 kDa to 120 kDa.

In some embodiments, PBRMs in this molecular weight range include, but are not limited to, for example, camelids, Fab2, scFvFc, and the like.

In some embodiments, for conjugation with one or more Linker-Drug moieties, the cysteine engineered PBRM has a molecular weight of about 140 kDa to about 180 kDa.

In some embodiments, for conjugation with one or more Linker-Drug moieties, the cysteine engineered PBRM has a molecular weight of 140 kDa to 180 kDa.

In some embodiments, PBRMs in this molecular weight range include, but are not limited to, for example, full length antibodies, such as, IgG, IgM.

In some embodiments, these cysteine engineered targeting ligands, the linkers and the drug or prodrug fragments described herein can be assembled into the conjugate or scaffold of the disclosure, for example according to the disclosed techniques and methods. Therapeutic and targeting conjugates of the disclosure, and methods for producing them, are described below by way of non-limiting example.

In some embodiments, the total number of sulfide bonds formed between the Linker-Drug moiety and the cysteine engineered PBRM (or total number of attachment points) is 2 or less.

In some embodiments, the total number of sulfide bonds formed between the Linker-Drug moiety and the cysteine engineered PBRM (or total number of attachment points) is 2.

In some embodiments, the total number of sulfide bonds formed between the Linker-Drug moiety and the cysteine engineered PBRM (or total number of attachment points) is 1.

In some embodiments, the total number of sulfide bonds formed between the Linker-Drug moiety and the cysteine engineered PBRM (or total number of attachment points) is 4 or less.

In some embodiments, the total number of sulfide bonds formed between the Linker-Drug moiety and the cysteine engineered PBRM (or total number of attachment points) is 4.

In some embodiments, the total number of sulfide bonds formed between the Linker-Drug moiety and the cysteine engineered PBRM (or total number of attachment points) is 3.

In some embodiments, the total number of sulfide bonds formed between the Linker-Drug moiety and the cysteine engineered PBRM (or total number of attachment points) is 6 or less.

In some embodiments, the total number of sulfide bonds formed between the Linker-Drug moiety and the cysteine engineered PBRM (or total number of attachment points) is 6.

In some embodiments, the total number of sulfide bonds formed between the Linker-Drug moiety and the cysteine engineered PBRM (or total number of attachment points) is 5.

In some embodiments, the ratio between Linker-Drug moiety and the PBRM is between about 1:1 and about 2:1.

In some embodiments, the ratio between Linker-Drug moiety and the PBRM is between about 1:1 and about 4:1.

In some embodiments, the ratio between Linker-Drug moiety and the PBRM is between about 1:1 and about 6:1.

In some embodiments, the ratio between Linker-Drug moiety and the PBRM is between about 2:1 and about 4:1.

In some embodiments, the ratio between Linker-Drug moiety and the PBRM is between about 2:1 and about 6:1.

In some embodiments, the ratio between Linker-Drug moiety and the PBRM is between about 4:1 and about 6:1.

In some embodiments, the disclosure also relates to a Linker-Drug moiety comprising at least two moieties, in which each moiety is capable of conjugation to a thiol group at the light chain V205C in a PBRM so as to form a cysteine engineered protein-Linker-Drug conjugate.

In some embodiments, one or more thiol groups of the one or more engineered cysteines of a PBRM are produced by reducing a protein. The one or more thiol groups of the one or more engineered cysteines the PBRM may then react with one or more Linker-Drug moieties that are capable of conjugation to a thiol group from the engineered cysteine so as to conjugate the cysteine engineered PBRM with the Linker-Drug moiety. In some embodiments, the at least two moieties connected to the cysteine engineered PBRM are maleimide groups.

In some embodiments, the cysteine engineered antibodies may be activated for conjugation with Linker-Drug moiety by treatment with a reducing agent such as DTT (Cleland's reagent, dithiothreitol) or TCEP (tris(2-carboxyethyl)phosphine hydrochloride). In some embodiments, full length, cysteine engineered monoclonal antibodies can be reduced with an excess of TCEP to reduce disulfide bonds (e.g., between the newly introduced engineered cysteine and the cysteine present in the corresponding parent antibodies) to yield a reduced form of the antibody. The interchain disulfide bonds between paired cysteine residues may be reformed under partial oxidation conditions, e.g., dilute aqueous copper sulfate (CuSO₄) or other oxidants known in the art. Such mild, partial reoxidation step may allow for forming intrachain disulfides efficiently with high fidelity. The newly introduced and unpaired engineered cysteine may remain available for reaction with Linker-Drug moiety to form the antibody conjugates of the present disclosure. In some embodiments, an excess of Linker-drug moiety is added to effect conjugation and form the cysteine engineered antibody-drug conjugate, and the conjugation mixture is purified to remove excess Linker-drug intermediate and other impurities.

In some embodiments, the free thiol groups of the engineered cysteine in the cysteine engineered PBRM, which are used for the conjugation, are derived from cysteine residues at the light chain V205C (Kabat numbering) of a native protein.

In some embodiments, for conjugating of the Linker-Drug moiety, a cysteine engineered PBRM has a molecular weight of 40 kDa or greater (e.g., 60 kDa or greater; 80 kDa or greater; or 100 kDa or greater; 120 kDa or greater; 140 kDa or greater; 160 kDa or greater or 180 kDa or greater). In some embodiments, the ratio of cysteine engineered PBRM per Linker-Drug moiety is between about 1:1 and about 1:6; between about 1:1 and about 1:5; between about 1:1 and about 1:4; between about 1:1 and about 1:3; or between about 1:1 and about 1:2.

PBRMs in this molecular weight range include, but are not limited to, for example, full length antibodies, such as, IgG, IgM.

In some embodiments, for conjugation with one or more Linker-Drug moieties a cysteine engineered PBRM has a molecular weight of 60 kDa to 120 kDa. In some embodiments, the ratio of PBRM per Linker-Drug moiety is between about 1:1 and about 1:6; between about 1:1 and about 1:5; between about 1:1 and about 1:4; between about 1:1 and about 1:3; or between about 1:1 and about 1:2.

Cysteine engineered PBRMs in this molecular weight range include, but are not limited to, for example, antibody fragments such as, for example Fab2, scFcFv and camelids.

In some embodiments, for conjugation with one or more Linker-Drug moieties a PBRM has a molecular weight of 40 kDa to 80 kDa. In some embodiments, the ratio of PBRM per Linker-Drug moiety is between about 1:1 and about 1:6; between about 1:1 and about 1:5; between 1:1 and about 1:4; between about 1:1 and about 1:3, or between about 1:1 and about 1:2.

In some embodiments, cysteine engineered PBRMs in this molecular weight range include, but are not limited to, for example, antibody fragments, such as, Fabs.

In some embodiments, the disclosure features a scaffold useful to conjugate with either or both of a cysteine engineered protein-based recognition-molecule (PBRM) and a therapeutic agent (D), e.g., the scaffold of any of Formulae (II)-(XXIX) disclosed herein.

In some embodiments, the drug-carrying scaffolds (i.e., without linking to a cysteine engineered PBRM), described herein each typically have a polydispersity index (PDI) of 1.

Conjugates and scaffolds disclosed herein can be purified (i.e., removal of any starting materials) by extensive diafiltration. If necessary, additional purification by size exclusion chromatography can be conducted to remove any aggregated conjugates. In general, the conjugates as purified typically contain less than 5% (e.g., <2% w/w) aggregated conjugates as determined by SEC; less than 0.5% (e.g., <0.1% w/w) free (unconjugated) drug as determined by RP-HPLC; less than 1% drug carrying-peptide-containing scaffolds as determined by SEC and less than 2% (e.g., <1% w/w) unconjugated cysteine engineered PBRM as determined by HIC-HPLC.

Tables B and C below provide examples of the drug carrying-peptide-containing scaffolds and the conjugates of the disclosure respectively.

TABLE B
Structure

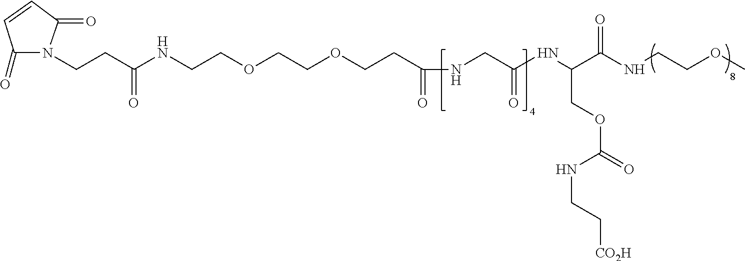

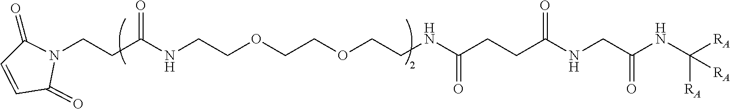

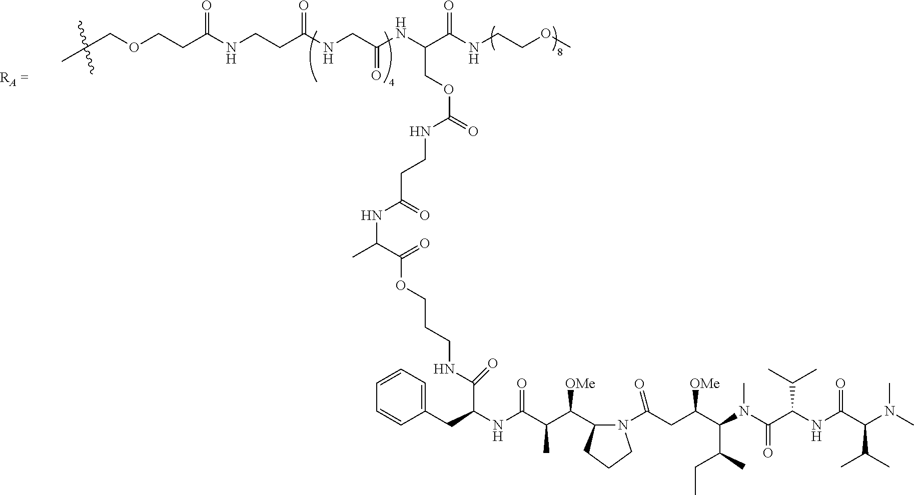

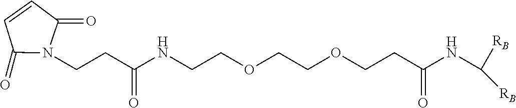

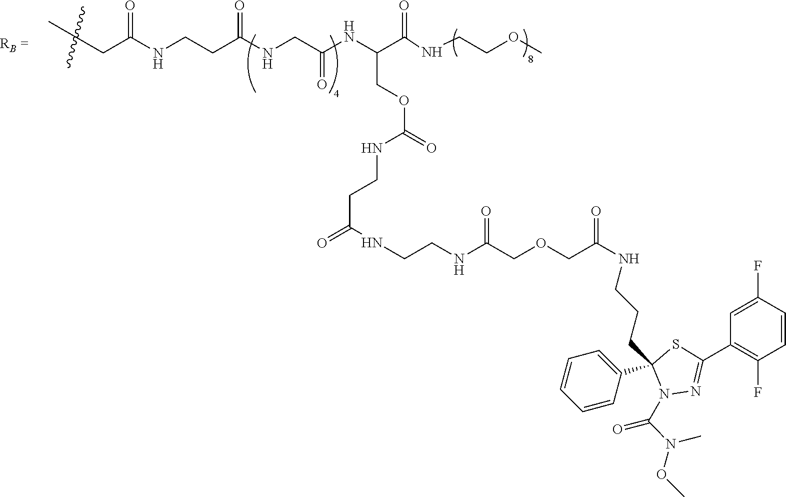

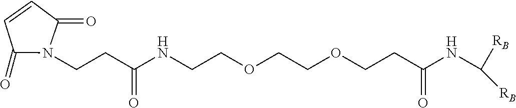

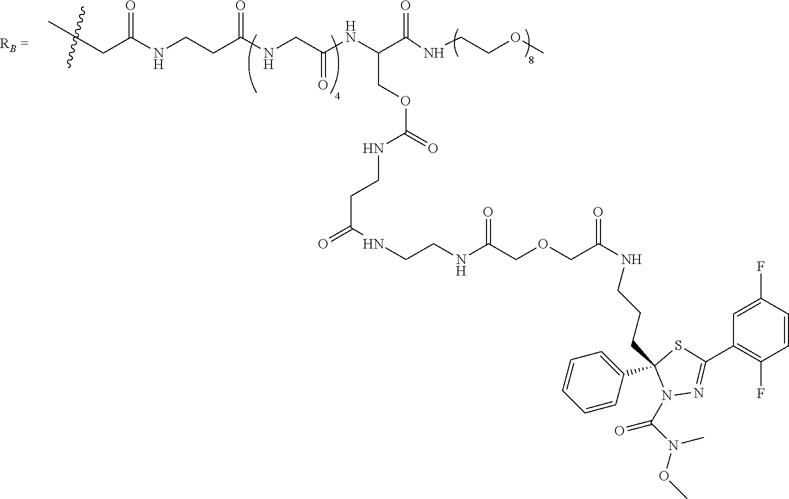

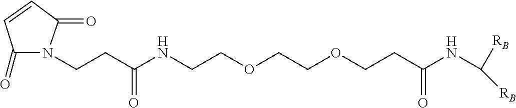

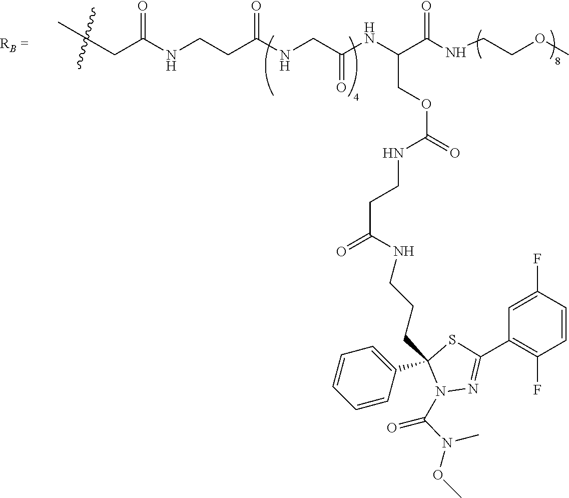

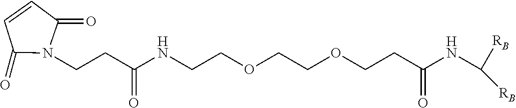

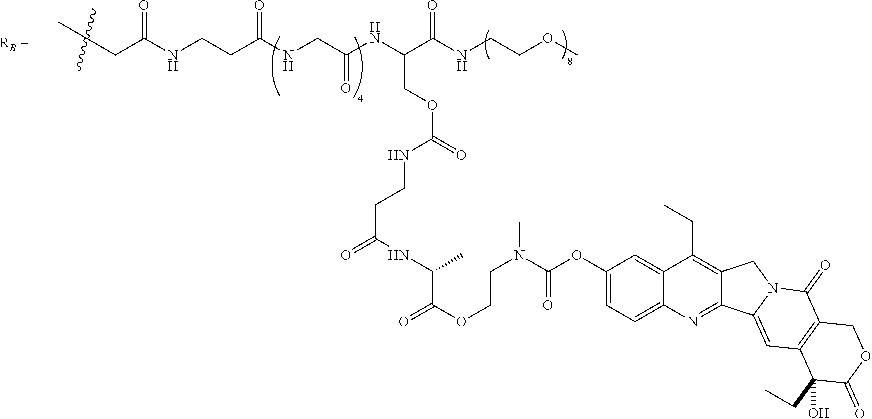

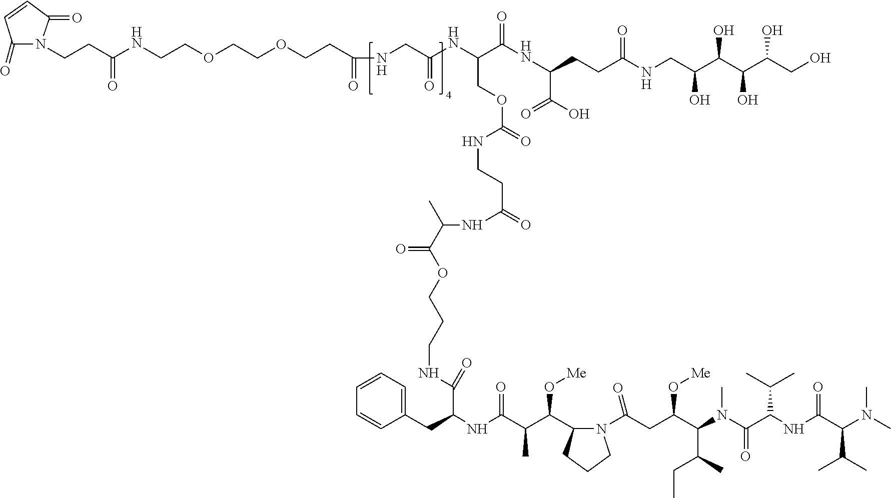

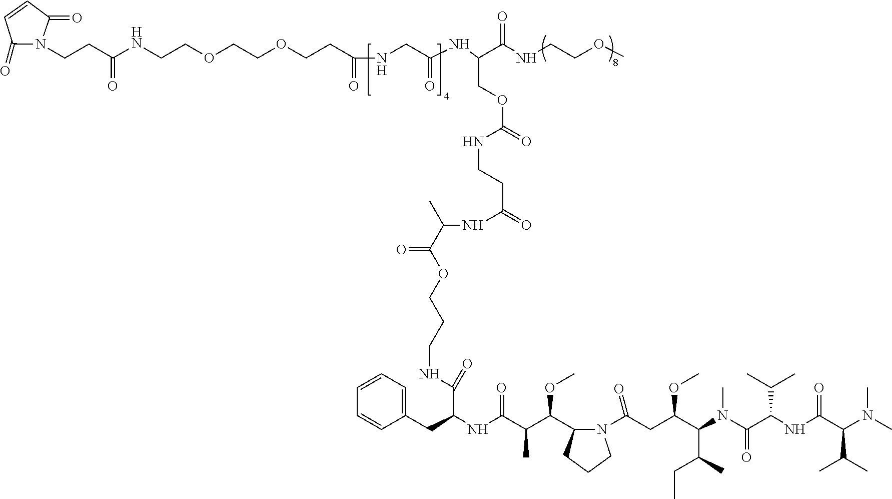

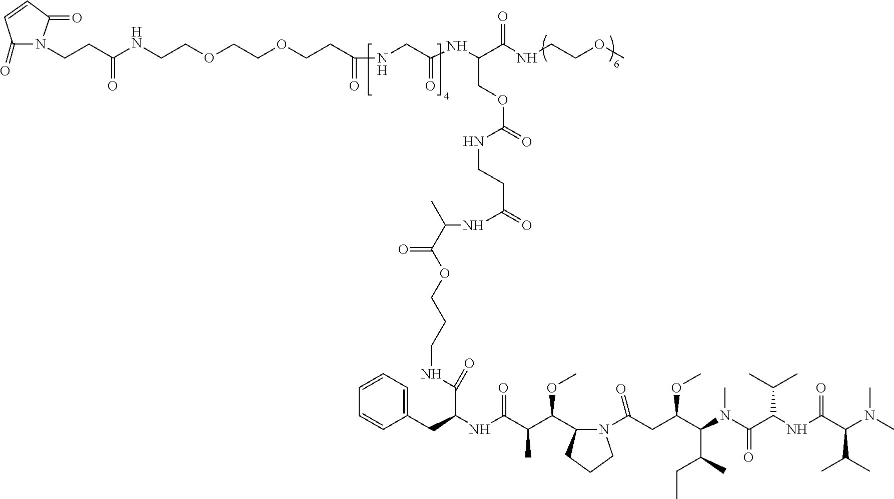

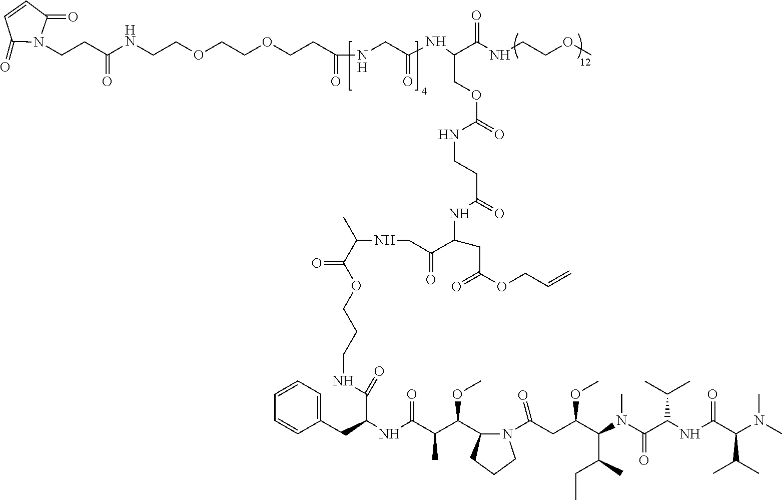

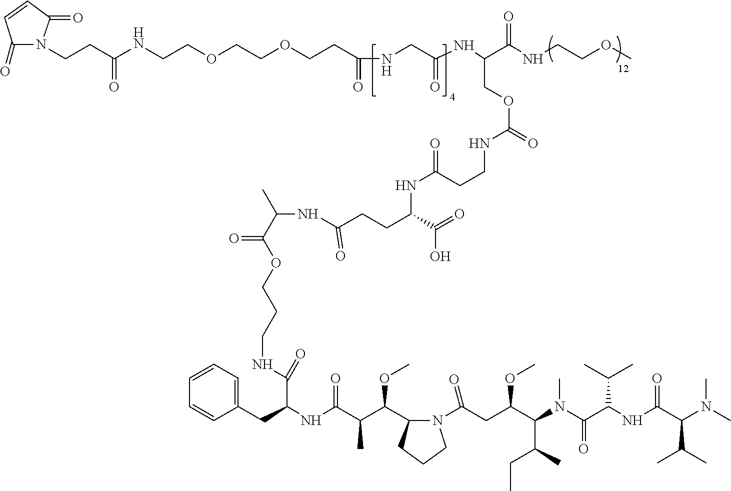

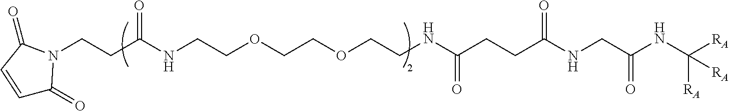

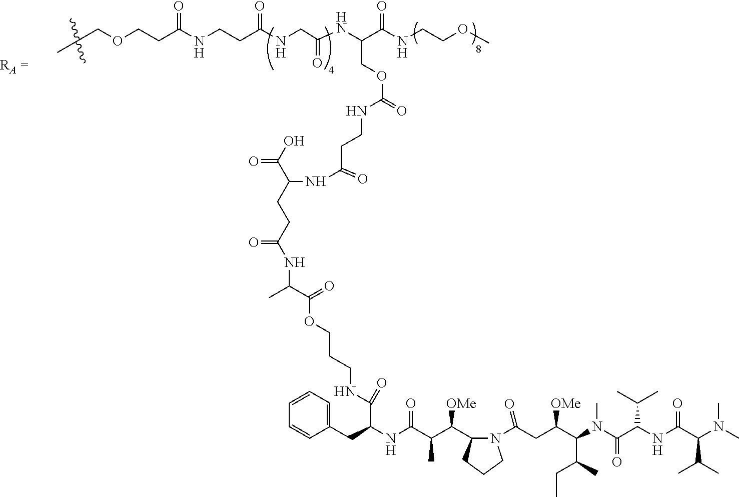

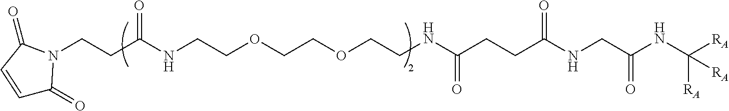

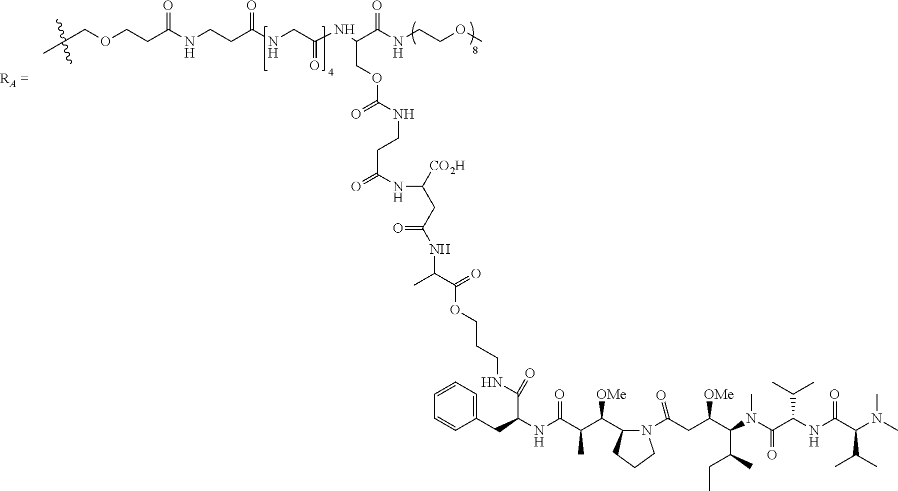

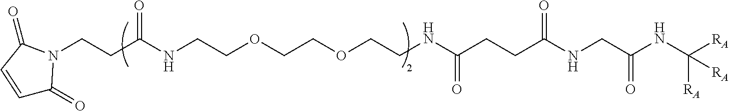

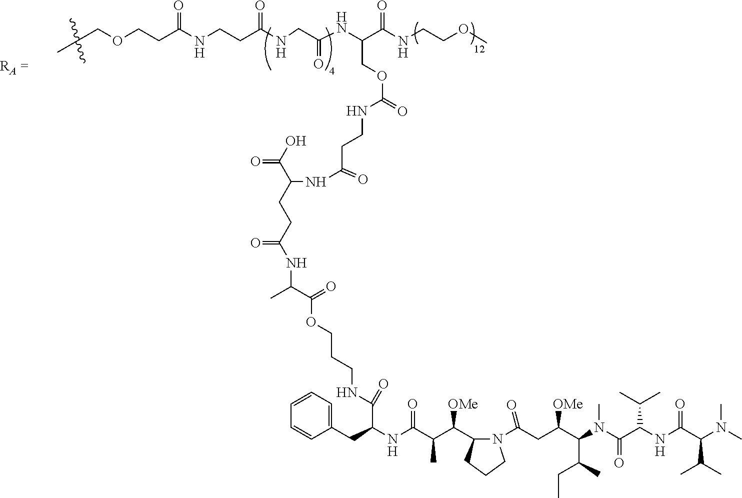

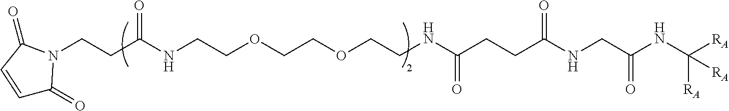

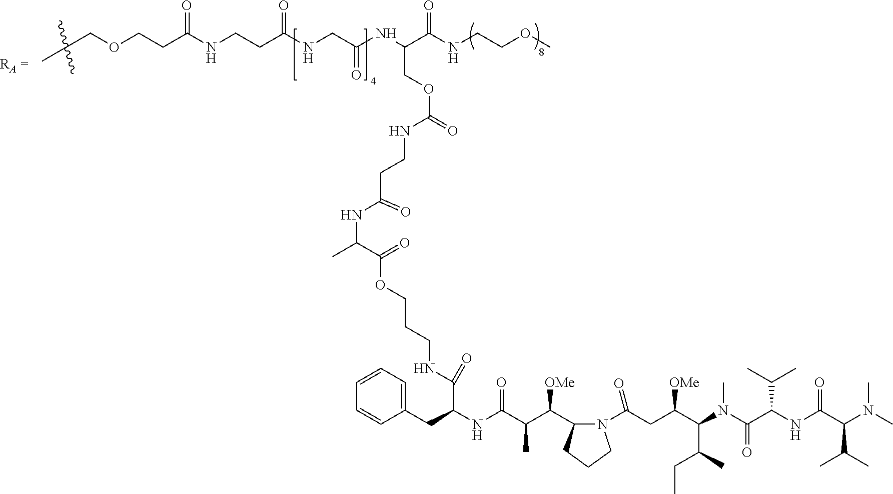

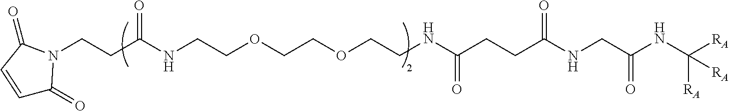

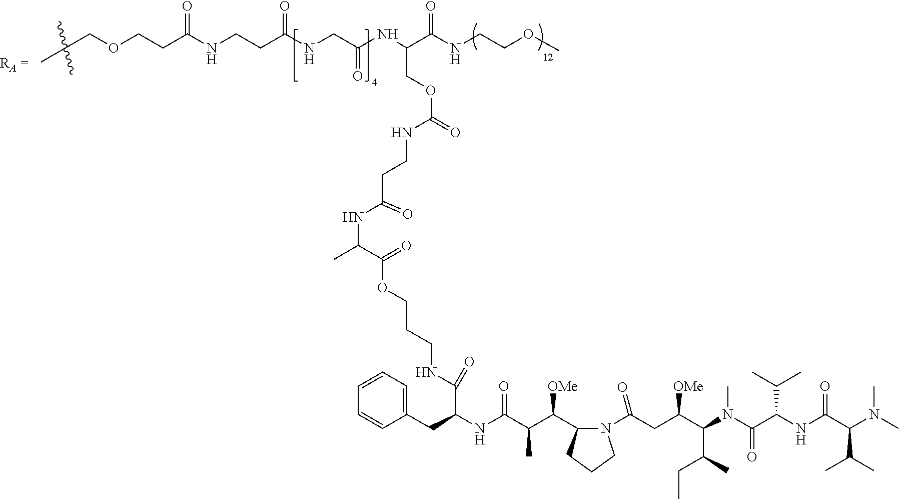

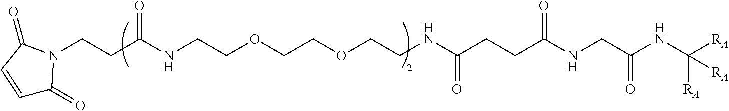

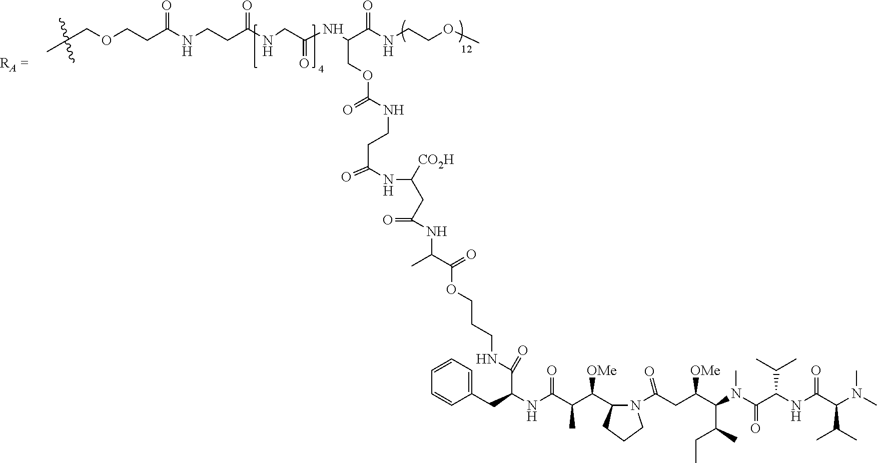

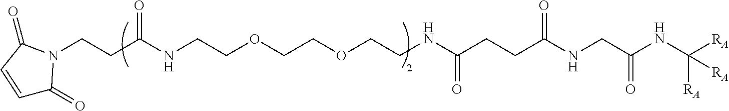

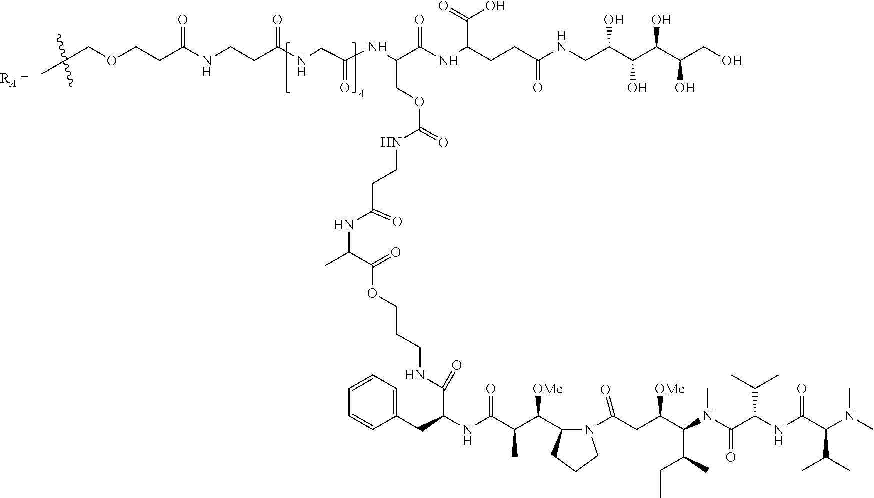

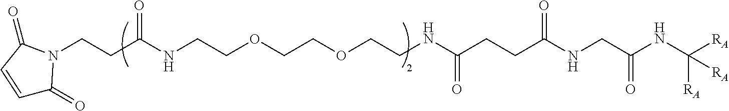

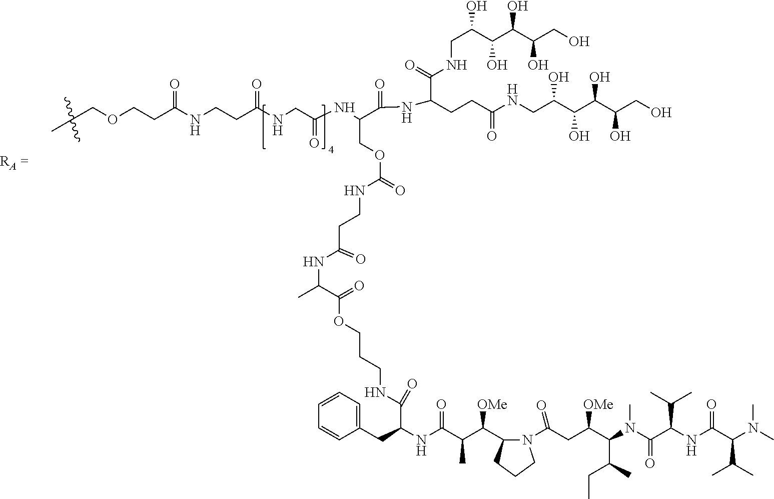

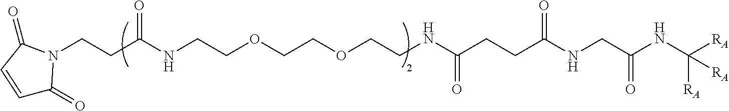

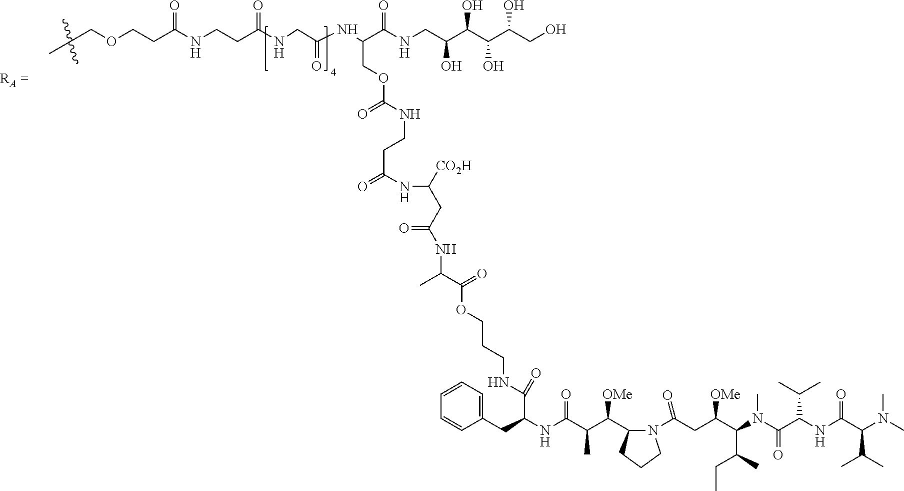

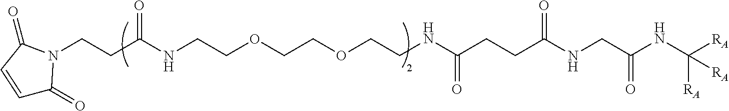

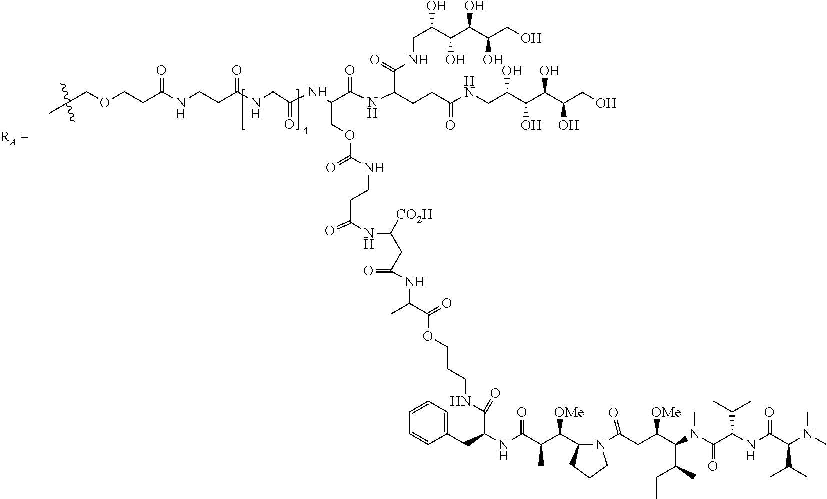

TABLE C
Structure

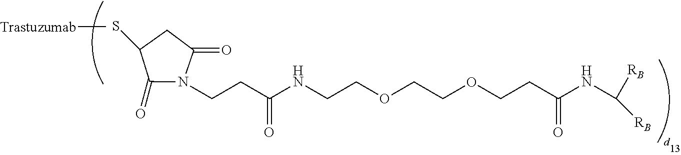

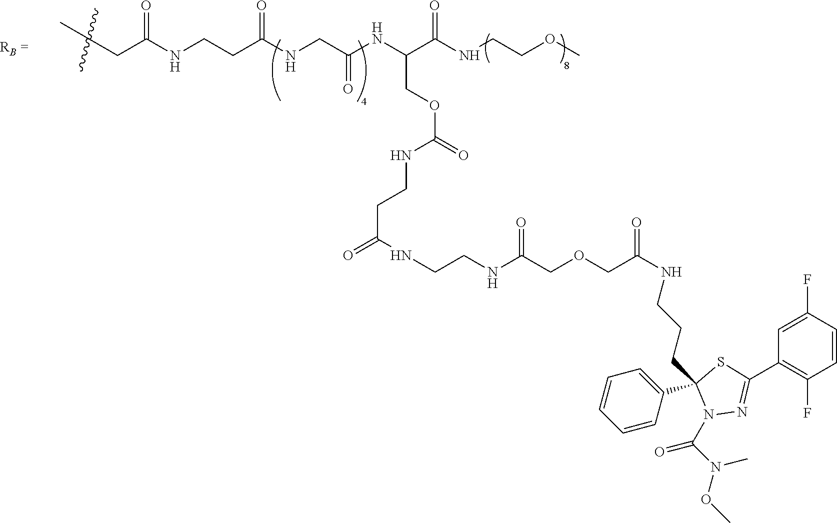

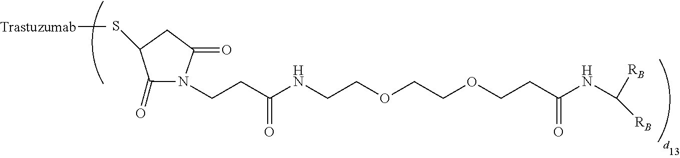

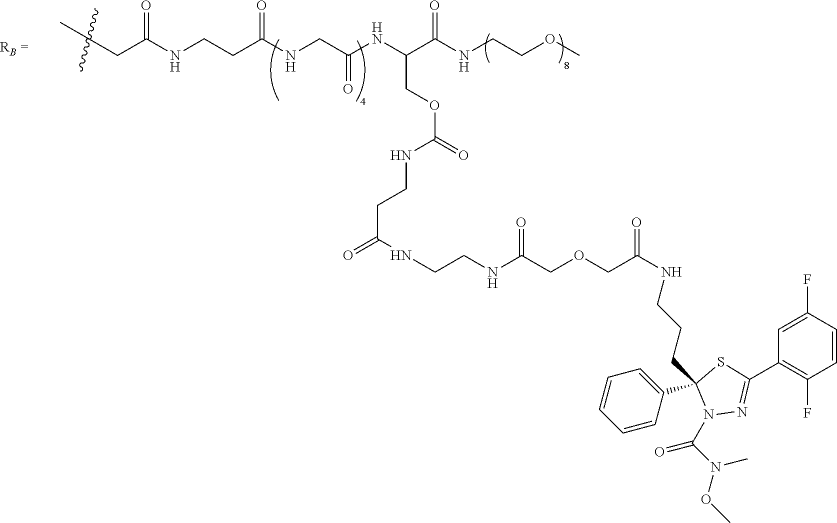

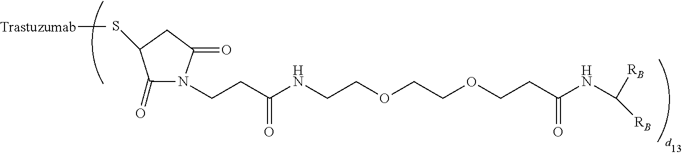

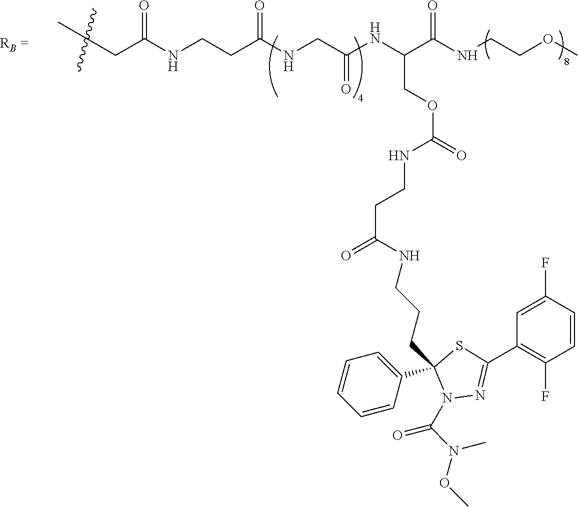

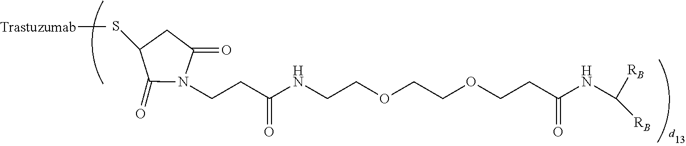

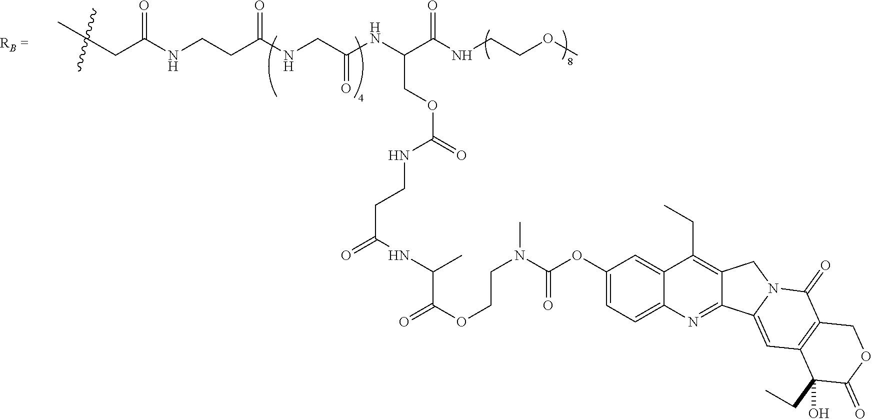

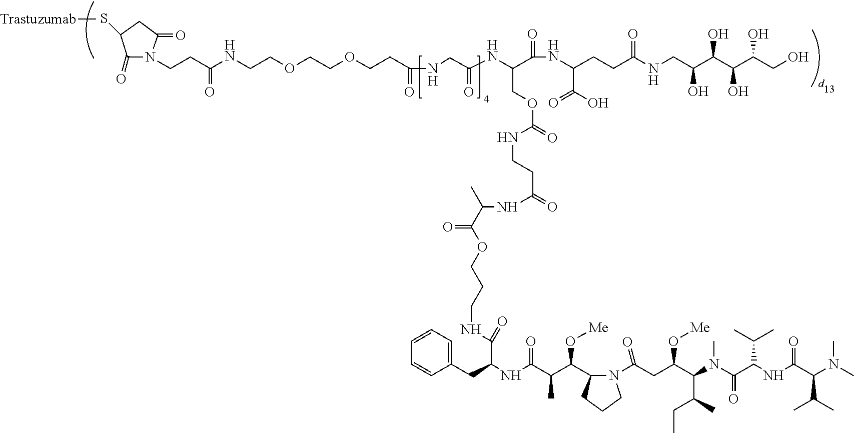

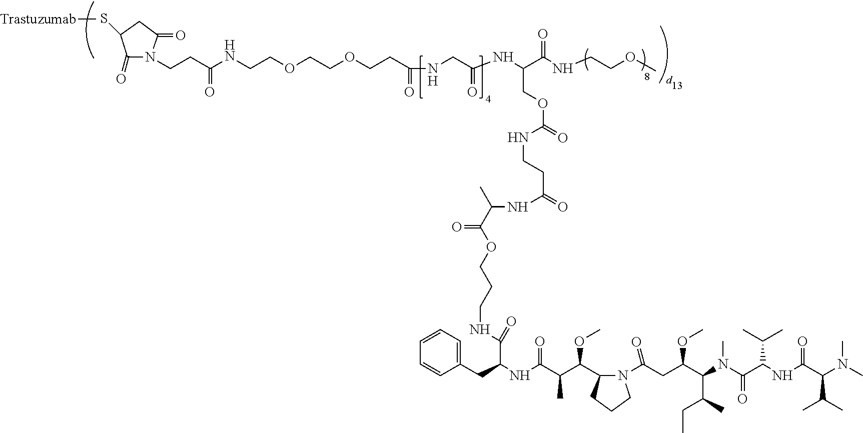

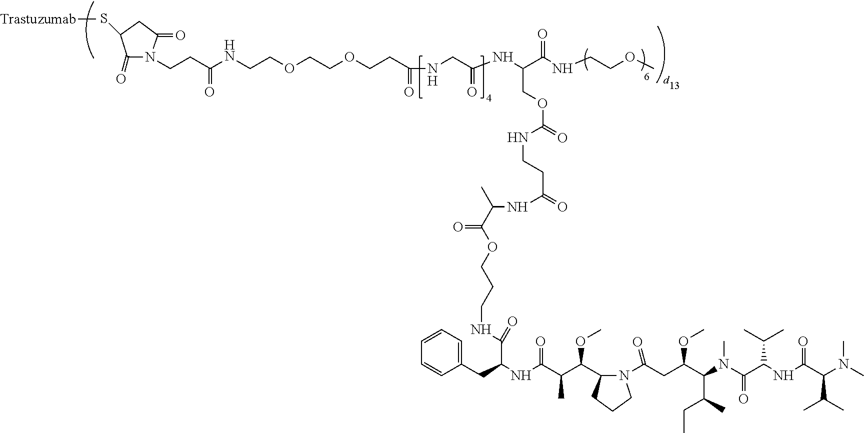

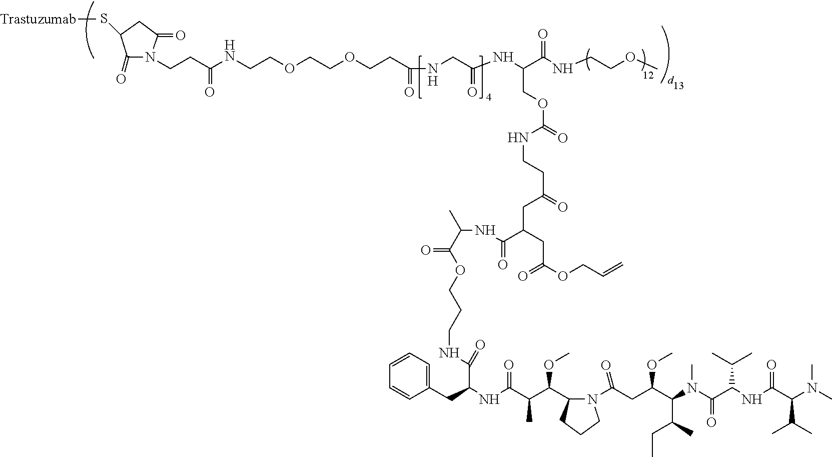

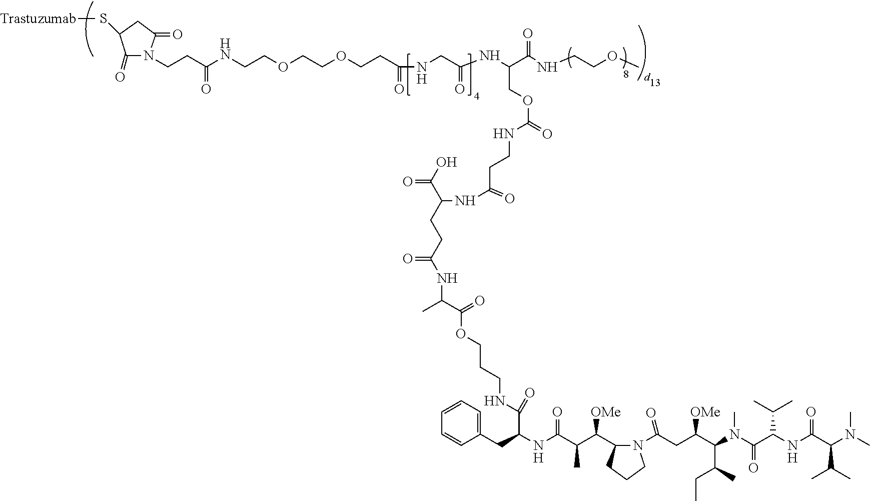

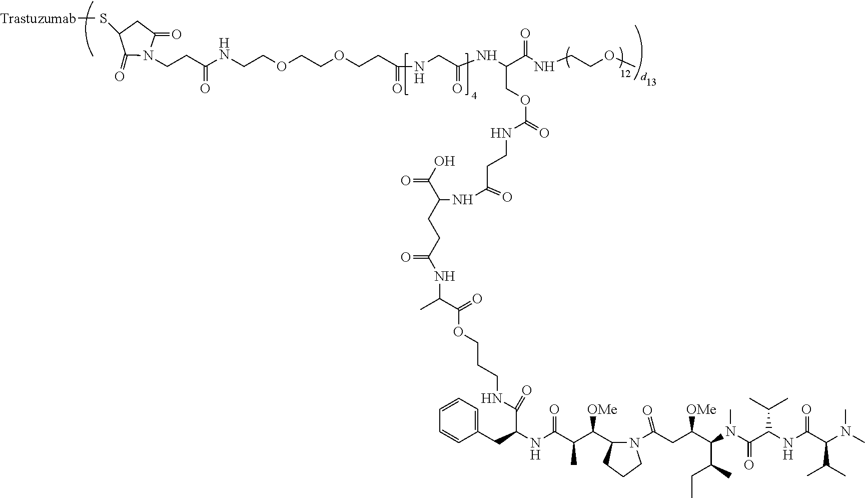

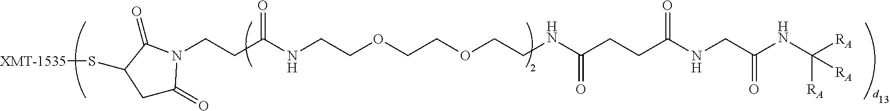

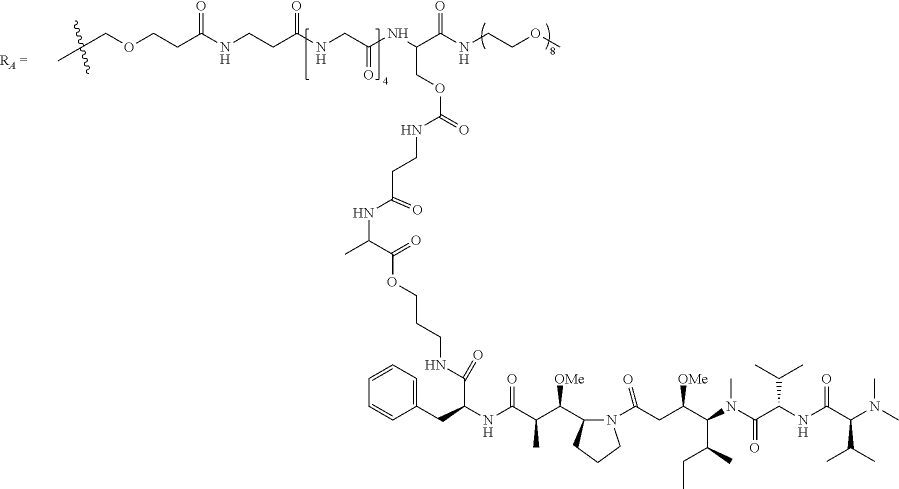

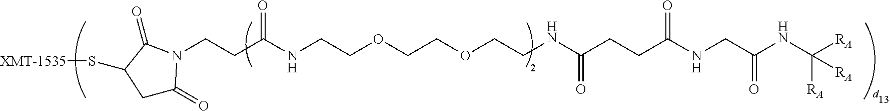

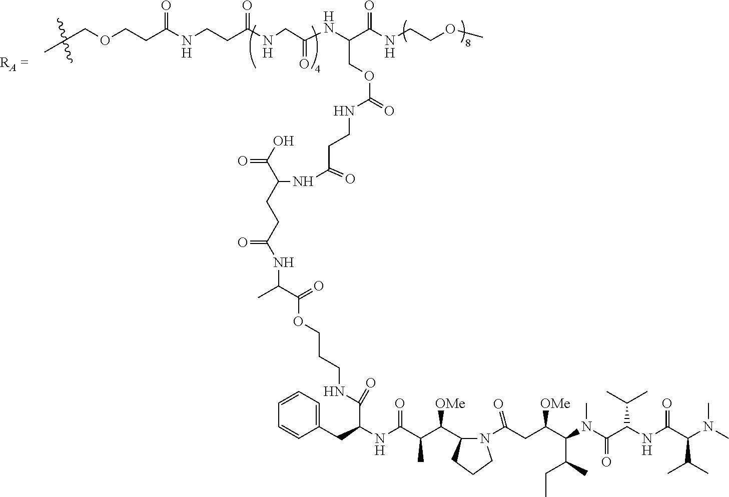

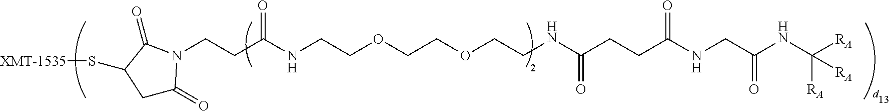

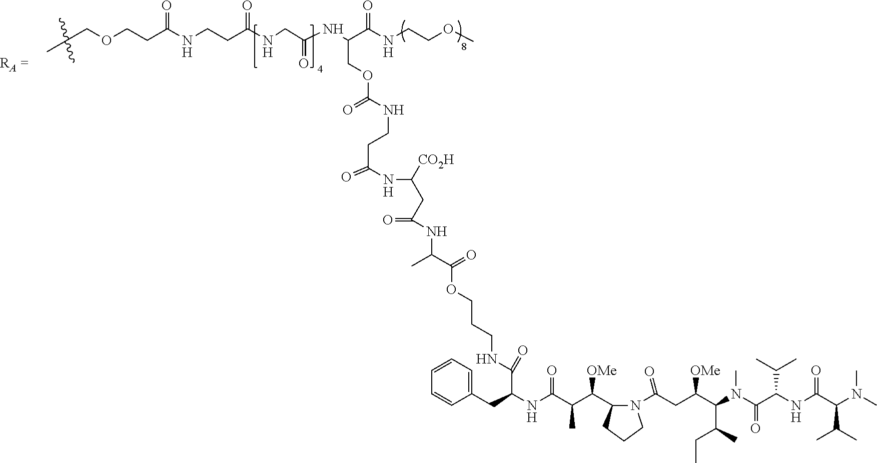

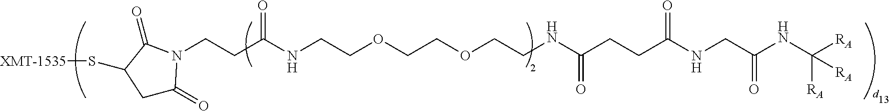

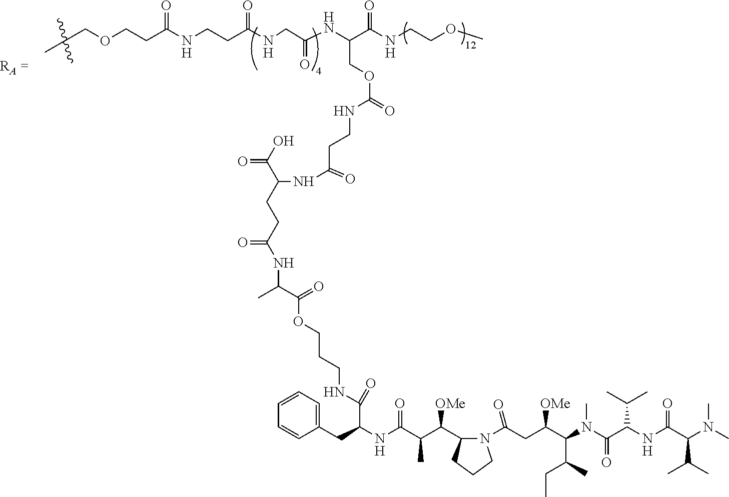

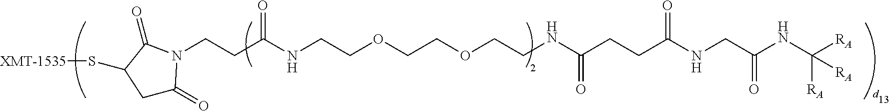

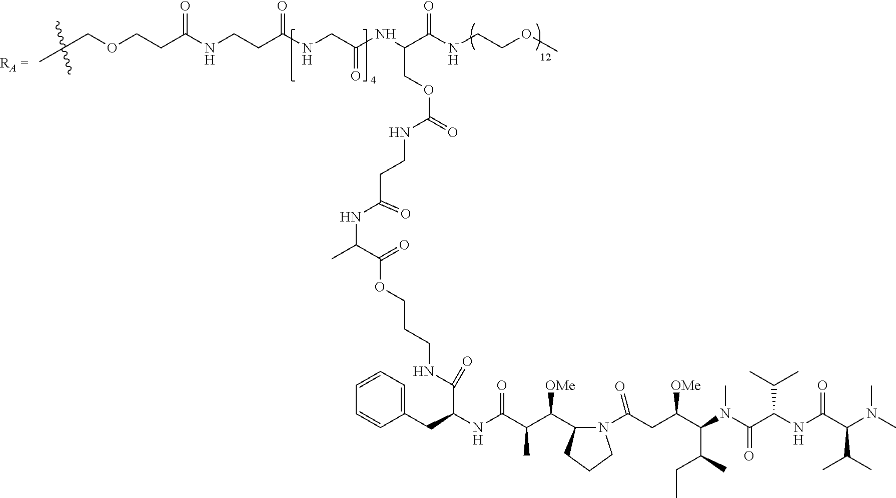

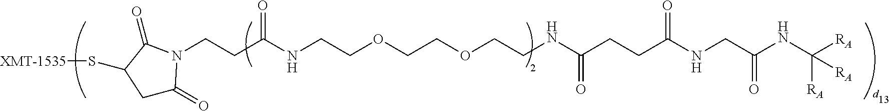

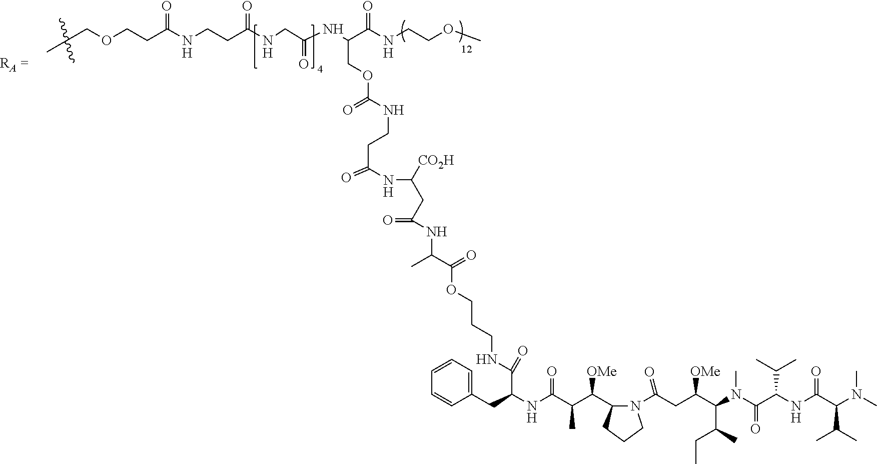

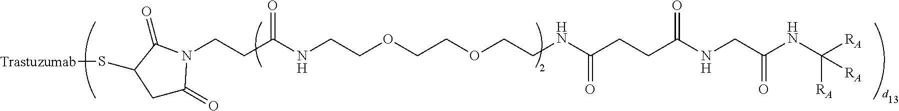

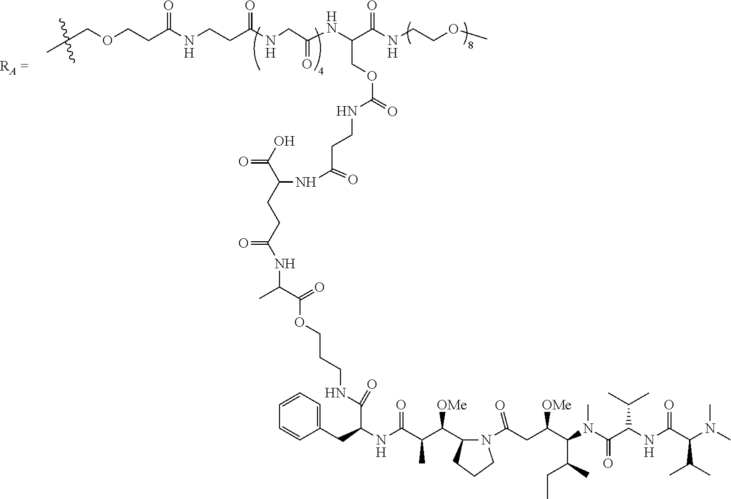

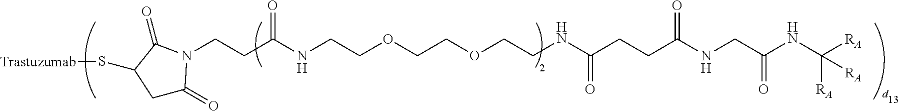

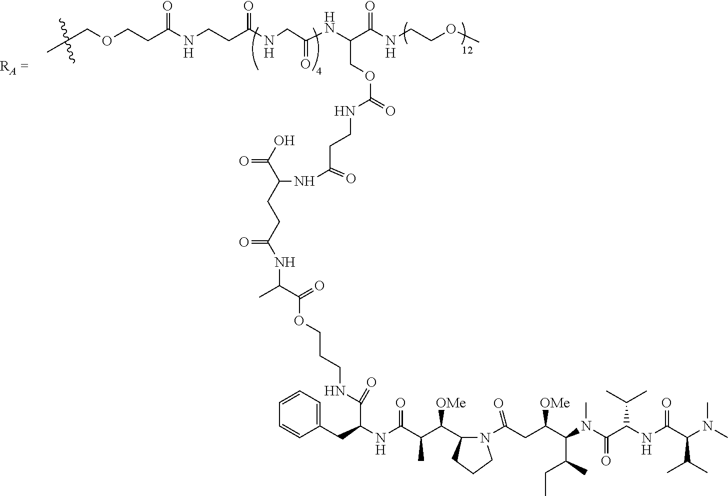

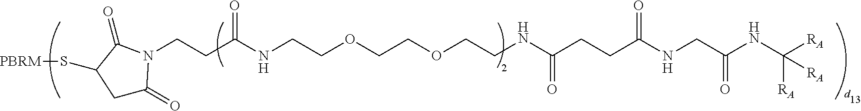

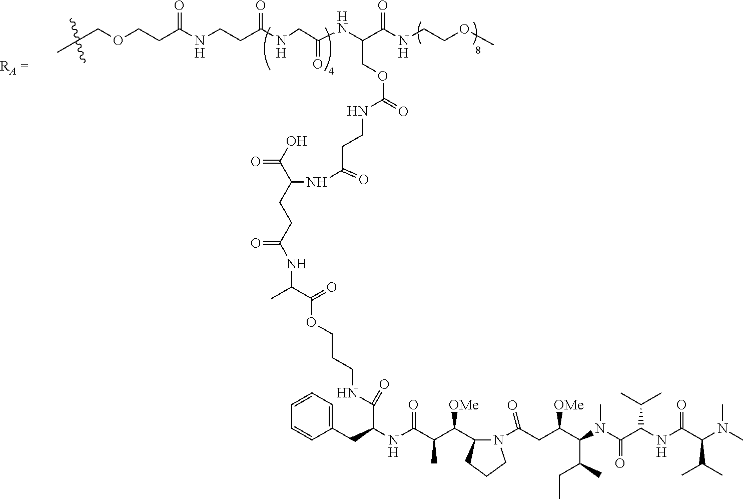

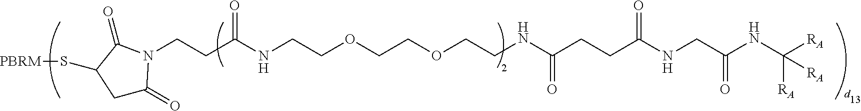

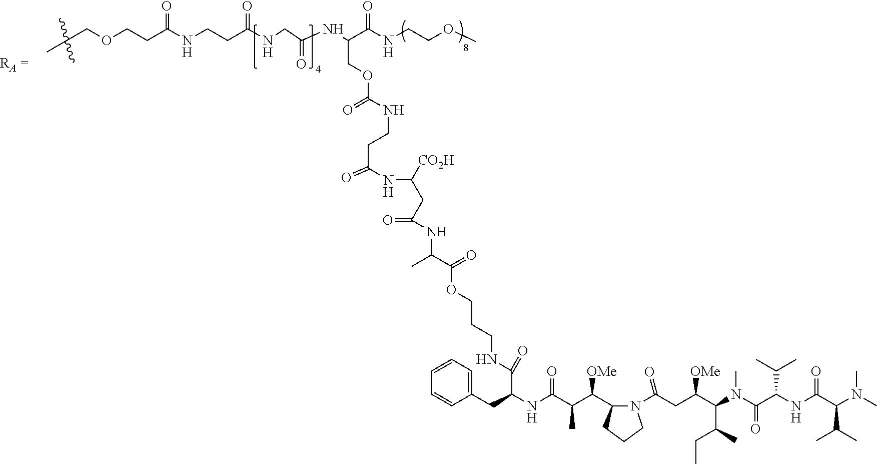

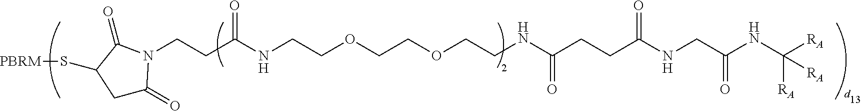

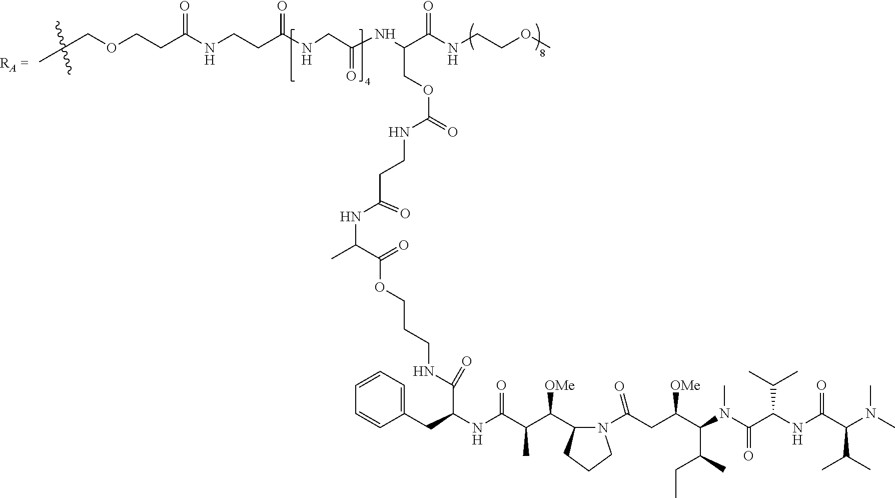

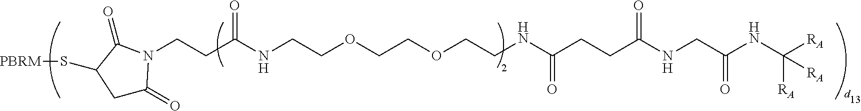

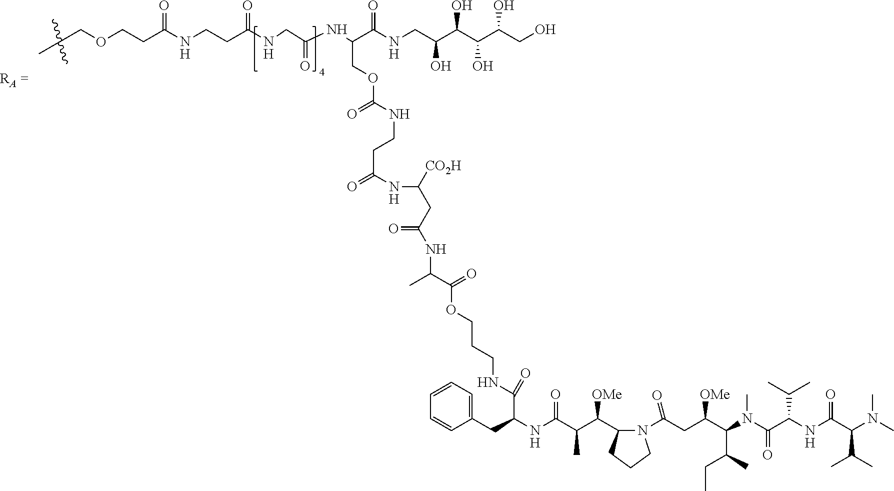

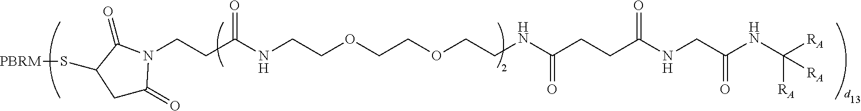

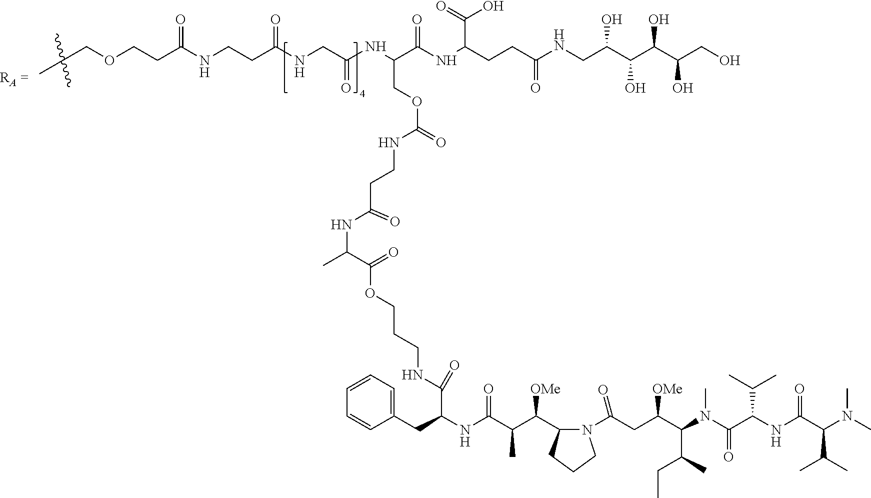

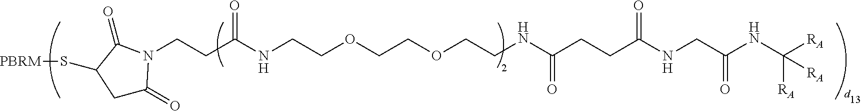

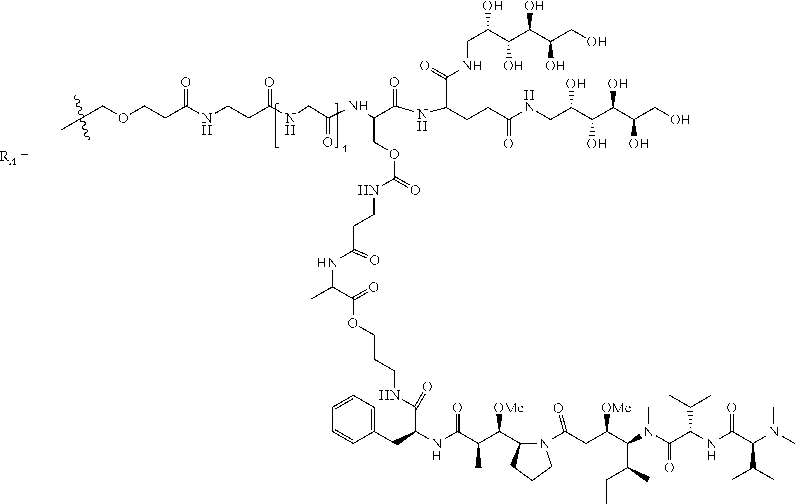

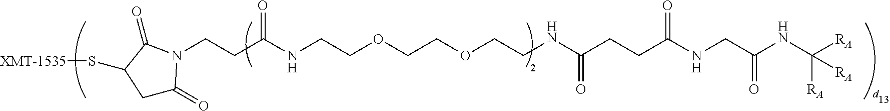

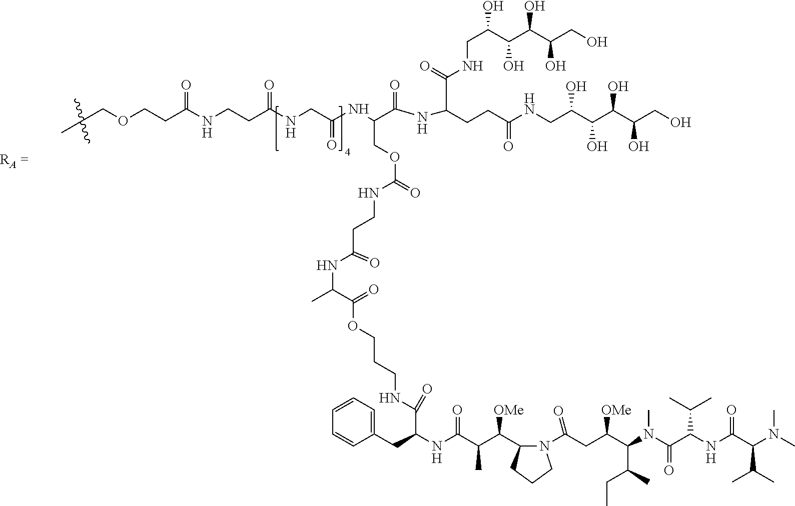

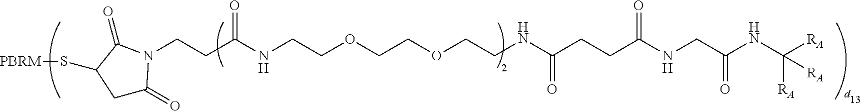

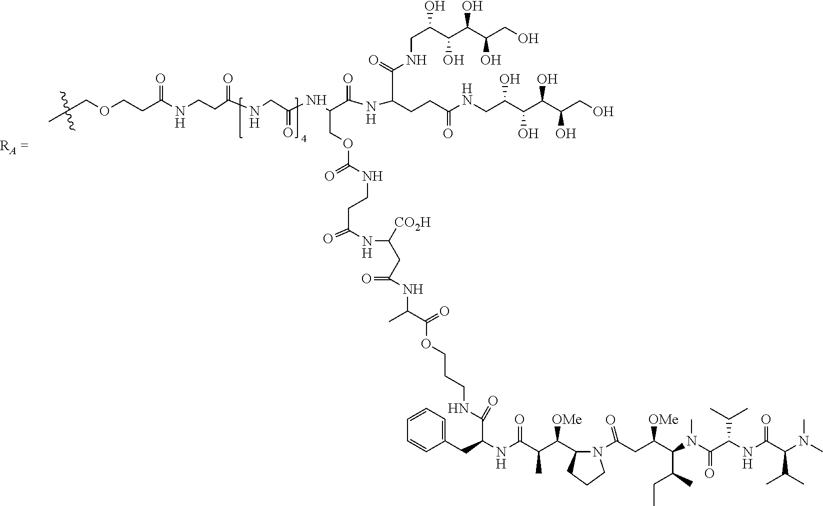

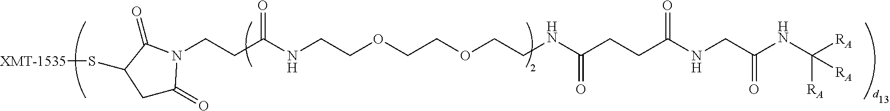

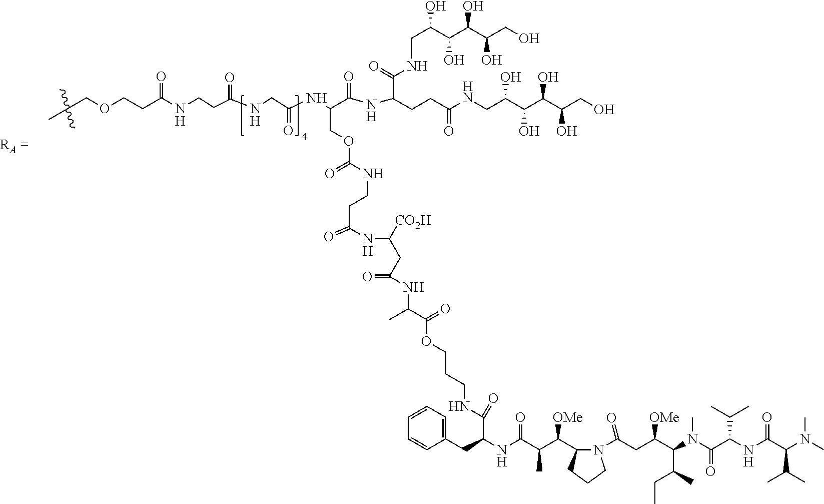

It is understood that the sequence and structure of XMT-1535 can be found at PCT Application No. PCT/US2017/022155 and U.S. application Ser. No. 15/457,574, the entire contents of which are incorporated herein by reference.

In some embodiments, the PBRM is Trastuzumab. In some embodiments, the PBRM is XMT-1535.

In some embodiments, the cysteine engineered protein-drug conjugates are conjugates of Formula (XXX):

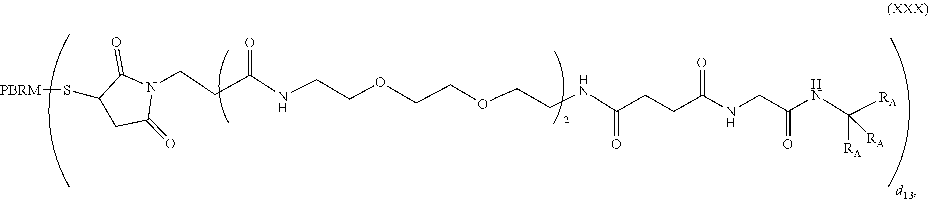

wherein each R_{A} is

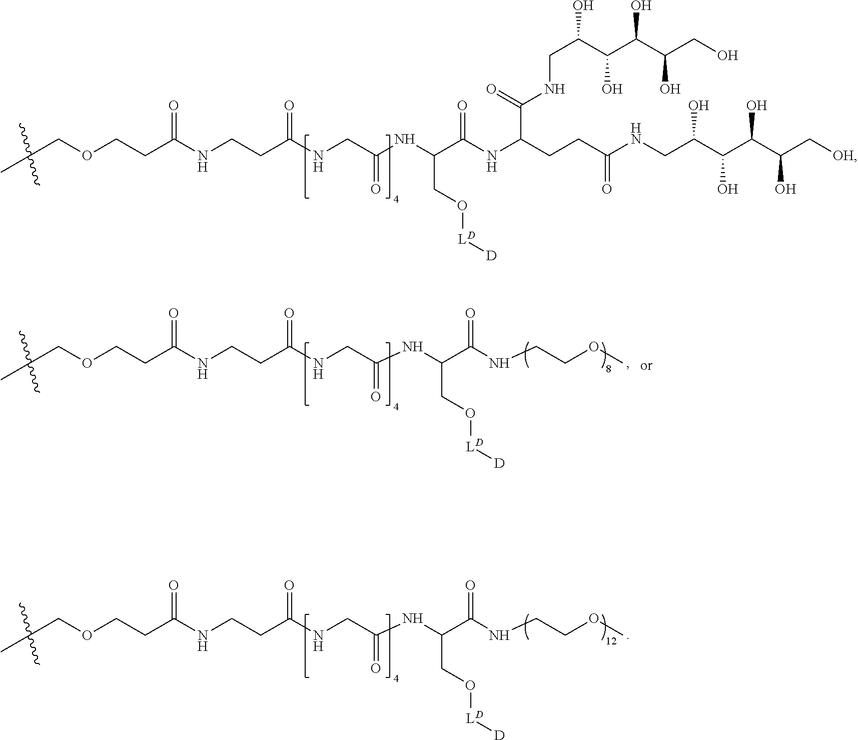

In other embodiments, the cysteine engineered protein-drug conjugates are conjugates of Formula (XXX):

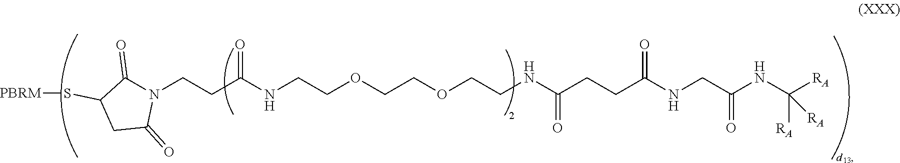

wherein each R_{A} is

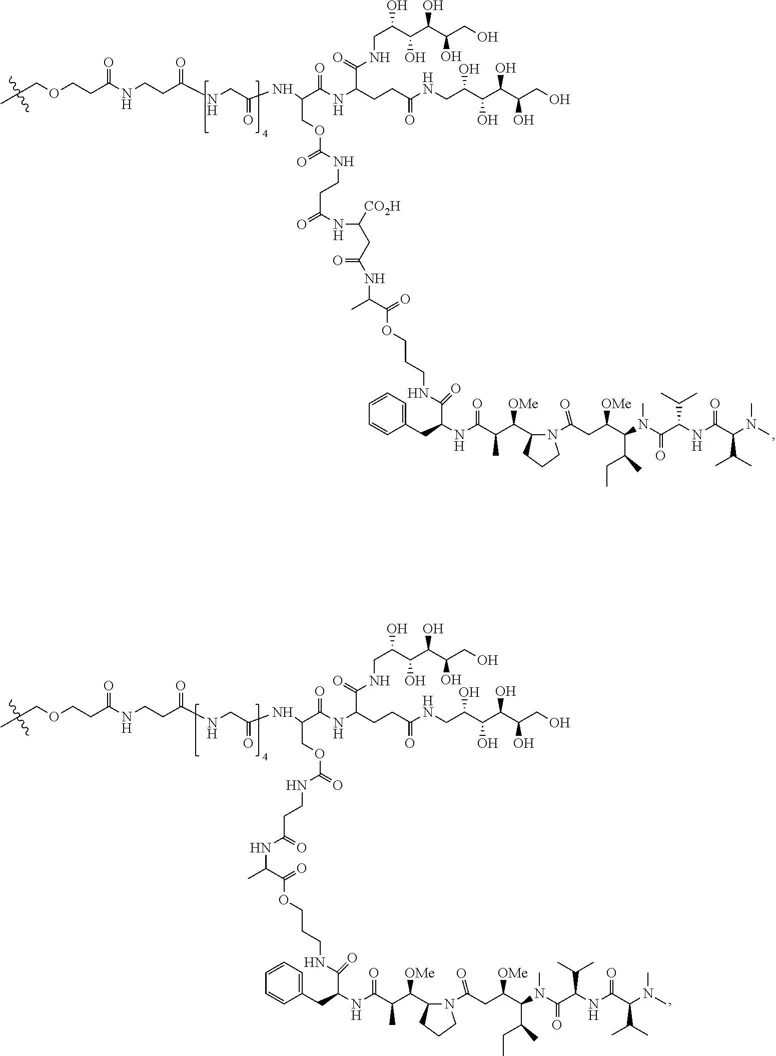

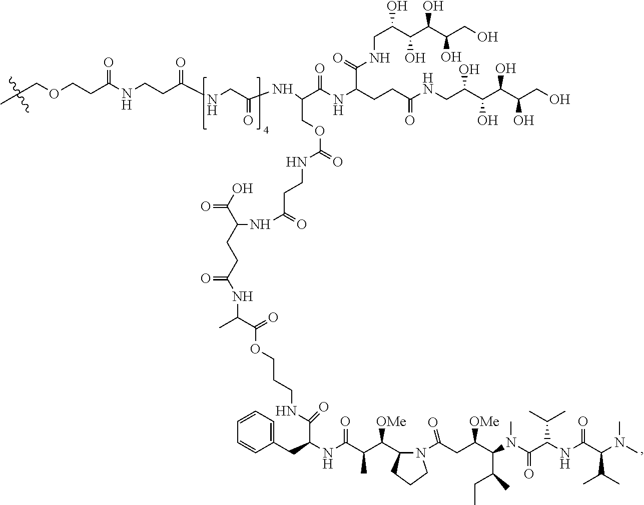

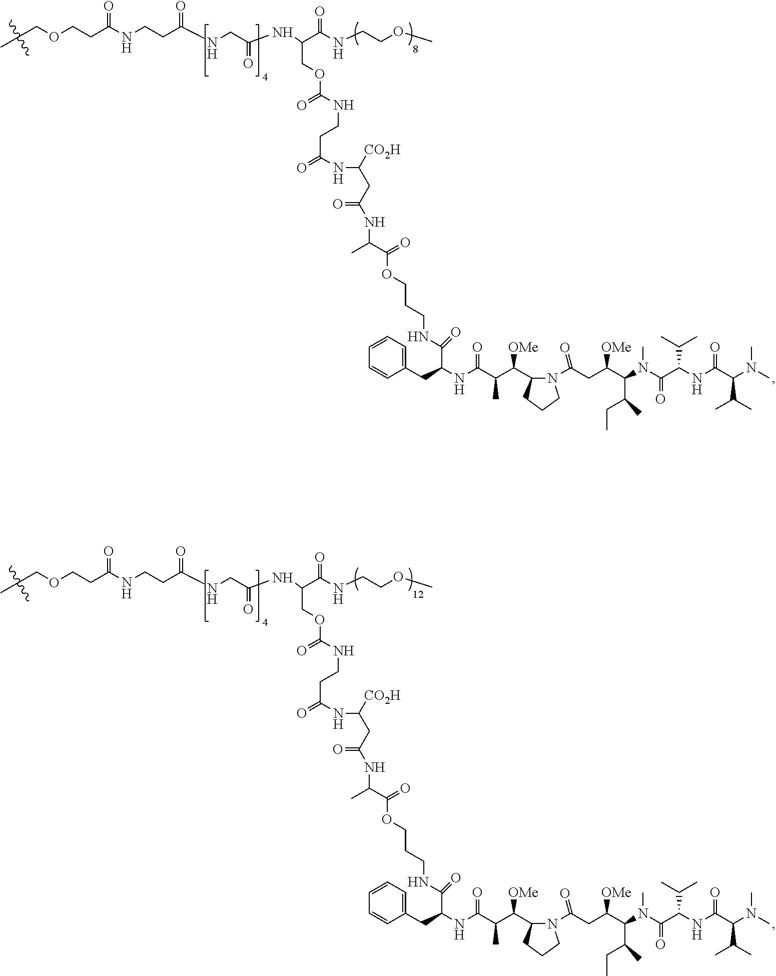

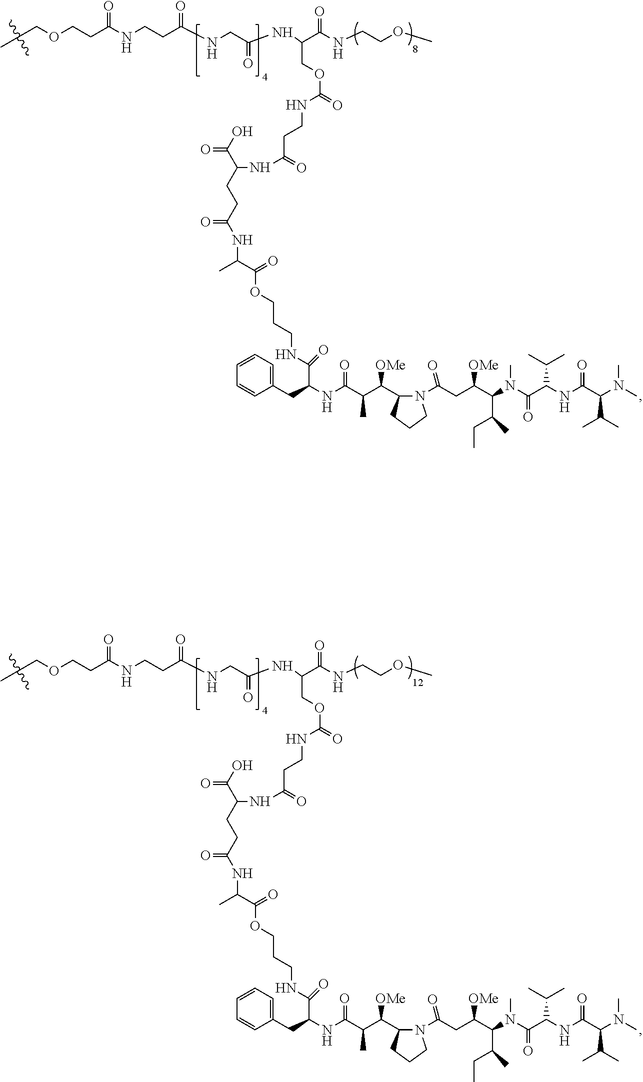

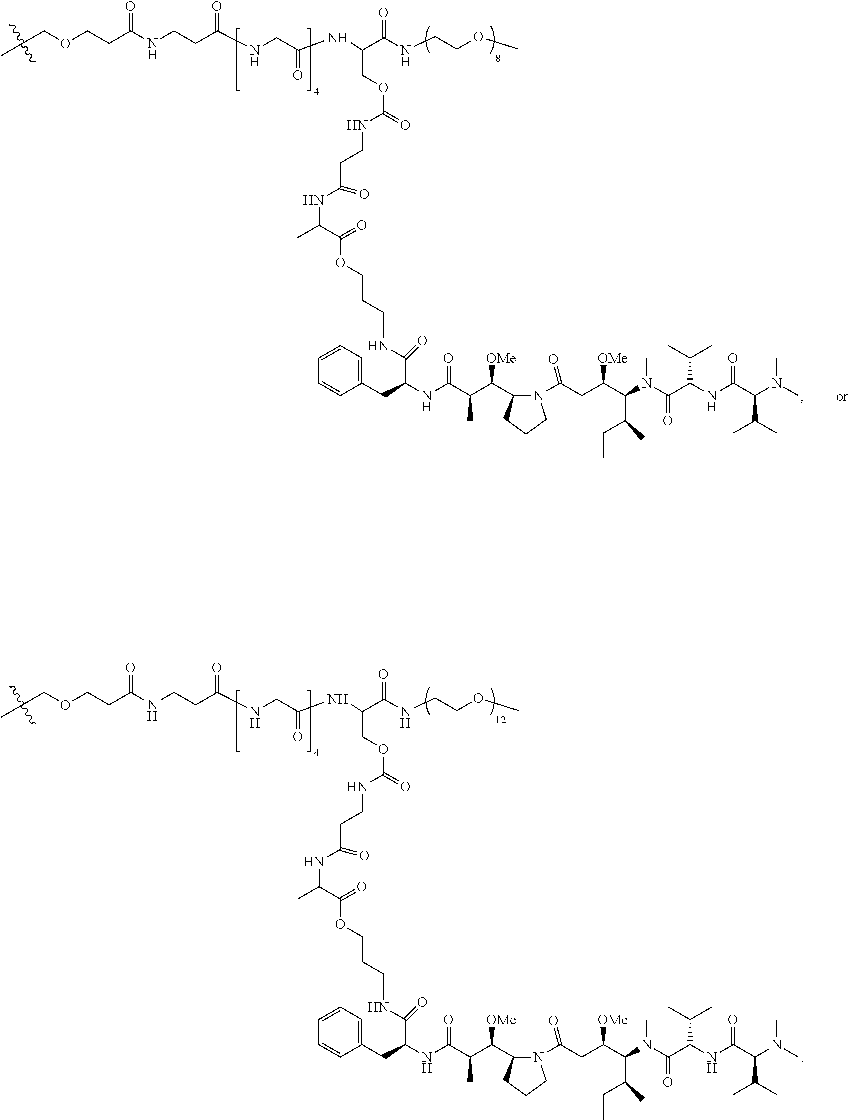

In some embodiments, the cysteine engineered protein-drug conjugate is of Formula (XXX), wherein each R_{A} is

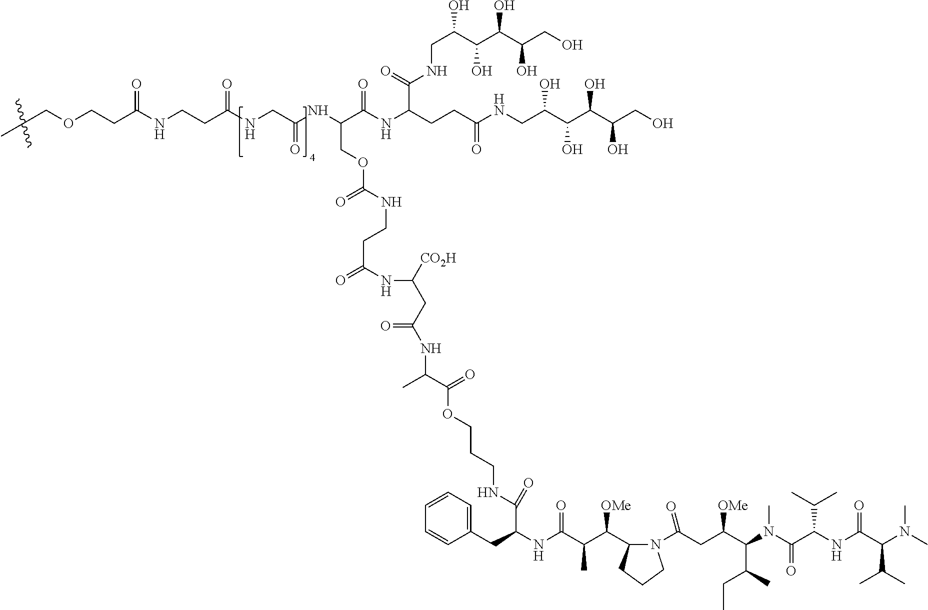

In some embodiments, the protein-drug conjugate is of Formula (XXX), wherein each R_{A} is

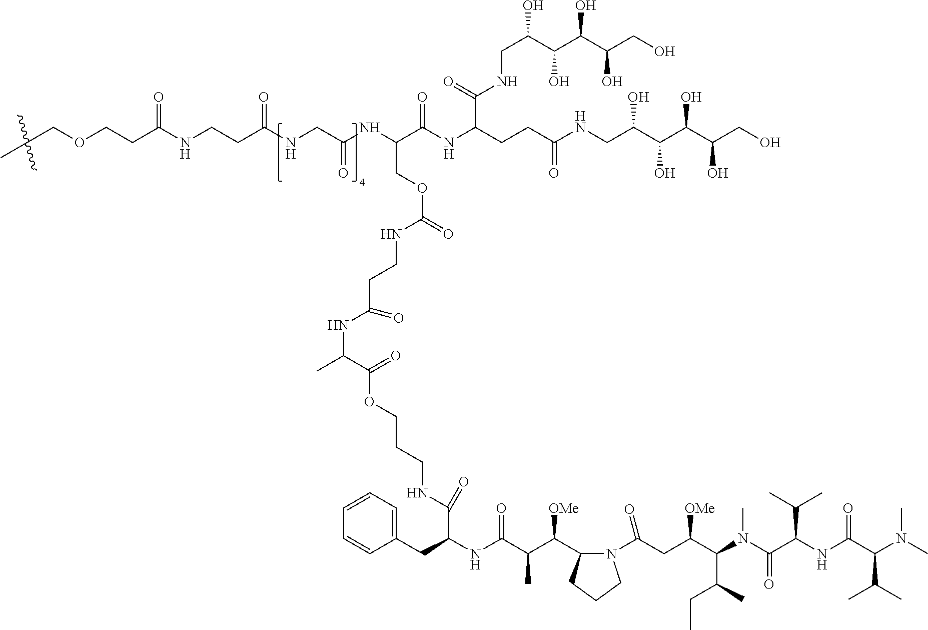

In some embodiments, the protein-drug conjugate is of Formula (XXX), wherein each R_{A} is

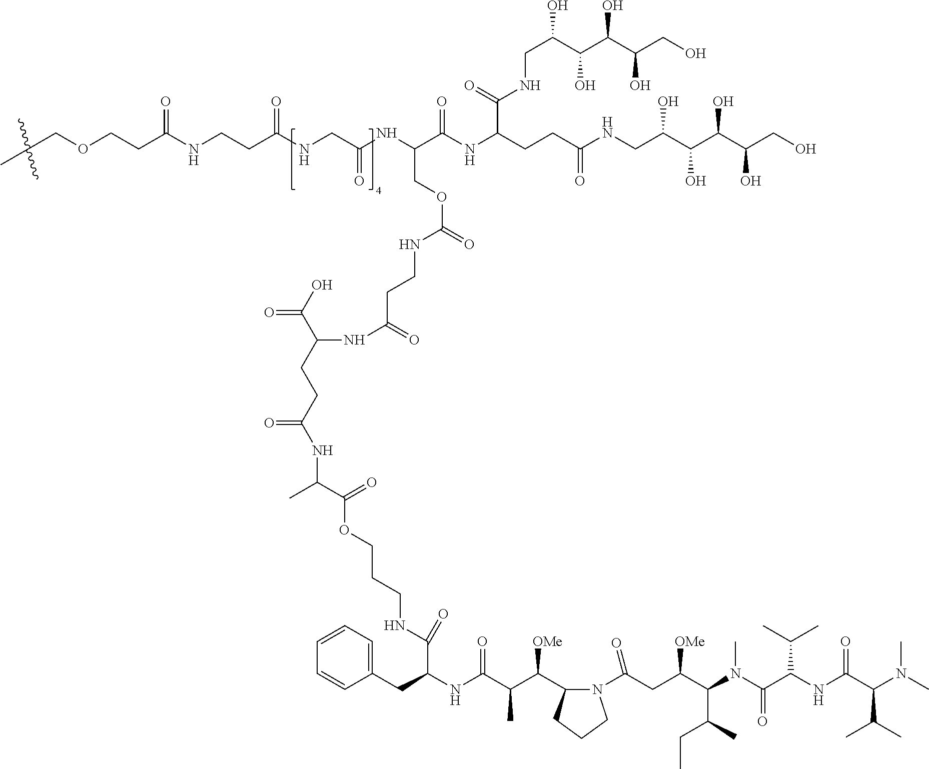

In some embodiments, the cysteine engineered protein-drug conjugate is of Formula (XXX), wherein each R_{A} is

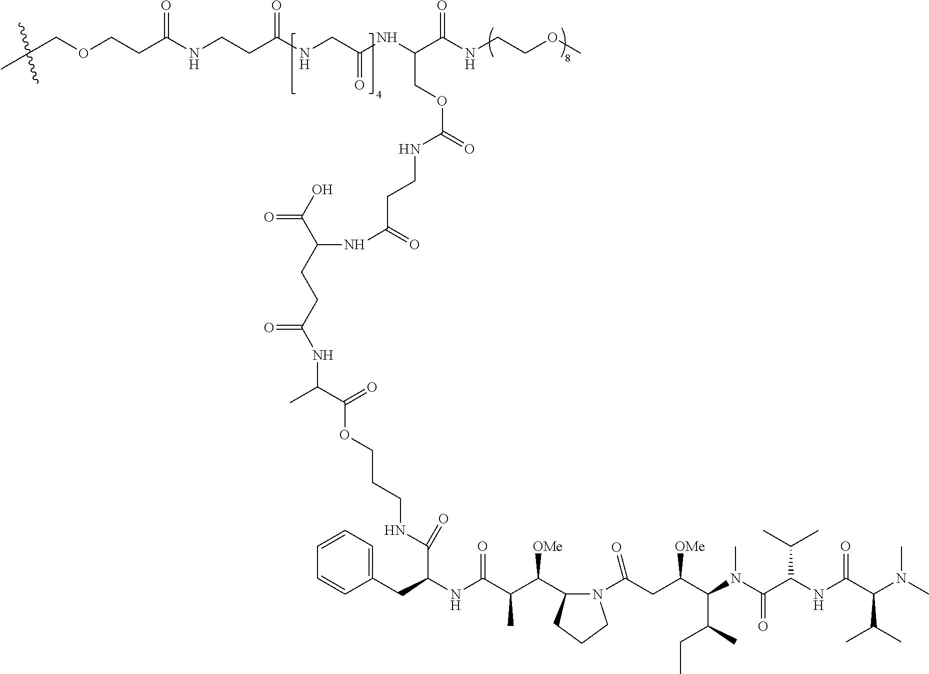

In some embodiments, the cysteine engineered protein-drug conjugate is of Formula (XXX), wherein each R_{A} is

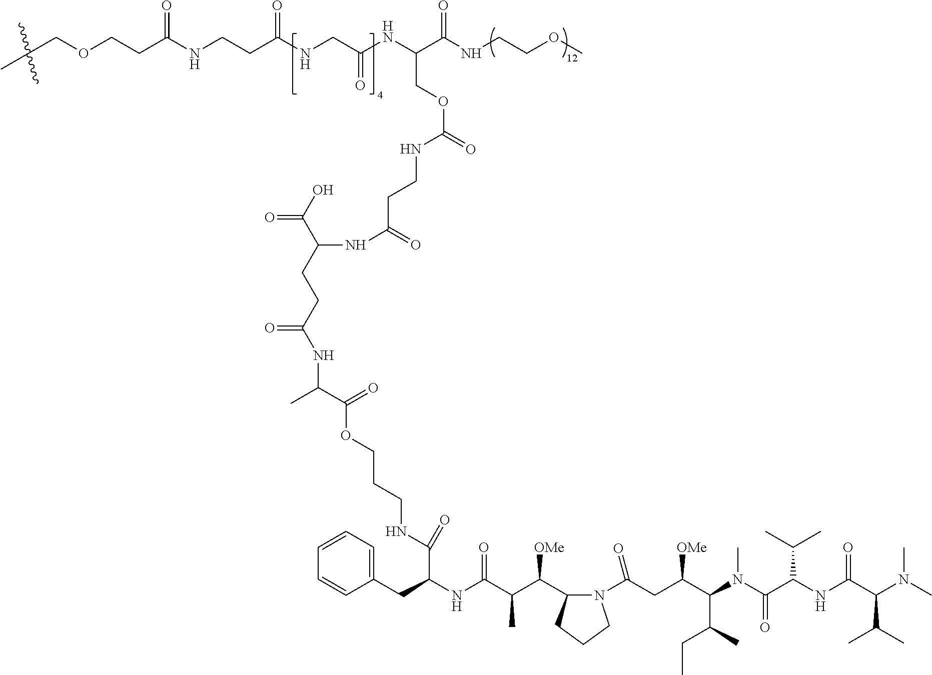

In some embodiments, the cysteine engineered protein-drug conjugate is of Formula (XXX), wherein each R_{A} is

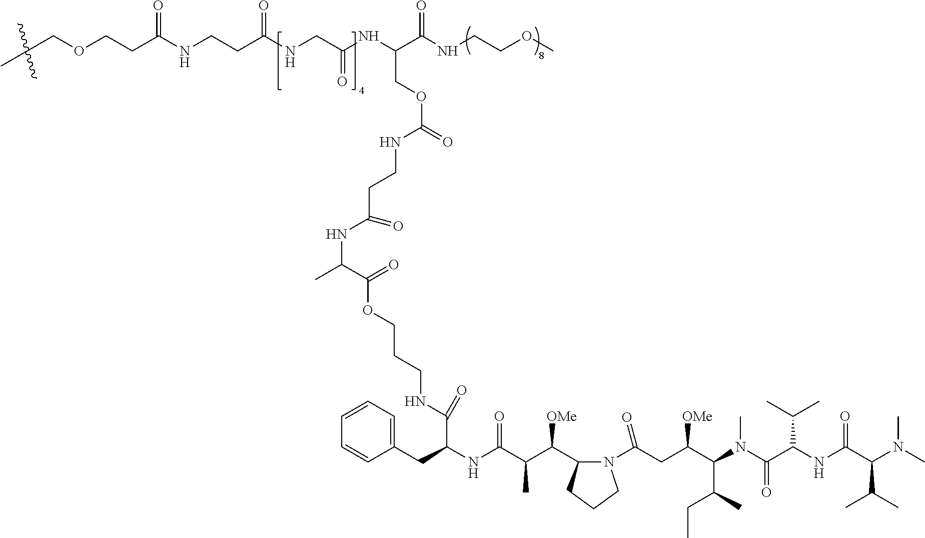

In some embodiments, the cysteine engineered protein-drug conjugate is of Formula (XXX), wherein each R_{A} is

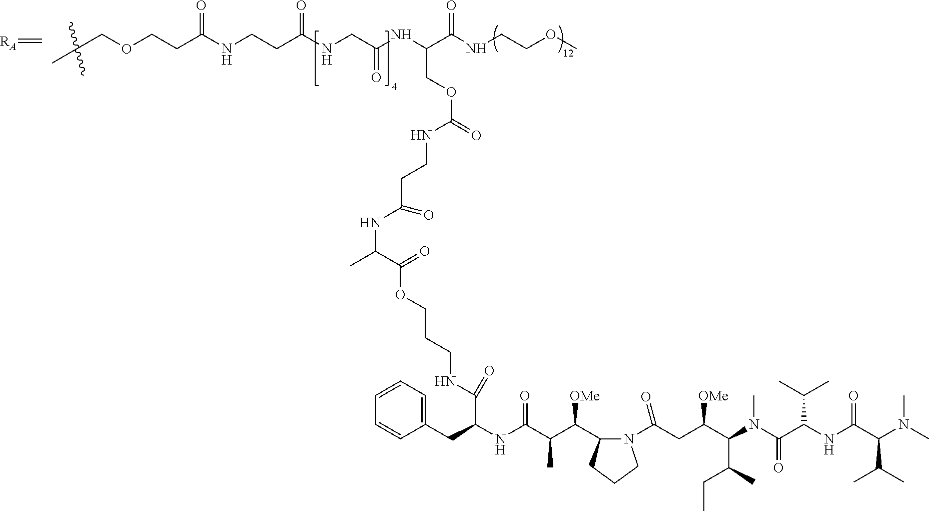

In some embodiments, the cysteine engineered protein-drug conjugate is of Formula (XXX), wherein each R_{A} is:

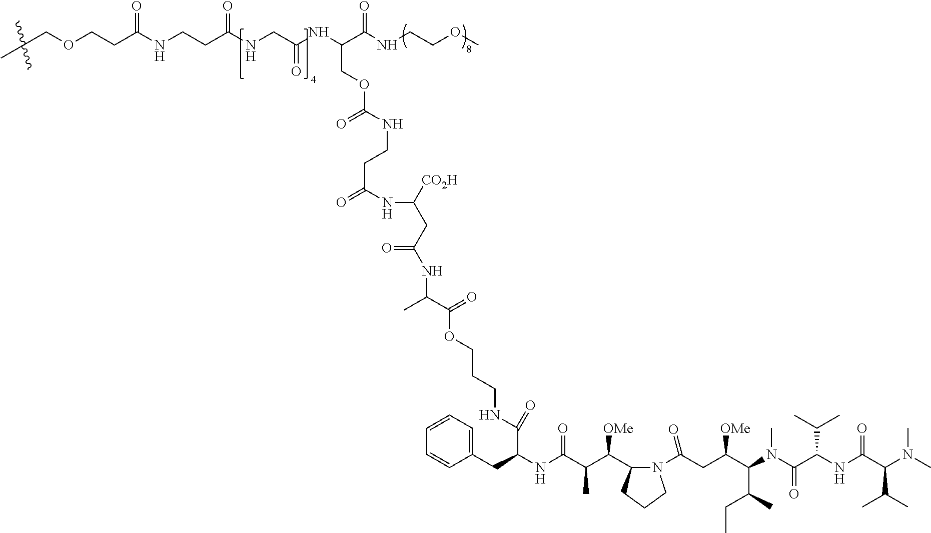

In some embodiments, the cysteine engineered protein-drug conjugate is of Formula (XXX), wherein each R_{A} is

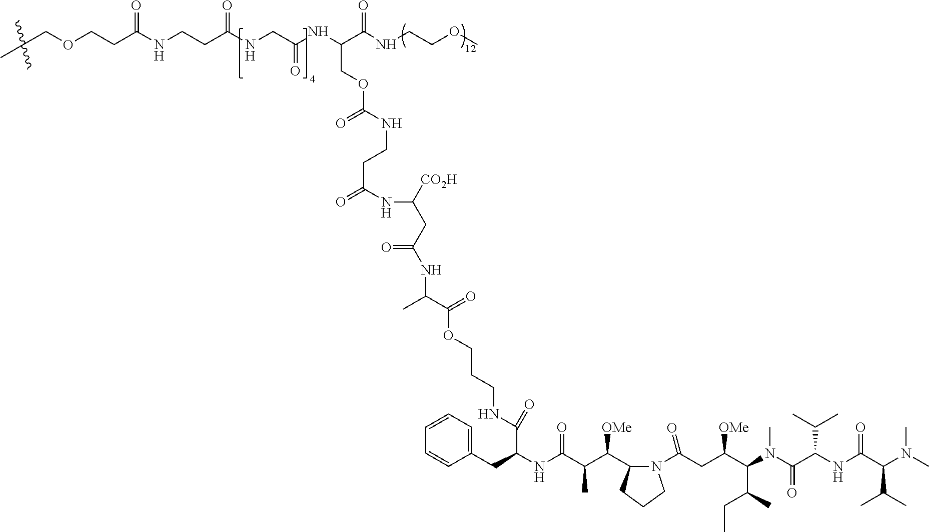

In some embodiments, the cysteine engineered protein-drug conjugates are conjugates of Formula (XXXI-1), (XXXI-2), (XXXI-3) or (XXXI-4):

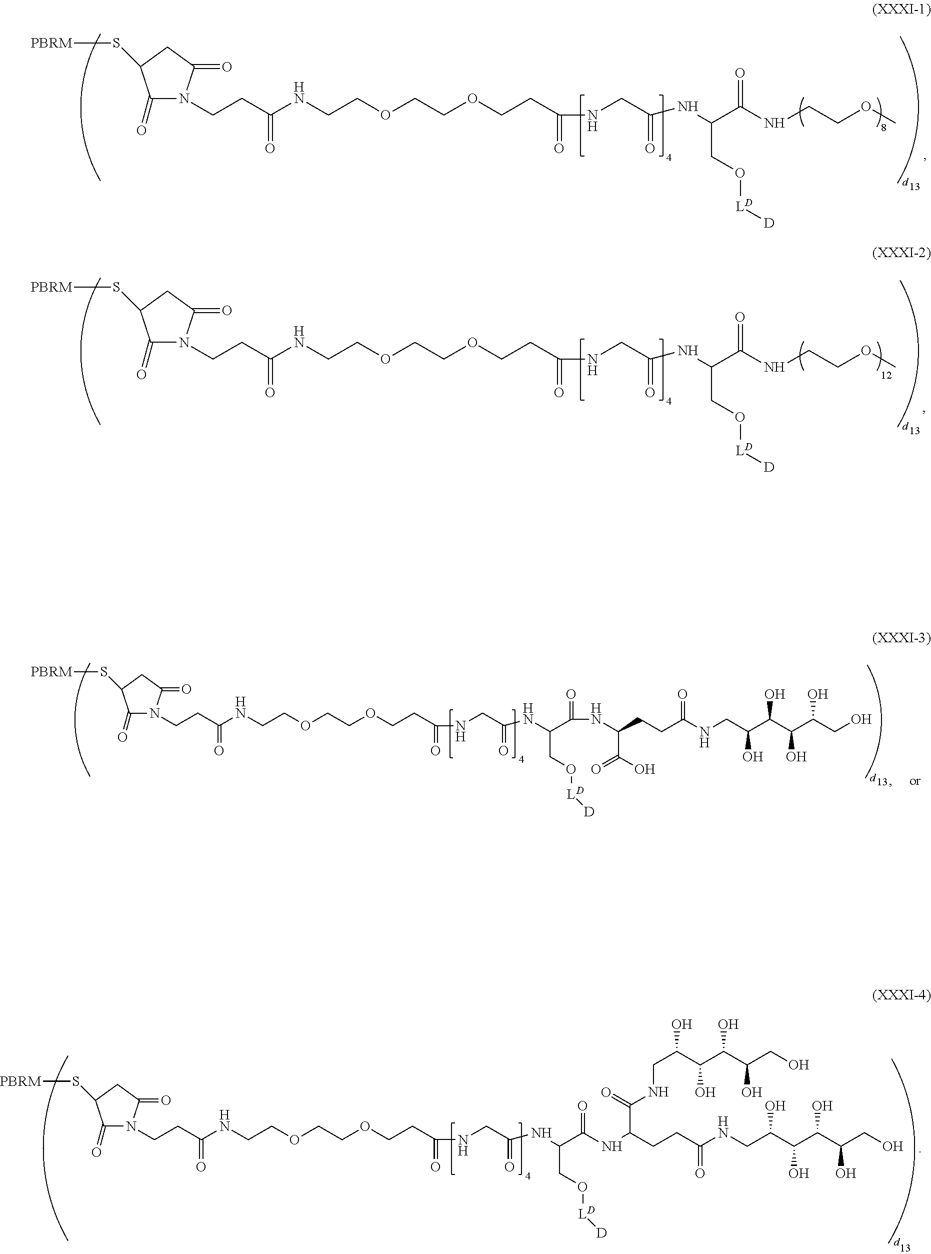

In some embodiments, the protein-drug conjugates are conjugates of Formula (XXXII):

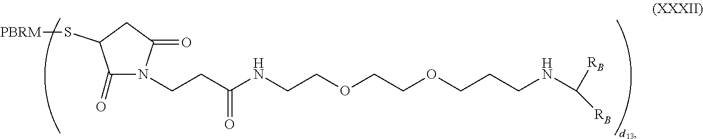

wherein each R_{B} is:

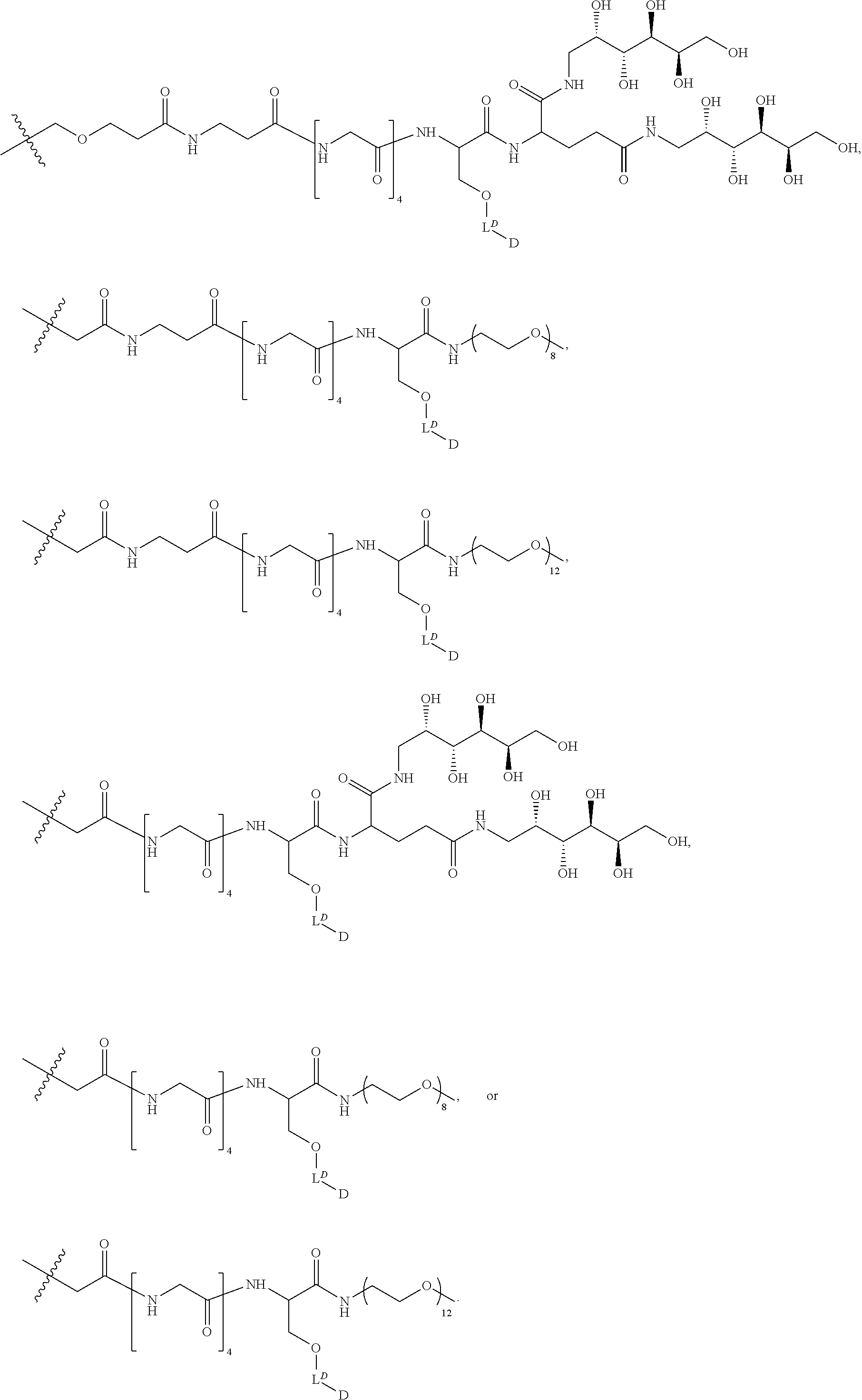

In some embodiments, in the protein-drug conjugates are conjugates of Formula (XXXI-1), (XXXI-2), (XXXI-3), (XXXI-4) or (XXXII), the variable -L^{D}-D is:

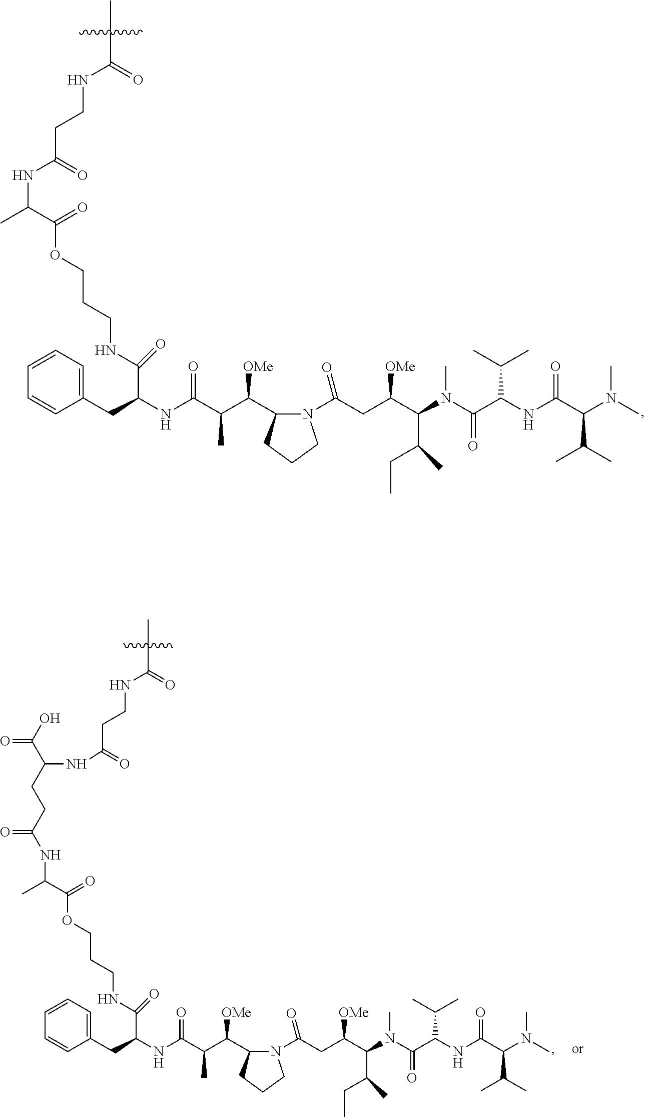

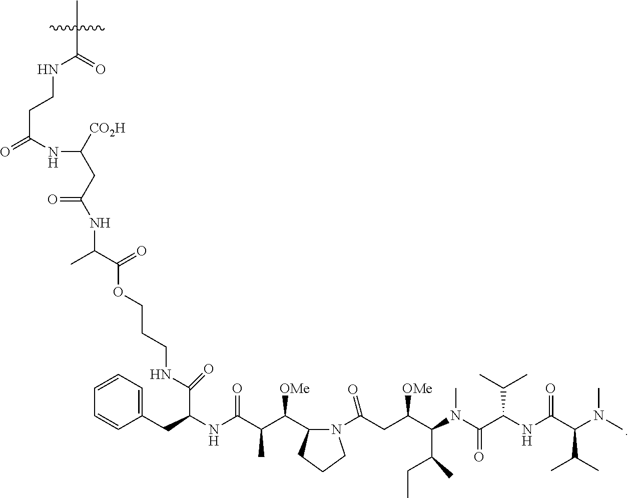 [SMILES: *C(=O)NCCC(=O)NC(CC(=O)NC(C)C(=O)OCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C)C(=O)O]

In some embodiments, the cysteine engineered protein-drug conjugates are conjugates of Formula (XXXIII-1) (XXXIII-2), (XXXXIII-3), or (XXXIII-4):

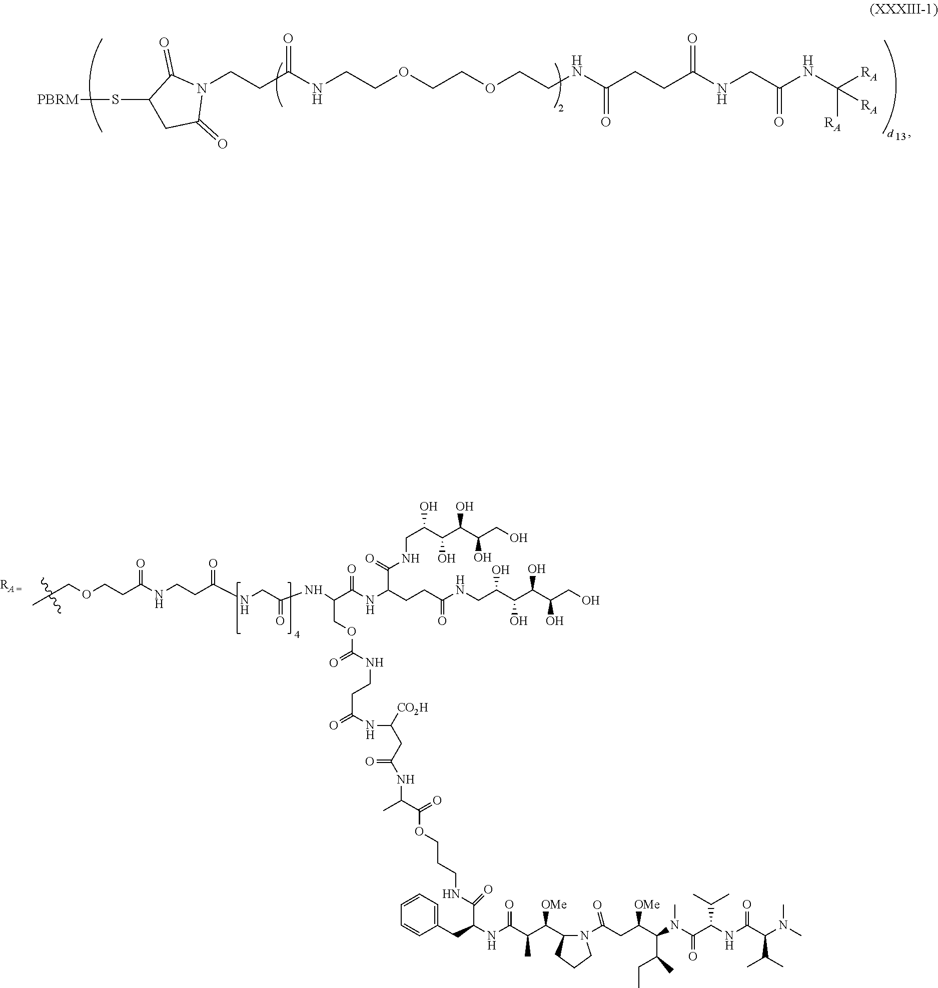

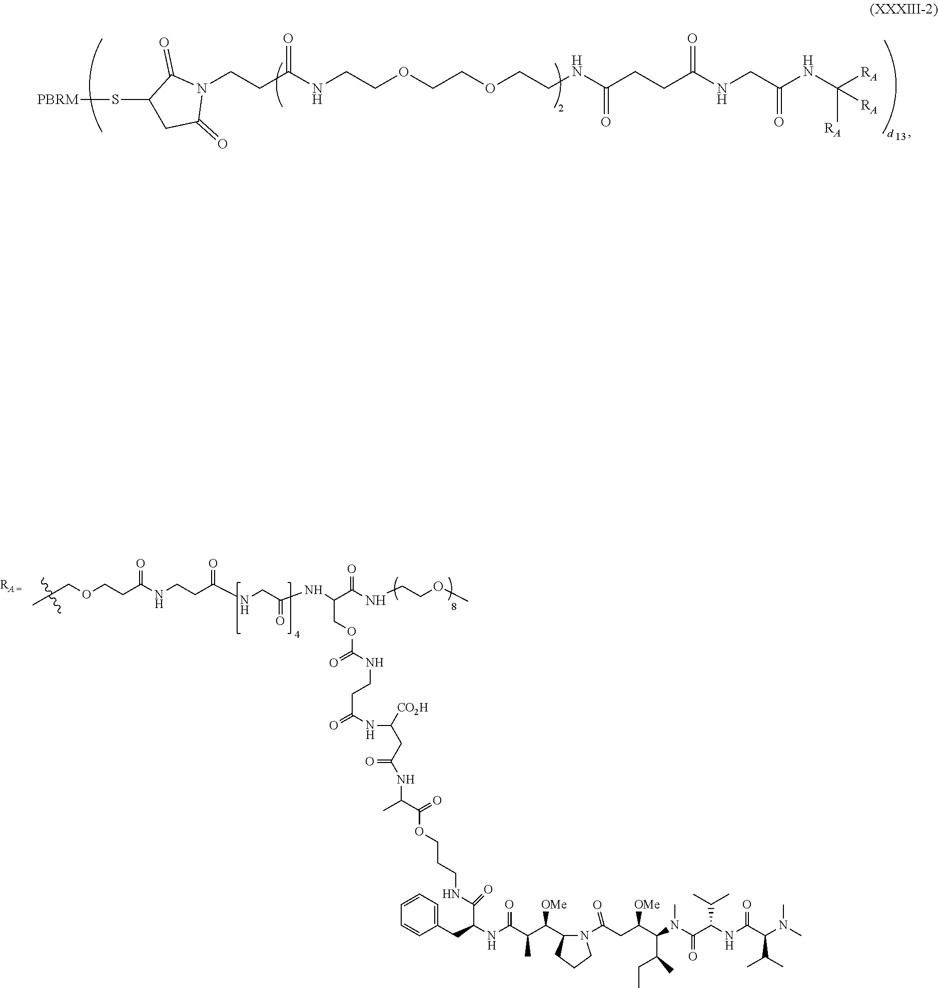

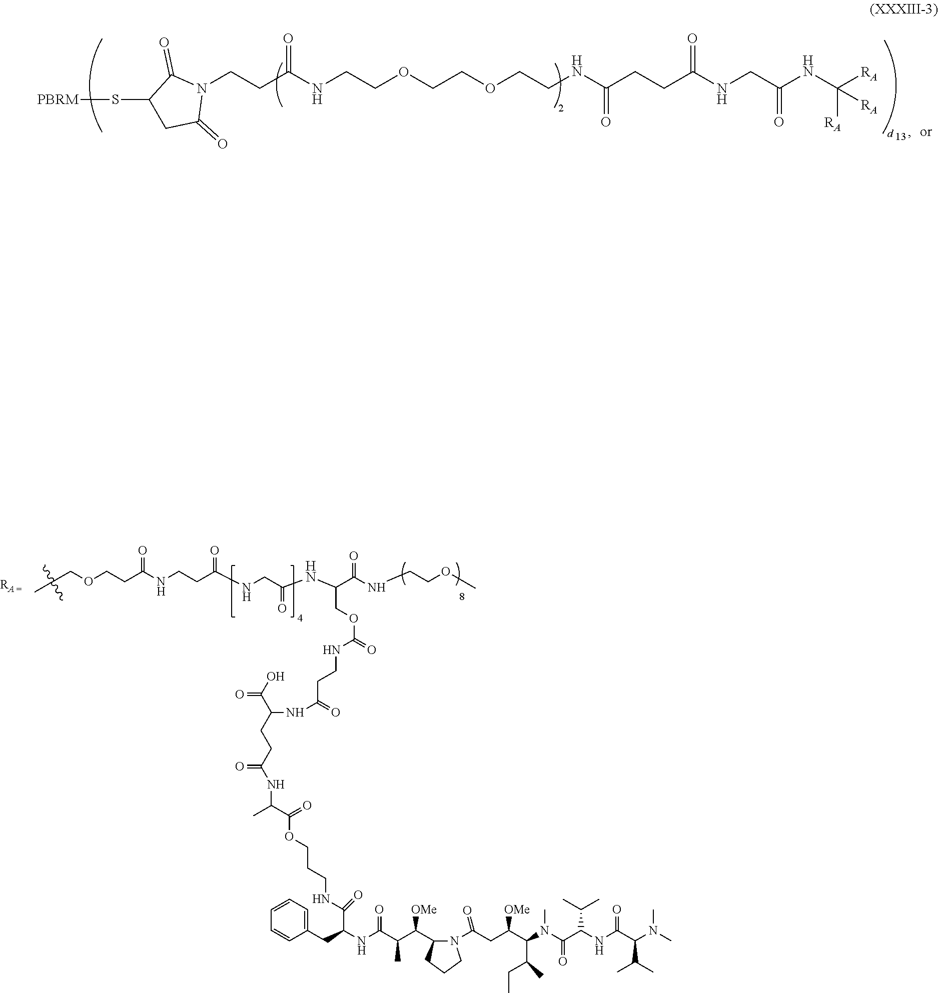

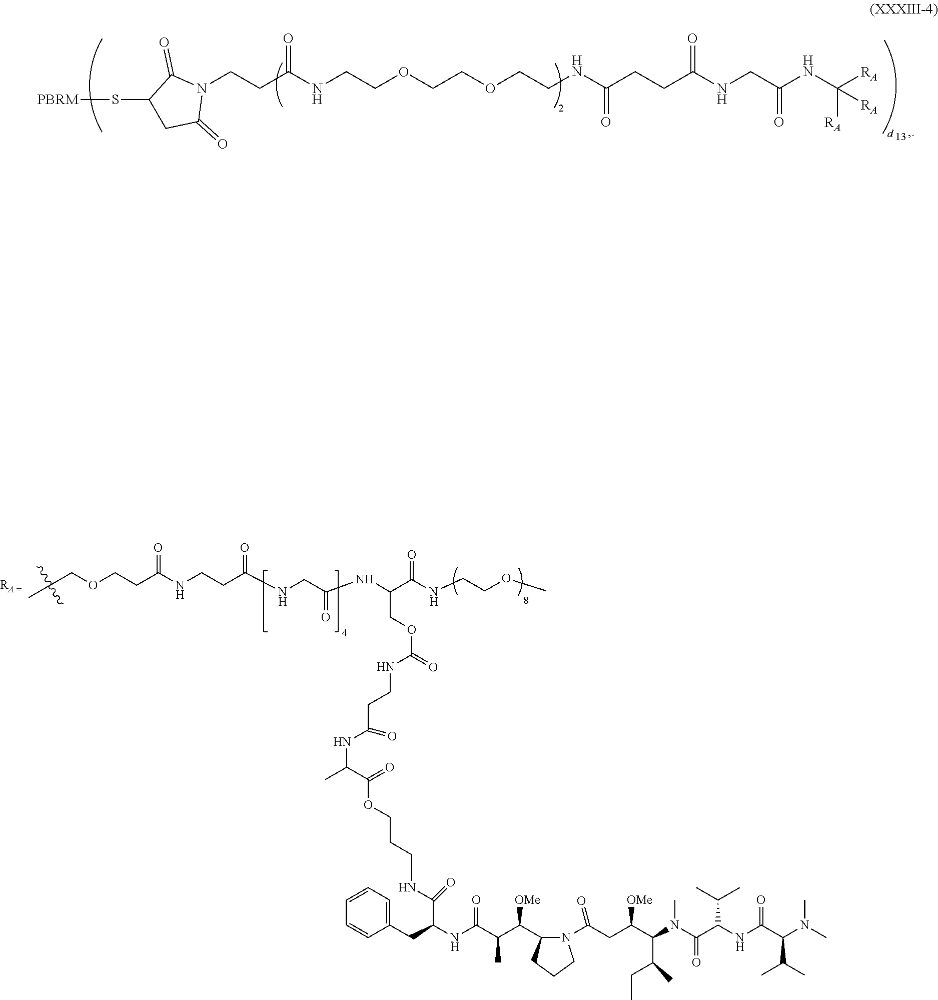

In some embodiments, the cysteine engineered protein-drug conjugates are conjugates of Formula (XXXIII-6) (XXXII-7), (XXXXIII-8) or (XXXIII-9):

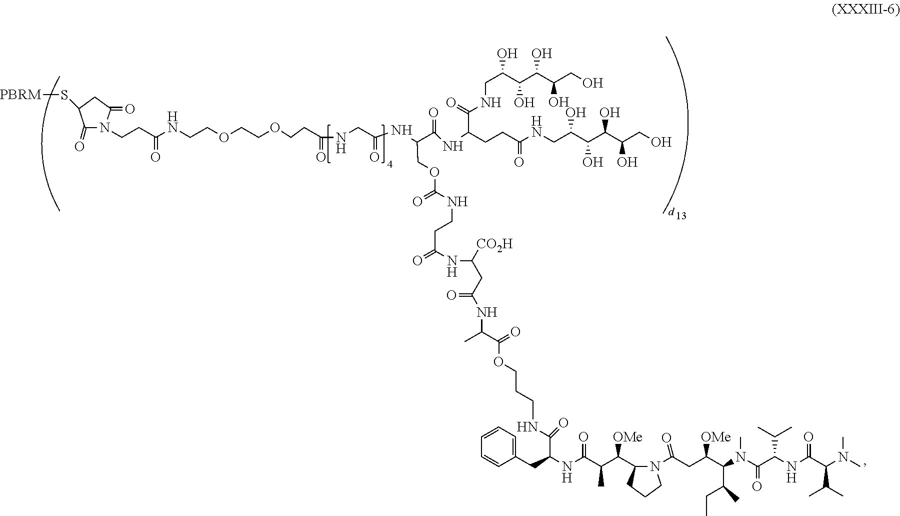

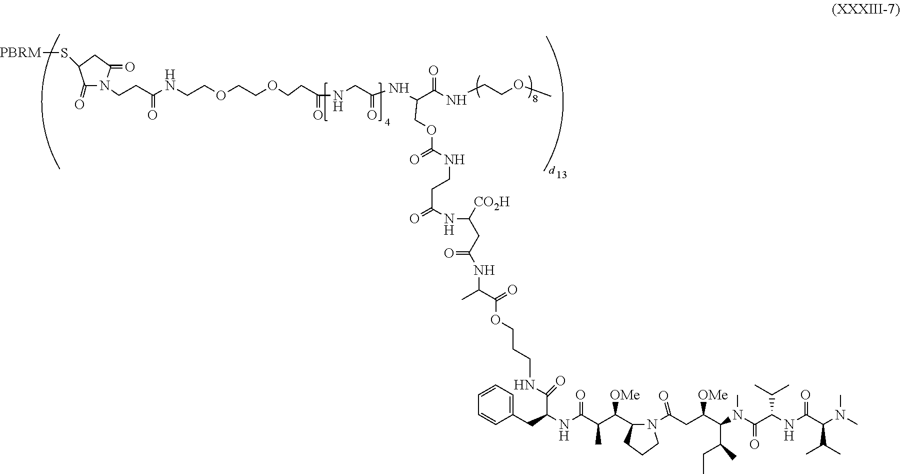

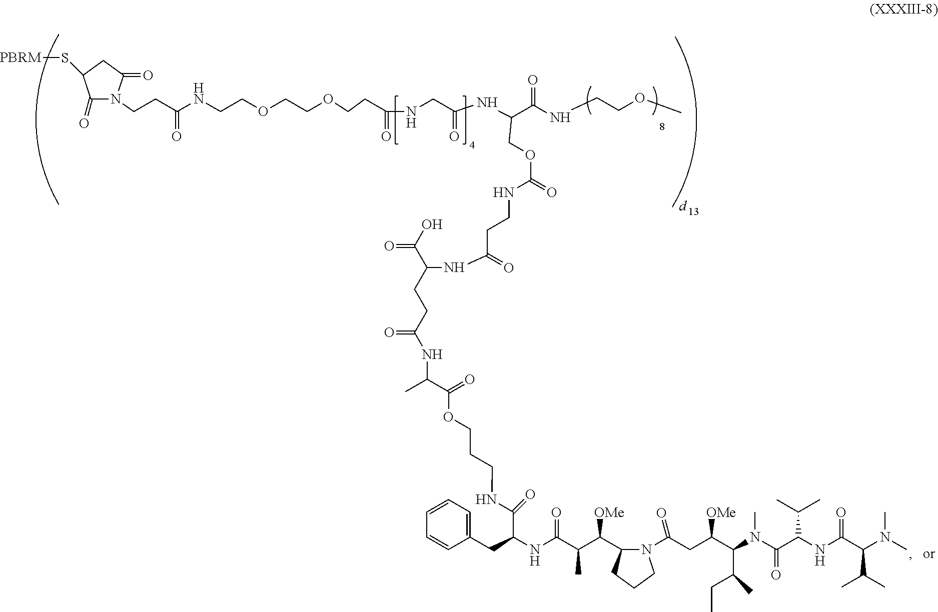

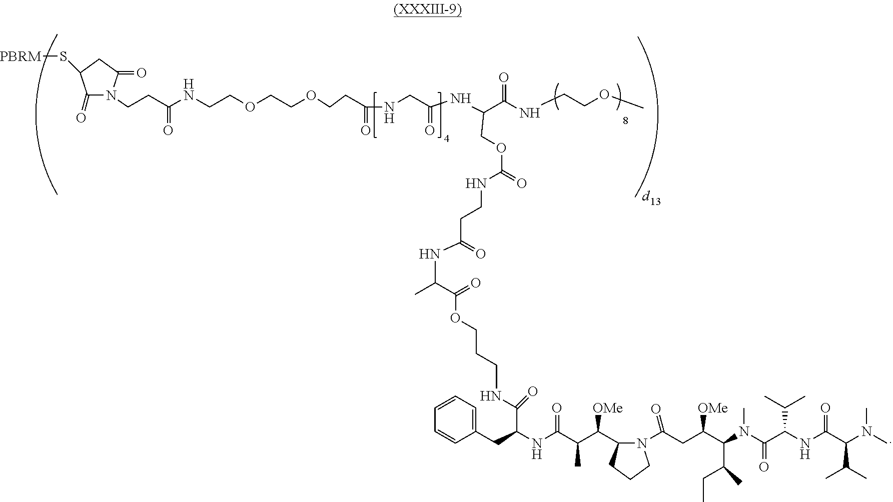

In some embodiments, the cysteine engineered protein-drug conjugates are conjugates of Formula (XXXIII-5):

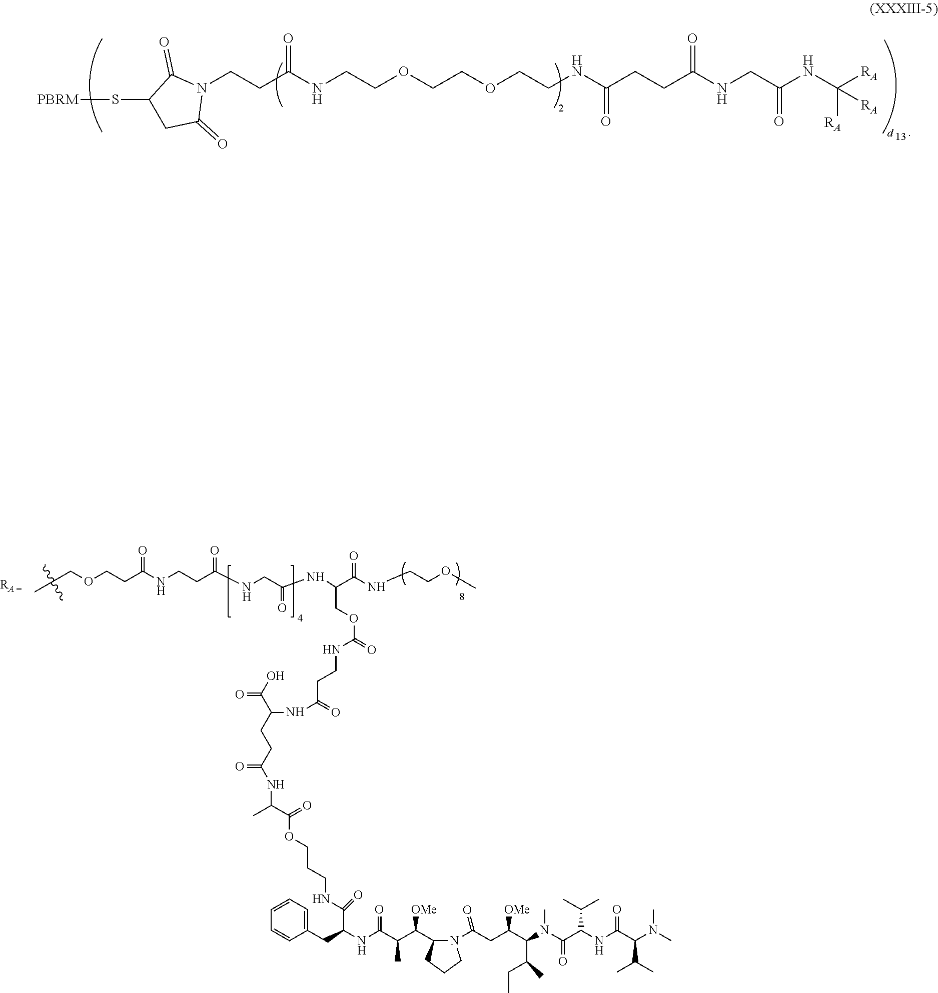

wherein PBRM is a cysteine engineered PBRM and d₁₃ is as defined herein.

### Pharmaceutical Compositions

Also included are pharmaceutical compositions comprising one or more conjugates as disclosed herein in an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). In some embodiments, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of the conjugate in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the conjugates to an accessible diseased tissue. The rate of entry of an active agent into the circulation has been shown to be a function of molecular weight or size. The use of a conjugate of this disclosure can localize the drug delivery in certain cells, such as cancer cells via the specificity of PBRMs.

A “pharmaceutically acceptable formulation” means a composition or formulation that allows for the effective distribution of the conjugates in the physical location most suitable for their desired activity. In some embodiments, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. In some embodiments, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/mL to 25 mg/mL human serum albumin, (2) 0.9% saline (0.9% w/v NaCl), and (3) 5% (w/v) dextrose.

The term “pharmaceutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition, and the therapeutic or combination of therapeutics selected for administration. Pharmaceutically effective amounts for a given situation can be determined by routine experimentation that is within the skill and judgment of the clinician. In a preferred aspect, the disease or condition to can be treated via gene silencing.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic and/or prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic and/or prophylactic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

In some embodiments, a drug or its derivatives, drug-conjugates or PBRM-drug conjugates can be evaluated for their ability to inhibit tumor growth in several cell lines using Cell titer Glo. Dose response curves can be generated using SoftMax Pro software and IC₅₀ values can be determined from four-parameter curve fitting. Cell lines employed can include those which are the targets of the PBRM and a control cell line that is not the target of the PBRM contained in the test conjugates.

In some embodiments, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.001 mg and about 140 mg/kg of body weight per day are useful in the treatment of the above-indicated conditions (between about 0.05 mg and about 7 g per subject per day). In some embodiments, the dosage administered to a patient is between about 0.001 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.001 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise ranges described in the preceding paragraphs, or from about 0.01 mg to about 200 mg of a conjugate per kg of the animal's body weight. In one aspect, the composition can include from about 1 to about 100 mg of a conjugate per kg of the animal's body weight. In some aspects, the amount administered will be in the range from about 0.1 mg/kg to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 mL of normal saline to which about 5 to about 10 mL of human serum albumin can be added. Continuous infusions are given in about 250 to about 500 mL of normal saline, to which about 25 to about 50 mL of human serum albumin can be added, per 24 hour period.

In some embodiments, about one to about four weeks after treatment, the patient can receive a second course of treatment. Specific clinical protocols with regard to route of administration, excipients, diluents, dosages, and times can be determined by the skilled artisan as the clinical situation warrants.

In some embodiments, the therapeutically effective amount may be provided on another regular schedule, i.e., daily, weekly, monthly, or yearly basis or on an irregular schedule with varying administration days, weeks, months, etc. Alternatively, the therapeutically effective amount to be administered may vary. In some embodiments, the therapeutically effective amount for the first dose is higher than the therapeutically effective amount for one or more of the subsequent doses. In some embodiments, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the recommendations of the relevant regulatory bodies and judgment of a health-care practitioner. The therapeutically effective amounts described herein refer to total amounts administered for a given time period; that is, if more than one different conjugate described herein is administered, the therapeutically effective amounts correspond to the total amount administered. It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

In some embodiments, a therapeutically effective amount of a conjugate disclosed herein relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of conjugates disclosed herein may be, by way of nonlimiting example, from about 0.1 mg/kg body weight to about 50 mg/kg body weight, from about 0.1 mg/kg body weight to about 100 mg/kg body weight or from about 0.1 mg/kg body weight to about 150 mg/kg body weight. Common dosing frequencies may range, for example, from twice daily to once a month (e.g., once daily, once weekly; once every other week; once every 3 weeks or monthly). In some embodiments, conjugates disclosed herein can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg). In some embodiments, conjugates disclosed herein can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 19 mg/kg, or 20 mg/kg) for treating cancer.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiments, the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine. The present disclosure also provides pharmaceutical kits comprising one or more containers filled with one or more of the conjugates and/or compositions of the present disclosure, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. The compositions described herein can be packaged as a single dose or for continuous or periodic discontinuous administration. For continuous administration, a package or kit can include the conjugates in each dosage unit (e.g., solution or other unit described above or utilized in drug delivery), and optionally instructions for administering the doses daily, weekly, or monthly, for a predetermined length of time or as prescribed. If varying concentrations of a composition, of the components of the composition, or the relative ratios of the conjugates or agents within a composition over time is desired, a package or kit may contain a sequence of dosage units which provide the desired variability.

A number of packages or kits are known in the art for dispensing pharmaceutical agents for periodic oral use. In some embodiments, the package has indicators for each period. In some embodiments, the package is a labeled blister package, dial dispenser package, or bottle. The packaging means of a kit may itself be geared for administration, such as a syringe, pipette, eye dropper, or other such apparatus, from which the formulation may be applied to an affected area of the body, injected into a subject, or even applied to and mixed with the other components of the kit.

### Methods of Use

### Methods of Treating

In some embodiments, the protein-drug conjugate of the disclosure is used in methods of treating animals. In some embodiments, the protein-drug conjugate of the disclosure is used in methods of treating mammals. In some embodiments, the protein-drug conjugate of the disclosure is used in methods of treating humans (e.g., males, females, infants, children, or adults). In some embodiments, the conjugates of the present disclosure may be used in a method of treating animals which comprises administering to the animal a biodegradable biocompatible conjugate of the disclosure. In some embodiments, conjugates of the disclosure can be administered in the form of soluble linear polymers, copolymers, conjugates, colloids, particles, gels, solid items, fibers, films, etc. Biodegradable biocompatible conjugates disclosed herein can be used as drug carriers and drug carrier components, in systems of controlled drug release, preparations for low-invasive surgical procedures, etc. Pharmaceutical formulations can be injectable, implantable, etc.

In some embodiments, the disclosure provides a method of treating a disease or disorder in a subject in need thereof, comprising administering to the subject an efficient amount of at least one conjugate of the disclosure; wherein said conjugate releases one or more therapeutic agents upon biodegradation.

In some embodiments, the particular types of cancers that can be treated with the conjugates include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, gall bladder cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, papillary thyroid cancer, endometrial cancer, papillary renal cell cancer, cholangiocarcinoma and salivary duct cancer, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, non-small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In some embodiments, the conjugates can be administered in vitro, in vivo and/or ex vivo to treat patients and/or to modulate the growth of selected cell populations in patients having anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal, or gastric cancer.

In some embodiments, the cancers are selected from the group consisting of breast cancer, gastric cancer, non-small cell lung cancer (NSCLC), prostate cancer, and ovarian cancer.

In some embodiments, the cancers are selected from the group consisting of ovarian cancer, non-small cell lung cancer (NSCLC), papillary thyroid cancer, endometrial cancer, papillary clear cell renal cell cancer, cholangiocarcinoma, breast cancer, kidney cancer, cervical cancer, and salivary duct cancer.

In some embodiments, the conjugates can be administered in vitro, in vivo and/or ex vivo to treat, prevent, reduce the risk of developing and/or delay onset of certain pathologies or disorders, for example, a cancer. In some embodiments, the conjugates of the disclosure are useful in treating, preventing, delaying the progression of or otherwise ameliorating a symptom of a cancer selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, gastric cancer, papillary thyroid cancer, endometrial cancer, papillary renal cell cancer, cholangiocarcinoma, and salivary duct cancer.

In some embodiments the conjugates can be administered in vitro, in vivo and/or ex vivo to treat autoimmune diseases, such as systemic lupus, rheumatoid arthritis, psoriasis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In some embodiments the conjugates can also be used for the manufacture of a medicament useful for treating or lessening the severity of disorders, such as, characterized by abnormal growth of cells (e.g., cancer).

In some embodiments, the therapeutic agent is locally delivered to a specific target cell, tissue, or organ.

In some embodiments, in practicing the method of the disclosure, the conjugate further comprises or is associated with a diagnostic label. In some embodiments, the diagnostic label is selected from the group consisting of: radiopharmaceutical or radioactive isotopes for gamma scintigraphy and positron emission tomography (PET), contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In some embodiments, the conjugate is further monitored in vivo.

Examples of diagnostic labels include, but are not limited to, diagnostic radiopharmaceutical or radioactive isotopes for gamma scintigraphy and positron emission tomography (PET), contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include T-emitting radionuclides, e.g., indium-111, technetium-99m and iodine-131, etc. Contrast agents for MRI (Magnetic Resonance Imaging) include magnetic compounds, e.g., paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photoimaging. In some embodiments a modifier comprises a paramagnetic ion or group.

In some embodiments, the present disclosure provides a method of treating a disease or disorder in a subject, comprising preparing an aqueous formulation of at least one conjugate of the disclosure and parenterally injecting said formulation in the subject.

In some embodiments, the disclosure provides a method of treating a disease or disorder in a subject, comprising preparing an implant comprising at least one conjugate of the disclosure, and implanting said implant into the subject. In certain exemplary embodiments, the implant is a biodegradable gel matrix.

In some embodiments, the disclosure provides a method for treating of an animal in need thereof, comprising administering a conjugate according to the methods described above.

In some embodiments, the disclosure provides a method for eliciting an immune response in an animal, comprising administering a conjugate as in the methods described above.

In some embodiments, the disclosure provides a method of diagnosing a disease in an animal, comprising steps of:

administering a conjugate as in the methods described above, wherein said conjugate comprises a detectable molecule; and

detecting the detectable molecule.

In some embodiments, the step of detecting the detectable molecule is performed non-invasively. In some embodiments, the step of detecting the detectable molecule is performed using suitable imaging equipment.

In some embodiments, a method for treating an animal comprises administering to the animal a biodegradable biocompatible conjugate of the disclosure as a packing for a surgical wound from which a tumor or growth has been removed. The biodegradable biocompatible conjugate packing will replace the tumor site during recovery and degrade and dissipate as the wound heals.

In some embodiments, the conjugate is associated with a diagnostic label for in vivo monitoring.

The conjugates described above can be used for therapeutic, preventative, and analytical (diagnostic) treatment of animals. The conjugates are intended, generally, for parenteral administration, but in some cases may be administered by other routes.

In some embodiments, soluble or colloidal conjugates are administered intravenously. In some embodiments, soluble or colloidal conjugates are administered via local (e.g., subcutaneous, intramuscular) injection. In some embodiments, solid conjugates (e.g., particles, implants, drug delivery systems) are administered via implantation or injection.

In some embodiments, conjugates comprising a detectable label are administered to study the patterns and dynamics of label distribution in animal body.

In some embodiments, any one or more of the conjugates disclosed herein may be used in practicing any of the methods described herein.

The pharmaceutical compositions of the conjugates described herein can be included in a container, pack, or dispenser together with instructions for administration.

In some embodiments, the compositions can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In some embodiments, the active compounds (e.g., conjugates or drugs of the disclosure) are administered in combination therapy, i.e., combined with other agents, e.g., therapeutic agents, that are useful for treating pathological conditions or disorders, such as various forms of cancer, autoimmune disorders and inflammatory diseases. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

In some embodiments, the combination therapy can include one or more conjugates disclosed herein coformulated with, and/or coadministered with, one or more additional antibodies, which can be the same as the antibody used to form the conjugate or a different antibody.

In some embodiments, the combination therapy can include one or more therapeutic agent and/or adjuvant. In some embodiments, the additional therapeutic agent is a small molecule inhibitor, another antibody-based therapy, a polypeptide or peptide-based therapy, a nucleic acid-based therapy, and/or other biologic.

In some embodiments, the additional therapeutic agent is a cytotoxic agent, a chemotherapeutic agent, a growth inhibitory agent, an angiogenesis inhibitor, a PARP (poly(ADP)-ribose polymerase) inhibitor, an alkylating agent, an anti-metabolite, an anti-microtubule agent, a topoisomerase inhibitor, a cytotoxic antibiotic, any other nucleic acid damaging agent or an immune checkpoint inhibitor. In some embodiments, the therapeutic agent used in the treatment of cancer, includes but is not limited to, a platinum compound (e.g., cisplatin or carboplatin); a taxane (e.g., paclitaxel or docetaxel); a topoisomerase inhibitor (e.g., irinotecan or topotecan); an anthracycline (e.g., doxorubicin (ADRIAMYCIN®) or liposomal doxorubicin (DOXIL®)); an anti-metabolite (e.g., gemcitabine, pemetrexed); cyclophosphamide; vinorelbine (NAVELBINE®); hexamethylmelamine; ifosfamide; etoposide; an angiogenesis inhibitor (e.g., Bevacizumab (Avastin®)), thalidomide, TNP-470, platelet factor 4, interferon or endostatin); a PARP inhibitor (e.g., Olaparib (Lynparza™)); an immune checkpoint inhibitor, such as for example, a monoclonal antibody that targets either PD-1 or PD-L ((Pembrolizumab (Keytruda®), atezolizumab (MPDL3280A) or Nivolumab (Opdivo®)) or CTA-4 (Ipilimumab (Yervoy®), a kinase inhibitor (e.g., sorafenib or erlotinib), a proteasome inhibitor (e.g., bortezomib or carfilzomib), an immune modulating agent (e.g., lenalidomide or IL-2), a radiation agent, an ALK inhibitor (e.g. crizotinib (Xalkori), ceritinib (Zykadia), alectinib (Alecensa), dalantercept (ACE-041), brigatinib (AP26113), entrectinib (NMS-E628), PF-06463922 TSR-011, CEP-37440 and X-396), and/or a biosimilar thereof and/or combinations thereof. Other suitable agents include an agent considered standard of care by those skilled in the art and/or a chemotherapeutic agent well known to those skilled in the art.

In some embodiments, the immune checkpoint inhibitor is an inhibitor of CTLA-4. In some embodiments, the immune checkpoint inhibitor is an antibody against CTLA-4. In some embodiments, the immune checkpoint inhibitor is a monoclonal antibody against CTLA-4. In other embodiments, the immune checkpoint inhibitor is a human or humanized antibody against CTLA-4. In some embodiments, the anti-CTLA-4 antibody blocks the binding of CTLA-4 to CD80 (B7-1) and/or CD86 (B7-2) expressed on antigen presenting cells. Exemplary antibodies against CTLA-4 include, but are not limited to, Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (herein incorporated by reference): WO 2001014424; WO 2004035607; US2005/0201994; EP 1212422 B1; WO2003086459; WO2012120125; WO2000037504; WO2009100140; WO200609649; WO2005092380; WO2007123737; WO2006029219; WO20100979597; WO200612168; and WO1997020574. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281, incorporated herein by reference). In some embodiments, the anti-CTLA-4 antibody is for example, those disclosed in: WO 98/42752; U.S. Pat. Nos. 6,682,736 and 6,207,156; Hurwitz et al, Proc. Natl. Acad. Sci. USA, 95(17): 10067-10071 (1998); Camacho et al, J. Clin. Oncol., 22(145): Abstract No. 2505 (2004) (antibody CP-675206); Mokyr et al, Cancer Res., 58:5301-5304 (1998) (incorporated herein by reference).

In some embodiments, the CTLA-4 inhibitor is a CTLA-4 ligand as disclosed in WO1996040915.

In some embodiments, the CTLA-4 inhibitor is a nucleic acid inhibitor of CTLA-4 expression. In some embodiments, anti-CTLA4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA4 RNAi molecules take the form of double stranded RNAi molecules described by Tuschl in European Patent No. EP 1309726 (incorporated herein by reference). In some instances, the anti-CTLA4 RNAi molecules take the form of double stranded RNAi molecules described by Tuschl in U.S. Pat. Nos. 7,056,704 and 7,078,196 (incorporated herein by reference). In some embodiments, the CTLA4 inhibitor is an aptamer described in PCT Publication No. WO2004081021.

Additionally, the anti-CTLA4 RNAi molecules of the present disclosure may take the form be RNA molecules described by Crooke in U.S. Pat. Nos. 5,898,031, 6,107,094, 7,432,249, and 7,432,250, and European Application No. EP 0928290 (incorporated herein by reference).

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In some embodiments, the immune checkpoint inhibitor is a human or humanized antibody against PD-L1. In some embodiments, the immune checkpoint inhibitor reduces the expression or activity of one or more immune checkpoint proteins, such as PD-L1. In some embodiments, the immune checkpoint inhibitor reduces the interaction between PD-1 and PD-L1. Exemplary immune checkpoint inhibitors include antibodies (e.g., an anti-PD-L1 antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. Antibodies include monoclonal antibodies, humanized antibodies, deimmunized antibodies, and Ig fusion proteins. An exemplary anti-PD-L1 antibody includes clone EH12. Exemplary antibodies against PD-L1 include: Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736. In some embodiments, the anti-PD-L1 antibody is an anti-PD-L1 antibody disclosed in any of the following patent publications (herein incorporated by reference): WO2013079174; CN101104640; WO2010036959; WO2013056716; WO2007005874; WO2010089411; WO2010077634; WO2004004771; WO2006133396; WO201309906; US 20140294898; WO2013181634 or WO2012145493.

In some embodiments, the PD-L1 inhibitor is a nucleic acid inhibitor of PD-L1 expression. In some embodiments, the PD-L1 inhibitor is disclosed in one of the following patent publications (incorporated herein by reference): WO2011127180 or WO2011000841. In some embodiments, the PD-L1 inhibitor is rapamycin.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In other or additional embodiments, the immune checkpoint inhibitor is a human, or humanized antibody against PD-L2. In some embodiments, the immune checkpoint inhibitor reduces the expression or activity of one or more immune checkpoint proteins, such as PD-L2. In other embodiments, the immune checkpoint inhibitor reduces the interaction between PD-1 and PD-L2. Exemplary immune checkpoint inhibitors include antibodies (e.g., an anti-PD-L2 antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

In some embodiments, the PD-L2 inhibitor is GlaxoSmithKline's AMP-224 (Amplimmune). In some embodiments, the PD-L2 inhibitor is rHIgMI2B7.

In some embodiments, the immune checkpoint inhibitor is an inhibitor of PD-L1. In some embodiments, the immune checkpoint inhibitor is an antibody against PD-1. In some embodiments, the immune checkpoint inhibitor is a monoclonal antibody against PD-1. In some embodiments, the immune checkpoint inhibitor is a human or humanized antibody against PD-1. In some embodiments, the inhibitors of PD-1 biological activity (or its ligands) disclosed in U.S. Pat. Nos. 7,029,674; 6,808,710; or U.S. Patent Application Nos: 20050250106 and 20050159351 can be used in the combinations provided herein. Exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab, h409A1 1); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011 or hBAT-1), CureTech Ltd.

Additional exemplary anti-PD-1 antibodies are described by Goldberg et al, Blood 1 10(1): 186-192 (2007), Thompson et al, Clin. Cancer Res. 13(6): 1757-1761 (2007), and Korman et al, International Application No. PCT/JP2006/309606 (publication no. WO 2006/121168 A1), each of which are expressly incorporated by reference herein. In some embodiments, the anti-PD-1 antibody is an anti-PD-1 antibody disclosed in any of the following patent publications (herein incorporated by reference): WO014557; WO2011110604; WO2008156712; US2012023752; WO2011110621; WO2004072286; WO2004056875; WO20100036959; WO2010029434; WO201213548; WO2002078731; WO2012145493; WO2010089411; WO2001014557; WO2013022091; WO2013019906; WO2003011911; US20140294898; and WO2010001617.

In some embodiments, the PD-1 inhibitor is a PD-1 binding protein as disclosed in WO200914335 (herein incorporated by reference).

In some embodiments, the PD-1 inhibitor is a peptidomimetic inhibitor of PD-1 as disclosed in WO2013132317 (herein incorporated by reference).

In some embodiments, the PD-1 inhibitor is an anti-mouse PD-1 mAb: clone J43, BioXCell (West Lebanon, N.H.).

In some embodiments, the PD-1 inhibitor is a PD-L1 protein, a PD-L2 protein, or fragments, as well as antibody MDX-1 106 (ONO-4538) tested in clinical studies for the treatment of certain malignancies (Brahmer et al., J Clin Oncol. 2010 28(19): 3167-75, Epub 2010 Jun. 1). Other blocking antibodies may be readily identified and prepared by the skilled person based on the known domain of interaction between PD-1 and PD-L1/PD-L2, as discussed above. In some embodiments, a peptide corresponding to the IgV region of PD-1 or PD-L1/PD-L2 (or to a portion of this region) could be used as an antigen to develop blocking antibodies using methods well known in the art.

In some embodiments, the immune checkpoint inhibitor is an inhibitor of IDO1. In some embodiments, the immune checkpoint inhibitor is a small molecule against IDO1. Exemplary small molecules against IDO1 include: Incyte's INCB024360, NSC-721782 (also known as 1-methyl-D-tryptophan), and Bristol Meyers Squibb's F001287.

In some embodiments, the immune checkpoint inhibitor is an inhibitor of LAG3 (CD223). In some embodiments, the immune checkpoint inhibitor is an antibody against LAG3. In some embodiments, the immune checkpoint inhibitor is a monoclonal antibody against LAG3. In some embodiments, the immune checkpoint inhibitor is a human or humanized antibody against LAG3. In some embodiments, an antibody against LAG3 blocks the interaction of LAG3 with major histocompatibility complex (MHC) class II molecules. Exemplary antibodies against LAG3 include: anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, the anti-LAG3 antibody is an anti-LAG3 antibody disclosed in any of the following patent publications (herein incorporated by reference): WO2010019570; WO2008132601; or WO2004078928.

In some embodiments, the immune checkpoint inhibitor is an antibody against TIM3 (also known as HAVCR2). In some embodiments, the immune checkpoint inhibitor is a monoclonal antibody against TIM3. In some embodiments, the immune checkpoint inhibitor is a human or humanized antibody against TIM3. In some embodiments, an antibody against TIM3 blocks the interaction of TIM3 with galectin-9 (Gal9). In some embodiments, the anti-TIM3 antibody is an anti-TIM3 antibody disclosed in any of the following patent publications (herein incorporated by reference): WO2013006490; WO201155607; WO2011159877; or WO200117057. In some embodiments, a TIM3 inhibitor is a TIM3 inhibitor disclosed in WO2009052623.

In some embodiments, the immune checkpoint inhibitor is an antibody against B7-H3. In some embodiments, the immune checkpoint inhibitor is MGA271.

In some embodiments, the immune checkpoint inhibitor is an antibody against MR. In some embodiments, the immune checkpoint inhibitor is Lirilumab (IPH2101). In some embodiments, an antibody against MR blocks the interaction of KIR with HLA.

In some embodiments, the immune checkpoint inhibitor is an antibody against CD137 (also known as 4-1BB or TNFRSF9). In some embodiments, the immune checkpoint inhibitor is urelumab (BMS-663513, Bristol-Myers Squibb), PF-05082566 (anti-4-1BB, PF-2566, Pfizer), or XmAb-5592 (Xencor). In some embodiments, an anti-CD137 antibody is an antibody disclosed in U.S. Published Application No. US 2005/0095244; an antibody disclosed in issued U.S. Pat. No. 7,288,638 (such as 20H4.9-IgG4 [1007 or BMS-663513] or 20H4.9-IgG1 [BMS-663031]); an antibody disclosed in issued U.S. Pat. No. 6,887,673 [4E9 or BMS-554271]; an antibody disclosed in issued U.S. Pat. No. 7,214,493; an antibody disclosed in issued U.S. Pat. No. 6,303,121; an antibody disclosed in issued U.S. Pat. No. 6,569,997; an antibody disclosed in issued U.S. Pat. No. 6,905,685; an antibody disclosed in issued U.S. Pat. No. 6,355,476: an antibody disclosed in issued U.S. Pat. No. 6,362,325 [1D8 or BMS-469492; 3H3 or BMS-469497; or 3E1]; an antibody disclosed in issued U.S. Pat. No. 6,974,863 (such as 53A2); or an antibody disclosed in issued U.S. Pat. No. 6,210,669 (such as 1D8, 3B8, or 3E1). In some embodiments, the immune checkpoint inhibitor is one disclosed in WO 2014036412. In some embodiments, an antibody against CD137 blocks the interaction of CD137 with CD137L.

In some embodiments, the immune checkpoint inhibitor is an antibody against PS. In some embodiments, the immune checkpoint inhibitor is Bavituximab.

In some embodiments, the immune checkpoint inhibitor is an antibody against CD52. In some embodiments, the immune checkpoint inhibitor is alemtuzumab.

In some embodiments, the immune checkpoint inhibitor is an antibody against CD30. In some embodiments, the immune checkpoint inhibitor is brentuximab vedotin. In some embodiments, an antibody against CD30 blocks the interaction of CD30 with CD30L.

In some embodiments, the immune checkpoint inhibitor is an antibody against CD33. In some embodiments, the immune checkpoint inhibitor is gemtuzumab ozogamicin.

In some embodiments, the immune checkpoint inhibitor is an antibody against CD20. In some embodiments, the immune checkpoint inhibitor is ibritumomab tiuxetan. In some embodiments, the immune checkpoint inhibitor is ofatumumab. In some embodiments, the immune checkpoint inhibitor is rituximab. In some embodiments, the immune checkpoint inhibitor is tositumomab.

In some embodiments, the immune checkpoint inhibitor is an antibody against CD27 (also known as TNFRSF7). In some embodiments, the immune checkpoint inhibitor is CDX-1127 (Celldex Therapeutics). In some embodiments, an antibody against CD27 blocks the interaction of CD27 with CD70.

In some embodiments, the immune checkpoint inhibitor is an antibody against OX40 (also known as TNFRSF4 or CD134). In some embodiments, the immune checkpoint inhibitor is anti-OX40 mouse IgG. In some embodiments, an antibody against 0×40 blocks the interaction of OX40 with OX40L.

In some embodiments, the immune checkpoint inhibitor is an antibody against glucocorticoid-induced tumor necrosis factor receptor (GITR). In some embodiments, the immune checkpoint inhibitor is TRX518 (GITR, Inc.). In some embodiments, an antibody against GITR blocks the interaction of GITR with GITRL.

In some embodiments, the immune checkpoint inhibitor is an antibody against inducible T-cell COStimulator (ICOS, also known as CD278). In some embodiments, the immune checkpoint inhibitor is MEDI570 (MedImmune, LLC) or AMG557 (Amgen). In some embodiments, an antibody against ICOS blocks the interaction of ICOS with ICOSL and/or B7-H4.

In some embodiments, the immune checkpoint inhibitor is an inhibitor against BTLA (CD272), CD160, 2B4, LAIR1, TIGHT, LIGHT, DR3, CD226, CD2, or SLAM As described elsewhere herein, an immune checkpoint inhibitor can be one or more binding proteins, antibodies (or fragments or variants thereof) that bind to immune checkpoint molecules, nucleic acids that downregulate expression of the immune checkpoint molecules, or any other molecules that bind to immune checkpoint molecules (i.e. small organic molecules, peptidomimetics, aptamers, etc.). In some embodiments, an inhibitor of BTLA (CD272) is HVEM. In some instances, an inhibitor of CD160 is HVEM. In some embodiments, an inhibitor of 2B4 is CD48. In some embodiments, an inhibitor of LAIR1 is collagen. In some embodiments, an inhibitor of TIGHT is CD112, CD113, or CD155. In some embodiments, an inhibitor of CD28 is CD80 or CD86. In some embodiments, an inhibitor of LIGHT is HVEM. In some embodiments, an inhibitor of DR3 is TL1A. In some embodiments, an inhibitor of CD226 is CD155 or CD 112. In some embodiments, an inhibitor of CD2 is CD48 or CD58. In some embodiments, SLAM is self-inhibitory and an inhibitor of SLAM is SLAM.

In some embodiments, the immune checkpoint inhibitor inhibits a checkpoint protein that include, but are not limited to CTLA4 (cytotoxic T-lymphocyte antigen 4, also known as CDJ152), PD-L1 (programmed cell death I ligand 1, also known as CD274), PDL2 programmed cell death protein 2), PD-1 (programmed cell death protein 1, also known as CD279), a B-7 family ligand (B7-H1, B7-H3, B7-H4) BTLA (B and T lymphocyte attenuator, also known as CD272), HVEM, TIM3 (T-cell membrane protein 3), GAL9, LAG-3 (lymphocyte activation gene-3; CD223), VISTA, KIR (killer immunoglobulin receptor), 2B4 (also known as CD244), CD160, CGEN-15049, CHK1 (Checkpoint kinase 1), CHK2 (Checkpoint kinase 2), A2aR (adenosine A2a receptor), CD2, CD27, CD28, CD30, CD40, CD70, CD80, CD86, CD137, CD226, CD276, DR3, GITR, HAVCR2, HVEM, IDO1 (indoleamine 2,3-dioxygenase 1), IDO2 (indoleamine 2,3-dioxygenase 2), ICOS (inducible T cell costimulator), LAIR1, LIGHT (also known as TNFSF14, a TNF family member), MARCO (macrophage receptor with collagenous structure), OX40 (also known as tumor necrosis factor receptor superfamily, member 4, TNFRSF4, and CD134) and its ligand OX40L (CD252), SLAM, TIGHT, VTCN1 or a combination thereof.

In some embodiments, the immune checkpoint inhibitor interacts with a ligand of a checkpoint protein that comprises CTLA-4, PDL1, PDL2, PD1, BTLA, HVEM, TIM3, GAL9, LAG3, VISTA, KR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

In some embodiments, the immune checkpoint inhibitor inhibits a checkpoint protein that comprises CTLA-4, PDL1, PD1 or a combination thereof.

In some embodiments, the immune checkpoint inhibitor inhibits a checkpoint protein that comprises CTLA-4 and PD1 or a combination thereof.

In some embodiments, the immune checkpoint inhibitor comprises pembrolizumab (MK-3475), nivolumab (BMS-936558), pidilizumab (CT-011), AMP-224, MDX-1 105, durvalumab (MEDI4736), MPDL3280A, BMS-936559, IPH2101, TSR-042, TSR-022, ipilimumab, lirilumab, atezolizumab, avelumab, tremelimumab, or a combination thereof.

In some embodiments, the immune checkpoint inhibitor is nivolumab (BMS-936558), ipilimumab, pembrolizumab, atezolizumab, tremelimumab, durvalumab, avelumab, or a combination thereof.

In some embodiments, the immune checkpoint inhibitor is pembrolizumab.

Throughout the description, where compounds, scaffolds, and compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

### Synthetic Methods

Any available techniques can be used to make the conjugates or compositions thereof, and intermediates and components (e.g., scaffolds) useful for making them. In some embodiments, semi-synthetic and fully synthetic methods may be used.

The general methods of producing the conjugates or scaffolds disclosed herein are illustrated in Schemes 1 and 2 below and in co-pending U.S. Ser. No. 15/819,650, the disclosure of which is incorporated herein in its entirety. The variables (e.g., M^{P}, M^{A}, L³, W^{D}, W^{M}, L^{D}, and L^{P′}, etc.) in these schemes have the same definitions as described herein unless otherwise specified.

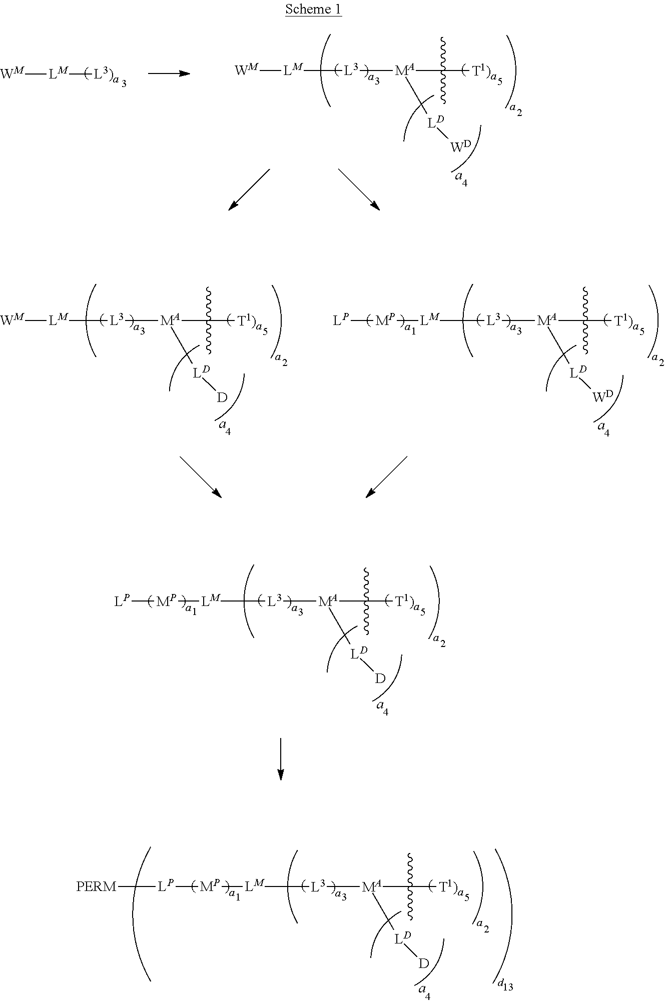

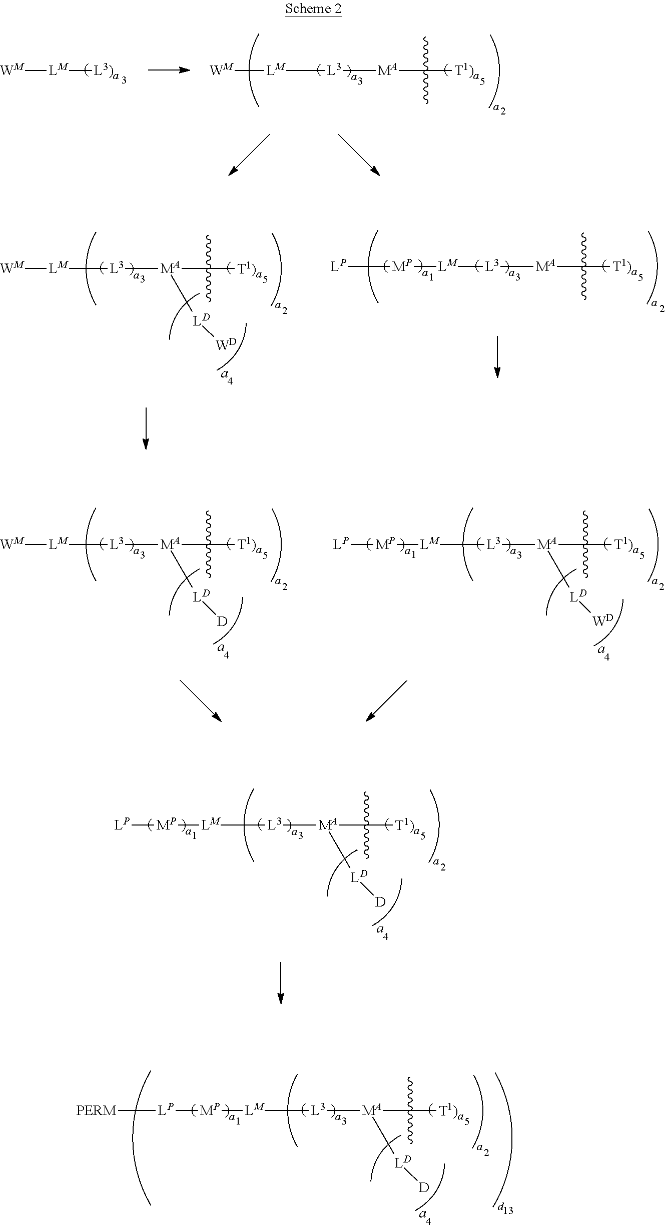

wherein PBRM is a cysteine engineered PBRM

The synthetic processes of the disclosure can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

Drug compounds used for the conjugates of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present disclosure.

Conjugates of the present disclosure and the drug compounds included therein can be prepared by a variety of methods familiar to those skilled in the art. The conjugates or compounds of the disclosure with each of the formulae described herein may be prepared according to the following procedures from commercially available starting materials or starting materials which can be prepared using literature procedures. These procedures show the preparation of representative conjugates of this disclosure.

Conjugates designed, selected and/or optimized by methods described above, once produced, can be characterized using a variety of assays known to those skilled in the art to determine whether the conjugates have biological activity. In some embodiments, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following working examples are illustrative of the linkers, drug molecules and antibodies or antibody fragments, and methods for preparing same. These are not intended to be limiting and it will be readily understood by one of skill in the art that other reagents or methods may be utilized.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples Abbreviations:

EDTA Ethylenediaminetetraacetic acid
TEAA Triethylammonium acetate
TCEP Tris[2-carboxyethyl] phosphine
MI Maleimide or maleimido
PDT Polydispersity index
RP-HPLC Reverse-phase high performance liquid chromatography
SEC Size exclusion chromatography
WCX Weak cation exchange chromatography

### General Information

Cysteine engineered trastuzumab was purchased from GenScript.

Tumor growth inhibition (% TGI) was defined as the percent difference in median tumor volumes (MTVs) between treated and control groups.

Treatment efficacy was determined from the incidence and magnitude of regression responses of the tumor size observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm3 for three consecutive measurements during the course of the study. An animal with a CR response at the termination of a study was additionally classified as a tumor-free survivor (TFS). Animals were monitored for regression responses.

HPLC purification was performed on a Phenomenex Gemini 5 μm 110 Å, 250×10 mm, 5 micron, semi-preparation column.

Whenever possible the drug content of the conjugates was determined quantitatively by chromatography.

The protein content of the protein-polymer drug conjugates was determined spectrophotometrically at 280 nm or by ELISA.

Antibody-polymer-drug conjugates, drug carrying-polymeric scaffolds, or antibody-carrying polymer scaffolds can be purified (i.e., removal of residual unreacted drug, antibody, or polymeric starting materials) by extensive diafiltration. If necessary, additional purification by size exclusion chromatography can be conducted to remove any aggregated antibody-polymer-drug conjugates. In general, the antibody-polymer-drug conjugates as purified typically contain <5% (e.g., <2% w/w) aggregated antibody-polymer-drug conjugates as determined by SEC; <0.5% (w/w) (e.g., <0.1% w/w) free (unconjugated) drug as determined by RP-HPLC or LC-MS/MS; <1% (w/w) of free polymer-drug conjugate as determined by SEC and/or RP-HPLC and <2% (w/w) (e.g., <1% w/w) unconjugated antibody or antibody fragment as determined by HIC-HPLC and/or WCX HPLC. Reduced or partially reduced antibodies were prepared using procedures described in the literature, see, for example, Francisco et al., Blood 102 (4): 1458-1465 (2003). The total drug (conjugated and unconjugated) concentration was determined by RP-HPLC or back-calculation from DAR measured by CE-SDS.

RP-HPLC, or CE-SDS were used to characterize the specificity and distribution of the cysteine bioconjugation sites in the PBRM-polymer-drug conjugates. The results gave the positional distribution of the drug-polymer conjugates on the heavy (H) and light (L) chains of the PBRM.

To determine the concentration of the free drug in a biological sample, an acidified sample was treated with acetonitrile. The free drug was extracted and the acetonitrile supernatant was analyzed. To determine the concentration of conjugated AF-HPA in a non-clinical sample, the sample was subjected to exhaustive basic hydrolysis followed by immunocapture using anti-IgG1 antibody magnetic beads. The acetonitrile supernatant containing the released AF-HPA was analyzed by LC-MS/MS. The total antibody in non-clinical samples were measured by LC-MS/MS after the immunocapture suing anti-IgG1 antibody using the unique peptide after tryptic digestion. For clinical samples, the same procedure could be followed except that an anti-idiotype antibody is used for immunocapture to avoid the interference of endogenous antibodies.

Analysis of free AF and AF-HPA was conducted by RP-HPLC using a C4 column, an acetonitrile gradient and UV detection. Peak areas are integrated and compared to AF and AF-HPA standards. The method is quantitative for AF-HPA and AF in plasma and tissue homogenates and linear over the concentration ranges of 0.1 to 150 ng/mL. The total drug (AF-HPA) released after hydrolysis with NaOH was measured under the same condition with the dynamic range from 1 ng/mL to 5000 ng/mL. The total antibody standards range from 0.1 μg/mL to 100 μg/mL.

The hydrophobicity of the PBRM-polymer-drug conjugates was determined by hydrophobic interaction chromatography (HIC) on a Shimadzu Prominence HPLC system equipped with a diode array detector (DAD). A TSKgel butyl-NPR column (4.6 mm×3.5 cm, 2.5 μm particle size) was held at 35° C. for these analyses. Mobile phase A consisted of 1.5 M ammonium sulfate, 25 mM sodium phosphate, pH 7.0, and mobile phase B was 25 mM sodium phosphate, 10% isopropyl alcohol, pH 7.0. Separations were performed with a 0-100% linear gradient of mobile phase B over 25 minutes followed by 100% mobile phase B for 5 minutes and then returning to 100% A over 5 minutes. The flow rate was 1 mL/min. Sample injections ranged from ˜10 to 100 μg.

The drug to antibody ratio (DAR) was determined by hydrolysis followed by RP-HPLC. The antibody-p-drug conjugates was subjected to exhaustive basic hydrolysis and the released AF-HPA was analyzed by RP-HPLC on a Shimadzu LC-20AD. The calculated free drug concentrations were normalized to the ADC antibody content to determine the DAR.

### Example 1: Synthesis of Stochastic Trastuzumab Conjugate 2 (DAR 13.3)

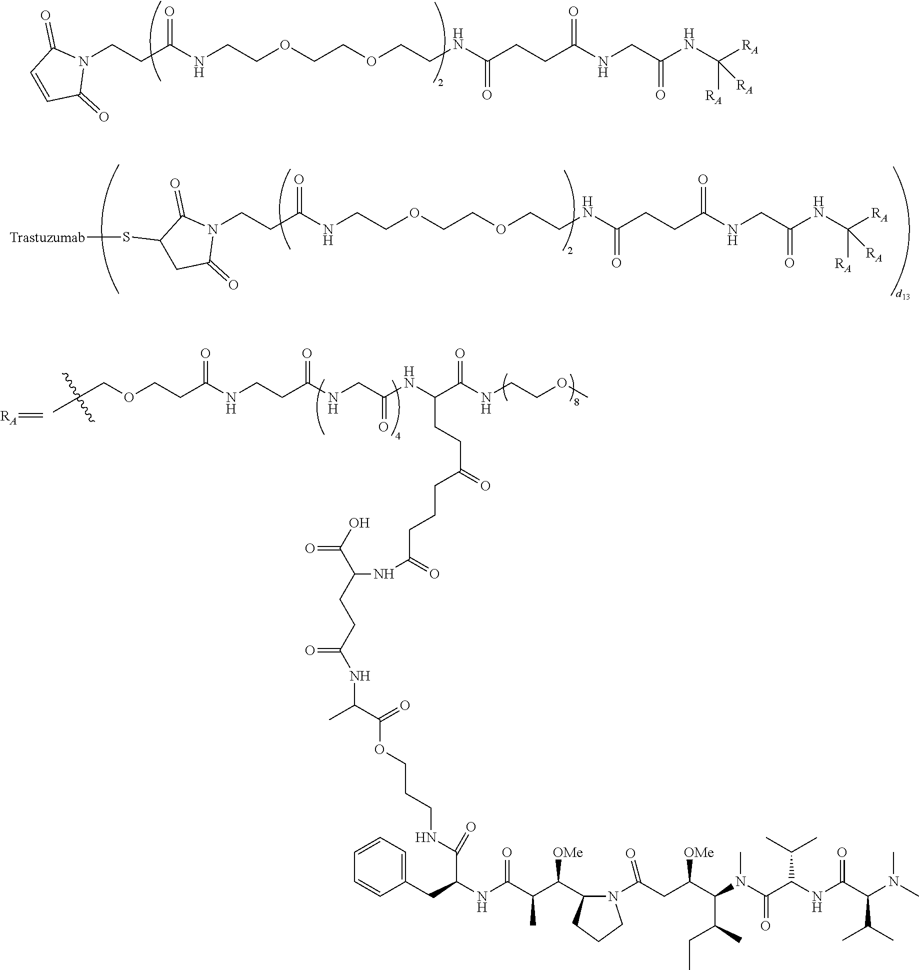

To a solution of trastuzumab (23 mg, 0.156 μmol), in TEAA buffer (50 mM, 1 mM EDTA, pH 7, 3.04 mL) was added a solution of TCEP (0.100 mg, 0.349 μmol) and the resulting mixture was incubated for 1 h at room temperature. The reaction mixture was diluted with TEAA buffer (0.29 mL). A solution of scaffold 1 (9.1 mg, 1.40 μmol, prepared as described in U.S. Ser. No. 15/819,650) dissolved in 1.0 mL TEAA buffer was then slowly added while vigorously stirring the reaction mixture. The reaction mixture was stirred at room temperature for 1 h. Cysteine (0.95 mg, 7.84 μmol) was added and the reaction mixture stirred for 30 minutes. The crude product was purified by WCX to give conjugate 2 (11.8 mg, 51% yield). The purified conjugate had a drug to trastuzumab ratio of 13.3 as determined by hydrolysis followed by RP-HPLC.

### Example 2: Synthesis of Stochastic Trastuzumab Conjugate 3 (DAR 6.4)

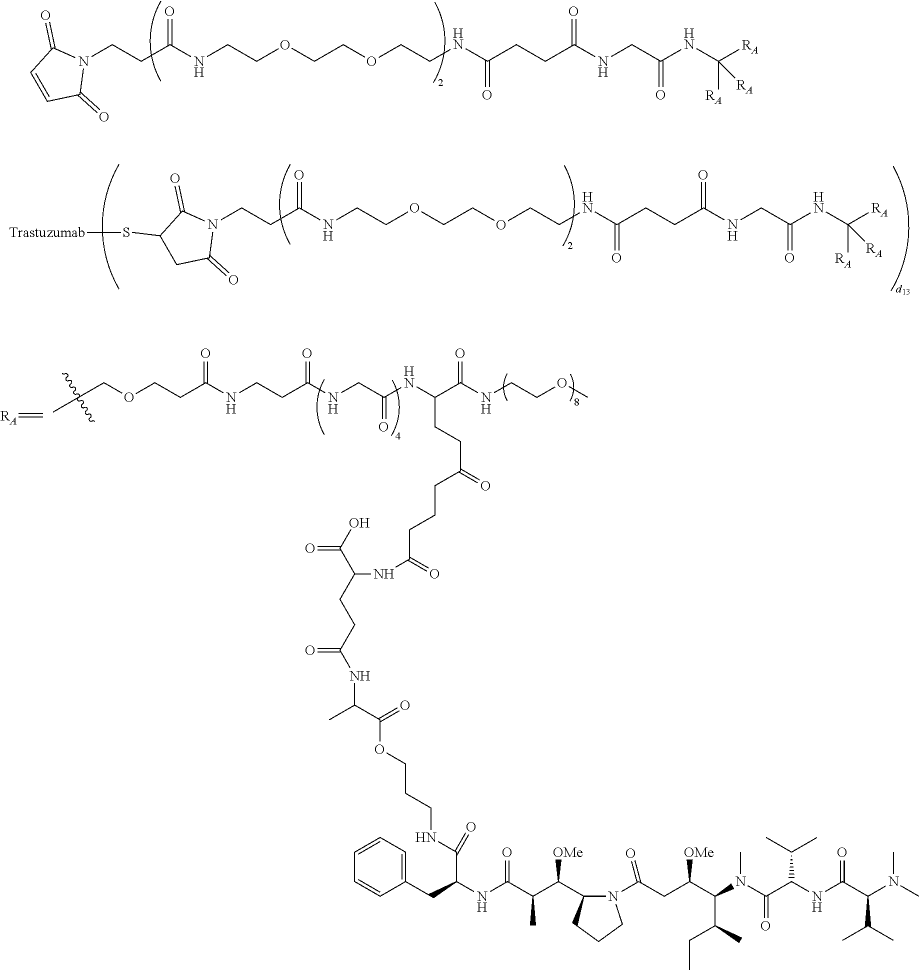

To a solution of trastuzumab (40 mg, 0.275 μmol), in TEAA buffer (50 mM, 1 mM EDTA, pH 7, 0.831 mL) was added a solution of TCEP-HCl (0.118 mg, 0.413 μmol). A solution of scaffold 1 (10.7 mg/mL, 1.65 mM, prepared as described in U.S. Ser. No. 15/819,650) in DMA was added and the resulting mixture was incubated for 1 h at room temperature. L-cysteine (16 mg/mL, 132 mM) was added and the reaction mixture stirred for 30 minutes. The crude reaction mixture was purified by HIC chromatography to give the conjugate 3 (6.7 mg, 11% yield). The purified conjugate had a drug to trastuzumab ratio of 6.4 as determined by hydrolysis followed by RP-HPLC.

### Example 3: Synthesis of Cysteine Engineered Trastuzumab Conjugate 4 (DAR 6.6)

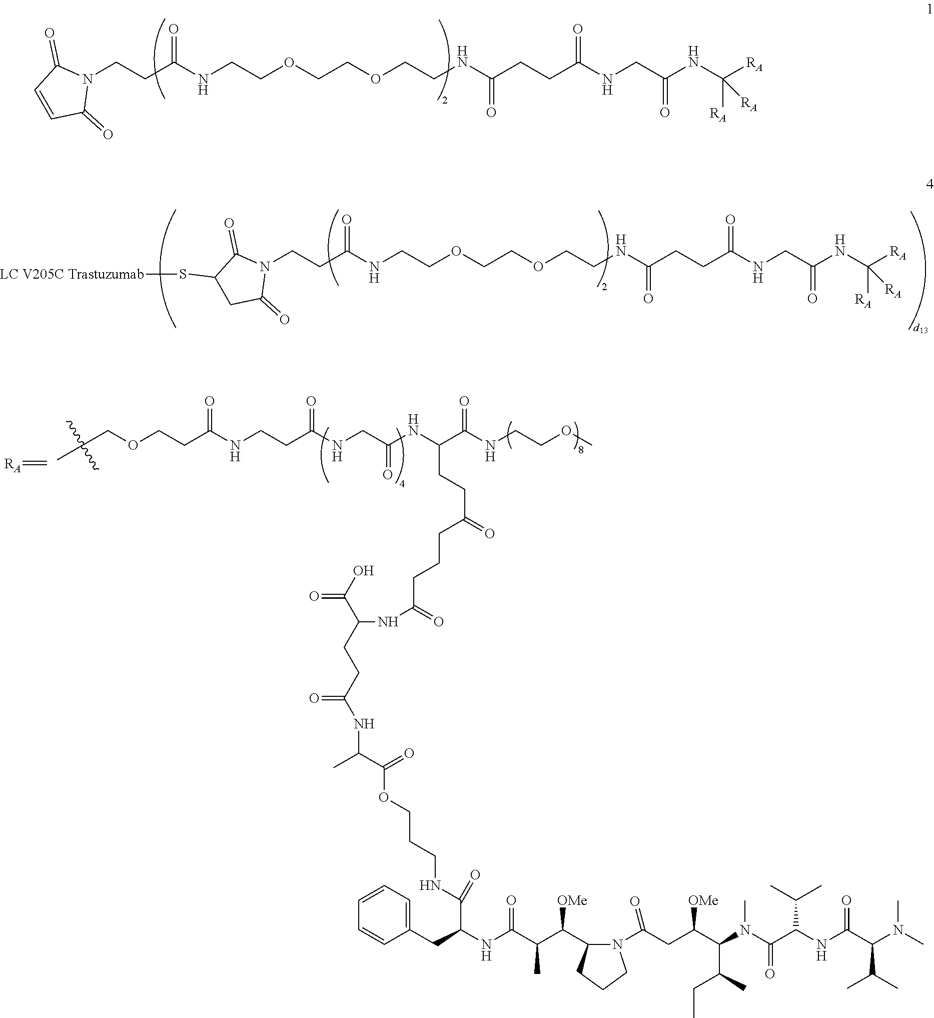

To a solution of cysteine engineered light chain trastuzumab L205C (30 mg, 0.21 μmol), in conjugation buffer (25 mM HEPES, 25 mM NaCl, 1 mM EDTA, pH 8, 5.84 mL, 5.14 mg/mL) was added a solution of TCEP-HCl (0.573 mg, 2.1 μmol) and the resulting mixture was shaken for 4 h at 37° C. the interchain disulfides were reoxidized by adding dehydroascorbic acid (dhAA) dissolved in reaction buffer (8.71 mg/mL, 50 mM) and the mixture was rotated for 2 h at room temperature. A solution of scaffold 1 (6.4 mg/mL, 1 mM, prepared as described in U.S. Ser. No. 15/819,650) in DMSO was added and the resulting mixture was stirred for 1.5 h at room temperature. The pH of the mixture was adjusted to -5.1 with 1 M acetic acid and the crude product was purified by HPLC to give conjugate 4 (5.9 mg, 12% yield). The purified conjugate had a drug to trastuzumab ratio of 6.6 as determined by hydrolysis followed by RP-HPLC.

### Example 4: Cell Viability Assay for the PBRM-Drug Conjugates

The conjugates were evaluated for their antiproliferation properties in tumor cell lines in vitro using CellTiter-Glo® (Promega Corp). SKBR3, cells (HER2 expressing cells), JIMT-1 cells (HER2 medium expression level cells) were plated at a density of 5,000 cells per well in a black walled 96-well plate and allowed to adhere overnight at 37° C. in a humidified atmosphere of 5% CO₂, and were plated. CellTiter-Glo® reagent was added to the wells at room temperature and the luminescent signal was measured after 10 min using a SpectraMax M5 plate reader (Molecular Devices). Dose response curves were generated using Graphpad Prism software. IC₅₀ values were determined from four-parameter curve fitting.

Table I gives illustrative results for the antiproliferation properties of the PBRM-drug conjugates: Conjugate 2, Conjugate 3 and Conjugate 4.

TABLE I

| | SKBR3 | JIMT-1 |
|---|---|---|
| Conjugate | IC₅₀ | IC₅₀ |
| No. | (nmol/L) | (nmol/L) |
| 2 | 0.09 | 1.3 |
| 3 | 0.09 | 0.98 |
| 4 | 0.10 | 0.95 |

As shown in Tables I, the PBRM-drug conjugates show activity in the tested cell lines.

### Example 5: Tumor Growth Response to Administration of PBRM-Drug Conjugates

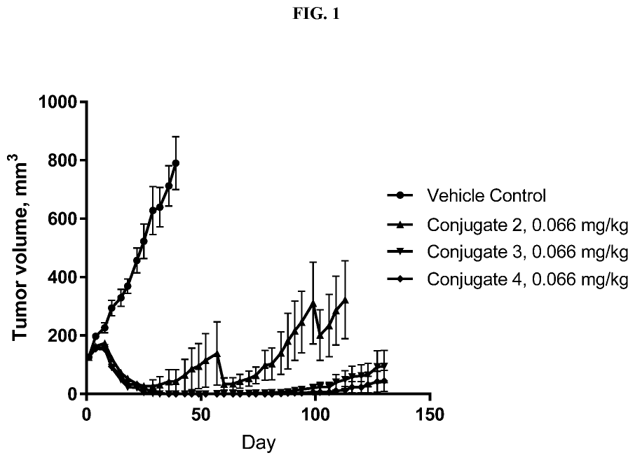

Female CB-17 SCID mice were inoculated subcutaneously with JIMT1 cells (n=10 for each group). Mice were randomized into groups of equal mean tumor volume 12 days post tumor implantation. Test compound or vehicle were dosed IV as a single dose on day 1. Tumor size was measured at the times indicated in FIG. 1 using digital calipers. Tumor volume was calculated and was used to determine the delay in tumor growth. Mice were sacrificed when tumors reached a size of 1000 mm³. Tumor volumes are reported as the mean±SEM for each group.

FIG. 1 provides the results for the tumor response in mice inoculated subcutaneously with JIMT-1 cells (n=10 for each group) after IV administration (12 days post tumor implantation) as a single dose on day 1 of vehicle and the Trastuzumab-drug conjugate, Conjugate 2, Example 1; Conjugate 3, Example 2; and Conjugate 4, Example 3; each at 0.066 mg/kg by payload. The results show that on day 130 at 0.066 mg/kg, Conjugate 2 resulted in 2 partial responses, 8 complete responses and 2 tumor free survivors, Conjugate 3 resulted in 10 complete responses and 6 tumor free survivors and Conjugate 4 resulted in 10 complete responses.

### Example 6. Mouse Exposure after Administration of PBRM-Polymer Drug Conjugates

Female CB-17 SCID mice were inoculated subcutaneously with JIMT1 cells (n=10 for each group). Mice were randomized into groups of equal mean tumor volume 12 days post tumor implantation. The mice were injected intravenously with vehicle (n=3) or with PBRM-polymer-drug conjugate (n=6), Conjugate 2, Example 1; Conjugate 3, Example 2; and Conjugate 4, Example 3; each at, at 0.199 mg/kg by payload. Plasma was collected at 10 min, 24 h, 96 h, 168 h and 336 h post dosing. Body weight was measured prior to dosing on day 1 and on days 1, 7 and 14. All animals were observed throughout the fourteen day period for mortality or morbidity.

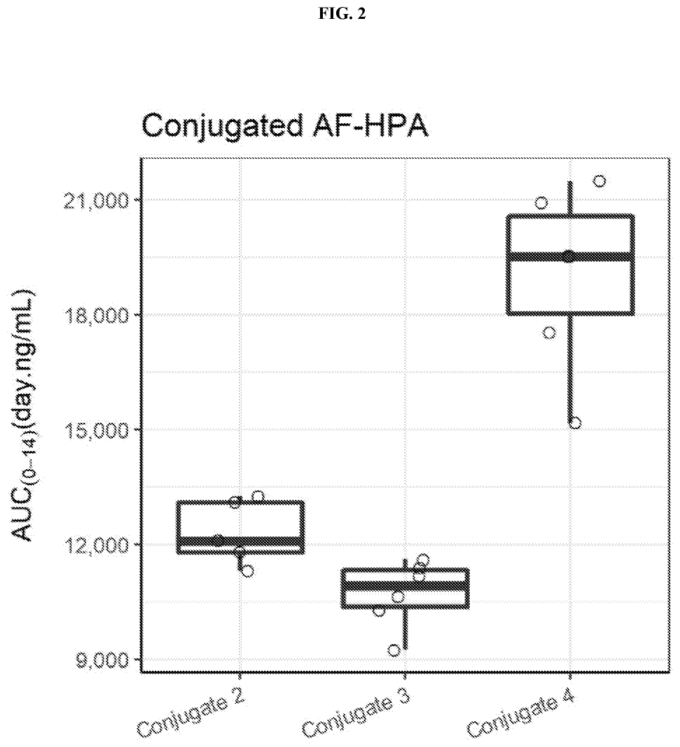

The conjugated AF-HPA concentrations were determined by LC-MS/MS analysis. FIG. 2 depicts the exposure data for Conjugate 2, Conjugate 3, and Conjugate 4. The results show that Conjugate 4 resulted in the highest exposure of the conjugated AF-HPA

All publications, including, e.g., non-patent literature, patent applications, and patents, cited in this specification are incorporated herein by reference for all purposes. The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A conjugate comprising a cysteine engineered targeting moiety and one or more Linker-Drug moieties covalently bonded to the targeting moiety, wherein:
each Linker-Drug moiety includes a Multifunctional Linker that connects the cysteine engineered targeting moiety to one or more Drug Units through intermediacy of a Releasable Assembly Unit for each Drug Unit, and connects a hydrophilic group to the Drug Units of each Linker-Drug moiety, wherein the Releasable Assembly units are capable of releasing free drug in proximity to a target site targeted by the cysteine engineered targeting moiety, and
wherein the Multifunctional Linker comprises a peptide moiety between the cysteine engineered targeting moiety and the hydrophilic group, wherein the peptide moiety includes at least two amino acids.

2. The conjugate of claim 1, wherein the cysteine engineered targeting moiety comprises a cysteine being connected to the Multifunctional Linker.

3. The conjugate of any one of the preceding claims, wherein the cysteine engineered targeting moiety is a protein-based recognition-molecule (PBRM).

4. The conjugate of any one of the preceding claims, wherein the PBRM is an antibody or antibody fragment.

5. The conjugate of any one of the preceding claims, wherein the PBRM is an antibody or antibody fragment comprises light chain V205C, and the PBRM is connected to the Multifunctional Linker through the light chain V205C.

6. The conjugate of any one of the preceding claims, wherein the peptide moiety comprises from three to about ten amino acids.

7. The conjugate of any one of the preceding claims, wherein the peptide moiety comprises at least four amino acids or at least five amino acids.

8. The conjugate of any one of the preceding claims, wherein the hydrophilic group comprises a polyether or a derivative thereof.

9. The conjugate of any one of the preceding claims, wherein the hydrophilic group comprises 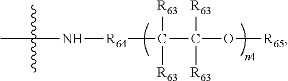
in which
n₄ is an integer from 1 to about 25;
each R₆₃ is independently —H or C₁₋₈ alkyl;
R₆₄ is a bond or a C₁₋₈ alkyl linker;
R₆₅ is —H, C₁₋₈ alkyl or —(CH₂)ₙ₂COOR₆₂;
R₆₂ is —H or C₁₋₈ alkyl; and
n₂ is an integer from 1 to about 5.

10. The conjugate of any one of the preceding claims, wherein the hydrophilic group comprises polyethylene glycol.

11. The conjugate of any one of the preceding claims, wherein the hydrophilic group comprises polyethylene glycol with from about 6 to about 24 PEG subunits,

12. The conjugate of any one of the preceding claims, wherein the hydrophilic group comprises polyethylene glycol with from about 6 to about 12 PEG subunits.

13. The conjugate of any one of the preceding claims, wherein the hydrophilic group comprises polyethylene glycol with from about 8 to about 12 PEG subunits.

14. A conjugate comprising a cysteine engineered targeting moiety and one or more Linker-Drug moieties covalently bonded to the targeting moiety, wherein
each Linker-Drug moiety includes a Multifunctional Linker that connects the cysteine engineered targeting moiety to one or more Drug Units through intermediacy of a Releasable Assembly Unit for each Drug Unit, and connects a polyalcohol or a derivative thereof to the Drug Units of each Linker-Drug moiety, wherein the Releasable Assembly units are capable of releasing free drug in proximity to a target site targeted by the cysteine engineered targeting moiety.

15. The conjugate of any one of the preceding claims, being of Formula (I): 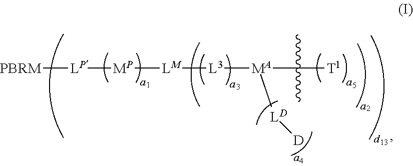
wherein
a₁, when present, is an integer from 0 to 1;
a₂ is 3;
a₃, when present, is an integer from 0 to 1;
a₄ is an integer from 1 to about 5;
a₅ is an integer from 1 to 3;
d₁₃ is an integer from 1 to about 6;
PBRM denotes a protein-based recognition-molecule, wherein the PBRM comprises an engineered cysteine;
L^{P′} is a divalent linker moiety connecting the engineered cysteine of the PBRM to M^{P}; of which the corresponding monovalent moiety L^{P} contains a functional group W^{P} that is capable of forming a covalent bond with a functional group of the engineered cysteine of the PBRM;
M^{P}, when present, is a Stretcher unit;
L^{M} is a tetravalent linker;
L³, when present, is a carbonyl-containing moiety;
M^{A} comprises a peptide moiety that contains at least two amino acids;
T¹ is a hydrophilic group and the [symbol] between T¹ and M^{A} denotes direct or indirect attachment of T¹ and M^{A};
each occurrence of D is independently a therapeutic agent having a molecular weight ≤about 5 kDa; and
each occurrence of L^{D} is independently a divalent linker moiety connecting D to M^{A} and comprises at least one cleavable bond such that when the bond is broken, D is released in an active form for its intended therapeutic effect.

16. A peptide-containing scaffold, being any of Formulae (II)-(V): 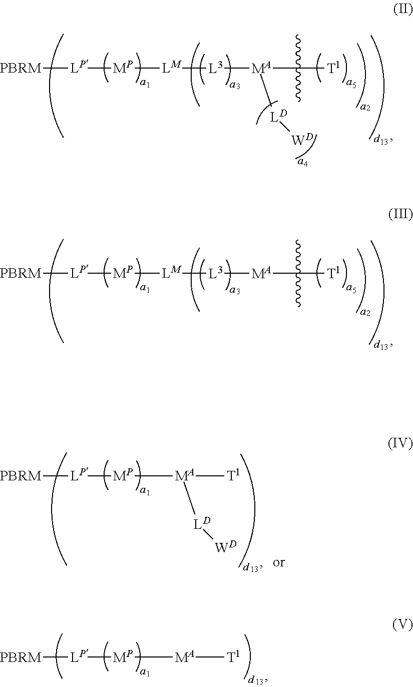
wherein:
a₁, when present, is an integer from 0 to 1;
a₂, when present, is 3;
a₃, when present, is an integer from 0 to 1;
a₄, when present, is an integer from 1 to about 5;
a_{5′} when present, is an integer from 1 to 3;
d₁₃ is an integer from 1 to 6;
PBRM denotes a protein-based recognition-molecule, wherein the PBRM comprises an engineered cysteine;
L^{P′} is a divalent linker moiety connecting the engineered cysteine of the PBRM to M^{P}; of which the corresponding monovalent moiety L^{P} contains a functional group W^{P} that is capable of forming a covalent bond with a functional group of the engineered cysteine of the PBRM;
M^{P}, when present, is a Stretcher unit;
L^{M} is a tetravalent linker, a₂ is 3;
L³, when present, is a carbonyl-containing moiety;
M^{A} comprises a peptide moiety that contains at least two amino acids;
T¹ is a hydrophilic group and the [symbol] between T¹ and M^{A} denotes direct or indirect attachment of T¹ and M^{A};
each occurrence of W^{D} is independently a functional group that is capable of forming a covalent bond with a functional group of a therapeutic agent (“D”) having a molecular weight ≤about 5 kDa; and
each occurrence of L^{D} is independently a divalent linker moiety connecting W^{D} or D to M^{A} and L^{D} comprises at least one cleavable bond such that when the bond is broken, D is released in an active form for its intended therapeutic effect.

17. The conjugate or scaffold of any one of the preceding claims, wherein the PBRM is an antibody or antibody fragment comprising light chain V205C, and wherein the PBRM is connected to Lf through the light chain V205C.

18. The conjugate or scaffold of any one of the preceding claims, wherein L³, when present, comprises —X—C₁₋₁₀ alkylene-C(O)—, with X directly connected to L^{M}, in which X is CH₂, O, or NR₅, and R₅ is —H, C₁₋₆ alkyl, C₆₋₁₀ aryl, C₃₋₈ cycloalkyl, COOH, or COO—C₁₋₆ alkyl.

19. The conjugate or scaffold of any one of the preceding claims, wherein L³, when present, is —NR₅—(CH₂)ᵥ—C(O)— or —CH₂—(CH₂)—C(O)—NR₅—(CH₂)ᵥ—C(O)—, in which each v independently is an integer from 1 to 10.

20. The conjugate or scaffold of any one of the preceding claims, wherein L³, when present, is —NH—(CH₂)₂—C(O)— or —(CH₂)₂—C(O)—NH—(CH₂)₂—C(O)—.

21. The conjugate or scaffold of any one of the preceding claims, wherein each v independently is an integer from 1 to 6, or from 2 to 4, or is 2.

22. The conjugate or scaffold of any one of the preceding claims, wherein a₄ is 1, 2, or 3.

23. The conjugate or scaffold of any one of the preceding claims, wherein d₁₃ is 2, 4 or 6.

24. The conjugate or scaffold of any one of the preceding claims, wherein each W^{P}, when present, is independently: 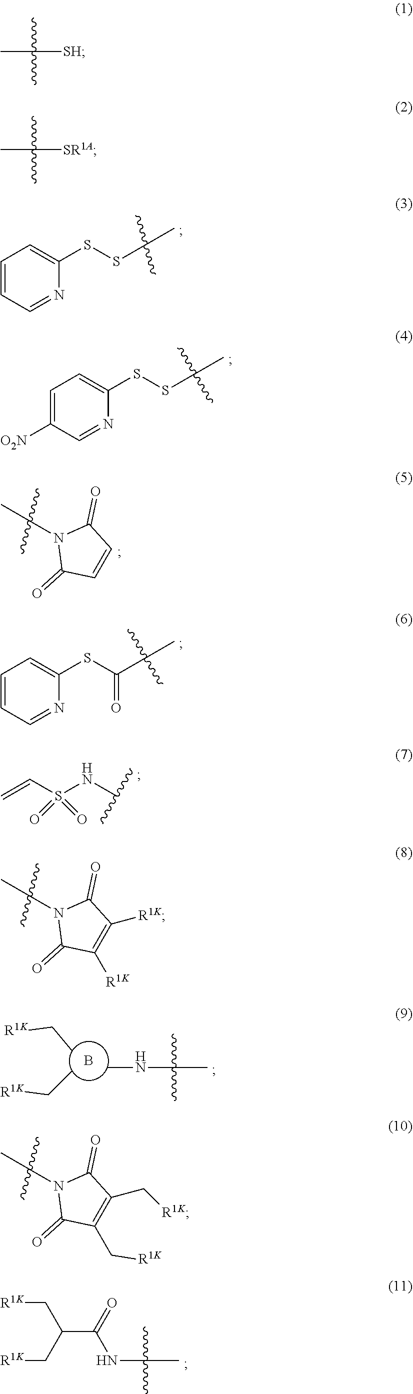 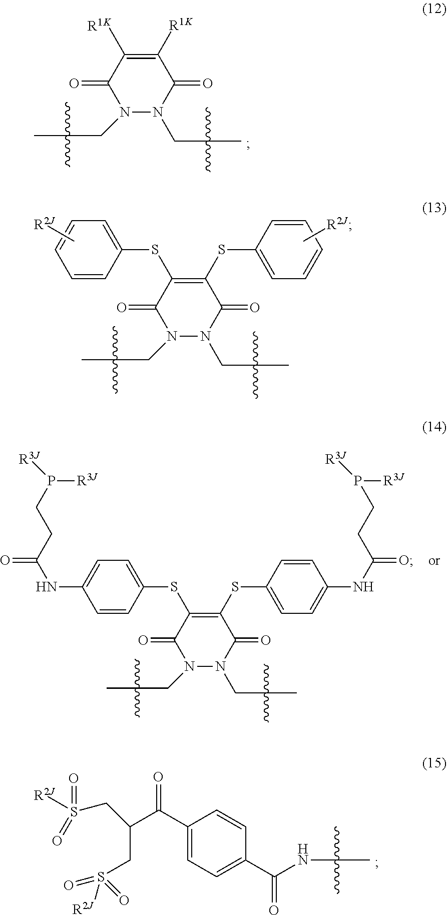
wherein
ring B is cycloalkyl, heterocycloalkyl, aryl, or heteroaryl;
R^{1K} is a leaving group;
R^{1A} is a sulfur protecting group;
R^{2J} is —H, an aliphatic, aryl, heteroaliphatic, or carbocyclic moiety; and
R^{3J} is C₁₋₆ alkyl and each of Z₁, Z₂, Z₃ and Z₇ is independently a carbon or nitrogen atom;

25. The conjugate or scaffold of any one of the preceding claims, wherein R^{1K} is halo or RC(O)O— in which R is —H, an aliphatic, heteroaliphatic, carbocyclic, or heterocycloalkyl moiety.

26. The conjugate or scaffold of any one of the preceding claims, wherein R^{1A} is 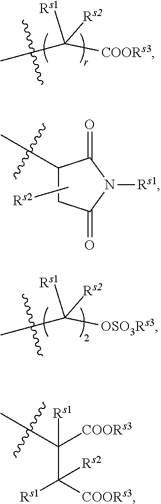
in which r is 1 or 2 and each of R^{s1}, R^{s2}, and R^{s3} is —H, an aliphatic, heteroaliphatic, carbocyclic, or heterocycloalkyl moiety.

27. The conjugate or scaffold of any one of the preceding claims, wherein M^{P}, when present, is —(Z₄)—[(Z₅)—(Z₆)]_{z}—, with Z₄ connected to L^{P′} or L^{P} and Z₆ connected to L^{M}; in which
z is 1, 2, or 3;
Z₄ is: 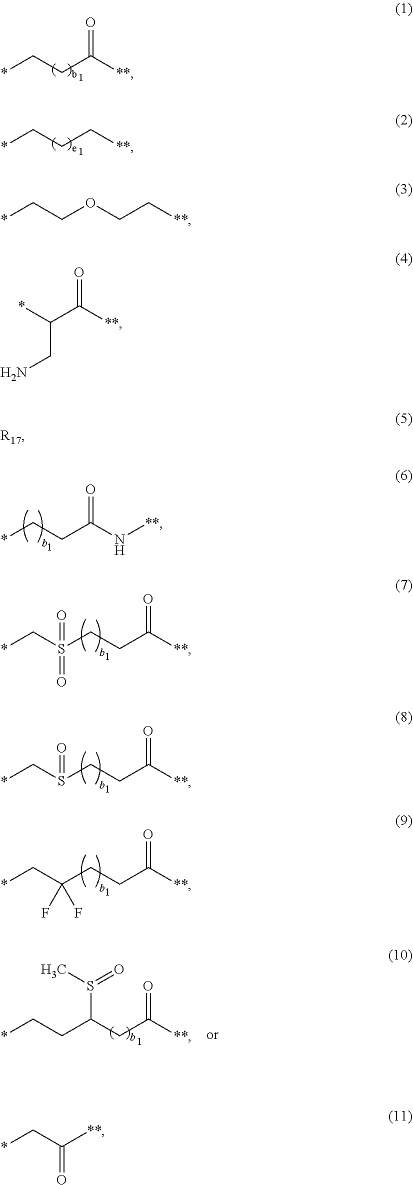
wherein * denotes attachment to L^{P′} or L^{P} and ** denotes attachment to Z₅ or Z₆ when present or to L^{M} when Z₅ and Z₆ are both absent;
b₁ is an integer from 0 to 6;
e₁ is an integer from 0 to 8,
R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4 to 14-membered heterocycloalkylene, —C₁₋₁₀ alkylene-(4 to 14-membered heterocycloalkylene)-, -(4 to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4 to 14-membered heterocycloalkylene-C(═O)—, —C₁₋₁₀ alkylene-(4 to 14-membered heterocycloalkylene)-C(═O)—, -(4 to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4 to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁-C₁₀ alkylene-S—;
each Z₅ independently is absent, R₅₇—R₁₇ or a polyether unit;
each R₅₇ independently is a bond, NR₂₃, S, or O;
each R₂₃ independently is —H, C₁₋₆ alkyl, C₆₋₁₀ aryl, C₃₋₈ cycloalkyl, —COOH, or —COO—C₁₋₆ alkyl; and
each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—;
each R₃ independently is —C(O)—NR₅— or —NR₅—C(O)—;
each R₅ independently is —H, C₁₋₆ alkyl, C₆₋₁₀ aryl, C₃₋₈ cycloalkyl, COOH, or COO—C₁₋₆ alkyl; and
g₁ is an integer from 1 to 4.

28. The conjugate or scaffold of any one of the preceding claims, wherein M^{P}, when present, is 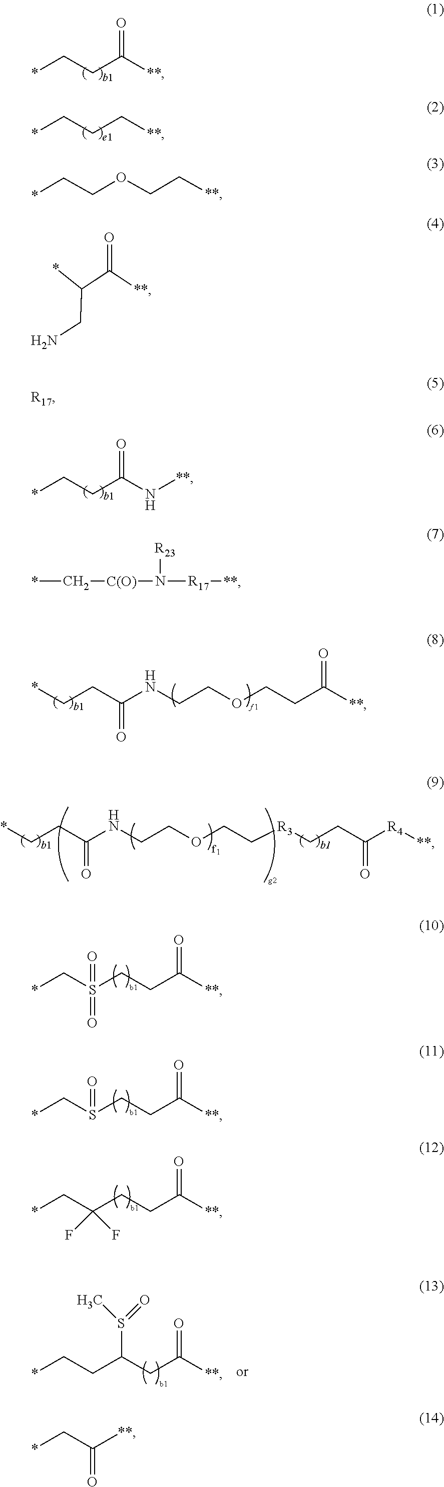
wherein * denotes attachment to L^{P′} or L^{P} and ** denotes attachment to L^{M};
R₃ is —C(O)—NR₅ or —NR₅—C(O)—;
R₄ is a bond or —NR₅—(CR₂₀R₂₁)—C(O)—;
R₅ is —H, C₁₋₆ alkyl, C₆₋₁₀ aryl, C₃₋₈ cycloalkyl, —COOH, or —COO—C₁₋₆ alkyl;
R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4 to 14-membered heterocycloalkylene, —C₁₋₁₀ alkylene-(4 to 14-membered heterocycloalkylene)-, -(4 to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4 to 14-membered heterocycloalkylene-C(═O)—, —C₁₋₁₀ alkylene-(4 to 14-membered heterocycloalkylene)-C(═O)—, -(4 to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4 to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁-C₁₀ alkylene-S—;
each R₂₀ and R₂₁ independently is —H, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
each R₂₃ independently is —H, C₁₋₆ alkyl, C₆₋₁₀ aryl, C₃₋₈ cycloalkyl, —COOH, or —COO—C₁₋₆ alkyl;
each b₁ independently is an integer from 0 to 6;
e₁ is an integer from 0 to 8;
each f₁ independently is an integer from 1 to 6; and
g₂ is an integer from 1 to 4.

29. The conjugate or scaffold of any one of the preceding claims, wherein M^{P}, when present, is 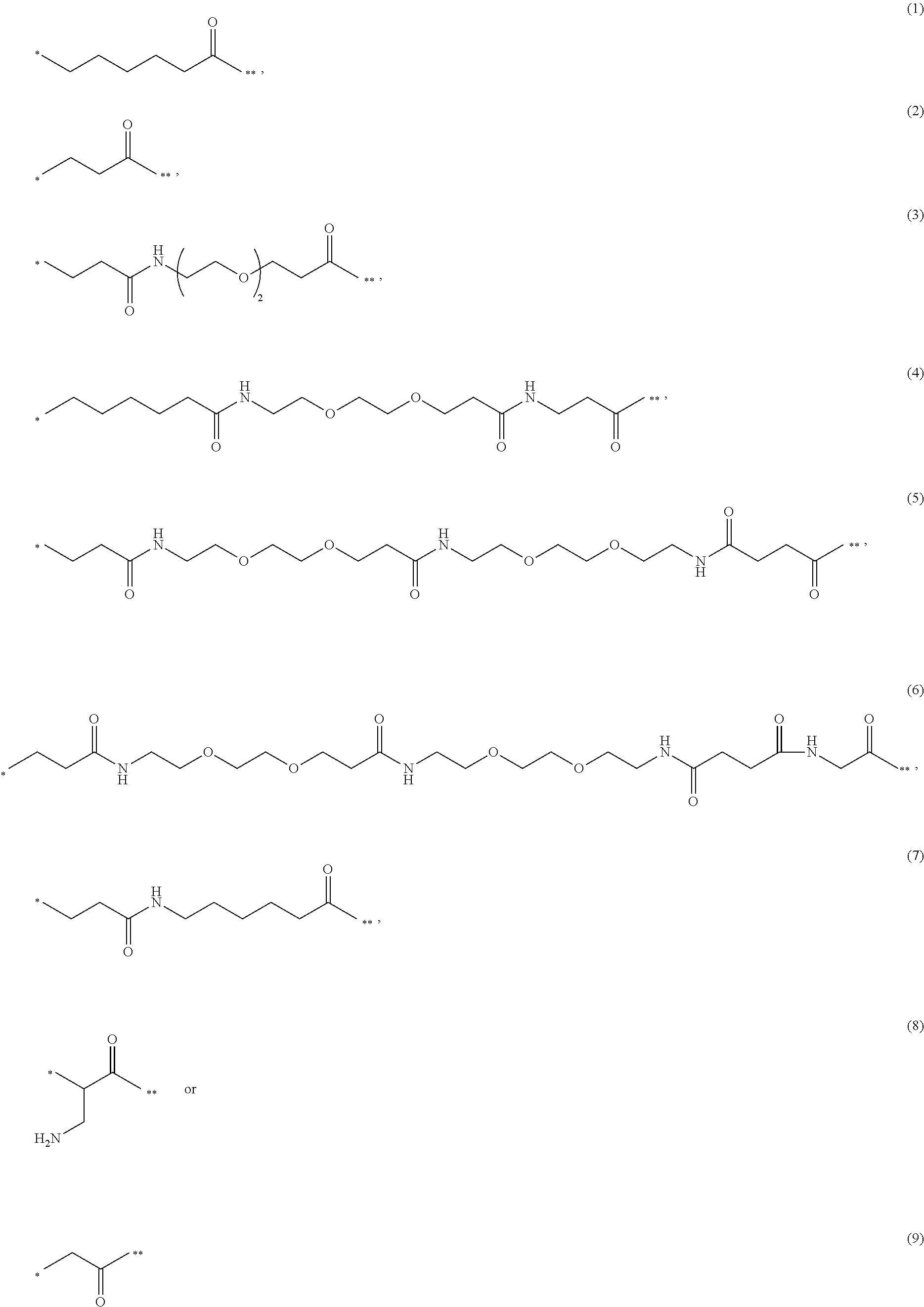
wherein * denotes attachment to L^{P′} or L^{P} and ** denotes attachment to L^{M}.

30. The conjugate or scaffold of any one of the preceding claims, wherein a₂ is 3 and L^{M} is; 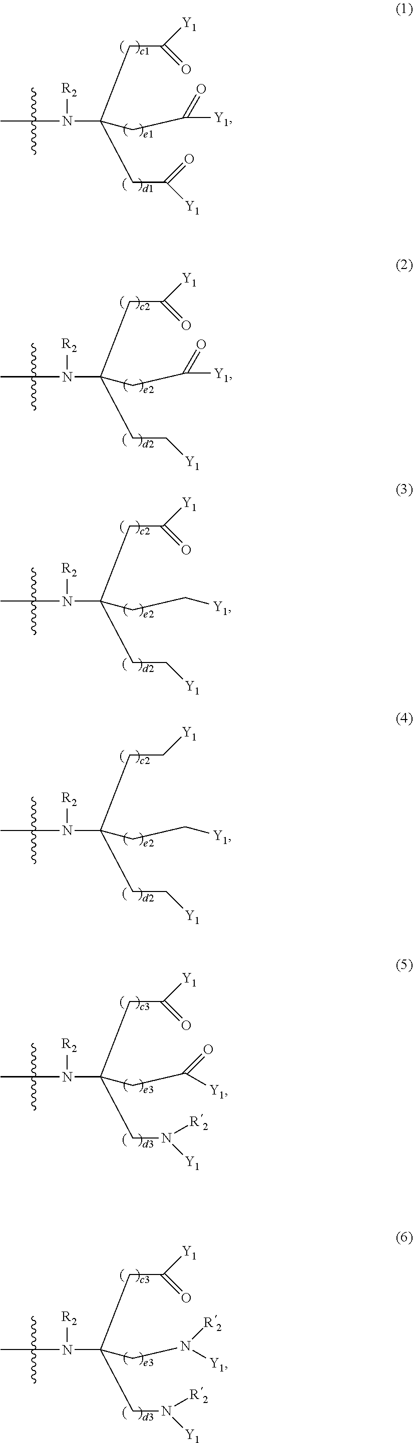 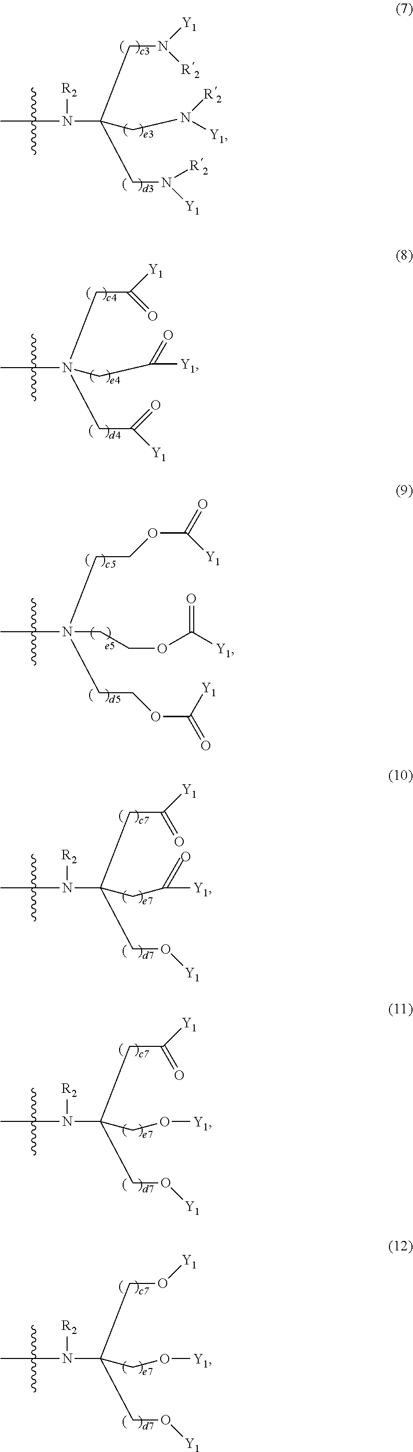 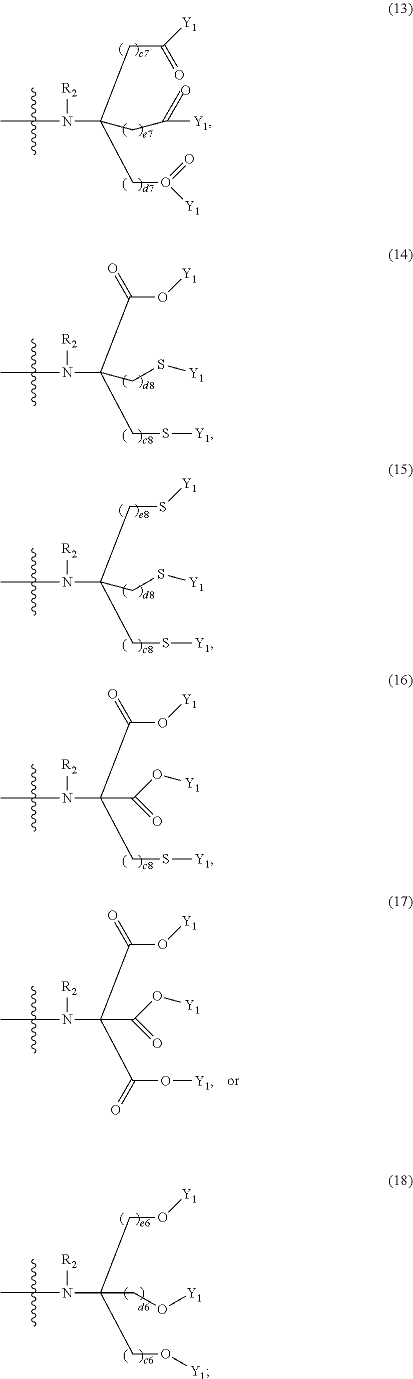
wherein: [symbol]
denotes attachment to M^{P} when present or attachment to L^{P} or L^{P′} when M^{P} is absent;
Y₁ denotes attachment to L³ when present or attachment to M^{A} when L³ is absent;
R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆alkoxycarbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, —COOH, or —COO—C₁₋₆ alkyl;
each of c₁, c₂, c₃, c₄, c₅, c₆, c₇, and c₈ is an integer independently ranging between 0 and 10;
each of d₁, d₂, d₃, d₄, d₅, d₆, d₇, and d₈ is an integer independently ranging between 0 and 10; and
each of e₁, e₂, e₃, e4, e₅, e₆, e₇, and e₈ is an integer independently ranging between 0 and 10.

31. The conjugate or scaffold of any one of the preceding claims, wherein a₂ is 3 and L^{M} is

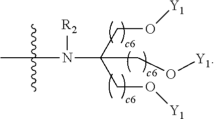

32. The conjugate or scaffold of any one of the preceding claims, wherein M^{A} comprises a peptide moiety that comprises at least about five amino acids.

33. The conjugate or scaffold of any one of the preceding claims, wherein M^{A} comprises a peptide moiety that comprises at most about ten amino acids.

34. The conjugate or scaffold of any one of the preceding claims, wherein M^{A} comprises a peptide moiety that comprises from three to about ten amino acids selected from glycine, serine, glutamic acid, aspartic acid, lysine, cysteine and a combination thereof.

35. The conjugate or scaffold of any one of the preceding claims, wherein M^{A} comprises a peptide moiety that comprises at least four glycines and at least one serine.

36. The conjugate or scaffold of any one of the preceding claims, wherein M^{A} comprises a peptide moiety that comprises at least four glycines and at least one glutamic acid.

37. The conjugate or scaffold of any one of the preceding claims, wherein M^{A} comprises a peptide moiety that comprises at least four glycines, at least one serine and at least one glutamic acid.

38. The conjugate of any one of the preceding claims, being of Formula (XXX): 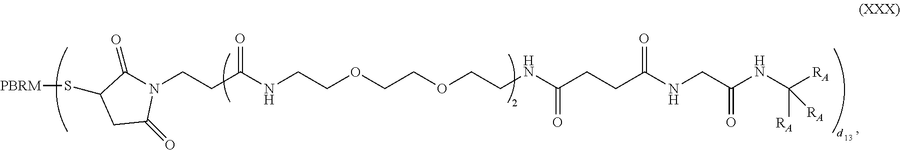
wherein each R_{A} is

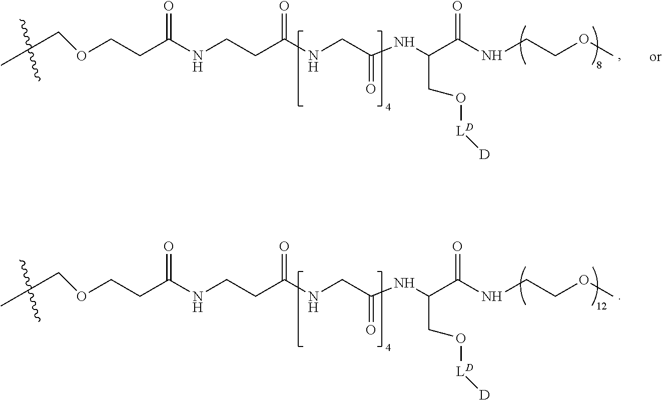

39. The conjugate of any one of the preceding claims, being of Formula (XXX): 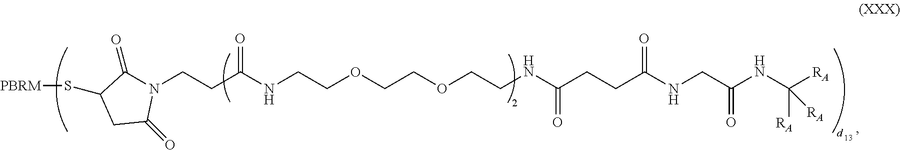
wherein each R_{A} is

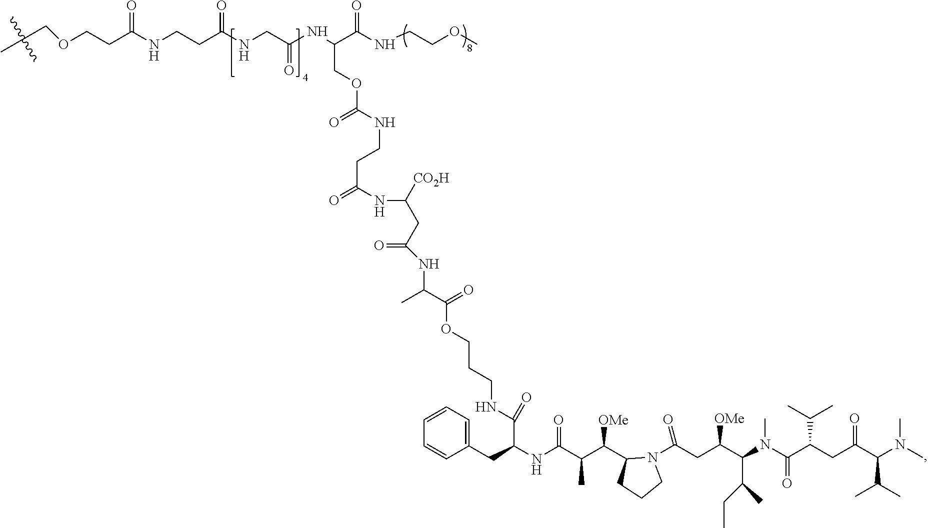

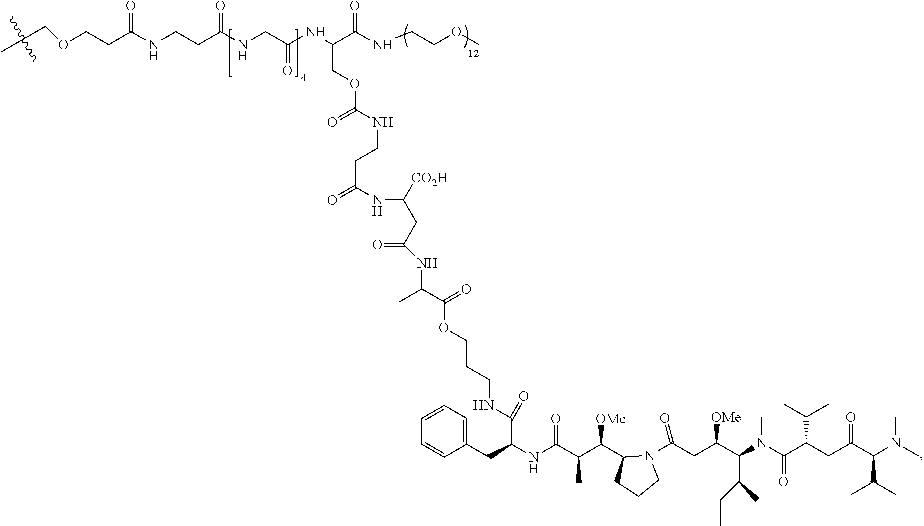

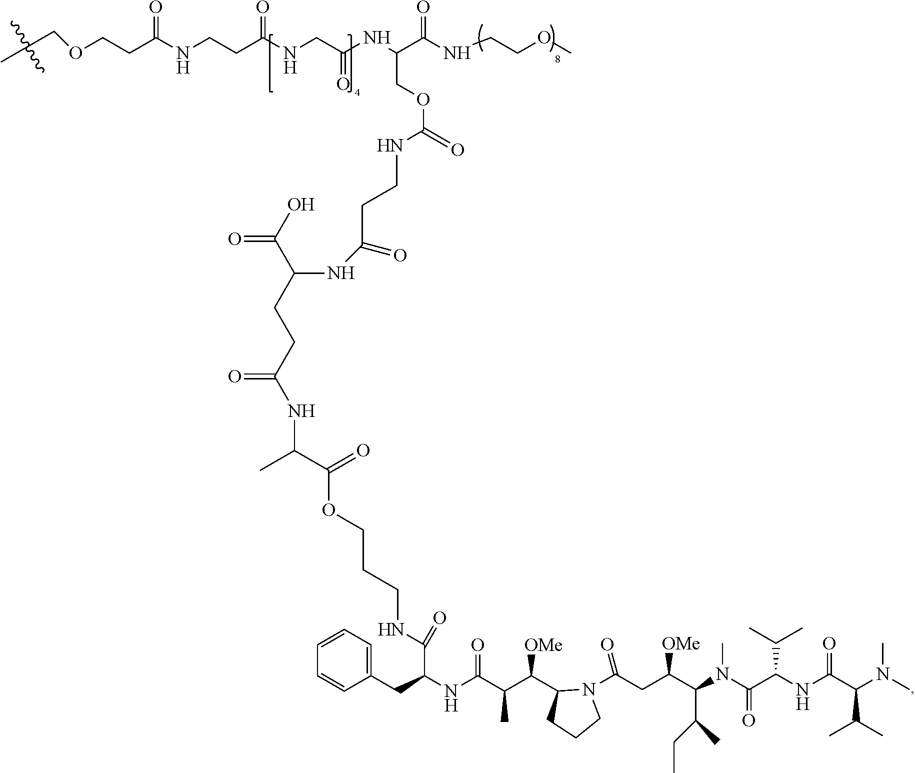

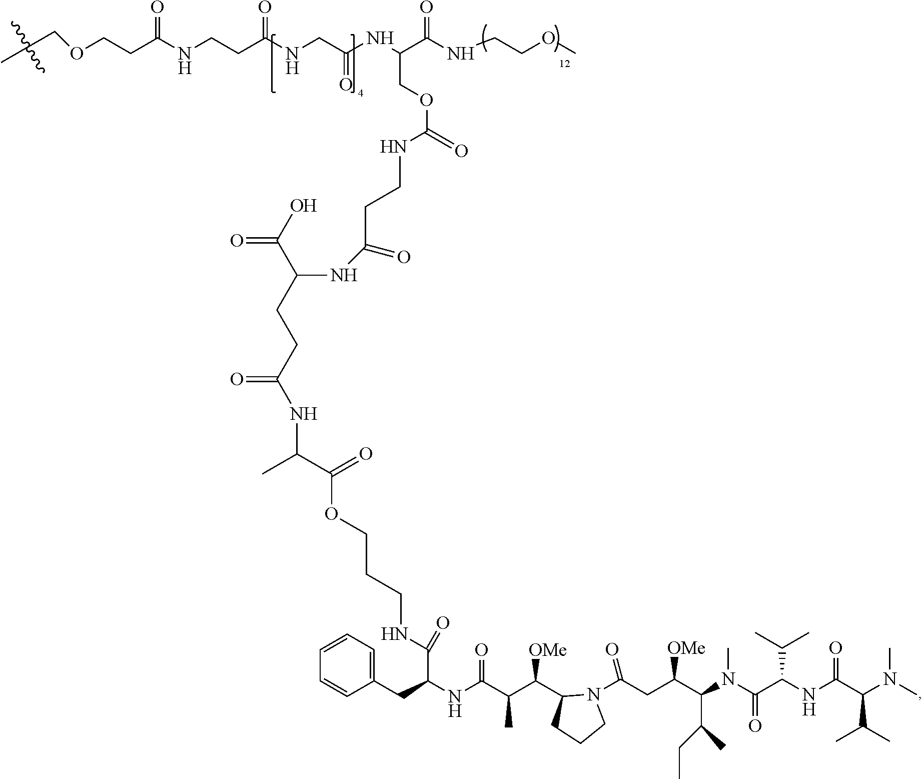

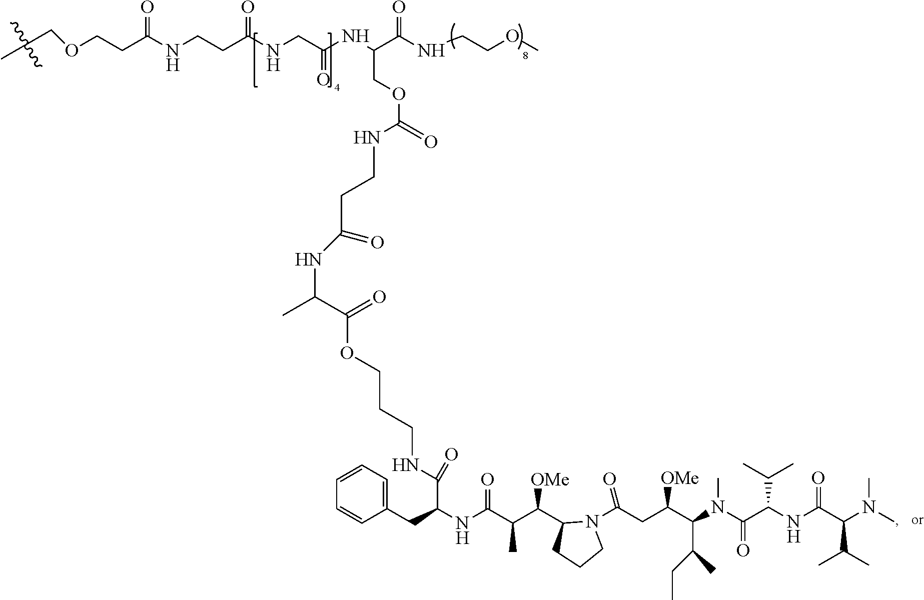

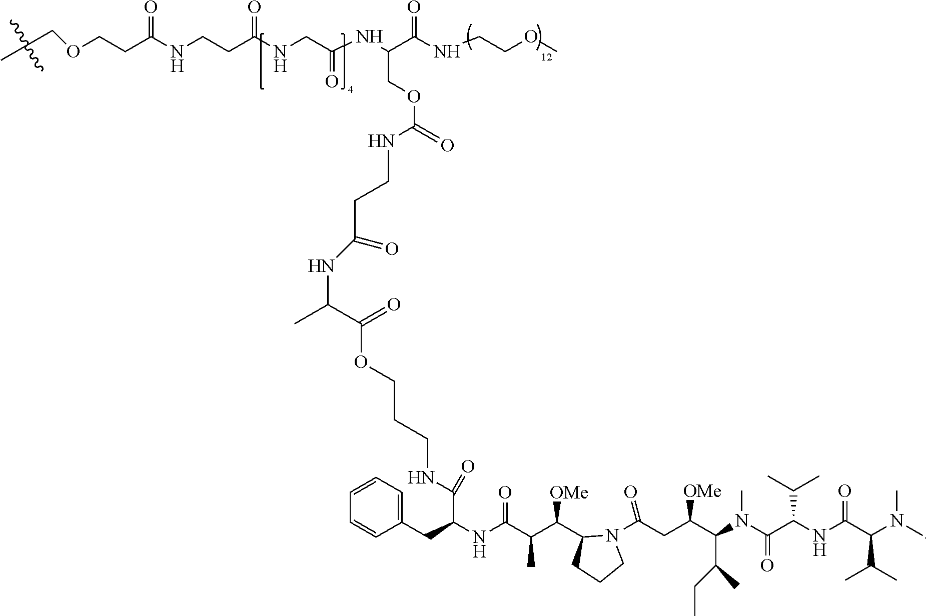

40. The conjugate of any one of the preceding claims, wherein each R_{A} is

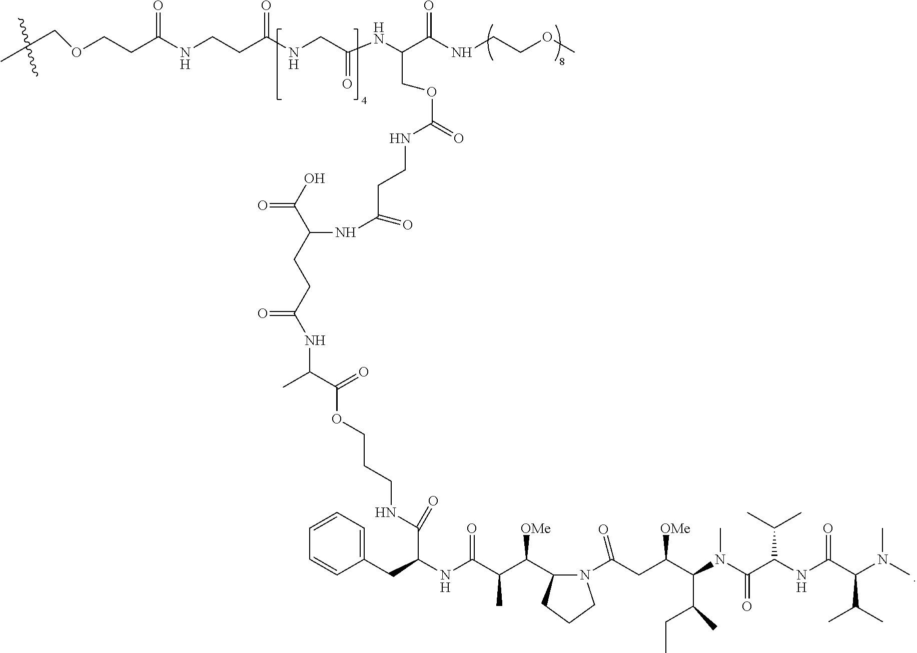

41. The conjugate of an one of the preceding claims wherein each is

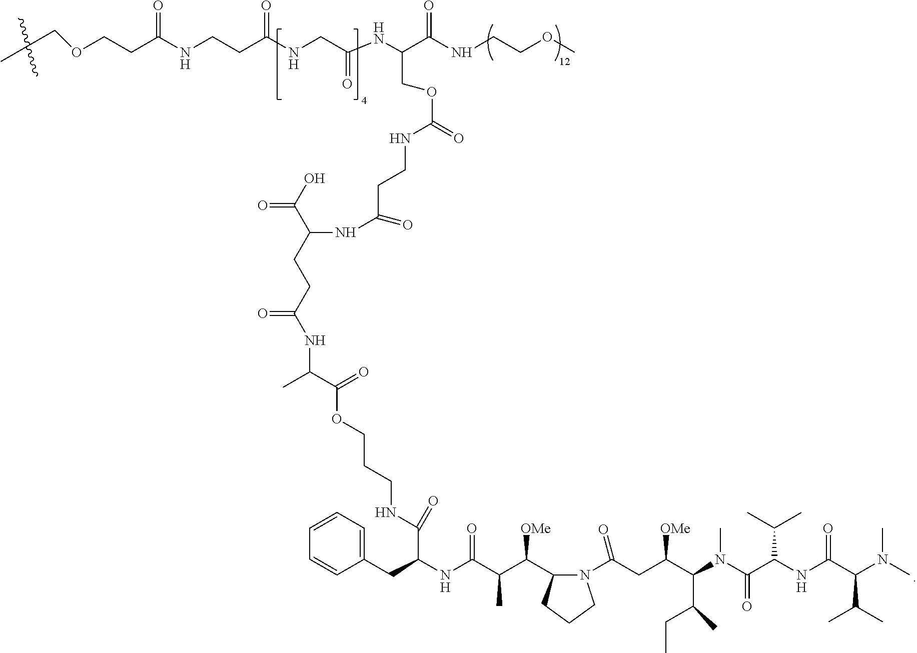

42. The conjugate of any one of the preceding claims, wherein each R_{A} is

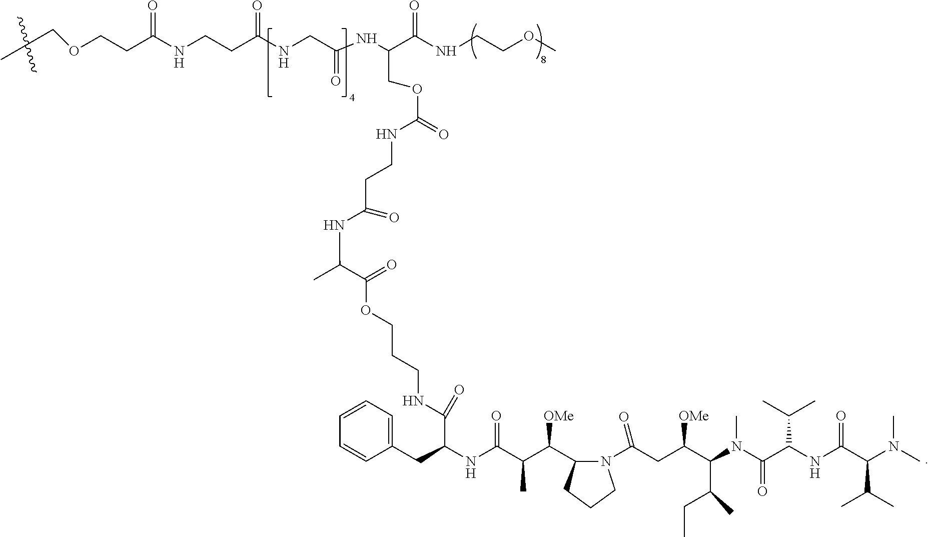

43. The conjugate of any one of the preceding claims, wherein each R_{A} is

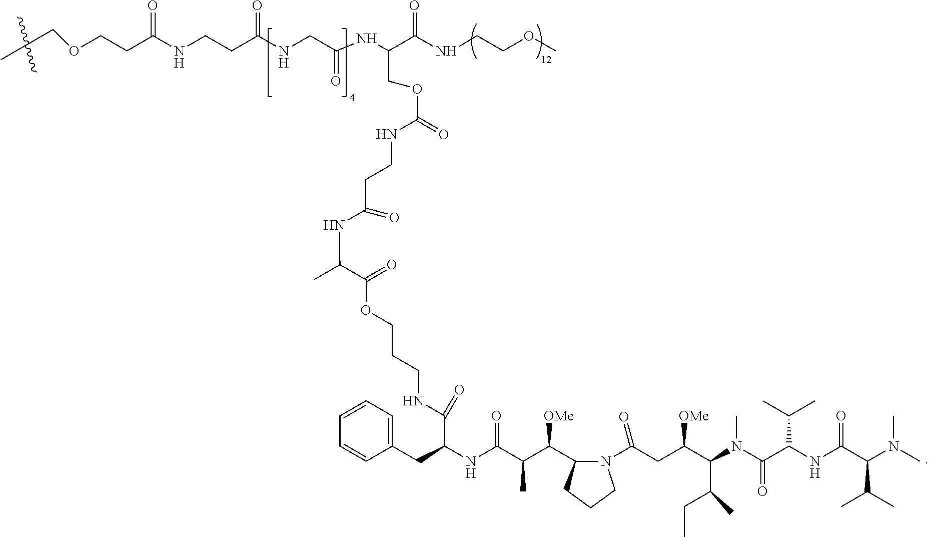

44. The conjugate of any one of the preceding claims, wherein each R_{A} is

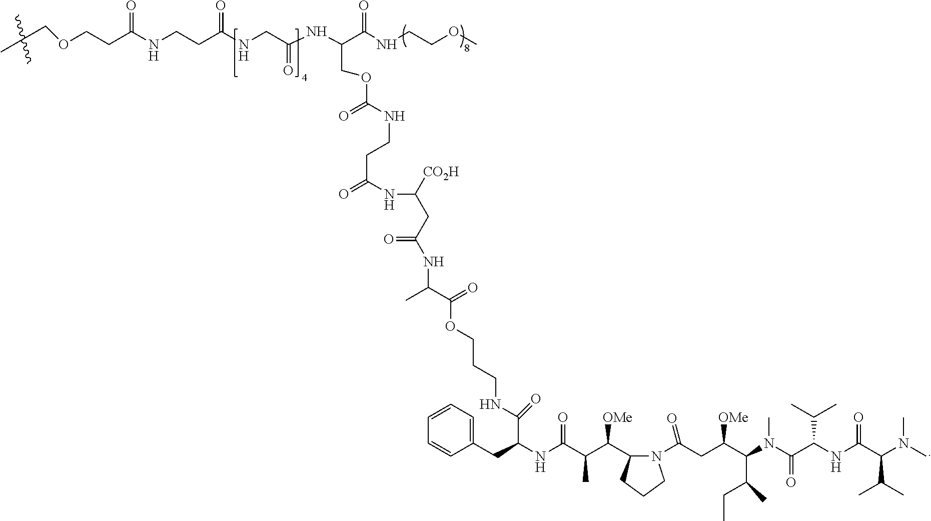

45. The conjugate of any one of the preceding claims, wherein each R_{A} is

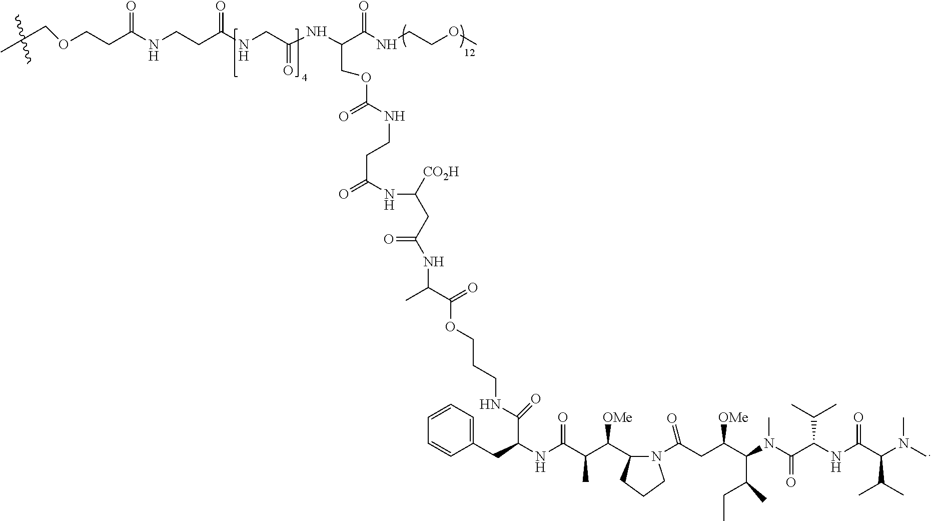

46. The conjugate of any one of the preceding claims, of Formula (XXXIII-5): 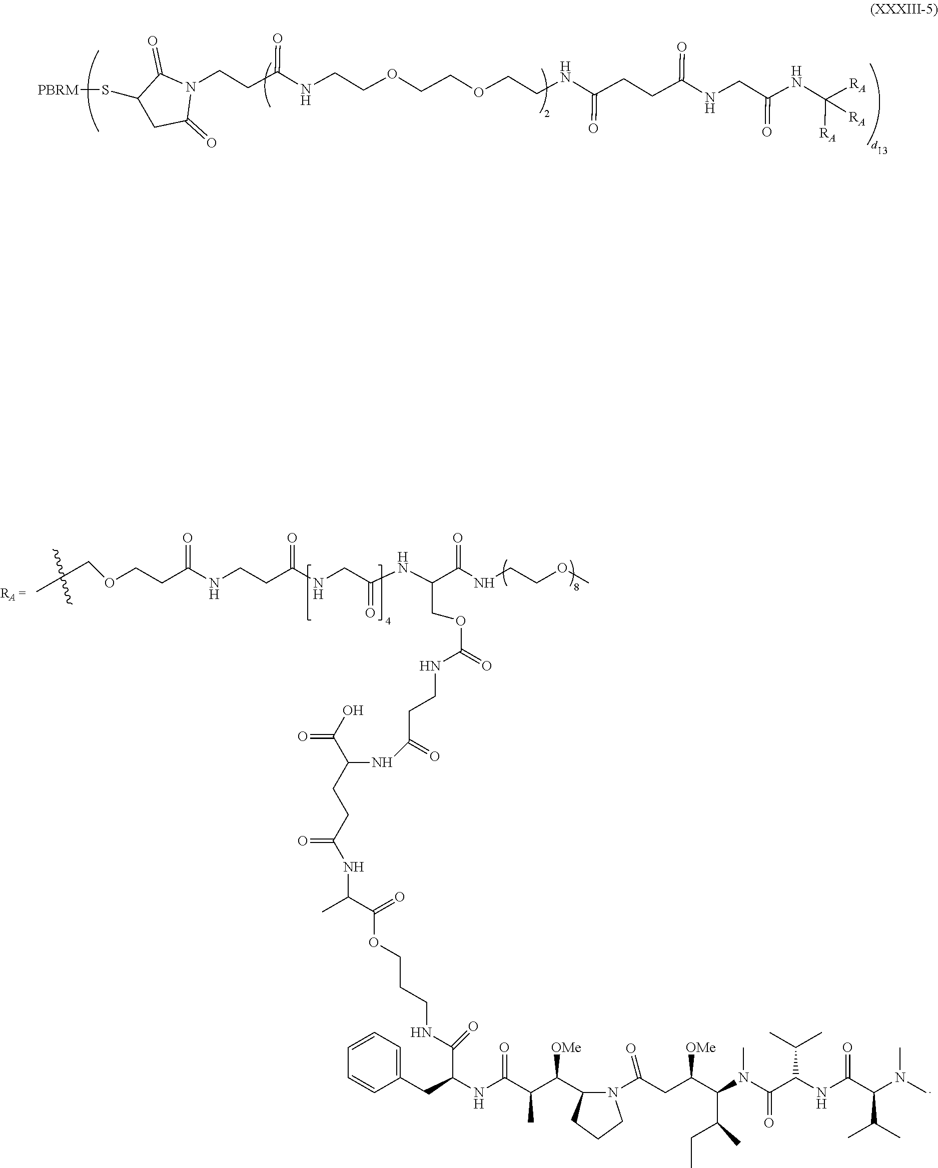
wherein PBRM is an antibody or antibody fragment comprising a light chain V205C, and d₁₃ is an integer from 1 to 2.

47. A pharmaceutical composition comprising a conjugate of any one of the preceding claims and a pharmaceutically acceptable carrier.

48. A method of treating a disorder in a subject in need thereof, comprising administering to the subject an effective amount of a conjugate of any one of the preceding claims.


SEQUENCE LISTING (48 sequences)

SEQ ID NO: 1 - protein, 4 aa, Artificial Sequence
  artificial peptide
GGGG

SEQ ID NO: 2 - protein, 5 aa, Artificial Sequence
  artificial peptide
SGGGG

SEQ ID NO: 3 - protein, 5 aa, Artificial Sequence
  artificial peptide
GSGGG

SEQ ID NO: 4 - protein, 5 aa, Artificial Sequence
  artificial peptide
GGSGG

SEQ ID NO: 5 - protein, 10 aa, Artificial Sequence
  CDRH1 sequence
GYTFTGYNIH

SEQ ID NO: 6 - protein, 17 aa, Artificial Sequence
  CDRH2 sequence
AIYPGNGDTSYKQKFRG

SEQ ID NO: 7 - protein, 10 aa, Artificial Sequence
  CDRH3 sequence
GETARATFAY

SEQ ID NO: 8 - protein, 11 aa, Artificial Sequence
  CDRL1 sequence
SASQDIGNFLN

SEQ ID NO: 9 - protein, 7 aa, Artificial Sequence
  CDRL2 sequence
YTSSLYS

SEQ ID NO: 10 - protein, 9 aa, Artificial Sequence
  CDRL3 sequence
QQYSKLPLT

SEQ ID NO: 11 - protein, 5 aa, Artificial Sequence
  artificial peptide
GGGSG

SEQ ID NO: 12 - protein, 5 aa, Artificial Sequence
  artificial peptide
GGGGS

SEQ ID NO: 13 - protein, 5 aa, Artificial Sequence
  artificial peptide
EGGGG

SEQ ID NO: 14 - protein, 5 aa, Artificial Sequence
  artificial peptide
GEGGG

SEQ ID NO: 15 - protein, 5 aa, Artificial Sequence
  artificial peptide
GGEGG

SEQ ID NO: 16 - protein, 5 aa, Artificial Sequence
  artificial peptide
GGGEG

SEQ ID NO: 17 - protein, 5 aa, Artificial Sequence
  artificial peptide
GGGGE

SEQ ID NO: 18 - protein, 6 aa, Artificial Sequence
  artificial peptide
  X (1)..(1): X is beta-alanine
XGGGGS

SEQ ID NO: 19 - protein, 6 aa, Artificial Sequence
  artificial peptide
  X (1)..(1): X is beta-alanine
XSGGGG

SEQ ID NO: 20 - protein, 6 aa, Artificial Sequence
  artificial peptide
  X (1)..(1): X is beta-alanine
XGSGGG

SEQ ID NO: 21 - protein, 7 aa, Artificial Sequence
  CDRL2 sequence
GASSRAT

SEQ ID NO: 22 - protein, 6 aa, Artificial Sequence
  artificial peptide
  X (1)..(1): X is beta-alanine
XGGSGG

SEQ ID NO: 23 - protein, 6 aa, Artificial Sequence
  artificial peptide
  X (1)..(1): X is beta-alanine
XGGGSG

SEQ ID NO: 24 - protein, 6 aa, Artificial Sequence
  artificial peptide
GGGGSE

SEQ ID NO: 25 - protein, 9 aa, Artificial Sequence
  CDRH1 sequence
FTFSSYSMN

SEQ ID NO: 26 - protein, 17 aa, Artificial Sequence
  CDRH2 sequence
YISSSSSTIYYADSVKG

SEQ ID NO: 27 - protein, 8 aa, Artificial Sequence
  CDRH3 sequence
GGHGYFDL

SEQ ID NO: 28 - protein, 12 aa, Artificial Sequence
  CDRL1 sequence
RASQSVSSSYLA

SEQ ID NO: 29 - protein, 9 aa, Artificial Sequence
  CDRL3 sequence
QQYHHSPLT

SEQ ID NO: 30 - protein, 6 aa, Artificial Sequence
  artificial peptide
SGGGGE

SEQ ID NO: 31 - protein, 6 aa, Artificial Sequence
  artificial peptide
GSGGGE

SEQ ID NO: 32 - protein, 6 aa, Artificial Sequence
  artificial peptide
GGSGGE

SEQ ID NO: 33 - protein, 6 aa, Artificial Sequence
  artificial peptide
GGGSGE

SEQ ID NO: 34 - protein, 6 aa, Artificial Sequence
  artificial peptide
GGGGSE

SEQ ID NO: 35 - protein, 7 aa, Artificial Sequence
  artificial peptide
  X (1)..(1): X is beta-alanine
XGGGGES

SEQ ID NO: 36 - protein, 7 aa, Artificial Sequence
  artificial peptide
  X (1)..(1): X is beta-alanine
XSGGGGE

SEQ ID NO: 37 - protein, 7 aa, Artificial Sequence
  artificial peptide
  X (1)..(1): X is beta-alanine
XGSGGGE

SEQ ID NO: 38 - protein, 7 aa, Artificial Sequence
  artificial peptide
  X (1)..(1): X is beta-alanine
XGGSGGE

SEQ ID NO: 39 - protein, 7 aa, Artificial Sequence
  artificial peptide
  X (1)..(1): X is beta-alanine
XGGGSGE

SEQ ID NO: 40 - protein, 4 aa, Artificial Sequence
  artificial peptide
GGGS

SEQ ID NO: 41 - protein, 4 aa, Artificial Sequence
  artificial peptide
  X (1)..(1): X is beta-alanine
XGGS

SEQ ID NO: 42 - protein, 5 aa, Artificial Sequence
  artificial peptide
  X (1)..(1): X is beta-alanine
XGGGS

SEQ ID NO: 43 - protein, 4 aa, Artificial Sequence
  artificial peptide
GGGE

SEQ ID NO: 44 - protein, 4 aa, Artificial Sequence
  artificial peptide
  X (1)..(1): X is beta-alanine
XGGE

SEQ ID NO: 45 - protein, 5 aa, Artificial Sequence
  artificial peptide
  X (1)..(1): X is beta-alanine
XGGGE

SEQ ID NO: 46 - protein, 6 aa, Artificial Sequence
  artificial peptide
  X (1)..(1): X is beta-alanine
XGGGGE

SEQ ID NO: 47 - protein, 4 aa, Artificial Sequence
  artificial peptide
  X (1)..(1): X is citrulline
XVGG

SEQ ID NO: 48 - protein, 4 aa, Artificial Sequence
  artificial peptide
GFGG